(19) United States
(12) Patent Application Publication
(10) Pub. No.: US 2026/0345832 A1
(43) Pub. Date: Nov. 5, 2026

(54) COMPOSITIONS AND METHODS FOR TREATING SENSORINEURAL HEARING LOSS USING OTOFERLIN DUAL VECTOR SYSTEMS

(71) Applicants: Decibel Therapeutics, Inc., Boston, MA (US); Regeneron Pharmaceuticals, Inc., Tarrytown, NY (US)

(72) Inventors: Joseph BURNS, Newton, MA (US); Kathryn ELLIS, Arlington, MA (US); Tyler GIBSON, Boston, MA (US); Adam PALERMO, Somerville, MA (US); Martin SCHWANDER, Auburndale, MA (US); Jonathon WHITTON, Cambridge, MA (US); Leah SABIN, Golden Bridge, NY (US); Christos KYRATSOUS, Irvington, NY (US); Meghan DRUMMOND SAMUELSON, Katonah, NY (US)

(21) Appl. No.: 19/564,648

(22) Filed: Mar. 12, 2026

Related U.S. Application Data
(63) Continuation of application No. 17/733,744, filed on Apr. 29, 2022, now Pat. No. 12,589,168.
Application No. 17/733,744 is a continuation of application No. PCT/US20/58265, filed on Oct. 30, 2020.
(60) Provisional application No. 62/928,290, filed on Oct. 30, 2019.
(60) Provisional application No. 62/965,776, filed on Jan. 24, 2020.
(60) Provisional application No. 62/971,504, filed on Feb. 7, 2020.
(60) Provisional application No. 63/023,058, filed on May 11, 2020.

(51) Int. Cl. A61K 48/00; C07K 14/47; C12N 15/86

(52) U.S. Cl. CPC A61K 48/0075; C07K 14/4716; C12N 15/86; A01K 2227/105; A01K 2267/0306; C12N 2750/14143; C12N 2800/40; C12N 2830/008; C12N 2840/44

(57) ABSTRACT

The disclosure features compositions and methods for the treatment of sensorineural hearing loss and auditory neuropathy, particularly forms of the disease that are associated with a mutation in otoferlin (OTOF), by way of OTOF gene therapy. The disclosure provides a variety of compositions that include a first nucleic acid vector that contains a polynucleotide encoding an N-terminal portion of an OTOF isoform 5 protein and a second nucleic acid vector that contains a polynucleotide encoding a C-terminal portion of an OTOF isoform 5 protein. These vectors can be used to increase the expression of OTOF in a subject, such as a human subject suffering from sensorineural hearing loss.

### SEQUENCE LISTING

The instant application contains a Sequence Listing which has been submitted electronically in XML format and is hereby incorporated by reference in its entirety. Said XML copy, created on Feb. 12, 2026, is named 51471-004007_Sequence_Listing_2_12_26.xml and is 188,940 bytes in size.

### FIELD OF THE INVENTION

Described herein are compositions and methods for the treatment of sensorineural hearing loss and auditory neuropathy, particularly forms of the disease that are associated with mutations in otoferlin (OTOF), by way of OTOF gene therapy. The disclosure provides dual vector systems that include a first nucleic acid vector that contains a polynucleotide encoding an N-terminal portion of an OTOF isoform 5 protein and a second nucleic acid vector that contains a polynucleotide encoding a C-terminal portion of an OTOF isoform 5 protein. These vectors can be used to increase the expression of or provide wild-type OTOF to a subject, such as a human subject suffering from sensorineural hearing loss.

### BACKGROUND

Sensorineural hearing loss is a type of hearing loss caused by defects in the cells of the inner ear or the neural pathways that project from the inner ear to the brain. Although sensorineural hearing loss is often acquired, and can be caused by noise, infections, head trauma, ototoxic drugs, or aging, there are also congenital forms of sensorineural hearing loss associated with autosomal recessive mutations. One such form of autosomal recessive sensorineural hearing loss is associated with mutation of the otoferlin (OTOF) gene, which is implicated in prelingual nonsyndromic hearing loss. In recent years, efforts to treat hearing loss have increasingly focused on gene therapy as a possible solution; however, OTOF is too large to allow for treatment using standard gene therapy approaches. There is a need for new therapeutics to treat OTOF-related sensorineural hearing loss.

### SUMMARY OF THE INVENTION

The present invention is based on the inventors' discovery that OTOF isoform 5 was preferentially expressed in the inner ear of non-human primates, and that human OTOF isoform 5, but not human OTOF isoform 1, was able to rescue hearing in genetically engineered, congenitally deaf mice with Otoferlin deficiency. Accordingly, the invention provides compositions and methods for treating sensorineural hearing loss or auditory neuropathy in a subject, such as a human subject. The compositions and methods of the disclosure pertain to dual vector systems for the delivery of a polynucleotide encoding an otoferlin (OTOF) isoform 5 protein to a subject having or at risk of developing sensorineural hearing loss or auditory neuropathy (e.g., a subject with a mutation in OTOF). For example, using the compositions and methods described herein, a first nucleic acid vector (e.g., a first adeno-associated virus (AAV) vector) and a second nucleic acid vector (e.g., a second AAV vector) that each encode a portion of a functional OTOF isoform 5 protein may be delivered to a subject by way of viral gene therapy. The compositions and methods described herein may also be used to increase expression of a WT OTOF protein (e.g., a full-length OTOF isoform 5 protein) in a cochlear hair cell (e.g., an inner hair cell) and/or to treat a subject having or at risk of developing sensorineural hearing loss, such as a subject having a mutation in OTOF.

In a first aspect, the invention provides a dual vector system including a first AAV vector comprising a Myo15 promoter operably linked to a first coding polynucleotide that encodes an N-terminal portion of an Otoferlin (OTOF) isoform 5 protein, a splice donor sequence positioned 3′ of the first coding polynucleotide, and a first recombinogenic region positioned 3′ of the splice donor sequence; and a second AAV vector comprising a second recombinogenic region, a splice acceptor sequence positioned 3′ of the second recombinogenic region, a second coding polynucleotide that encodes a C-terminal portion of the OTOF isoform 5 protein positioned 3′ of the splice acceptor sequence, and a poly(A) sequence positioned 3′ of the second coding polynucleotide; in which the first coding polynucleotide and the second coding polynucleotide that encode the OTOF isoform 5 protein do not overlap, and in which neither the first nor the second AAV vector encodes the full-length OTOF isoform 5 protein.

In some embodiments, the first AAV vector and the second AAV vector include an AAV1 capsid.

In some embodiments, the first AAV vector and the second AAV vector include an AAV9 capsid.

In some embodiments, the Myo15 promoter comprises a first region having at least 85% sequence identity (e.g., 85%, 90%, 95%, 96%, 97%, 98%, 99%, or more, sequence identity) to SEQ ID NO: 7 or a functional portion or derivative thereof including the sequence of SEQ ID NO: 9 and/or SEQ ID NO: 10 joined (e.g., operably linked) to a second region having at least 85% sequence identity (e.g., 85%, 90%, 95%, 96%, 97%, 98%, 99%, or more, sequence identity) to SEQ ID NO: 8 or a functional portion or derivative thereof including the sequence of SEQ ID NO: 14 and/or SEQ ID NO: 15, optionally containing a linker comprising one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region comprises or consists of the sequence of SEQ ID NO: 7. In some embodiments, the second region comprises or consists of the sequence of SEQ ID NO: 8. In some embodiments, the Myo15 promoter comprises or consists of the sequence of SEQ ID NO: 19. In some embodiments, the Myo15 promoter comprises or consists of the sequence of SEQ ID NO: 21. In some embodiments, the Myo15 promoter comprises or consists of the sequence of SEQ ID NO: 22. In some embodiments, the Myo15 promoter comprises or consists of the sequence of SEQ ID NO: 36. In some embodiments, the Myo15 promoter comprises or consists of the sequence of SEQ ID NO: 37. In some embodiments, the Myo15 promoter comprises or consists of the sequence of SEQ ID NO: 42. In some embodiments, the Myo15 promoter comprises or consists of the sequence of SEQ ID NO: 43.

In some embodiments, the Myo15 promoter comprises a first region having at least 85% sequence identity (e.g., 85%, 90%, 95%, 96%, 97%, 98%, 99%, or more, sequence identity) to SEQ ID NO: 8 or a functional portion or derivative thereof including the sequence of SEQ ID NO: 14 and/or SEQ ID NO: 15, joined (e.g., operably linked) to a second region having at least 85% sequence identity (e.g., 85%, 90%, 95%, 96%, 97%, 98%, 99%, or more, sequence identity) to SEQ ID NO: 7 or a functional portion or derivative thereof including the sequence of SEQ ID NO: 9 and/or SEQ ID NO: 10, optionally containing a linker including one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region comprises or consists of the sequence of SEQ ID NO: 8. In some embodiments, the second region comprises or consists of the sequence of SEQ ID NO: 7. In some embodiments, the Myo15 promoter comprises or consists of the sequence of SEQ ID NO: 20. In some embodiments, the Myo15 promoter comprises or consists of the sequence of SEQ ID NO: 41.

In some embodiments, the Myo15 promoter comprises a region having at least 85% sequence identity (e.g., 85%, 90%, 95%, 96%, 97%, 98%, 99%, or more, sequence identity) to SEQ ID NO: 7 or a functional portion or derivative thereof including the sequence of SEQ ID NO: 9 and/or SEQ ID NO: 10. In some embodiments, the region comprises or consists of the sequence of SEQ ID NO: 7.

In some embodiments, the Myo15 promoter comprises a region having at least 85% sequence identity (e.g., 85%, 90%, 95%, 96%, 97%, 98%, 99%, or more, sequence identity) to SEQ ID NO: 8 or a functional portion or derivative thereof including the sequence of SEQ ID NO: 14 and/or SEQ ID NO: 15. In some embodiments, the region comprises or consists of the sequence of SEQ ID NO: 8.

In some embodiments, the functional portion of SEQ ID NO: 7 contains the sequence of SEQ ID NO: 9. In some embodiments, the functional portion of SEQ ID NO: 7 contains the sequence of SEQ ID NO: 10. In some embodiments, the functional portion of SEQ ID NO: 7 contains the sequence of SEQ ID NO: 9 and the sequence of SEQ ID NO: 10. In some embodiments, the functional portion of SEQ ID NO: 7 contains the sequence of SEQ ID NO: 11. In some embodiments, the functional portion of SEQ ID NO: 7 contains the sequence of SEQ ID NO: 12. In some embodiments, the functional portion of SEQ ID NO: 7 contains the sequence of SEQ ID NO: 13. In some embodiments, the functional portion of SEQ ID NO: 7 contains the sequence of SEQ ID NO: 33.

In some embodiments, the functional portion of SEQ ID NO: 8 contains the sequence of SEQ ID NO: 14. In some embodiments, the functional portion of SEQ ID NO: 8 contains the sequence of SEQ ID NO: 15. In some embodiments, the functional portion of SEQ ID NO: 8 contains the sequence of SEQ ID NO: 34. In some embodiments, the functional portion of SEQ ID NO: 8 contains the sequence of SEQ ID NO: 35. In some embodiments, the functional portion of SEQ ID NO: 8 contains the sequence of SEQ ID NO: 14 and the sequence of SEQ ID NO: 15. In some embodiments, the functional portion of SEQ ID NO: 8 contains the sequence of SEQ ID NO: 16. In some embodiments, the functional portion of SEQ ID NO: 8 contains the sequence of SEQ ID NO: 17. In some embodiments, the functional portion of SEQ ID NO: 8 contains the sequence of SEQ ID NO: 18. In some embodiments, the functional portion of SEQ ID NO: 8 contains the sequence of SEQ ID NO: 38.

In some embodiments, the Myo15 promoter has at least 85% sequence identity (e.g., 85%, 90%, 95%, 96%, 97%, 98%, 99%, or more, sequence identity) to the nucleic acid sequence of any one of SEQ ID NOs: 33-41. In some embodiments, the Myo15 promoter comprises or consists of the sequence of SEQ ID NO: 33. In some embodiments, the Myo15 promoter comprises or consists of the sequence of SEQ ID NO: 34. In some embodiments, the Myo15 promoter comprises or consists of the sequence of SEQ ID NO: 35. In some embodiments, the Myo15 promoter comprises or consists of the sequence of SEQ ID NO: 36. In some embodiments, the Myo15 promoter comprises or consists of the sequence of SEQ ID NO: 37. In some embodiments, the Myo15 promoter comprises or consists of the sequence of SEQ ID NO: 38. In some embodiments, the Myo15 promoter comprises or consists of the sequence of SEQ ID NO: 39. In some embodiments, the Myo15 promoter comprises or consists of the sequence of SEQ ID NO: 40. In some embodiments, the Myo15 promoter comprises or consists of the sequence of SEQ ID NO: 41.

In some embodiments, the Myo15 promoter comprises a first region having at least 85% sequence identity (e.g., 85%, 90%, 95%, 96%, 97%, 98%, 99%, or more, sequence identity) to SEQ ID NO: 23 or a functional portion or derivative thereof including the sequence of SEQ ID NO: 25 joined (e.g., operably linked) to a second region having at least 85% sequence identity (e.g., 85%, 90%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, optionally containing a linker comprising one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region comprises or consists of the sequence of SEQ ID NO: 23. In some embodiments, the second region comprises or consists of the sequence of SEQ ID NO: 24. In some embodiments, the Myo15 promoter comprises or consists of the sequence of SEQ ID NO: 31. In some embodiments, the Myo15 promoter comprises or consists of the sequence of SEQ ID NO: 32.

In some embodiments, the Myo15 promoter comprises a first region having at least 85% sequence identity (e.g., 85%, 90%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, joined (e.g., operably linked) to a second region having at least 85% sequence identity (e.g., 85%, 90%, 95%, 96%, 97%, 98%, 99%, or more, sequence identity) to SEQ ID NO: 23 or a functional portion or derivative thereof including the sequence of SEQ ID NO: 25, optionally containing a linker including one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region comprises or consists of the sequence of SEQ ID NO: 24. In some embodiments, the second region comprises or consists of the sequence of SEQ ID NO: 23.

In some embodiments, the Myo15 promoter comprises a region having at least 85% sequence identity (e.g., 85%, 90%, 95%, 96%, 97%, 98%, 99%, or more, sequence identity) to SEQ ID NO: 23 or a functional portion or derivative thereof including the sequence of SEQ ID NO: 25. In some embodiments, the region comprises or consists of the sequence of SEQ ID NO: 23.

In some embodiments, the Myo15 promoter comprises a region having at least 85% sequence identity (e.g., 85%, 90%, 95%, 96%, 97%, 98%, 99%, or more, sequence identity) to SEQ ID NO: 24 or a functional portion or derivative thereof including the sequence of SEQ ID NO: 26 and/or SEQ ID NO: 27. In some embodiments, the region comprises or consists of the sequence of SEQ ID NO: 24.

In some embodiments, the functional portion of SEQ ID NO: 23 contains the sequence of SEQ ID NO: 25.

In some embodiments, the functional portion of SEQ ID NO: 24 contains the sequence of SEQ ID NO: 26. In some embodiments, the functional portion of SEQ ID NO: 24 contains the sequence of SEQ ID NO: 27. In some embodiments, the functional portion of SEQ ID NO: 24 contains the sequence of SEQ ID NO: 26 and the sequence of SEQ ID NO: 27. In some embodiments, the functional portion of SEQ ID NO: 24 contains the sequence of SEQ ID NO: 28. In some embodiments, the functional portion of SEQ ID NO: 24 contains the sequence of SEQ ID NO: 29. In some embodiments, the functional portion of SEQ ID NO: 24 contains the sequence of SEQ ID NO: 30.

In another aspect, the invention provides a dual vector system including a first AAV1 vector containing a ubiquitous promoter operably linked to a first coding polynucleotide that encodes an N-terminal portion of an OTOF isoform 5 protein, a splice donor sequence positioned 3′ of the first coding polynucleotide, and a first recombinogenic region positioned 3′ of the splice donor sequence; and a second AAV1 vector containing a second recombinogenic region, a splice acceptor sequence positioned 3′ of the second recombinogenic region, a second coding polynucleotide that encodes a C-terminal portion of the OTOF isoform 5 protein positioned 3′ of the splice acceptor sequence, and a poly(A) sequence positioned 3′ of the second coding polynucleotide; in which the first coding polynucleotide and the second coding polynucleotide that encode the OTOF isoform 5 protein do not overlap, and in which neither the first nor second AAV1 vector encodes the full-length OTOF isoform 5 protein.

In some embodiments, the ubiquitous promoter is selected from the group consisting of a CAG promoter, a cytomegalovirus (CMV) promoter, and a truncated CMV-chicken β-actin promoter (smCBA). In some embodiments, the ubiquitous promoter is the smCBA promoter. In some embodiments, the smCBA promoter comprises or consists of the sequence of SEQ ID NO: 44.

In some embodiments of any of the foregoing aspects, the first and second recombinogenic regions are the same.

In some embodiments of any of the foregoing aspects, the first recombinogenic region and/or the second recombinogenic region is an AK recombinogenic region. In some embodiments, the AK recombinogenic region comprises or consists of the sequence of SEQ ID NO: 47.

In some embodiments of any of the foregoing aspects, the first recombinogenic region and/or the second recombinogenic region is an AP gene fragment. In some embodiments, the AP gene fragment comprises or consists of the sequence of any one of SEQ ID NOs: 48-53. In some embodiments, the AP gene fragment comprises or consists of the sequence of SEQ ID NO: 51.

In some embodiments of any of the foregoing aspects, each of the first and second coding polynucleotides encode about half of the OTOF isoform 5 protein sequence.

In some embodiments of any of the foregoing aspects, the first coding polynucleotide encodes amino acids 1-802 of SEQ ID NO: 1. In some embodiments of any of the foregoing aspects, the second coding polynucleotide encodes amino acids 803-1997 of SEQ ID NO: 1.

In some embodiments of any of the foregoing aspects, the first and second coding polynucleotides are divided at an OTOF exon boundary. In some embodiments, the first and second coding polynucleotides are divided at the boundary between exons 20 and 21 of OTOF.

In some embodiments of any of the foregoing aspects, the first coding polynucleotide consists of exons 1-20 of a polynucleotide encoding the OTOF isoform 5 protein and the second coding polynucleotide consists of exons 21-45 and 47 of a polynucleotide encoding the OTOF isoform 5 protein (e.g., a polynucleotide encoding a human OTOF isoform 5 protein).

In some embodiments of any of the foregoing aspects, the first and second coding polynucleotides that encode the OTOF isoform 5 protein do not comprise introns.

In some embodiments of any of the foregoing aspects, the OTOF isoform 5 protein is a human OTOF isoform 5 protein (e.g., the protein having the sequence of SEQ ID NO: 1).

In some embodiments of any of the foregoing aspects, the OTOF isoform 5 protein comprises the sequence of SEQ ID NO: 1 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the OTOF isoform 5 protein variant are conservative amino acid substitutions. In some embodiments, the OTOF isoform 5 protein consists of the sequence of SEQ ID NO: 1.

In some embodiments of any of the foregoing aspects, the OTOF isoform 5 protein is encoded by the sequence of SEQ ID NO: 2.

In some embodiments of any of the foregoing aspects, the OTOF isoform 5 protein is encoded by the sequence of SEQ ID NO: 3.

In some embodiments of any of the foregoing aspects, the N-terminal portion of the OTOF isoform 5 protein consists of the sequence of SEQ ID NO: 58 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the N-terminal portion of the OTOF isoform 5 protein variant are conservative amino acid substitutions. In some embodiments, the N-terminal portion of the OTOF isoform 5 protein consists of the sequence of SEQ ID NO: 58. In some embodiments, the N-terminal portion of the OTOF isoform 5 protein is encoded by the sequence of SEQ ID NO: 56.

In some embodiments of any of the foregoing aspects, the C-terminal portion of the OTOF isoform 5 protein consists of the sequence of SEQ ID NO: 59 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the C-terminal portion of the OTOF isoform 5 protein variant are conservative amino acid substitutions. In some embodiments, the C-terminal portion of the OTOF isoform 5 protein consists of the sequence of SEQ ID NO: 59. In some embodiments, the C-terminal portion of the OTOF isoform 5 protein is encoded by the sequence of SEQ ID NO: 57.

In some embodiments of any of the foregoing aspects, the first vector includes a first inverted terminal repeat (ITR) sequence 5′ of the promoter and a second ITR sequence 3′ of the recombinogenic region, and the second vector includes a first ITR sequence 5′ of the recombinogenic region and a second ITR sequence 3′ of the poly(A) sisequence. In some embodiments, the ITRs in the first vector and second vector are AAV2 ITRs. In some embodiments, the ITRs in the first vector and second vector have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.

In some embodiments of any of the foregoing aspects, the poly(A) sisequence is a bovine growth hormone (bGH) poly(A) signal sequence.

In some embodiments of any of the foregoing aspects, the splice donor sequence in the first vector comprises or consists of the sequence of SEQ ID NO: 54.

In some embodiments of any of the foregoing aspects, the splice acceptor sequence in the second vector comprises or consists of the sequence of SEQ ID NO: 55.

In some embodiments of any of the foregoing aspects, the first AAV vector comprises a Kozak sequence 3′ of the promoter and 5′ of the first coding polynucleotide that encodes the N-terminal portion of the OTOF isoform 5 protein.

In some embodiments of any of the foregoing aspects, the first AAV vector contains a polynucleotide sequence comprising the sequence of nucleotides 2272 to 6041 of SEQ ID NO: 60. In some embodiments of any of the foregoing aspects, the first AAV vector contains a polynucleotide sequence comprising or consisting of the sequence of nucleotides 2049 to 6264 of SEQ ID NO: 60.

In some embodiments of any of the foregoing aspects, the first AAV vector contains a polynucleotide sequence comprising the sequence of nucleotides 182 to 3949 of SEQ ID NO: 62. In some embodiments of any of the foregoing aspects, the first AAV vector contains a polynucleotide sequence comprising or consisting of the sequence of nucleotides 19 to 4115 of SEQ ID NO: 62.

In some embodiments of any of the foregoing aspects, the first AAV vector contains a polynucleotide sequence comprising the sequence of positions 2267 to 6014 of SEQ ID NO: 64. In some embodiments of any of the foregoing aspects, the first AAV vector contains a polynucleotide sequence comprising or consisting of the sequence of positions 2049 to 6237 of SEQ ID NO: 64.

In some embodiments of any of the foregoing aspects, the first AAV vector contains a polynucleotide sequence comprising the sequence of positions 177 to 3924 of SEQ ID NO: 65. In some embodiments of any of the foregoing aspects, the first AAV vector contains a polynucleotide sequence comprising or consisting of the sequence of positions 19 to 4090 of SEQ ID NO: 65.

In some embodiments of any of the foregoing aspects, the second AAV vector contains a polynucleotide sequence comprising the sequence of nucleotides 2267 to 6476 of SEQ ID NO: 61. In some embodiments of any of the foregoing aspects, the second AAV vector contains a polynucleotide sequence comprising or consisting of the sequence of nucleotides 2049 to 6693 of SEQ ID NO: 61.

In some embodiments of any of the foregoing aspects, the second AAV vector contains a polynucleotide sequence comprising the sequence of nucleotides 187 to 4396 of SEQ ID NO: 63. In some embodiments of any of the foregoing aspects, the second AAV vector contains a polynucleotide sequence comprising or consisting of the sequence of nucleotides 19 to 4589 of SEQ ID NO: 63.

In some embodiments of any of the foregoing aspects, the first AAV vector contains a polynucleotide sequence comprising the sequence of nucleotides 235 to 4004 of SEQ ID NO: 66. In some embodiments of any of the foregoing aspects, the first AAV vector contains a polynucleotide sequence comprising or consisting of the sequence of nucleotides 12 to 4227 of SEQ ID NO: 66.

In some embodiments of any of the foregoing aspects, the first AAV vector contains a polynucleotide sequence comprising the sequence of nucleotides 230 to 3977 of SEQ ID NO: 68. In some embodiments of any of the foregoing aspects, the first AAV vector contains a polynucleotide sequence comprising or consisting of the sequence of nucleotides 12 to 4200 of SEQ ID NO: 68.

In some embodiments of any of the foregoing aspects, the second AAV vector contains a polynucleotide sequence comprising the sequence of nucleotides 229 to 4438 of SEQ ID NO: 67. In some embodiments of any of the foregoing aspects, the second AAV vector contains a polynucleotide sequence comprising or consisting of the sequence of nucleotides 12 to 4655 of SEQ ID NO: 67.

In another aspect, the invention provides a pharmaceutical composition containing a dual vector system of the invention and a pharmaceutically acceptable excipient. In some embodiments, the pharmaceutical composition is formulated for administration to the inner ear.

In another aspect, the invention provides a kit containing a dual vector system or pharmaceutical composition of the invention.

In another aspect, the invention provides a method of increasing OTOF expression (e.g., wild-type OTOF expression, e.g., full-length OTOF isoform 5 expression) in a subject in need thereof by administering to the subject a therapeutically effective amount of a dual vector system of the invention.

In another aspect, the invention provides a method of treating a subject having or at risk of developing sensorineural hearing loss by administering to the subject a therapeutically effective amount of a dual vector system of the invention.

In another aspect, the invention provides a method of treating a subject having or at risk of developing auditory neuropathy by administering to the subject a therapeutically effective amount of a dual vector system of the invention.

In some embodiments of any of the foregoing aspects, the subject has a mutation in OTOF.

In some embodiments of any of the foregoing aspects, the subject has been identified as having a mutation in OTOF.

In some embodiments of any of the foregoing aspects, the method further includes identifying the subject as having a mutation in OTOF prior to administering the composition.

In some embodiments of any of the foregoing aspects, the subject has or has been identified as having Deafness, Autosomal Recessive 9 (DFNB9).

In some embodiments of any of the foregoing aspects, the method further includes the step of evaluating the hearing of the subject prior to administering the dual vector system.

In some embodiments of any of the foregoing aspects, the dual vector system is administered locally to the ear. In some embodiments, the dual vector system is administered by injection through the round window membrane, injection into a semicircular canal, canalostomy, insertion of a catheter through the round window membrane, transtympanic injection, or intratympanic injection.

In some embodiments of any of the foregoing aspects, the method increases OTOF expression in a cochlear hair cell. In some embodiments, the cochlear hair cell is an inner hair cell.

In some embodiments of any of the foregoing aspects, the subject is a mammal. In some embodiments, the subject is a human.

In some embodiments of any of the foregoing aspects, the method further comprises evaluating the hearing of the subject after administering the dual vector system.

In some embodiments of any of the foregoing aspects, the dual vector system increases OTOF expression in a cell (e.g., a cochlear hair cell), improves hearing (e.g., as assessed by standard tests, such as audiometry, auditory brainstem response (ABR), electrocochleography (ECOG), and otoacoustic emissions), prevents or reduces hearing loss, delays the development of hearing loss, slows the progression of hearing loss, improves speech discrimination, or improves hair cell function.

In some embodiments of any of the foregoing aspects, the dual vector system is administered in an amount sufficient to increase OTOF expression in a cochlear hair cell, prevent or reduce hearing loss, delay the development of hearing loss, slow the progression of hearing loss, improve hearing (e.g., as assessed by standard tests, such as audiometry, ABR, ECOG, and otoacoustic emissions), improve speech discrimination, or improve hair cell function.

In another aspect, the invention provides a method of increasing OTOF expression in a cell by introducing a dual vector system of the invention into the cell.

In some embodiments, the cell is a cochlear hair cell. In some embodiments, the cell is an inner hair cell. In some embodiments, the cell is a mammalian cell. In some embodiments, the cell is a human cell.

In some embodiments of any of the foregoing aspects, the first vector and the second vector are administered concurrently.

In some embodiments of any of the foregoing aspects, the first vector and the second vector are administered sequentially.

In some embodiments of any of the foregoing aspects, the first vector and the second vector are administered at a concentration of 1×10⁷ vector genomes (VG)/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

### DEFINITIONS

As used herein, the term “about” refers to a value that is within 10% above or below the value being described.

As used herein, “administration” refers to providing or giving a subject a therapeutic agent (e.g., a composition containing a first nucleic acid vector containing a polynucleotide that encodes an N-terminal portion of an otoferlin protein and a second nucleic acid vector containing a polynucleotide that encodes a C-terminal portion of an otoferlin protein), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring
amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms | |
| | | | | in equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a composition, vector construct, or viral vector described herein refer to a quantity sufficient to, when administered to the subject in need thereof, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. Note that when a combination of active ingredients is administered, the effective amount of the combination may or may not include amounts of each ingredient that would have been effective if administered individually. As defined herein, a therapeutically effective amount of a composition, vector construct, viral vector or cell of the present disclosure may be readily determined by one of ordinary skill by routine methods known in the art. Dosage regime may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear hair cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear hair cell). Exogenous materials include those that are provided from an external source to an organism or to cultured matter extracted there from.

As used herein, the term “hair cell-specific expression” refers to production of an RNA transcript or polypeptide primarily within hair cells (e.g., cochlear hair cells) as compared to other cell types of the inner ear (e.g., spiral ganglion neurons, glia, or other inner ear cell types). Hair cell-specific expression of a transgene can be confirmed by comparing transgene expression (e.g., RNA or protein expression) between various cell types of the inner ear (e.g., hair cells vs. non-hair cells) using any standard technique (e.g., quantitative RT PCR, immunohistochemistry, Western Blot analysis, or measurement of the fluorescence of a reporter (e.g., GFP) operably linked to a promoter). A hair cell-specific promoter induces expression (e.g., RNA or protein expression) of a transgene to which it is operably linked that is at least 50% greater (e.g., 50%, 75%, 100%, 125%, 150%, 175%, 200% greater or more) in hair cells (e.g., cochlear hair cells) compared to at least 3 (e.g., 3, 4, 5, 6, 7, 8, 9, 10, or more) of the following inner ear cell types: Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., OTOF expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “intron” refers to a region within the coding region of a gene, the nucleotide sequence of which is not translated into the amino acid sequence of the corresponding protein. The term intron also refers to the corresponding region of the RNA transcribed from a gene. Introns are transcribed into pre-mRNA, but are removed during processing, and are not included in the mature mRNA.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule that can be joined to a second molecule, wherein the molecules are so arranged that the first molecule affects the function of the second molecule. The term “operably linked” includes the juxtaposition of two or more components (e.g., a promoter and another sequence element) such that both components function normally and allow for the possibility that at least one of the components can mediate a function that is exerted upon at least one of the other components. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. In additional embodiments, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the terms “otoferlin isoform 5” and “OTOF isoform 5” refer to an isoform of the gene associated with nonsyndromic recessive deafness DFNB9. The human isoform of the gene is associated with reference sequence NM_001287489, and the transcript includes exons 1-45 and 47 of human otoferlin, but lacks exon 46 of the OTOF gene. The human OTOF isoform 5 protein is also known as Otoferlin isoform e. The terms “otoferlin isoform 5” and “OTOF isoform 5” also refer to variants of the wild-type OTOF isoform 5 protein and polynucleotides encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type OTOF isoform 5 protein (e.g., SEQ ID NO: 1) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the polynucleotide sequence of a wild-type OTOF isoform 5 gene, provided that the OTOF isoform 5 analog encoded retains the therapeutic function of wild-type OTOF isoform 5. OTOF isoform 5 protein variants can have one or more (e.g., 1, 2, 3, 4, 5, 6, 7, 8, 9, 10, 11, 12, 13, 14, 15, 16, 17, 18, 19, 20, or more) conservative amino acid substitutions relative to a wild-type OTOF isoform 5 (e.g., SEQ ID NO: 1), provided that the that the OTOF isoform 5 variant retains the therapeutic function of wild-type OTOF isoform 5 and has no more than 10% amino acid substitutions in an N-terminal portion of the amino acid sequence and no more than 10% amino acid substitutions in a C-terminal portion of the amino acid sequence. As used herein, OTOF isoform 5 may refer to the protein localized to inner hair cells or to the gene encoding this protein, depending upon the context, as will be appreciated by one of skill in the art. OTOF isoform 5 may refer to human OTOF isoform 5 or to a homolog from another mammalian species. Murine otoferlin contains one additional exon relative to human otoferlin (48 exons in murine otoferlin), and the exons of murine otoferlin that correspond to those that encode human OTOF isoform 5 are 1-5, 7-46, and 48. The exon numbering convention used herein is based on the exons currently understood to be present in the consensus transcripts of human OTOF.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules containing nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the terms “complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41 (% G+C), although other known Tm computations take into account nucleic acid structural characteristics.

As used herein, the term “promoter” refers to a recognition site on DNA that is bound by an RNA polymerase. The polymerase drives transcription of the transgene. Exemplary promoters suitable for use with the compositions and methods described herein include ubiquitous promoters (e.g., the CAG promoter, cytomegalovirus (CMV) promoter, and smCBA promoter) and cochlear hair cell-specific promoters (e.g., the Myosin 15 (Myo15) promoter).

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full-length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
   where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “derivative” as used herein refers to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “recombinogenic region” refers to a region of homology that mediates recombination between two different sequences.

As used herein, the term “regulatory sequence” includes promoters, enhancers and other expression control elements (e.g., polyadenylation signals) that control the transcription or translation of the polynucleotides that encode OTOF. Such regulatory sequences are described, for example, in Goeddel, Gene Expression Technology: Methods in Enzymology 185 (Academic Press, San Diego, CA, 1990); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection and the like.

As used herein, the terms “subject” and “patient” refer to an animal (e.g., a mammal, such as a human), veterinary animals (e.g., cats, dogs, cows, horses, sheep, pigs, etc.) and experimental animal models of diseases (e.g., mice, rats). A subject to be treated according to the methods described herein may be one who has been diagnosed with hearing loss (e.g., hearing loss associated with a mutation in OTOF), or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” of a state, disorder or condition can include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO94/11026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of OTOF as described herein include vectors that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIG. 1A is a graph showing the analysis of the auditory brainstem response (ABR) in OTOF homozygous mutant (OTOF^{Q828X/Q828X}) mice administered an OTOF dual vector system demonstrating that AAV-mediated gene transfer of human OTOF isoform 5 but not human OTOF isoform 1 improved auditory function. ABR thresholds to pure tone stimuli at 22.6 kHz were recorded from mice that received OTOF dual vector systems carrying either codon-optimized (CO) or native (native) sequences encoding human OTOF isoform 1 (V1) (SEQ ID NO: 4) or isoform 5 (V5) (SEQ ID NO: 1). The dose of virus, in vector genomes (vg), delivered to the ear is indicated below the description of the sequence administered to each treatment group. OTOF virus was injected through the round window membrane of four- to seven-week-old OTOF^{Q828X/Q828X} mice (a mouse model of the human OTOF mutation p.Gln828Ter). ABR responses were measured approximately four weeks after injection of the viruses. ABR responses significantly improved in mice treated with human OTOF isoform 5, irrespective of codon-optimization. No ABR response was detected in mice treated with human OTOF isoform 1. The range of ABR thresholds observed in wild-type and heterozygous OTOF mutant mice (OTOF^{Q828X/+}) are indicated by a box.

FIG. 1B is a series of fluorescent images of mouse cochleae transduced with OTOF dual vector systems carrying codon-optimized (CO) sequences encoding human OTOF isoform 1 (V1) or isoform 5 (V5). Mice were injected with OTOF dual vector systems carrying codon-optimized (CO) sequences of human OTOF isoform 1 (V1) (SEQ ID NO: 6) or isoform 5 (V5) (SEQ ID NO: 3). After physiology testing was completed, (see data in FIG. 1A), mice were euthanized and perfused with 10% neutral buffered formalin. The inner ear temporal bone was harvested and decalcified in 8% EDTA for 3 days. The cochlea was dissected from the de-calcified temporal bone, immunostained with OTOF antibody to detect expression of the OTOF transgene, and mounted on a slide for fluorescence imaging. Cochleae injected with dual vector systems encoding either human OTOF isoform 1 (V1) or isoform 5 (V5) showed comparable levels of OTOF expression (FIG. 1B). Since functional recovery was only observed for human OTOF isoform 5 (V5) but not isoform 1 (V1) (see FIG. 1A), these data indicate that human OTOF isoform 5 (V5) is the functionally relevant OTOF isoform in cochlear inner hair cells.

FIG. 2 is a graph showing the analysis of ABR in in OTOF homozygous mutant (OTOF^{Q828X/Q828X}) mice demonstrating that AAV-mediated gene transfer of human OTOF isoform 5 improves auditory function to wild-type levels when delivered at higher dose. ABR thresholds to pure tone stimuli at 22.6 kHz were recorded from mice that received OTOF dual vectors carrying either codon-optimized (CO) or native (native) sequences of human OTOF isoform 5 (V5) (SEQ ID NOs: 3 and 2, respectively). For each treatment group, the dose of virus, in vector genomes (vg), delivered to the ear is indicated below the description of the sequence administered to each treatment group. OTOF virus was injected through the round window membrane of four- to seven-week-old OTOF^{Q828X/Q828X} mice. ABR responses were measured approximately four weeks after injection of the viruses. Relative to animals dosed with 7E9 or 6E9 vector genomes/ear (FIG. 1A), these animals showed even stronger ABR responses, irrespective of codon-optimization (FIG. 2). The range of ABR thresholds observed in wild-type and heterozygous OTOF mutant mice (OTOF^{Q828X/+}) are indicated by a box.

FIGS. 3A-3C are a series of graphs showing electrophysiological signatures of hearing function in mice treated with viral vectors expressing OTOF via dual hybrid vector systems. Homozygous OTOF-Q828X mice were treated with an AAV1-Myo15 (SEQ ID NO: 21)-hOTOF (isoform 5, SEQ ID NO: 1) dual hybrid vector system by injection through the round window membrane and auditory brainstem response (ABR) thresholds were used to assess hearing function (FIG. 3A). Untreated animals (untreated Otof HOM) showed no detectable recovery in hearing function, whereas treated animals exhibited robust recovery, which was consistent from four weeks post-treatment (Otof HOM at 4 weeks after treatment) to eight weeks post-treatment (Otof HOM at 8-11 weeks after treatment). ABR thresholds from heterozygous mice (Otof HET) were also tested.

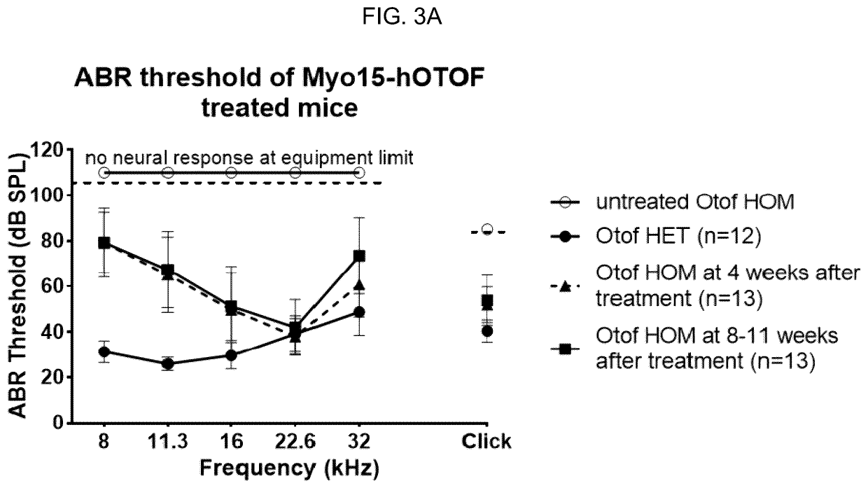

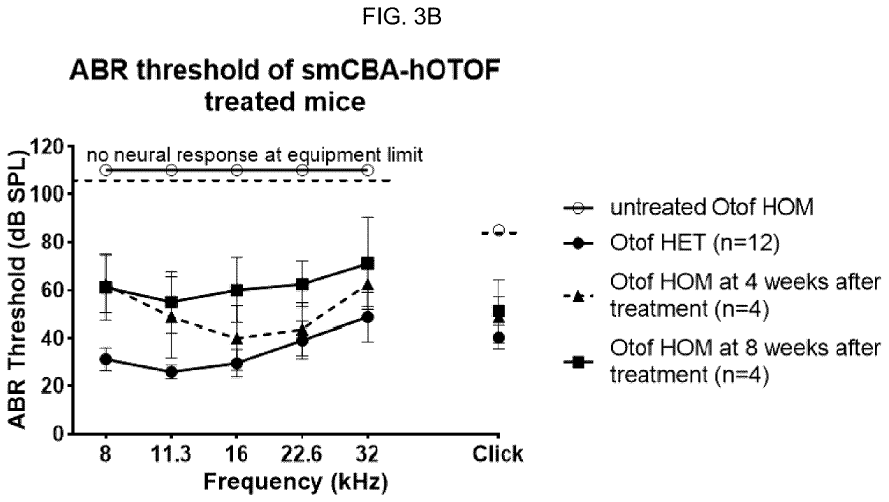

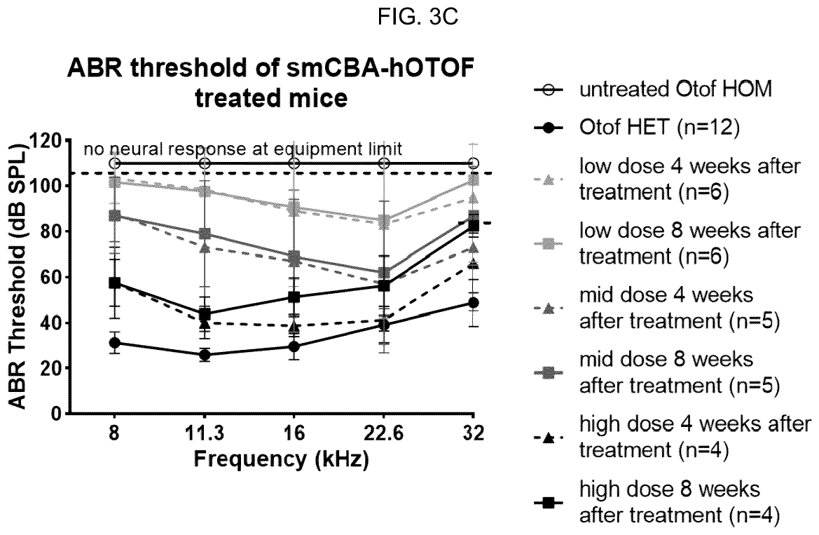

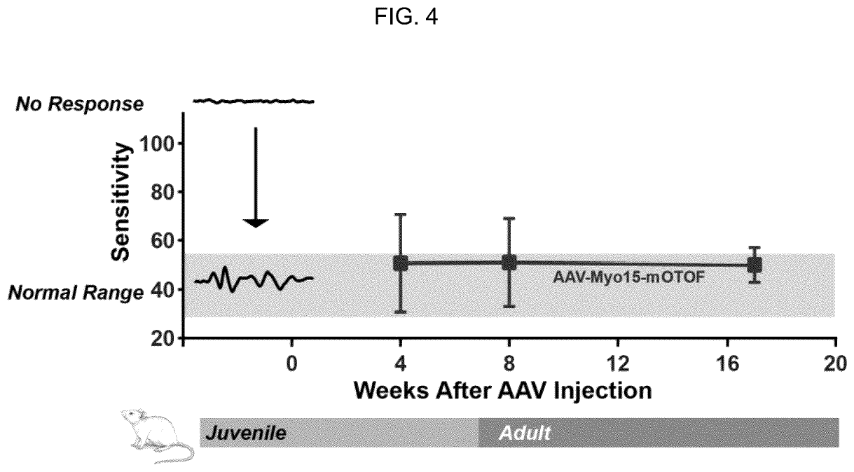

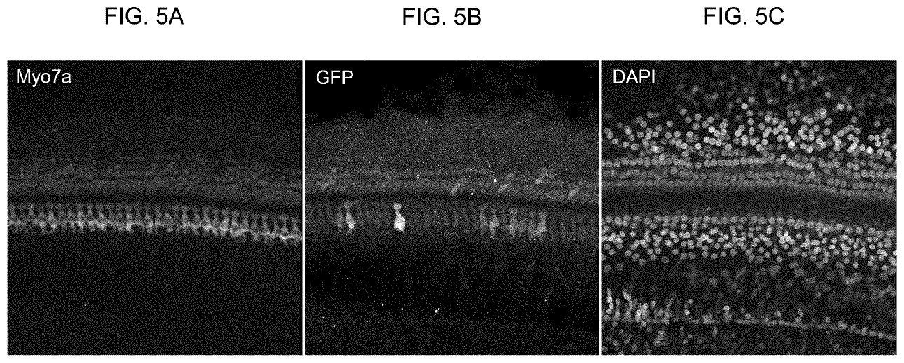

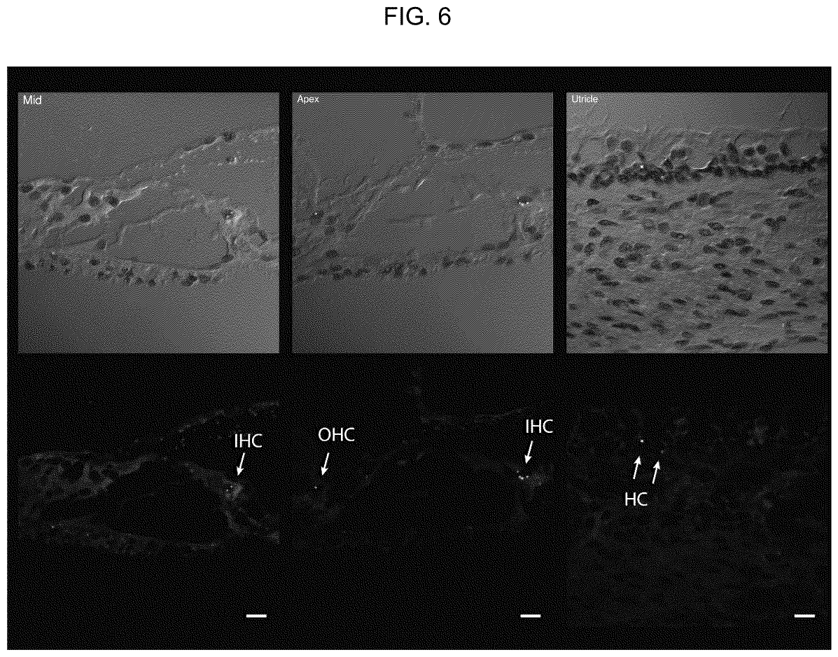

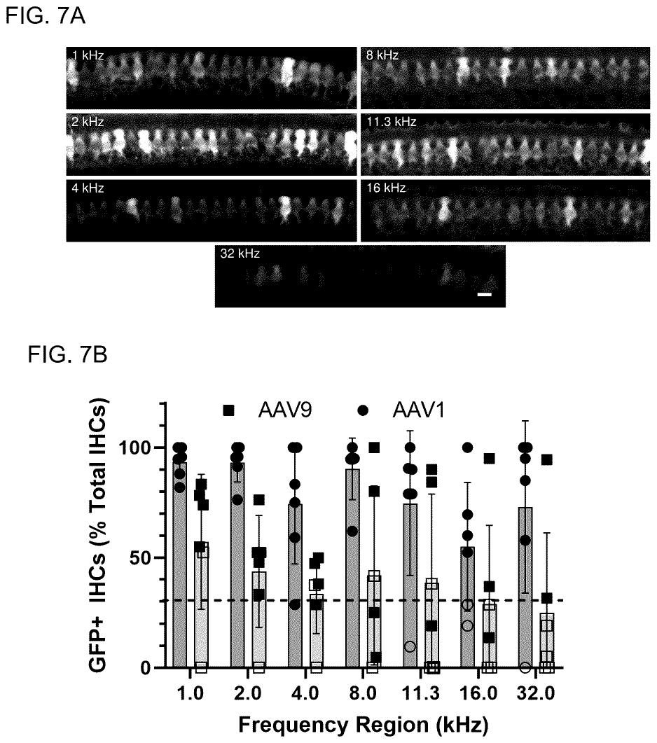

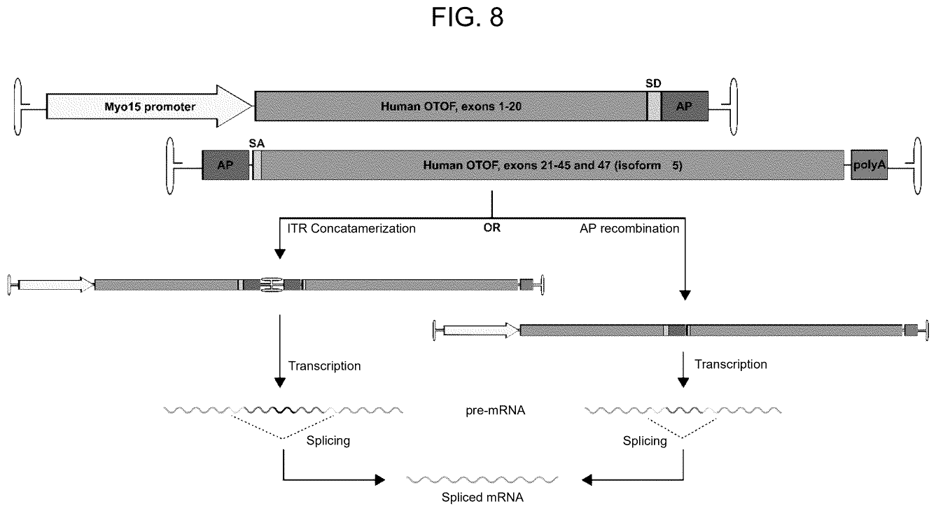

In another set of experiments, Homozygous OTOF-Q828X mice were treated with an AAV1-truncated chimeric CMV-chicken β-actin (smCBA; SEQ ID NO: 44)-hOTOF (isoform 5, SEQ ID NO: 1) dual hybrid vector system by injection through the round window membrane and ABR thresholds were used to assess hearing function as described above (FIG. 3B). Untreated animals showed no detectable recovery in hearing function, while treated animals exhibited a robust recovery at four weeks post-treatment (Otof HOM at 4 weeks after treatment). When the same mice were evaluated at eight weeks post-treatment (Otof HOM at 8 weeks after treatment), ABR thresholds increased, suggesting less durable recovery with the smCBA promoter. ABR thresholds from heterozygous mice were also tested.

In yet another set of experiments, homozygous OTOF-Q828X mice were treated with an AAV1-smCBA (SEQ ID NO: 44)-hOTOF (isoform 5, SEQ ID NO: 1) dual hybrid vector system by injection through the round window membrane at low, medium (mid), and high doses and ABR thresholds were used to assess hearing function at four weeks and eight weeks post-treatment (FIG. 3C). A dose-dependent recovery in ABR thresholds was observed at both timepoints. When comparing the eight weeks versus the four weeks timepoints, recovery was steady for the low and mid doses, but decreased for the high dose animals. ABR thresholds for HET animals were also tested.

FIG. 4 is a graph showing the durability of hearing recovery in mice treated with viral vectors expressing OTOF via dual hybrid vector systems. Homozygous OTOF-Q828X mice were treated with AAV2quad(Y-F)-Myo15 (SEQ ID NO: 21)-murine OTOF (mOTOF, transcript variant 1, RefSeq NM_001100395) and AAV2quad(Y-F)-Myo15 (SEQ ID NO: 31)-mOTOF (transcript variant 1, RefSeq NM_001100395) dual hybrid vector systems by injection through the round window membrane and ABR thresholds were used to assess hearing function. Untreated animals showed no detectable recovery in hearing function, whereas treated animals exhibited robust recovery, which was consistent from four weeks post-treatment to seventeen weeks post-treatment (FIG. 4). The graph depicts mean hearing thresholds at 22.6 KHz+/−standard deviation.

FIGS. 5A-5C are a series of fluorescent images of non-human primate cochlea showing that dual hybrid vectors can be used to express full length, functional GFP in hair cells. A non-human primate (NHP) received a local injection to the round window of the inner ear at a flow rate of 6 μL/min with an AAV1-Myo15 (SEQ ID NO: 21)-GFP (viral titer of 3.18E13 vg/mL for the 5′ vector and 3.42E13 vg/mL for the 3′ vector) dual hybrid vector system. Four weeks post-injection, inner ears were removed, and a surface preparation of the basilar membranes was made. Dual hybrid vectors resulted in GFP expression in hair cells across the entire baso-apical axis of the cochlea. High magnification images at 4 kHz showed GFP expression was observed within inner hair cells (IHCs) (FIG. 5B). Immunohistochemistry for Myo7A was used to visualize hair cells (FIG. 5A) and nuclei were stained with DAPI (FIG. 5C).

FIG. 6 is a series of images of sections with differential interference contrast images (top three panels) and corresponding BaseScope™ fluorescent staining specific for the exon 20/exon 21 junction of human OTOF (bottom three panels) from various areas of NHP ears four weeks after being treated with a dual hybrid viral vector system described in Example 6 herein to express full length OTOF. The fluorescent staining is specific for full length human OTOF transcript generated from the dual vectors. “IHC” refers to inner hair cells. “OHC” refers to outer hair cells. “HC” refers to hair cells. The scale bars seen in the lower panels represent a scale of 10 μm.

FIGS. 7A-7B represent the expression of GFP in inner hair cells at various locations in the organ of Corti in NHP treated with a dual vector system expressing eGFP under the control of a Myo15 promoter in either an AAV1 or AAV9 vector as described in Example 6. FIG. 7A shows a series of confocal images of inner hair cells at different frequency locations across the organ of Corti and the corresponding eGFP-signal after AAV1 dual hybrid GFP transduction. FIG. 7B is a graph showing a quantification of the number of inner hair cells expressing eGFP in various regions of the organ of Corti.

FIG. 8 is a schematic depicting an exemplary dual vector system of the invention. The 5′ vector contains, from 5′ to 3′ a Myo15 promoter, the first 20 exons of the 5′ half of the human otoferlin isoform 5 coding sequence, a splice donor site (SD), and an alkaline phosphatase (AP)-derived homology region. The 3′ vector contains, from 5′ to 3′, a copy of the AP homology region, a complementary splice acceptor site (SA), the remaining 3′ portion of the human otoferlin isoform 5 coding sequence (exons 21-45 and 47), and a polyadenylation (polyA) signal. Both the 5′ and 3′ vectors contain flanking inverted terminal repeats (ITRs). These vectors are designed to reconstitute a functional human OTOF isoform 5 gene cassette when co-transduced into the same target cell and vector recombination can occur through two recombinogenic mechanisms in vivo. The ITRs naturally associate during second strand DNA synthesis and extension and the identical AP sequences in each vector can undergo homologous recombination, forming a functional human OTOF isoform 5 cassette in either case. The resultant double-stranded DNA can express RNA from the Myo15 promoter, and the SD/SA sites facilitate the removal by splicing of the AP region and ITR sequences to generate a mature mRNA encoding the full-length human OTOF isoform 5 coding sequence.

### DETAILED DESCRIPTION

Described herein are compositions and methods for the treatment of sensorineural hearing loss or auditory neuropathy in a subject (such as a mammalian subject, for instance, a human) by administering a first nucleic acid vector (e.g., an AAV vector) containing a promoter and a polynucleotide encoding an N-terminal portion of an otoferlin (OTOF) isoform 5 protein (e.g., a wild-type (WT) human OTOF isoform 5 protein) and a second nucleic acid vector (e.g., an AAV vector) containing a polynucleotide encoding a C-terminal portion of an OTOF isoform 5 protein and a polyadenylation (poly(A)) sequence. When introduced into a mammalian cell, such as a cochlear hair cell, the polynucleotides encoded by the two nucleic acid vectors can combine to form a nucleic acid molecule that encodes the full-length OTOF isoform 5 protein. The compositions and methods described herein can, therefore, be used to induce or increase expression of WT OTOF isoform 5 in cochlear hair cells of a subject who has an OTOF deficiency (e.g., low OTOF expression or an OTOF mutation that impairs OTOF expression or function).

### Otoferlin

OTOF is a 230 kDa membrane protein that contains at least six C2 domains implicated in calcium, phospholipid, and protein binding. Human OTOF is encoded by a gene that contains 47 exons, and the full-length protein is made up of 1,997 amino acids. OTOF is located at ribbon synapses in inner hair cells, where it is believed to function as a calcium sensor in synaptic vesicle fusion, triggering the fusion of neurotransmitter-containing vesicles with the plasma membrane. It has also been implicated in vesicle replenishment and clathrin-mediated endocytosis, and has been shown to interact with Myosin VI, Rab8b, SNARE proteins, calcium channel Cav1.3, Ergic2, and AP-2. The mechanism by which OTOF mediates exocytosis and the physiological significance of its interactions with its binding partners remain to be determined.

There are multiple long and short isoforms of the Otoferlin gene. Studies of human genetic deafness have suggested that long isoforms are important for inner ear function. However, the role of these individual long isoforms and other protein variants in inner ear function is not understood. To develop effective gene transfer therapies for patients who experience deafness secondary to genetically driven Otoferlin deficiency, a cDNA sequence that encodes functional OTOF isoforms in the ear must be identified.

The present invention is based, in part, on the discovery that OTOF isoform 5 was preferentially expressed in the inner ear of non-human primates, and that human OTOF isoform 5, but not human OTOF isoform 1, was able to rescue hearing in genetically engineered, congenitally deaf mice with Otoferlin deficiency. Accordingly, the dual vector systems described herein (e.g., dual vector systems for the expression of OTOF isoform 5) may be used to treat sensorineural hearing loss or auditory neuropathy in human subjects having a deficiency (e.g., mutation) in the OTOF gene.

### Otoferlin-Associated Hearing Loss

OTOF was first identified by a study investigating the genetics of a non-syndromic form of deafness, autosomal recessive deafness-9 (DFNB9). Mutations in OTOF have since been found to cause sensorineural hearing loss in patients throughout the world, with many patients carrying OTOF mutations having auditory neuropathy, a disorder in which the inner ear detects sound, but is unable to properly transmit sound from the ear to the brain. These patients have an abnormal auditory brainstem response (ABR) and impaired speech discrimination with initially normal otoacoustic emissions. Patients carrying homozygous or compound heterozygous mutations often develop hearing loss in early childhood, and the severity of hearing impairment has been found to vary with the location and type of mutation in OTOF.

The compositions and methods described herein can be used to treat sensorineural hearing loss or auditory neuropathy by administering a first nucleic acid vector containing a polynucleotide encoding an N-terminal portion of an OTOF isoform 5 protein and a second nucleic acid vector containing a polynucleotide encoding a C-terminal portion of an OTOF isoform 5 protein. The full-length OTOF isoform 5 coding sequence is too large to include in the type of vector that is commonly used for gene therapy (e.g., an adeno-associated virus (AAV) vector, which is thought to have a packaging limit of 5 kb). The compositions and methods described herein overcome this problem by dividing the OTOF isoform 5 coding sequence between two different nucleic acid vectors (e.g., AAV vectors) that can combine in a cell to reconstitute the full-length OTOF isoform 5 sequence. These compositions and methods can be used to treat subjects having one or more mutations in the OTOF gene, e.g., an OTOF mutation that reduces OTOF expression, reduces OTOF function, or is associated with hearing loss (e.g., a frameshift mutation, a nonsense mutation, a deletion, or a missense substitution). When the first and second nucleic acid vectors are administered in a composition, the polynucleotides encoding the N-terminal and C-terminal portions of OTOF isoform 5 can combine within a cell (e.g., a human cell, e.g., a cochlear hair cell) to form a single nucleic acid molecule that contains the full-length OTOF isoform 5 coding sequence (e.g., through homologous recombination and/or splicing).

The nucleic acid vectors (e.g., AAV vectors) used in the compositions and methods described herein include polynucleotide sequences that encode wild-type OTOF isoform 5, or a variant thereof, such as a polynucleotide sequences that, when combined, encode a protein having at least 85% sequence identity (e.g., 85%, 90%, 95%, 96%, 97%, 98%, 99%, or more, sequence identity) to the amino acid sequence of wild-type mammalian (e.g., human or mouse) OTOF isoform 5. The polynucleotides used in the nucleic acid vectors described herein can encode an N-terminal portion and a C-terminal portion of an OTOF isoform 5 amino acid sequence in Table 2 below (e.g., two portions that, when combined, encode a full-length OTOF isoform 5 amino acid sequence listed in Table 2, e.g., SEQ ID NO: 1).

According to the methods described herein, a subject can be administered a composition containing a first nucleic acid vector and a second nucleic acid vector that contain an N-terminal and C-terminal portion, respectively, of a polynucleotide sequence encoding the amino acid sequence of SEQ ID NO: 1, or a polynucleotide sequence encoding an amino acid sequence having at least 85% sequence identity (e.g., 85%, 90%, 95%, 96%, 97%, 98%, 99%, or more, sequence identity) to the amino acid sequence of SEQ ID NO: 1, or a polynucleotide sequence encoding an amino acid sequence that contains one or more conservative amino acid substitutions relative to SEQ ID NO: 1 (e.g., 1, 2, 3, 4, 5, 6, 7, 8, 9, 10, 11, 12, 13, 14, 15, 16, 17, 18, 19, 20 or more conservative amino acid substitutions), provided that the OTOF analog encoded retains the therapeutic function of wild-type OTOF isoform 5 (e.g., the ability to regulate exocytosis at ribbon synapses or rescue or improve ABR response in an animal model of hearing loss related to Otoferlin gene deficiency (e.g., OTOF mutation)). No more than 10% of the amino acids in the N-terminal portion of the human OTOF isoform 5 protein and no more than 10% of the amino acids in the C-terminal portion of the human OTOF isoform 5 protein may be replaced with conservative amino acid substitutions. The OTOF isoform 5 protein may be encoded by a polynucleotide having the sequence of SEQ ID NO: 2 or SEQ ID NO: 3. The OTOF isoform 5 protein may also be encoded by a polynucleotide having single nucleotide variants (SNVs) that have been found to be non-pathogenic in human subjects. The OTOF isoform 5 protein may be a human OTOF isoform 5 protein or may be a homolog of the human isoform 5 protein from another mammalian species (e.g., mouse, rat, cow, horse, goat, sheep, donkey, cat, dog, rabbit, guinea pig, or other mammal).

TABLE 2
OTOF Sequences

| SEQ ID | | |
|---|---|---|
| NO. | Sequence Name | Sequence |
| 1 | Human OTOF | MALLIHLKTVSELRGRGDRIAKVTFRGQSFYSRVLENCEDVADFDE |
| | isoform 5 protein | TFRWPVASSIDRNEMLEIQVFNYSKVFSNKLIGTFRMVLQKVVEES |
| | (NP_001274418.1), | HVEVTDTLIDDNNAIIKTSLCVEVRYQATDGTVGSWDDGDFLGDES |
| | also called human | LQEEEKDSQETDGLLPGSRPSSRPPGEKSFRRAGRSVFSAMKLGK |
| | otoferlin isoform e, | NRSHKEEPQRPDEPAVLEMEDLDHLAIRLGDGLDPDSVSLASVTAL |
| | 1997 aa | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLCNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTAFVWFLNPLKSIKYLICTRYKWLIIKIVLAL |
| | | LGLLMLGLFLYSLPGYMVKKLLGA |
| 2 | DNA sequence | ATGGCCTTGCTCATCCACCTCAAGACAGTCTCGGAGCTGCGGG |
| | encoding the human | GCAGGGGCGACCGGATCGCCAAAGTGACTTTCCGAGGGCAATC |
| | otoferlin isoform 5 | CTTCTACTCTCGGGTCCTGGAGAACTGTGAGGATGTGGCTGACT |
| | protein (SEQ ID NO: | TTGATGAGACATTTCGGTGGCCGGTGGCCAGCAGCATCGACAG |
| | 1), 5994 bp, | AAATGAGATGCTGGAGATTCAGGTTTTCAACTACAGCAAAGTCTT |
| | corresponds to the | CAGCAACAAGCTCATCGGGACCTTCCGCATGGTGCTGCAGAAG |
| | coding sequence | GTGGTAGAGGAGAGCCATGTGGAGGTGACTGACACGCTGATTG |
| | documented in | ATGACAACAATGCTATCATCAAGACCAGCCTGTGCGTGGAGGTC |
| | NM_001287489 | CGGTATCAGGCCACTGACGGCACAGTGGGCTCCTGGGACGATG |
| | | GGGACTTCCTGGGAGATGAGTCTCTTCAAGAGGAAGAGAAGGA |
| | | CAGCCAAGAGACGGATGGACTGCTCCCAGGCTCCCGGCCCAGC |
| | | TCCCGGCCCCCAGGAGAGAAGAGCTTCCGGAGAGCCGGGAGG |
| | | AGCGTGTTCTCCGCCATGAAGCTCGGCAAAAACCGGTCTCACAA |
| | | GGAGGAGCCCCAAAGACCAGATGAACCGGCGGTGCTGGAGAT |
| | | GGAAGACCTTGACCATCTGGCCATTCGGCTAGGAGATGGACTG |
| | | GATCCCGACTCGGTGTCTCTAGCCTCAGTCACAGCTCTCACCAC |
| | | TAATGTCTCCAACAAGCGATCTAAGCCAGACATTAAGATGGAGC |
| | | CAAGTGCTGGGCGGCCCATGGATTACCAGGTCAGCATCACGGT |
| | | GATCGAGGCCCGGCAGCTGGTGGGCTTGAACATGGACCCTGTG |
| | | GTGTGCGTGGAGGTGGGTGACGACAAGAAGTACACATCCATGA |
| | | AGGAGTCCACTAACTGCCCCTATTACAACGAGTACTTCGTCTTC |
| | | GACTTCCATGTCTCTCCGGATGTCATGTTTGACAAGATCATCAAG |
| | | ATTTCGGTGATTCACTCCAAGAACCTGCTGCGCAGTGGCACCCT |
| | | GGTGGGCTCCTTCAAAATGGACGTGGGAACCGTGTACTCGCAG |
| | | CCAGAGCACCAGTTCCATCACAAGTGGGCCATCCTGTCTGACCC |
| | | CGATGACATCTCCTCGGGGCTGAAGGGCTACGTGAAGTGTGAC |
| | | GTTGCCGTGGTGGGCAAAGGGGACAACATCAAGACGCCCCACA |
| | | AGGCCAATGAGACCGACGAAGATGACATTGAGGGGAACTTGCT |
| | | GCTCCCCGAGGGGGTGCCCCCCGAACGCCAGTGGGCCCGGTT |
| | | CTATGTGAAAATTTACCGAGCAGAGGGGCTGCCCCGTATGAACA |
| | | CAAGCCTCATGGCCAATGTAAAGAAGGCTTTCATCGGTGAAAAC |
| | | AAGGACCTCGTGGACCCCTACGTGCAAGTCTTCTTTGCTGGCCA |
| | | GAAGGGCAAGACTTCAGTGCAGAAGAGCAGCTATGAGCCCCTG |
| | | TGGAATGAGCAGGTCGTCTTTACAGACCTCTTCCCCCCACTCTG |
| | | CAAACGCATGAAGGTGCAGATCCGAGACTCGGACAAGGTCAAC |
| | | GACGTGGCCATCGGCACCCACTTCATTGACCTGCGCAAGATTTC |
| | | TAATGACGGAGACAAAGGCTTCCTGCCCACACTGGGCCCAGCC |
| | | TGGGTGAACATGTACGGCTCCACACGTAACTACACGCTGCTGGA |
| | | TGAGCATCAGGACCTGAACGAGGGCCTGGGGGAGGGTGTGTC |
| | | CTTCCGGGCCCGGCTCCTGCTGGGCCTGGCTGTGGAGATCGTA |
| | | GACACCTCCAACCCTGAGCTCACCAGCTCCACAGAGGTGCAGG |
| | | TGGAGCAGGCCACGCCCATCTCGGAGAGCTGTGCAGGTAAAAT |
| | | GGAAGAATTCTTTCTCTTTGGAGCCTTCCTGGAGGCCTCAATGA |
| | | TCGACCGGAGAAACGGAGACAAGCCCATCACCTTTGAGGTCAC |
| | | CATAGGCAACTATGGGAACGAAGTTGATGGCCTGTCCCGGCCC |
| | | CAGCGGCCTCGGCCCCGGAAGGAGCCGGGGGATGAGGAAGAA |
| | | GTAGACCTGATTCAGAACGCAAGTGATGACGAGGCCGGTGATG |
| | | CCGGGGACCTGGCCTCAGTCTCCTCCACTCCACCAATGCGGCC |
| | | CCAGGTCACCGACAGGAACTACTTCCATCTGCCCTACCTGGAGC |
| | | GAAAGCCCTGCATCTACATCAAGAGCTGGTGGCCGGACCAGCG |
| | | CCGCCGCCTCTACAATGCCAACATCATGGACCACATTGCCGACA |
| | | AGCTGGAAGAAGGCCTGAACGACATACAGGAGATGATCAAAAC |
| | | GGAGAAGTCCTACCCTGAGCGTCGCCTGCGGGGCGTCCTGGA |
| | | GGAGCTGAGCTGTGGCTGCTGCCGCTTCCTCTCCCTCGCTGAC |
| | | AAGGACCAGGGCCACTCATCCCGCACCAGGCTTGACCGGGAGC |
| | | GCCTCAAGTCCTGCATGAGGGAGCTGGAAAACATGGGGCAGCA |
| | | GGCCAGGATGCTGCGGGCCCAGGTGAAGCGGCACACGGTGCG |
| | | GGACAAGCTGAGGCTGTGCCAGAACTTCCTGCAGAAGCTGCGC |
| | | TTCCTGGCGGACGAGCCCCAGCACAGCATTCCCGACATCTTCAT |
| | | CTGGATGATGAGCAACAACAAGCGTGTCGCCTATGCCCGTGTG |
| | | CCCTCCAAGGACCTGCTCTTCTCCATCGTGGAGGAGGAGACTG |
| | | GCAAGGACTGCGCCAAGGTCAAGACGCTCTTCCTTAAGCTGCC |
| | | AGGGAAGCGGGGCTTCGGCTCGGCAGGCTGGACAGTGCAGGC |
| | | CAAGGTGGAGCTGTACCTGTGGCTGGGCCTCAGCAAACAGCGC |
| | | AAGGAGTTCCTGTGCGGCCTGCCCTGTGGCTTCCAGGAGGTCA |
| | | AGGCAGCCCAGGGCCTGGGCCTGCATGCCTTCCCACCCGTCAG |
| | | CCTGGTCTACACCAAGAAGCAGGCGTTCCAGCTCCGAGCGCAC |
| | | ATGTACCAGGCCCGCAGCCTCTTTGCCGCCGACAGCAGCGGAC |
| | | TCTCAGACCCCTTTGCCCGCGTCTTCTTCATCAATCAGAGTCAG |
| | | TGCACAGAGGTGCTGAATGAGACCCTGTGTCCCACCTGGGACC |
| | | AGATGCTGGTGTTCGACAACCTGGAGCTCTATGGTGAAGCTCAT |
| | | GAGCTGAGGGACGATCCGCCCATCATTGTCATTGAAATCTATGA |
| | | CCAGGATTCCATGGGCAAAGCTGACTTCATGGGCCGGACCTTC |
| | | GCCAAACCCCTGGTGAAGATGGCAGACGAGGCGTACTGCCCAC |
| | | CCCGCTTCCCACCTCAGCTCGAGTACTACCAGATCTACCGTGGC |
| | | AACGCCACAGCTGGAGACCTGCTGGCGGCCTTCGAGCTGCTGC |
| | | AGATTGGACCAGCAGGGAAGGCTGACCTGCCCCCCATCAATGG |
| | | CCCGGTGGACGTGGACCGAGGTCCCATCATGCCCGTGCCCATG |
| | | GGCATCCGGCCCGTGCTCAGCAAGTACCGAGTGGAGGTGCTGT |
| | | TCTGGGGCCTACGGGACCTAAAGCGGGTGAACCTGGCCCAGGT |
| | | GGACCGGCCACGGGTGGACATCGAGTGTGCAGGGAAGGGGGT |
| | | GCAGTCGTCCCTGATCCACAATTATAAGAAGAACCCCAACTTCA |
| | | ACACCCTCGTCAAGTGGTTTGAAGTGGACCTCCCAGAGAACGA |
| | | GCTGCTGCACCCGCCCTTGAACATCCGTGTGGTGGACTGCCGG |
| | | GCCTTCGGTCGCTACACACTGGTGGGCTCCCATGCCGTCAGCT |
| | | CCCTGCGACGCTTCATCTACCGGCCCCCAGACCGCTCGGCCCC |
| | | CAGCTGGAACACCACGGTCAGGCTTCTCCGGCGCTGCCGTGTG |
| | | CTGTGCAATGGGGGCTCCTCCTCTCACTCCACAGGGGAGGTTG |
| | | TGGTGACTATGGAGCCAGAGGTACCCATCAAGAAACTGGAGAC |
| | | CATGGTGAAGCTGGACGCGACTTCTGAAGCTGTTGTCAAGGTG |
| | | GATGTGGCTGAGGAGGAGAAGGAGAAGAAGAAGAAGAAGAAGG |
| | | GCACTGCGGAGGAGCCAGAGGAGGAGGAGCCAGACGAGAGCA |
| | | TGCTGGACTGGTGGTCCAAGTACTTTGCCTCCATTGACACCATG |
| | | AAGGAGCAACTTCGACAACAAGAGCCCTCTGGAATTGACTTGGA |
| | | GGAGAAGGAGGAAGTGGACAATACCGAGGGCCTGAAGGGGTC |
| | | AATGAAGGGCAAGGAGAAGGCAAGGGCTGCCAAAGAGGAGAAG |
| | | AAGAAGAAAACTCAGAGCTCTGGCTCTGGCCAGGGGTCCGAGG |
| | | CCCCCGAGAAGAAGAAACCCAAGATTGATGAGCTTAAGGTATAC |
| | | CCCAAAGAGCTGGAGTCCGAGTTTGATAACTTTGAGGACTGGCT |
| | | GCACACTTTCAACTTGCTTCGGGGCAAGACCGGGGATGATGAG |
| | | GATGGCTCCACCGAGGAGGAGCGCATTGTGGGACGCTTCAAGG |
| | | GCTCCCTCTGCGTGTACAAAGTGCCACTCCCAGAGGACGTGTC |
| | | CCGGGAAGCCGGCTACGACTCCACCTACGGCATGTTCCAGGGC |
| | | ATCCCGAGCAATGACCCCATCAATGTGCTGGTCCGAGTCTATGT |
| | | GGTCCGGGCCACGGACCTGCACCCTGCTGACATCAACGGCAAA |
| | | GCTGACCCCTACATCGCCATCCGGCTAGGCAAGACTGACATCC |
| | | GCGACAAGGAGAACTACATCTCCAAGCAGCTCAACCCTGTCTTT |
| | | GGGAAGTCCTTTGACATCGAGGCCTCCTTCCCCATGGAATCCAT |
| | | GCTGACGGTGGCTGTGTATGACTGGGACCTGGTGGGCACTGAT |
| | | GACCTCATTGGGGAAACCAAGATCGACCTGGAGAACCGCTTCTA |
| | | CAGCAAGCACCGCGCCACCTGCGGCATCGCCCAGACCTACTCC |
| | | ACACATGGCTACAATATCTGGCGGGACCCCATGAAGCCCAGCC |
| | | AGATCCTGACCCGCCTCTGCAAAGACGGCAAAGTGGACGGCCC |
| | | CCACTTTGGGCCCCCTGGGAGAGTGAAGGTGGCCAACCGCGTC |
| | | TTCACTGGGCCCTCTGAGATTGAGGACGAGAACGGTCAGAGGA |
| | | AGCCCACAGACGAGCATGTGGCGCTGTTGGCCCTGAGGCACTG |
| | | GGAGGACATCCCCCGCGCAGGCTGCCGCCTGGTGCCAGAGCA |
| | | TGTGGAGACGAGGCCGCTGCTCAACCCCGACAAGCCGGGCATC |
| | | GAGCAGGGCCGCCTGGAGCTGTGGGTGGACATGTTCCCCATGG |
| | | ACATGCCAGCCCCTGGGACGCCTCTGGACATCTCACCTCGGAA |
| | | GCCCAAGAAGTACGAGCTGCGGGTCATCATCTGGAACACAGAT |
| | | GAGGTGGTCTTGGAGGACGACGACTTCTTCACAGGGGAGAAGT |
| | | CCAGTGACATCTTCGTGAGGGGGTGGCTGAAGGGCCAGCAGGA |
| | | GGACAAGCAGGACACAGACGTCCACTACCACTCCCTCACTGGC |
| | | GAGGGCAACTTCAACTGGCGCTACCTGTTCCCCTTCGACTACCT |
| | | GGCGGCGGAGGAGAAGATCGTCATCTCCAAGAAGGAGTCCATG |
| | | TTCTCCTGGGACGAGACCGAGTACAAGATCCCCGCGCGGCTCA |
| | | CCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGG |
| | | CCTTCGTCTGGTTCCTCAACCCTCTCAAGTCCATCAAGTACCTCA |
| | | TCTGCACCCGGTACAAGTGGCTCATCATCAAGATCGTGCTGGCG |
| | | CTGTTGGGGCTGCTCATGTTGGGGCTCTTCCTCTACAGCCTCCC |
| | | TGGCTACATGGTCAAAAAGCTCCTTGGGGCATGA |
| 3 | Codon-optimized | ATGGCACTGCTGATCCACCTGAAAACCGTCTCCGAACTGAGAGG |
| | (CO) DNA sequence | CAGAGGGGACAGAATCGCTAAAGTCACCTTCCGGGGACAGAGC |
| | encoding the human | TTTTACAGCAGGGTGCTGGAGAACTGCGAGGACGTGGCCGACT |
| | otoferlin isoform 5 | TTGACGAGACATTCAGGTGGCCCGTGGCCAGCTCCATCGATCG |
| | protein (SEQ ID NO: | CAATGAGATGCTGGAGATCCAGGTGTTTAACTATAGCAAGGTGT |
| | 1), 5994 bp | TCTCCAATAAGCTGATCGGCACCTTCCGGATGGTGCTGCAGAAG |
| | | GTGGTGGAGGAGTCCCACGTGGAGGTGACCGACACACTGATCG |
| | | ACGATAACAATGCCATCATCAAGACATCCCTGTGCGTGGAGGTG |
| | | CGCTACCAGGCCACCGATGGCACAGTGGGCTCTTGGGACGATG |
| | | GCGACTTCCTGGGCGATGAGTCCCTGCAGGAGGAGGAGAAGGA |
| | | CTCTCAGGAGACAGATGGCCTGCTGCCTGGCTCCCGGCCATCT |
| | | AGCCGCCCCCCTGGCGAGAAGTCTTTTAGGAGAGCCGGCAGGT |
| | | CCGTGTTCTCTGCCATGAAGCTGGGCAAGAACAGGAGCCACAA |
| | | GGAGGAGCCTCAGAGGCCCGACGAGCCAGCCGTGCTGGAGAT |
| | | GGAGGACCTGGATCACCTGGCCATCAGACTGGGCGATGGCCTG |
| | | GACCCTGATAGCGTGTCCCTGGCCTCCGTGACCGCCCTGACCA |
| | | CAAACGTGTCTAATAAGCGGAGCAAGCCAGACATCAAGATGGAG |
| | | CCATCTGCCGGCAGGCCCATGGATTACCAGGTGAGCATCACAG |
| | | TGATCGAGGCCAGACAGCTGGTGGGCCTGAACATGGACCCCGT |
| | | GGTGTGCGTGGAAGTGGGCGACGATAAGAAGTACACCTCCATG |
| | | AAGGAGTCTACAAACTGTCCATACTACAACGAGTACTTCGTGTTT |
| | | GATTTCCACGTGAGCCCCGACGTGATGTTCGATAAGATCATCAA |
| | | GATCAGCGTGATCCACTCCAAGAATCTGCTGCGGTCTGGCACC |
| | | CTGGTGGGAAGCTTTAAGATGGACGTGGGCACAGTGTACTCTCA |
| | | GCCTGAGCACCAGTTCCACCACAAGTGGGCCATCCTGAGCGAT |
| | | CCAGACGATATCTCCTCTGGCCTGAAGGGCTATGTGAAGTGCGA |
| | | CGTGGCAGTGGTGGGCAAGGGCGATAACATCAAGACCCCACAC |
| | | AAGGCCAATGAGACAGACGAGGACGATATCGAGGGAAACCTGC |
| | | TGCTGCCAGAGGGAGTGCCACCCGAGAGGCAGTGGGCCAGGT |
| | | TCTACGTGAAGATCTATAGGGCAGAGGGCCTGCCTAGGATGAA |
| | | CACCAGCCTGATGGCCAATGTGAAGAAGGCCTTCATCGGCGAG |
| | | AACAAGGACCTGGTGGATCCCTACGTGCAGGTGTTCTTTGCCG |
| | | GCCAGAAGGGCAAGACCTCCGTGCAGAAGAGCTCCTATGAGCC |
| | | TCTGTGGAATGAGCAGGTGGTGTTTACAGACCTGTTCCCTCCAC |
| | | TGTGCAAGAGGATGAAGGTGCAGATCAGAGACTCTGATAAGGT |
| | | GAACGACGTGGCCATCGGCACCCACTTTATCGATCTGAGGAAG |
| | | ATCAGCAATGACGGCGATAAGGGCTTCCTGCCCACCCTGGGCC |
| | | CCGCCTGGGTGAACATGTACGGCAGCACCAGAAATTATACACTG |
| | | CTGGACGAGCACCAGGATCTGAACGAGGGCCTGGGCGAGGGC |
| | | GTGAGCTTTAGAGCCAGGCTGCTGCTGGGCCTGGCCGTGGAGA |
| | | TCGTGGACACCTCCAATCCCGAGCTGACCTCTAGCACAGAGGT |
| | | GCAGGTGGAGCAGGCCACACCTATCTCTGAGAGCTGTGCCGGC |
| | | AAGATGGAGGAGTTCTTTCTGTTTGGCGCCTTCCTGGAGGCCTC |
| | | CATGATCGACCGGCGCAACGGCGATAAGCCTATCACCTTCGAG |
| | | GTGACAATCGGCAACTACGGCAATGAGGTGGACGGCCTGTCTC |
| | | GGCCCCAGCGCCCAAGGCCCAGAAAGGAGCCTGGCGACGAGG |
| | | AGGAGGTGGATCTGATCCAGAACGCCAGCGACGATGAGGCAGG |
| | | CGACGCAGGCGATCTGGCCTCCGTGTCCTCTACCCCCCCTATG |
| | | CGGCCACAGGTGACAGACCGCAATTACTTTCACCTGCCTTATCT |
| | | GGAGCGCAAGCCATGCATCTACATCAAGTCTTGGTGGCCCGAT |
| | | CAGAGGAGACGGCTGTATAACGCCAATATCATGGACCACATCGC |
| | | CGATAAGCTGGAGGAGGGCCTGAATGACATCCAGGAGATGATC |
| | | AAGACCGAGAAGTCCTATCCAGAGCGCAGGCTGAGGGGCGTGC |
| | | TGGAGGAGCTGAGCTGTGGCTGCTGTAGATTCCTGTCCCTGGC |
| | | CGACAAGGATCAGGGGCACTCATCACGGACACGGCTGGACCGG |
| | | GAGCGGCTGAAATCATGTATGCGGGAGCTGGAAAATATGGGAC |
| | | AGCAGGCAAGGATGCTGCGCGCCCAGGTGAAGAGGCACACCG |
| | | TGAGAGACAAGCTGCGGCTGTGCCAGAACTTCCTGCAGAAGCT |
| | | GCGCTTTCTGGCCGATGAGCCACAGCACAGCATCCCCGACATC |
| | | TTCATCTGGATGATGTCCAACAATAAGAGAGTGGCCTACGCCCG |
| | | GGTGCCCTCTAAGGATCTGCTGTTTAGCATCGTGGAGGAGGAG |
| | | ACAGGCAAGGACTGTGCCAAGGTGAAGACCCTGTTCCTGAAGC |
| | | TGCCTGGCAAGAGAGGCTTTGGCAGCGCCGGATGGACCGTGCA |
| | | GGCAAAGGTGGAGCTGTATCTGTGGCTGGGCCTGTCTAAGCAG |
| | | CGGAAGGAGTTCCTGTGCGGCCTGCCCTGTGGCTTTCAGGAGG |
| | | TGAAGGCAGCACAGGGACTGGGACTGCACGCCTTCCCCCCCGT |
| | | GAGCCTGGTGTACACCAAGAAGCAGGCCTTTCAGCTGAGGGCC |
| | | CATATGTACCAGGCCAGGTCTCTGTTCGCCGCCGATAGCTCCG |
| | | GACTGAGCGACCCTTTTGCCAGGGTGTTCTTTATCAATCAGAGC |
| | | CAGTGCACAGAGGTGCTGAACGAGACCCTGTGCCCAACATGGG |
| | | ATCAGATGCTGGTGTTCGACAACCTGGAGCTGTACGGAGAGGC |
| | | ACACGAGCTGAGGGACGATCCACCCATCATCGTGATCGAGATCT |
| | | ATGATCAGGACTCCATGGGCAAGGCCGATTTCATGGGCAGGAC |
| | | CTTTGCCAAGCCCCTGGTGAAGATGGCCGACGAGGCCTACTGC |
| | | CCTCCAAGATTCCCCCCTCAGCTCGAGTACTATCAGATCTATAG |
| | | GGGAAATGCAACCGCCGGAGACCTGCTGGCCGCCTTTGAGCTG |
| | | CTGCAGATCGGCCCCGCCGGAAAGGCAGACCTGCCACCCATCA |
| | | ACGGCCCAGTGGATGTGGACAGAGGCCCCATCATGCCTGTGCC |
| | | AATGGGCATCAGACCAGTGCTGTCCAAGTACAGGGTGGAGGTG |
| | | CTGTTCTGGGGACTGCGCGACCTGAAGAGGGTGAATCTGGCCC |
| | | AGGTGGATAGGCCCAGAGTGGACATCGAGTGCGCCGGAAAGG |
| | | GCGTGCAGTCTAGCCTGATCCACAACTATAAGAAGAACCCAAAT |
| | | TTCAACACCCTGGTGAAGTGGTTTGAGGTGGATCTGCCCGAGAA |
| | | TGAGCTGCTGCACCCTCCACTGAACATCCGGGTGGTGGACTGT |
| | | AGAGCCTTCGGCAGGTACACCCTGGTGGGCAGCCACGCCGTGA |
| | | GCAGCCTGAGGAGGTTCATCTACAGGCCCCCTGACAGGTCCGC |
| | | CCCTTCTTGGAATACCACAGTGAGACTGCTGCGGCGCTGCAGG |
| | | GTGCTGTGCAACGGAGGCAGCTCCTCTCACTCTACCGGCGAGG |
| | | TGGTGGTGACAATGGAGCCTGAGGTACCCATCAAGAAGCTGGA |
| | | GACCATGGTGAAGCTGGATGCCACAAGCGAGGCAGTGGTGAAG |
| | | GTGGACGTGGCAGAGGAGGAGAAGGAGAAGAAGAAGAAGAAG |
| | | AAGGGAACCGCCGAGGAGCCTGAGGAAGAGGAGCCAGATGAG |
| | | AGCATGCTGGACTGGTGGTCCAAGTACTTCGCCTCTATCGACAC |
| | | AATGAAGGAGCAGCTGAGACAGCAGGAGCCTAGCGGCATCGAT |
| | | CTGGAGGAGAAGGAGGAGGTGGACAATACCGAGGGCCTGAAG |
| | | GGCTCCATGAAGGGCAAGGAGAAGGCAAGGGCAGCAAAGGAA |
| | | GAGAAGAAGAAGAAGACCCAGAGCAGCGGCTCTGGACAGGGCA |
| | | GCGAGGCACCAGAGAAGAAGAAGCCTAAGATCGATGAGCTGAA |
| | | GGTGTACCCAAAGGAGCTGGAGTCCGAGTTCGATAATTTTGAGG |
| | | ACTGGCTGCACACCTTCAACCTGCTGCGCGGCAAGACAGGCGA |
| | | CGATGAGGACGGCAGCACCGAGGAGGAGAGAATCGTGGGCCG |
| | | GTTTAAGGGCTCCCTGTGCGTGTACAAGGTGCCACTGCCTGAG |
| | | GACGTGAGCAGGGAGGCCGGATACGACTCTACCTATGGCATGT |
| | | TCCAGGGCATCCCCTCTAATGATCCTATCAACGTGCTGGTGCGC |
| | | GTGTATGTGGTGAGGGCCACAGATCTGCACCCCGCCGACATCA |
| | | ACGGCAAGGCCGACCCTTACATCGCCATCCGCCTGGGCAAGAC |
| | | CGATATCAGGGACAAGGAGAATTATATCTCCAAGCAGCTGAACC |
| | | CCGTGTTCGGCAAGTCTTTTGACATCGAGGCCAGCTTCCCTATG |
| | | GAGTCCATGCTGACCGTGGCCGTGTACGATTGGGACCTGGTGG |
| | | GCACCGACGATCTGATCGGCGAGACAAAGATCGATCTGGAGAA |
| | | TCGCTTTTATTCTAAGCACAGGGCAACCTGCGGAATCGCACAGA |
| | | CCTACAGCACACACGGCTATAACATCTGGCGCGACCCCATGAA |
| | | GCCTAGCCAGATCCTGACAAGGCTGTGCAAGGATGGCAAGGTG |
| | | GACGGACCACACTTCGGACCACCCGGCAGAGTGAAGGTGGCCA |
| | | ATCGGGTGTTTACAGGCCCTTCCGAGATCGAGGATGAGAACGG |
| | | CCAGCGCAAGCCAACCGACGAGCACGTGGCCCTGCTGGCCCT |
| | | GAGGCACTGGGAGGATATCCCAAGGGCCGGATGTAGGCTGGTG |
| | | CCTGAGCACGTGGAGACCAGACCACTGCTGAATCCAGACAAGC |
| | | CAGGAATCGAGCAGGGCAGGCTGGAGCTGTGGGTGGATATGTT |
| | | CCCAATGGACATGCCAGCCCCAGGAACACCCCTGGATATCTCC |
| | | CCTAGAAAGCCAAAGAAGTACGAGCTGAGAGTGATCATCTGGAA |
| | | CACAGACGAGGTGGTGCTGGAGGACGATGACTTCTTTACCGGC |
| | | GAGAAGTCTAGCGATATCTTTGTGCGCGGATGGCTGAAGGGAC |
| | | AGCAGGAGGACAAGCAGGATACAGACGTGCACTACCACTCCCT |
| | | GACCGGCGAGGGCAATTTCAACTGGAGATACCTGTTCCCTTTTG |
| | | ATTATCTGGCCGCCGAGGAGAAGATCGTGATCTCTAAGAAGGAG |
| | | AGCATGTTTTCCTGGGACGAGACAGAGTATAAGATCCCAGCCAG |
| | | ACTGACCCTGCAGATCTGGGATGCCGACCACTTCAGCGCCGAT |
| | | GACTTTCTGGGCGCCATCGAGCTGGACCTGAACCGGTTCCCAA |
| | | GAGGCGCCAAGACCGCCAAGCAGTGCACAATGGAGATGGCAAC |
| | | CGGAGAGGTGGACGTGCCTCTGGTGTCTATCTTCAAGCAGAAG |
| | | CGGGTGAAGGGATGGTGGCCACTGCTGGCCAGGAACGAGAAT |
| | | GATGAGTTTGAGCTGACAGGCAAGGTGGAGGCAGAGCTGCACC |
| | | TGCTGACCGCCGAGGAGGCAGAGAAGAACCCAGTGGGCCTGG |
| | | CCAGGAATGAGCCCGACCCTCTGGAGAAGCCAAACAGGCCCGA |
| | | TACAGCCTTCGTGTGGTTTCTGAATCCTCTGAAGAGCATCAAGT |
| | | ACCTGATCTGTACCAGGTATAAGTGGCTGATCATCAAGATCGTG |
| | | CTGGCCCTGCTGGGACTGCTGATGCTGGGCCTGTTTCTGTACTC |
| | | CCTGCCCGGCTATATGGTGAAGAAGCTGCTGGGCGCCTGA |
| 4 | Human OTOF | MALLIHLKTVSELRGRGDRIAKVTFRGQSFYSRVLENCEDVADFDE |
| | isoform 1 protein | TFRWPVASSIDRNEMLEIQVFNYSKVFSNKLIGTFRMVLQKVVEES |
| | (NP_919224.1), also | HVEVTDTLIDDNNAIIKTSLCVEVRYQATDGTVGSWDDGDFLGDES |
| | called human | LQEEEKDSQETDGLLPGSRPSSRPPGEKSFRRAGRSVFSAMKLGK |
| | otoferlin isoform a, | NRSHKEEPQRPDEPAVLEMEDLDHLAIRLGDGLDPDSVSLASVTAL |
| | 1997 aa | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLCNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTSFIWFLNPLKSARYFLWHTYRWLLLKLL |
| | | LLLLLLLLLALFLYSVPGYLVKKILGA |
| 5 | DNA sequence | ATGGCCCTGATTGTTCACCTCAAGACTGTCTCAGAGCTCCGAGG |
| | encoding the human | CAAAGGTGACCGGATTGCCAAAGTCACTTTCCGAGGGCAGTCTT |
| | otoferlin isoform 1 | TCTACTCCCGGGTCCTGGAGAACTGCGAGGGTGTGGCTGACTT |
| | protein (SEQ ID NO: | TGATGAGACGTTCCGGTGGCCAGTGGCCAGCAGCATCGACCGG |
| | 4), 5979 bp, | AATGAAGTGTTGGAGATTCAGATTTTCAACTACAGCAAAGTCTTC |
| | corresponds to the | AGCAACAAGCTGATAGGGACCTTCTGCATGGTGCTGCAGAAAGT |
| | coding sequence | GGTGGAGGAGAATCGGGTAGAGGTGACCGACACGCTGATGGAT |
| | documented in | GACAGCAATGCTATCATCAAGACCAGCCTGAGCATGGAGGTCC |
| | NM_001100395 | GGTATCAGGCCACAGATGGCACTGTGGGCCCCTGGGATGATGG |
| | | AGACTTCCTGGGAGATGAATCCCTCCAGGAGGAGAAGGACAGC |
| | | CAGGAGACAGATGGGCTGCTACCTGGTTCCCGACCCAGCACCC |
| | | GGATATCTGGCGAGAAGAGCTTTCGCAGCAAAGGCAGAGAGAA |
| | | GACCAAGGGAGGCAGAGATGGCGAGCACAAAGCGGGAAGGAG |
| | | TGTGTTCTCGGCCATGAAACTCGGCAAAACTCGGTCCCACAAAG |
| | | AGGAGCCCCAAAGACAAGATGAGCCAGCAGTGCTGGAGATGGA |
| | | GGACCTGGACCACCTAGCCATTCAGCTGGGGGATGGGCTGGAT |
| | | CCTGACTCCGTGTCTCTAGCCTCGGTCACCGCTCTCACCAGCAA |
| | | TGTCTCCAACAAACGGTCTAAGCCAGATATTAAGATGGAGCCCA |
| | | GTGCTGGAAGGCCCATGGATTACCAGGTCAGCATCACAGTGATT |
| | | GAGGCTCGGCAGCTGGTGGGCTTGAACATGGACCCTGTGGTGT |
| | | GTGTGGAGGTGGGTGATGACAAGAAATACACGTCAATGAAGGA |
| | | GTCCACAAACTGCCCTTACTACAACGAGTACTTTGTCTTCGACTT |
| | | CCATGTCTCTCCTGATGTCATGTTTGACAAGATCATCAAGATCTC |
| | | GGTTATCCATTCTAAGAACCTGCTTCGGAGCGGCACCCTGGTGG |
| | | GTTCCTTCAAAATGGATGTGGGGACTGTGTATTCCCAGCCTGAA |
| | | CACCAGTTCCATCACAAATGGGCCATCCTGTCAGACCCCGATGA |
| | | CATCTCTGCTGGGTTGAAGGGTTATGTAAAGTGTGATGTCGCTG |
| | | TGGTGGGCAAGGGAGACAACATCAAGACACCCCACAAGGCCAA |
| | | CGAGACGGATGAGGACGACATTGAAGGGAACTTGCTGCTCCCC |
| | | GAGGGCGTGCCCCCCGAACGGCAGTGGGCACGGTTCTATGTGA |
| | | AAATTTACCGAGCAGAGGGACTGCCCCGGATGAACACAAGCCT |
| | | CATGGCCAACGTGAAGAAGGCGTTCATCGGTGAGAACAAGGAC |
| | | CTCGTCGACCCCTATGTGCAAGTCTTCTTTGCTGGACAAAAGGG |
| | | CAAAACATCAGTGCAGAAGAGCAGCTATGAGCCGCTATGGAATG |
| | | AGCAGGTCGTCTTCACAGACTTGTTCCCCCCACTCTGCAAACGC |
| | | ATGAAGGTGCAGATCCGGGACTCTGACAAGGTCAATGATGTGG |
| | | CCATCGGCACCCACTTCATCGACCTGCGCAAGATTTCCAACGAT |
| | | GGAGACAAAGGCTTCCTGCCTACCCTCGGTCCAGCCTGGGTGA |
| | | ACATGTACGGCTCCACGCGCAACTACACACTGCTGGACGAGCA |
| | | CCAGGACTTGAATGAAGGCCTGGGGGAGGGTGTGTCCTTCCGG |
| | | GCCCGCCTCATGTTGGGACTAGCTGTGGAGATCCTGGACACCT |
| | | CCAACCCAGAGCTCACCAGCTCCACGGAGGTGCAGGTGGAGCA |
| | | GGCCACGCCTGTCTCGGAGAGCTGCACAGGGAGAATGGAAGAA |
| | | TTTTTTCTATTTGGAGCCTTCTTGGAAGCCTCAATGATTGACCGG |
| | | AAAAATGGGGACAAGCCAATTACCTTTGAGGTGACCATAGGAAA |
| | | CTACGGCAATGAAGTCGATGGTATGTCCCGGCCCCTGAGGCCT |
| | | CGGCCCCGGAAAGAGCCTGGGGATGAAGAAGAGGTAGACCTGA |
| | | TTCAGAACTCCAGTGACGATGAAGGTGACGAAGCCGGGGACCT |
| | | GGCCTCGGTGTCCTCCACCCCACCTATGCGGCCCCAGATCACG |
| | | GACAGGAACTATTTCCACCTGCCCTACCTGGAGCGCAAGCCCT |
| | | GCATCTATATCAAGAGCTGGTGGCCTGACCAGAGGCGGCGCCT |
| | | CTACAATGCCAACATCATGGATCACATTGCTGACAAGCTGGAAG |
| | | AAGGCCTGAATGATGTACAGGAGATGATCAAAACGGAGAAGTCC |
| | | TACCCGGAGCGCCGCCTGCGGGGTGTGCTAGAGGAACTCAGCT |
| | | GTGGCTGCCACCGCTTCCTCTCCCTCTCGGACAAGGACCAGGG |
| | | CCGCTCGTCCCGCACCAGGCTGGATCGAGAGCGTCTTAAGTCC |
| | | TGTATGAGGGAGTTGGAGAGCATGGGACAGCAGGCCAAGAGCC |
| | | TGAGGGCTCAGGTGAAGCGGCACACTGTTCGGGACAAGCTGAG |
| | | GTCATGCCAGAACTTTCTGCAGAAGCTACGCTTCCTGGCGGATG |
| | | AGCCCCAGCACAGCATTCCTGATGTGTTCATTTGGATGATGAGC |
| | | AACAACAAACGTATCGCCTATGCCCGCGTGCCTTCCAAAGACCT |
| | | GCTCTTCTCCATCGTGGAGGAGGAACTGGGCAAGGACTGCGCC |
| | | AAAGTCAAGACCCTCTTCCTGAAGCTGCCAGGGAAGAGGGGCT |
| | | TCGGCTCGGCAGGCTGGACAGTACAGGCCAAGCTGGAGCTCTA |
| | | CCTGTGGCTGGGCCTCAGCAAGCAGCGAAAGGACTTCCTGTGT |
| | | GGTCTGCCCTGTGGCTTCGAGGAGGTCAAGGCAGCCCAAGGCC |
| | | TGGGCCTGCATTCCTTTCCGCCCATCAGCCTAGTCTACACCAAG |
| | | AAGCAAGCCTTCCAGCTCCGAGCACACATGTATCAGGCCCGAA |
| | | GCCTCTTTGCTGCTGACAGCAGTGGGCTCTCTGATCCCTTTGCC |
| | | CGTGTCTTCTTCATCAACCAGAGCCAATGCACTGAGGTTCTAAA |
| | | CGAGACACTGTGTCCCACCTGGGACCAGATGCTGGTATTTGACA |
| | | ACCTGGAGCTGTACGGTGAAGCTCACGAGTTACGAGATGATCC |
| | | CCCCATCATTGTCATTGAAATCTACGACCAGGACAGCATGGGCA |
| | | AAGCCGACTTCATGGGCCGGACCTTCGCCAAGCCCCTGGTGAA |
| | | GATGGCAGATGAAGCATACTGCCCACCTCGCTTCCCGCCGCAG |
| | | CTTGAGTACTACCAGATCTACCGAGGCAGTGCCACTGCCGGAG |
| | | ACCTACTGGCTGCCTTCGAGCTGCTGCAGATTGGGCCATCAGG |
| | | GAAGGCTGACCTGCCACCCATCAATGGCCCAGTGGACATGGAC |
| | | AGAGGGCCCATCATGCCTGTGCCCGTGGGAATCCGGCCAGTGC |
| | | TCAGCAAGTACCGAGTGGAGGTGCTGTTCTGGGGCCTGAGGGA |
| | | CCTAAAGAGGGTGAACCTGGCCCAGGTGGACCGACCACGGGTG |
| | | GACATCGAGTGTGCAGGAAAGGGGGTACAATCCTCCCTGATTCA |
| | | CAATTATAAGAAGAACCCCAACTTCAACACGCTGGTCAAGTGGT |
| | | TTGAAGTGGACCTCCCGGAGAATGAGCTCCTGCACCCACCCTT |
| | | GAACATCCGAGTGGTAGATTGCCGGGCCTTTGGACGATACACC |
| | | CTGGTGGGTTCCCACGCAGTCAGCTCACTGAGGCGCTTCATCTA |
| | | CCGACCTCCAGACCGCTCAGCCCCCAACTGGAACACCACAGGG |
| | | GAGGTTGTAGTAAGCATGGAGCCTGAGGAGCCAGTTAAGAAGC |
| | | TGGAGACCATGGTGAAACTGGATGCGACTTCTGATGCTGTGGTC |
| | | AAGGTGGATGTGGCTGAAGATGAGAAGGAAAGGAAGAAGAAGA |
| | | AAAAGAAAGGCCCGTCAGAGGAGCCAGAGGAGGAAGAGCCCG |
| | | ATGAGAGCATGCTGGATTGGTGGTCCAAGTACTTCGCCTCCATC |
| | | GACACAATGAAGGAGCAACTTCGACAACATGAGACCTCTGGAAC |
| | | TGACTTGGAAGAGAAGGAAGAGATGGAAAGCGCTGAGGGCCTG |
| | | AAGGGACCAATGAAGAGCAAGGAGAAGTCCAGAGCTGCAAAGG |
| | | AGGAGAAAAAGAAGAAAAACCAGAGCCCTGGCCCTGGCCAGGG |
| | | ATCGGAGGCTCCTGAGAAGAAGAAAGCCAAGATCGATGAGCTTA |
| | | AGGTGTACCCCAAGGAGCTGGAATCGGAGTTTGACAGCTTTGA |
| | | GGACTGGCTGCACACCTTCAACCTGTTGAGGGGCAAGACGGGA |
| | | GATGATGAGGATGGCTCCACAGAGGAGGAGCGCATAGTAGGCC |
| | | GATTCAAGGGCTCCCTCTGTGTGTACAAAGTGCCACTCCCAGAA |
| | | GATGTATCTCGAGAAGCTGGCTATGATCCCACCTATGGAATGTT |
| | | CCAGGGCATCCCAAGCAATGACCCCATCAATGTGCTGGTCCGA |
| | | ATCTATGTGGTCCGGGCCACAGACCTGCACCCGGCCGACATCA |
| | | ATGGCAAAGCTGACCCCTATATTGCCATCAAGTTAGGCAAGACC |
| | | GACATCCGAGACAAGGAGAACTACATCTCCAAGCAGCTCAACCC |
| | | TGTGTTTGGGAAGTCCTTTGACATTGAGGCCTCCTTCCCCATGG |
| | | AGTCCATGTTGACAGTGGCCGTGTACGACTGGGATCTGGTGGG |
| | | CACTGATGACCTCATCGGAGAAACCAAGATTGACCTGGAAAACC |
| | | GCTTCTACAGCAAGCATCGCGCCACCTGCGGCATCGCACAGAC |
| | | CTATTCCATACATGGCTACAATATCTGGAGGGACCCCATGAAGC |
| | | CCAGCCAGATCCTGACACGCCTCTGTAAAGAGGGCAAAGTGGA |
| | | CGGCCCCCACTTTGGTCCCCATGGGAGAGTGAGGGTTGCCAAC |
| | | CGTGTCTTCACGGGGCCTTCAGAAATAGAGGATGAGAATGGTCA |
| | | GAGGAAGCCCACAGATGAGCACGTGGCACTGTCTGCTCTGAGA |
| | | CACTGGGAGGACATCCCCCGGGTGGGCTGCCGCCTTGTGCCG |
| | | GAACACGTGGAGACCAGGCCGCTGCTCAACCCTGACAAGCCAG |
| | | GCATTGAGCAGGGCCGCCTGGAGCTGTGGGTGGACATGTTCCC |
| | | CATGGACATGCCAGCCCCTGGGACACCTCTGGATATATCCCCCA |
| | | GGAAACCCAAGAAGTACGAGCTGCGGGTCATCGTGTGGAACAC |
| | | AGACGAGGTGGTCCTGGAAGACGATGATTTCTTCACGGGAGAG |
| | | AAGTCCAGTGACATTTTTGTGAGGGGGTGGCTGAAGGGCCAGC |
| | | AGGAGGACAAACAGGACACAGATGTCCACTATCACTCCCTCACG |
| | | GGGGAGGGCAACTTCAACTGGAGATACCTCTTCCCCTTCGACTA |
| | | CCTAGCGGCCGAAGAGAAGATCGTTATGTCCAAAAAGGAGTCTA |
| | | TGTTCTCCTGGGATGAGACGGAGTACAAGATCCCTGCGCGGCT |
| | | CACCCTGCAGATCTGGGACGCTGACCACTTCTCGGCTGACGAC |
| | | TTCCTGGGGGCTATCGAGCTGGACCTGAACCGGTTCCCGAGGG |
| | | GCGCTAAGACAGCCAAGCAGTGCACCATGGAGATGGCCACCGG |
| | | GGAGGTGGACGTACCCCTGGTTTCCATCTTTAAACAGAAACGTG |
| | | TCAAAGGCTGGTGGCCCCTCCTGGCCCGCAATGAGAATGATGA |
| | | GTTTGAGCTCACAGGCAAAGTGGAGGCGGAGCTACACCTACTC |
| | | ACGGCAGAGGAGGCAGAGAAGAACCCTGTGGGCCTGGCTCGC |
| | | AATGAACCTGATCCCCTAGAAAAACCCAACCGGCCTGACACGGC |
| | | ATTCGTCTGGTTCCTGAACCCACTCAAATCTATCAAGTACCTCAT |
| | | CTGCACCCGGTACAAGTGGCTGATCATCAAGATCGTGCTGGCG |
| | | CTGCTGGGGCTGCTCATGCTGGCCCTCTTCCTTTACAGCCTCCC |
| | | AGGCTACATGGTCAAGAAGCTCCTAGGGGCCTGA |
| 6 | Codon-optimized | ATGGCACTGCTGATCCACCTGAAAACCGTCTCCGAACTGAGAGG |
| | (CO) DNA sequence | CAGAGGGGACAGAATCGCTAAAGTCACCTTCCGGGGACAGAGC |
| | encoding the human | TTTTACAGCAGGGTGCTGGAGAACTGCGAGGACGTGGCCGACT |
| | otoferlin isoform 1 | TTGACGAGACATTCAGGTGGCCCGTGGCCAGCTCCATCGATCG |
| | protein (SEQ ID NO: | CAATGAGATGCTGGAGATCCAGGTGTTTAACTATAGCAAGGTGT |
| | 4), 5994 bp | TCTCCAATAAGCTGATCGGCACCTTCCGGATGGTGCTGCAGAAG |
| | | GTGGTGGAGGAGTCCCACGTGGAGGTGACCGACACACTGATCG |
| | | ACGATAACAATGCCATCATCAAGACATCCCTGTGCGTGGAGGTG |
| | | CGCTACCAGGCCACCGATGGCACAGTGGGCTCTTGGGACGATG |
| | | GCGACTTCCTGGGCGATGAGTCCCTGCAGGAGGAGGAGAAGGA |
| | | CTCTCAGGAGACAGATGGCCTGCTGCCTGGCTCCCGGCCATCT |
| | | AGCCGCCCCCCTGGCGAGAAGTCTTTTAGGAGAGCCGGCAGGT |
| | | CCGTGTTCTCTGCCATGAAGCTGGGCAAGAACAGGAGCCACAA |
| | | GGAGGAGCCTCAGAGGCCCGACGAGCCAGCCGTGCTGGAGAT |
| | | GGAGGACCTGGATCACCTGGCCATCAGACTGGGCGATGGCCTG |
| | | GACCCTGATAGCGTGTCCCTGGCCTCCGTGACCGCCCTGACCA |
| | | CAAACGTGTCTAATAAGCGGAGCAAGCCAGACATCAAGATGGAG |
| | | CCATCTGCCGGCAGGCCCATGGATTACCAGGTGAGCATCACAG |
| | | TGATCGAGGCCAGACAGCTGGTGGGCCTGAACATGGACCCCGT |
| | | GGTGTGCGTGGAAGTGGGCGACGATAAGAAGTACACCTCCATG |
| | | AAGGAGTCTACAAACTGTCCATACTACAACGAGTACTTCGTGTTT |
| | | GATTTCCACGTGAGCCCCGACGTGATGTTCGATAAGATCATCAA |
| | | GATCAGCGTGATCCACTCCAAGAATCTGCTGCGGTCTGGCACC |
| | | CTGGTGGGAAGCTTTAAGATGGACGTGGGCACAGTGTACTCTCA |
| | | GCCTGAGCACCAGTTCCACCACAAGTGGGCCATCCTGAGCGAT |
| | | CCAGACGATATCTCCTCTGGCCTGAAGGGCTATGTGAAGTGCGA |
| | | CGTGGCAGTGGTGGGCAAGGGCGATAACATCAAGACCCCACAC |
| | | AAGGCCAATGAGACAGACGAGGACGATATCGAGGGAAACCTGC |
| | | TGCTGCCAGAGGGAGTGCCACCCGAGAGGCAGTGGGCCAGGT |
| | | TCTACGTGAAGATCTATAGGGCAGAGGGCCTGCCTAGGATGAA |
| | | CACCAGCCTGATGGCCAATGTGAAGAAGGCCTTCATCGGCGAG |
| | | AACAAGGACCTGGTGGATCCCTACGTGCAGGTGTTCTTTGCCG |
| | | GCCAGAAGGGCAAGACCTCCGTGCAGAAGAGCTCCTATGAGCC |
| | | TCTGTGGAATGAGCAGGTGGTGTTTACAGACCTGTTCCCTCCAC |
| | | TGTGCAAGAGGATGAAGGTGCAGATCAGAGACTCTGATAAGGT |
| | | GAACGACGTGGCCATCGGCACCCACTTTATCGATCTGAGGAAG |
| | | ATCAGCAATGACGGCGATAAGGGCTTCCTGCCCACCCTGGGCC |
| | | CCGCCTGGGTGAACATGTACGGCAGCACCAGAAATTATACACTG |
| | | CTGGACGAGCACCAGGATCTGAACGAGGGCCTGGGCGAGGGC |
| | | GTGAGCTTTAGAGCCAGGCTGCTGCTGGGCCTGGCCGTGGAGA |
| | | TCGTGGACACCTCCAATCCCGAGCTGACCTCTAGCACAGAGGT |
| | | GCAGGTGGAGCAGGCCACACCTATCTCTGAGAGCTGTGCCGGC |
| | | AAGATGGAGGAGTTCTTTCTGTTTGGCGCCTTCCTGGAGGCCTC |
| | | CATGATCGACCGGCGCAACGGCGATAAGCCTATCACCTTCGAG |
| | | GTGACAATCGGCAACTACGGCAATGAGGTGGACGGCCTGTCTC |
| | | GGCCCCAGCGCCCAAGGCCCAGAAAGGAGCCTGGCGACGAGG |
| | | AGGAGGTGGATCTGATCCAGAACGCCAGCGACGATGAGGCAGG |
| | | CGACGCAGGCGATCTGGCCTCCGTGTCCTCTACCCCCCCTATG |
| | | CGGCCACAGGTGACAGACCGCAATTACTTTCACCTGCCTTATCT |
| | | GGAGCGCAAGCCATGCATCTACATCAAGTCTTGGTGGCCCGAT |
| | | CAGAGGAGACGGCTGTATAACGCCAATATCATGGACCACATCGC |
| | | CGATAAGCTGGAGGAGGGCCTGAATGACATCCAGGAGATGATC |
| | | AAGACCGAGAAGTCCTATCCAGAGCGCAGGCTGAGGGGCGTGC |
| | | TGGAGGAGCTGAGCTGTGGCTGCTGTAGATTCCTGTCCCTGGC |
| | | CGACAAGGATCAGGGGCACTCATCACGGACACGGCTGGACCGG |
| | | GAGCGGCTGAAATCATGTATGCGGGAGCTGGAAAATATGGGAC |
| | | AGCAGGCAAGGATGCTGCGCGCCCAGGTGAAGAGGCACACCG |
| | | TGAGAGACAAGCTGCGGCTGTGCCAGAACTTCCTGCAGAAGCT |
| | | GCGCTTTCTGGCCGATGAGCCACAGCACAGCATCCCCGACATC |
| | | TTCATCTGGATGATGTCCAACAATAAGAGAGTGGCCTACGCCCG |
| | | GGTGCCCTCTAAGGATCTGCTGTTTAGCATCGTGGAGGAGGAG |
| | | ACAGGCAAGGACTGTGCCAAGGTGAAGACCCTGTTCCTGAAGC |
| | | TGCCTGGCAAGAGAGGCTTTGGCAGCGCCGGATGGACCGTGCA |
| | | GGCAAAGGTGGAGCTGTATCTGTGGCTGGGCCTGTCTAAGCAG |
| | | CGGAAGGAGTTCCTGTGCGGCCTGCCCTGTGGCTTTCAGGAGG |
| | | TGAAGGCAGCACAGGGACTGGGACTGCACGCCTTCCCCCCCGT |
| | | GAGCCTGGTGTACACCAAGAAGCAGGCCTTTCAGCTGAGGGCC |
| | | CATATGTACCAGGCCAGGTCTCTGTTCGCCGCCGATAGCTCCG |
| | | GACTGAGCGACCCTTTTGCCAGGGTGTTCTTTATCAATCAGAGC |
| | | CAGTGCACAGAGGTGCTGAACGAGACCCTGTGCCCAACATGGG |
| | | ATCAGATGCTGGTGTTCGACAACCTGGAGCTGTACGGAGAGGC |
| | | ACACGAGCTGAGGGACGATCCACCCATCATCGTGATCGAGATCT |
| | | ATGATCAGGACTCCATGGGCAAGGCCGATTTCATGGGCAGGAC |
| | | CTTTGCCAAGCCCCTGGTGAAGATGGCCGACGAGGCCTACTGC |
| | | CCTCCAAGATTCCCCCCTCAGCTCGAGTACTATCAGATCTATAG |
| | | GGGAAATGCAACCGCCGGAGACCTGCTGGCCGCCTTTGAGCTG |
| | | CTGCAGATCGGCCCCGCCGGAAAGGCAGACCTGCCACCCATCA |
| | | ACGGCCCAGTGGATGTGGACAGAGGCCCCATCATGCCTGTGCC |
| | | AATGGGCATCAGACCAGTGCTGTCCAAGTACAGGGTGGAGGTG |
| | | CTGTTCTGGGGACTGCGCGACCTGAAGAGGGTGAATCTGGCCC |
| | | AGGTGGATAGGCCCAGAGTGGACATCGAGTGCGCCGGAAAGG |
| | | GCGTGCAGTCTAGCCTGATCCACAACTATAAGAAGAACCCAAAT |
| | | TTCAACACCCTGGTGAAGTGGTTTGAGGTGGATCTGCCCGAGAA |
| | | TGAGCTGCTGCACCCTCCACTGAACATCCGGGTGGTGGACTGT |
| | | AGAGCCTTCGGCAGGTACACCCTGGTGGGCAGCCACGCCGTGA |
| | | GCAGCCTGAGGAGGTTCATCTACAGGCCCCCTGACAGGTCCGC |
| | | CCCTTCTTGGAATACCACAGTGAGACTGCTGCGGCGCTGCAGG |
| | | GTGCTGTGCAACGGAGGCAGCTCCTCTCACTCTACCGGCGAGG |
| | | TGGTGGTGACAATGGAGCCTGAGGTACCCATCAAGAAGCTGGA |
| | | GACCATGGTGAAGCTGGATGCCACAAGCGAGGCAGTGGTGAAG |
| | | GTGGACGTGGCAGAGGAGGAGAAGGAGAAGAAGAAGAAGAAG |
| | | AAGGGAACCGCCGAGGAGCCTGAGGAAGAGGAGCCAGATGAG |
| | | AGCATGCTGGACTGGTGGTCCAAGTACTTCGCCTCTATCGACAC |
| | | AATGAAGGAGCAGCTGAGACAGCAGGAGCCTAGCGGCATCGAT |
| | | CTGGAGGAGAAGGAGGAGGTGGACAATACCGAGGGCCTGAAG |
| | | GGCTCCATGAAGGGCAAGGAGAAGGCAAGGGCAGCAAAGGAA |
| | | GAGAAGAAGAAGAAGACCCAGAGCAGCGGCTCTGGACAGGGCA |
| | | GCGAGGCACCAGAGAAGAAGAAGCCTAAGATCGATGAGCTGAA |
| | | GGTGTACCCAAAGGAGCTGGAGTCCGAGTTCGATAATTTTGAGG |
| | | ACTGGCTGCACACCTTCAACCTGCTGCGCGGCAAGACAGGCGA |
| | | CGATGAGGACGGCAGCACCGAGGAGGAGAGAATCGTGGGCCG |
| | | GTTTAAGGGCTCCCTGTGCGTGTACAAGGTGCCACTGCCTGAG |
| | | GACGTGAGCAGGGAGGCCGGATACGACTCTACCTATGGCATGT |
| | | TCCAGGGCATCCCCTCTAATGATCCTATCAACGTGCTGGTGCGC |
| | | GTGTATGTGGTGAGGGCCACAGATCTGCACCCCGCCGACATCA |
| | | ACGGCAAGGCCGACCCTTACATCGCCATCCGCCTGGGCAAGAC |
| | | CGATATCAGGGACAAGGAGAATTATATCTCCAAGCAGCTGAACC |
| | | CCGTGTTCGGCAAGTCTTTTGACATCGAGGCCAGCTTCCCTATG |
| | | GAGTCCATGCTGACCGTGGCCGTGTACGATTGGGACCTGGTGG |
| | | GCACCGACGATCTGATCGGCGAGACAAAGATCGATCTGGAGAA |
| | | TCGCTTTTATTCTAAGCACAGGGCAACCTGCGGAATCGCACAGA |
| | | CCTACAGCACACACGGCTATAACATCTGGCGCGACCCCATGAA |
| | | GCCTAGCCAGATCCTGACAAGGCTGTGCAAGGATGGCAAGGTG |
| | | GACGGACCACACTTCGGACCACCCGGCAGAGTGAAGGTGGCCA |
| | | ATCGGGTGTTTACAGGCCCTTCCGAGATCGAGGATGAGAACGG |
| | | CCAGCGCAAGCCAACCGACGAGCACGTGGCCCTGCTGGCCCT |
| | | GAGGCACTGGGAGGATATCCCAAGGGCCGGATGTAGGCTGGTG |
| | | CCTGAGCACGTGGAGACCAGACCACTGCTGAATCCAGACAAGC |
| | | CAGGAATCGAGCAGGGCAGGCTGGAGCTGTGGGTGGATATGTT |
| | | CCCAATGGACATGCCAGCCCCAGGAACACCCCTGGATATCTCC |
| | | CCTAGAAAGCCAAAGAAGTACGAGCTGAGAGTGATCATCTGGAA |
| | | CACAGACGAGGTGGTGCTGGAGGACGATGACTTCTTTACCGGC |
| | | GAGAAGTCTAGCGATATCTTTGTGCGCGGATGGCTGAAGGGAC |
| | | AGCAGGAGGACAAGCAGGATACAGACGTGCACTACCACTCCCT |
| | | GACCGGCGAGGGCAATTTCAACTGGAGATACCTGTTCCCTTTTG |
| | | ATTATCTGGCCGCCGAGGAGAAGATCGTGATCTCTAAGAAGGAG |
| | | AGCATGTTTTCCTGGGACGAGACAGAGTATAAGATCCCAGCCAG |
| | | ACTGACCCTGCAGATCTGGGATGCCGACCACTTCAGCGCCGAT |
| | | GACTTTCTGGGCGCCATCGAGCTGGACCTGAACCGGTTCCCAA |
| | | GAGGCGCCAAGACCGCCAAGCAGTGCACAATGGAGATGGCAAC |
| | | CGGAGAGGTGGACGTGCCTCTGGTGTCTATCTTCAAGCAGAAG |
| | | AGGGTGAAGGGCTGGTGGCCACTGCTGGCCAGAAACGAGAATG |
| | | ATGAGTTTGAGCTGACAGGCAAGGTGGAGGCAGAGCTGCACCT |
| | | GCTGACCGCCGAGGAGGCAGAGAAGAACCCAGTGGGCCTGGC |
| | | CAGGAATGAGCCCGACCCTCTGGAGAAGCCAAACAGGCCCGAC |
| | | ACCAGCTTCATCTGGTTTCTGAATCCTCTGAAGTCCGCCCGGTA |
| | | CTTCCTGTGGCACACCTATCGCTGGCTGCTGCTGAAGCTGTTAT |
| | | TACTGTTATTACTGCTGCTGCTGCTGGCCCTGTTTCTGTACAGC |
| | | GTGCCCGGCTATCTGGTGAAGAAGATCCTGGGCGCCTGA |

### Expression of OTOF in Mammalian Cells

Mutations in OTOF have been linked to sensorineural hearing loss and auditory neuropathy. The compositions and methods described herein increase the expression of WT OTOF isoform 5 protein by administering a first nucleic acid vector that contains a polynucleotide encoding an N-terminal portion of an OTOF isoform 5 protein and a second nucleic acid vector that contains a polynucleotide encoding a C-terminal portion of an OTOF isoform 5 protein. In order to utilize nucleic acid vectors for therapeutic application in the treatment of sensorineural hearing loss and auditory neuropathy, they can be directed to the interior of the cell, and, in particular, to specific cell types. A wide array of methods has been established for the delivery of proteins to mammalian cells and for the stable expression of genes encoding proteins in mammalian cells.

### Polynucleotides Encoding OTOF

One platform that can be used to achieve therapeutically effective intracellular concentrations of OTOF isoform 5 in mammalian cells is via the stable expression of the gene encoding OTOF isoform 5 (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

OTOF isoform 5 can also be introduced into a mammalian cell by targeting vectors containing portions of a gene encoding an OTOF isoform 5 protein to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding an OTOF isoform 5 protein by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode an OTOF protein downstream of a mammalian promoter (e.g., a polynucleotide that encodes an N-terminal portion of an OTOF isoform 5 protein downstream of a mammalian promoter). Promoters that are useful for the expression of an OTOF protein in mammalian cells include ubiquitous promoters and cochlear hair cell-specific promoters. Ubiquitous promoters include the CAG promoter, the cytomegalovirus (CMV) promoter, and a truncated form of the chimeric CMV-chicken β-actin promoter (CBA), in which the hybrid chicken β-actin/rabbit β-globin intron is greatly shortened to produce a smaller version of the promoter called smCBA. Cochlear hair cell-specific promoters include the Myosin 15 (Myo15) promoter. Myo15 promoter sequences for use in the methods and compositions described herein are described below and in Table 3. Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of Moloney virus, Epstein barr virus (EBV) promoter, and the Rous sarcoma virus (RSV) promoter.

### Murine Myosin 15 Promoters

In some embodiments, the Myo15 promoter for use in the compositions and methods described herein includes polynucleotide sequences from regions of the murine Myo15 locus that are capable of expressing a transgene specifically in hair cells, or variants thereof, such as a polynucleotide sequences that have at least 85% sequence identity (e.g., 85%, 90%, 95%, 96%, 97%, 98%, 99%, or more, sequence identity) to regions of the murine Myo15 locus that are capable of expressing a transgene specifically in hair cells. These regions include polynucleotide sequences immediately preceding the murine Myo 15 translation start site and an upstream regulatory element that is located over 5 kb from the murine Myo 15 translation start site. The murine Myo15 promoter for use in the compositions and methods described herein can optionally include a linker operably linking the regions of the murine Myo15 locus that are capable of expressing a transgene specifically in hair cells, or the regions of the murine Myo15 locus can be joined directly without an intervening linker.

In some embodiments, the murine Myo15 promoter for use in the compositions and methods described herein contains a first region (an upstream regulatory element) having at least 85% sequence identity (e.g., 85%, 90%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 7) or a functional portion or derivative thereof joined (e.g., operably linked) to a second region having at least 85% sequence identity (e.g., 85%, 90%, 95%, 96%, 97%, 98%, 99%, or more, sequence identity) to the polynucleotide sequence immediately preceding the murine Myo15 translation start site (nucleic acids from −1 to −1157 with respect to the murine Myo15 translation start site, the sequence of which is set forth in SEQ ID NO: 8) or a functional portion or derivative thereof. The functional portion of SEQ ID NO: 7 may have the sequence of nucleic acids from −7166 to −7091 with respect to the murine Myo15 translation start site (set forth in SEQ ID NO: 9) and/or the sequence of nucleic acids from −7077 to −6983 with respect to the murine Myo15 translation start site (set forth in SEQ ID NO: 10). The first region may contain the polynucleotide sequence of SEQ ID NO: 9 fused to the polynucleotide sequence of SEQ ID NO: 10 with no intervening nucleic acids, as set forth in SEQ ID NO: 11, or the first region may contain the polynucleotide sequence of SEQ ID NO: 10 fused to the polynucleotide sequence of SEQ ID NO: 9 with no intervening nucleic acids, as set forth in SEQ ID NO: 12. Alternatively, the first region may contain the sequences of SEQ ID NO: 9 and SEQ ID NO: 10 joined by the endogenous intervening polynucleotide sequence (e.g., the first region may have or include the sequence of nucleic acids from −7166 to −6983 with respect to the murine Myo15 translation start site, as set forth in SEQ ID NO: 13 and SEQ ID NO: 33) or a nucleic acid linker. In a murine Myo15 promoter in which the first region contains both SEQ ID NO: 9 and SEQ ID NO: 10, the two sequences can be included in any order (e.g., SEQ ID NO: 9 may be joined to (e.g., precede) SEQ ID NO: 10, or SEQ ID NO: 10 may be joined to (e.g., precede) SEQ ID NO: 9). The functional portion of SEQ ID NO: 8 may have the sequence of nucleic acids from −590 to −509 with respect to the murine Myo15 translation start site (set forth in SEQ ID NO: 14) and/or the sequence of nucleic acids from −266 to −161 with respect to the murine Myo15 translation start site (set forth in SEQ ID NO: 15). In some embodiments, the sequence containing SEQ ID NO: 14 has the sequence of SEQ ID NO: 34. In some embodiments, the sequence containing SEQ ID NO: 15 has the sequence of SEQ ID NO: 35. The second region may contain the polynucleotide sequence of SEQ ID NO: 14 fused to the polynucleotide sequence of SEQ ID NO: 15 with no intervening nucleic acids, as set forth in SEQ ID NO: 16, or the second region may contain the polynucleotide sequence of SEQ ID NO: 15 fused to the polynucleotide sequence of SEQ ID NO: 14 with no intervening nucleic acids, as set forth in SEQ ID NO: 17. The second region may contain the nucleic acid sequence of SEQ ID NO: 34 fused to the nucleic acid sequence of SEQ ID NO: 35 with no intervening nucleic acids, as set forth in SEQ ID NO: 38, or the second region may contain the nucleic acid sequence of SEQ ID NO: 35 fused to the nucleic acid sequence of SEQ ID NO: 34 with no intervening nucleic acids. Alternatively, the second region may contain the sequences of SEQ ID NO: 14 and SEQ ID NO: 15 joined by the endogenous intervening polynucleotide sequence (e.g., the second region may have the sequence of nucleic acids from −590 to −161 with respect to the murine Myo15 translation start site, as set forth in SEQ ID NO: 18) or a nucleic acid linker. In a murine Myo15 promoter in which the second region contains both SEQ ID NO: 14 and SEQ ID NO: 15, the two sequences can be included in any order (e.g., SEQ ID NO: 14 may be joined to (e.g., precede) SEQ ID NO: 15, or SEQ ID NO: 15 may be joined to (e.g., precede) SEQ ID NO: 14).

The first region and the second region of the murine Myo15 promoter can be joined directly or can be joined by a nucleic acid linker. For example, the murine Myo15 promoter can contain the sequence of SEQ ID NO: 7 or a functional portion or derivative thereof (e.g., any one or more of SEQ ID NOs: 9-13 and 33, e.g., SEQ ID NOs 9 and 10) fused to the sequence of SEQ ID NO: 8 or a functional portion or derivative thereof (e.g., any one or more of SEQ ID NOs: 14-18, 34, 35, and 38, e.g., SEQ ID NOs 14 and 15) with no intervening nucleic acids. For example, the polynucleotide sequence of the murine Myo15 promoter that results from direct fusion of SEQ ID NO: 7 to SEQ ID NO: 8 is set forth in SEQ ID NO: 19. Alternatively, a linker can be used to join the sequence of SEQ ID NO: 7 or a functional portion or derivative thereof (e.g., any one or more of SEQ ID NOs: 9-13 and 33, e.g., SEQ ID NOs 9 and 10) to the sequence of SEQ ID NO: 8 or a functional portion or derivative thereof (e.g., any one or more of SEQ ID NOs: 14-18, 34, 35, and 38, e.g., SEQ ID NOs 14 and 15). Exemplary Myo15 promoters containing functional portions of both SEQ ID NO: 7 and SEQ ID NO: 8 are provided in SEQ ID NOs: 21, 22, 36, 37, 42, and 43.

The length of a nucleic acid linker for use in a murine Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the murine Myo15 promoter described herein do not disrupt the ability of the murine Myo15 promoter of the invention to induce transgene expression in hair cells.

In some embodiments, the sequence of SEQ ID NO: 7 or a functional portion or derivative thereof (e.g., any one or more of SEQ ID NOs: 9-13 and 33, e.g., SEQ ID NOs 9 and 10) is joined (e.g., operably linked) to the sequence of SEQ ID NO: 8 or a functional portion or derivative thereof (e.g., any one or more of SEQ ID NOs: 14-18, 34, 35, and 38, e.g., SEQ ID NOs 14 and 15), and, in some embodiments, the order of the regions is reversed (e.g., the sequence of SEQ ID NO: 8 or a functional portion or derivative thereof (e.g., any one or more of SEQ ID NOs: 14-18, 34, 35, and 38, e.g., SEQ ID NOs 14 and 15) is joined (e.g., operably linked) to the sequence of SEQ ID NO: 7 or a functional portion or derivative thereof (e.g., any one or more of SEQ ID NOs: 9-13 and 33, e.g., SEQ ID NOs 9 and 10)). For example, the polynucleotide sequence of a murine Myo15 promoter that results from direct fusion of SEQ ID NO: 8 to SEQ ID NO: 7 is set forth in SEQ ID NO: 20. An example of a murine Myo15 promoter in which a functional portion or derivative of SEQ ID NO: 8 precedes a functional portion or derivative of SEQ ID NO: 7 is provided in SEQ ID NO: 41. Regardless of order, the sequence of SEQ ID NO: 7 or a functional portion or derivative thereof and the sequence of SEQ ID NO: 8 or a functional portion or derivative thereof can be joined by direct fusion or a nucleic acid linker, as described above.

In some embodiments, the murine Myo15 promoter for use in the compositions and methods described herein contains a region having at least 85% sequence identity (e.g., 85%, 90%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 7) or a functional portion or derivative thereof. The functional portion of SEQ ID NO: 7 may have the sequence of nucleic acids from −7166 to −7091 with respect to the murine Myo15 translation start site (set forth in SEQ ID NO: 9) and/or the sequence of nucleic acids from −7077 to −6983 with respect to the murine Myo15 translation start site (set forth in SEQ ID NO: 10). The murine Myo15 promoter may contain the polynucleotide sequence of SEQ ID NO: 9 fused to the polynucleotide sequence of SEQ ID NO: 10 with no intervening nucleic acids, as set forth in SEQ ID NO: 11, or the murine Myo15 promoter may contain the polynucleotide sequence of SEQ ID NO: 10 fused to the polynucleotide sequence of SEQ ID NO: 9 with no intervening nucleic acids, as set forth in SEQ ID NO: 12. Alternatively, the murine Myo15 promoter may contain the sequences of SEQ ID NO: 9 and SEQ ID NO: 10 joined by the endogenous intervening polynucleotide sequence (e.g., the first region may have or include the sequence of nucleic acids from −7166 to −6983 with respect to the murine Myo15 translation start site, as set forth in SEQ ID NO: 13 and SEQ ID NO: 33) or a polynucleotide linker. In a murine Myo15 promoter that contains both SEQ ID NO: 9 and SEQ ID NO: 10, the two sequences can be included in any order (e.g., SEQ ID NO: 9 may be joined to (e.g., precede) SEQ ID NO: 10, or SEQ ID NO: 10 may be joined to (e.g., precede) SEQ ID NO: 9).

In some embodiments, the murine Myo15 promoter for use in the compositions and methods described herein contains a region having at least 85% sequence identity (e.g., 85%, 90%, 95%, 96%, 97%, 98%, 99%, or more, sequence identity) to the polynucleotide sequence immediately upstream of the murine Myo15 translation start site (nucleic acids from −1 to −1157 with respect to the murine Myo15 translation start site, the sequence of which is set forth in SEQ ID NO: 8) or a functional portion or derivative thereof. The functional portion of SEQ ID NO: 8 may have the sequence of nucleic acids from −590 to −509 with respect to the murine Myo15 translation start site (set forth in SEQ ID NO: 14) and/or the sequence of nucleic acids from −266 to −161 with respect to the murine Myo15 translation start site (set forth in SEQ ID NO: 15). In some embodiments, the sequence containing SEQ ID NO: 14 has the sequence of SEQ ID NO: 34. In some embodiments, the sequence containing SEQ ID NO: 15 has the sequence of SEQ ID NO: 35. The murine Myo15 promoter may contain the polynucleotide sequence of SEQ ID NO: 14 fused to the polynucleotide sequence of SEQ ID NO: 15 with no intervening nucleic acids, as set forth in SEQ ID NO: 16, or the murine Myo15 promoter may contain the polynucleotide sequence of SEQ ID NO: 15 fused to the polynucleotide sequence of SEQ ID NO: 14 with no intervening nucleic acids, as set forth in SEQ ID NO: 17. The murine Myo15 promoter may contain the nucleic acid sequence of SEQ ID NO: 34 fused to the nucleic acid sequence of SEQ ID NO: 35 with no intervening nucleic acids, as set forth in SEQ ID NO: 38, or the murine Myo15 promoter may contain the nucleic acid sequence of SEQ ID NO: 35 fused to the nucleic acid sequence of SEQ ID NO: 41 with no intervening nucleic acids. Alternatively, the murine Myo15 promoter may contain the sequences of SEQ ID NO: 14 and SEQ ID NO: 15 joined by the endogenous intervening polynucleotide sequence (e.g., the second region may have the sequence of nucleic acids from −590 to −161 with respect to the murine Myo 15 translation start site, as set forth in SEQ ID NO: 18) or a nucleic acid linker. In a murine Myo 15 promoter that contains both SEQ ID NO: 14 and SEQ ID NO: 15, the two sequences can be included in any order (e.g., SEQ ID NO: 14 may be joined to (e.g., precede) SEQ ID NO: 15, or SEQ ID NO: 15 may be joined to (e.g., precede) SEQ ID NO: 14).

In some embodiments, the murine Myo15 promoter for use in the compositions and methods described herein contains a functional portion or derivative of a region having at least 85% sequence identity (e.g., 85%, 90%, 95%, 96%, 97%, 98%, 99%, or more, sequence identity) to a region containing the first non-coding exon of the Myo15 gene (nucleic acids from −6755 to −7209 with respect to the murine Myo15 translation start site, the sequence of which is set forth in SEQ ID NO: 7) flanked on either side by a functional portion or derivative of a region having at least 85% sequence identity (e.g., 85%, 90%, 95%, 96%, 97%, 98%, 99%, or more, sequence identity) to the nucleic acid sequence immediately upstream of the murine Myo 15 translation start site (nucleic acids from −1 to −1157 with respect to the murine Myo 15 translation start site, the sequence of which is set forth in SEQ ID NO: 8). For example, a functional portion or derivative of SEQ ID NO: 8, such as SEQ ID NO: 14 or 34 may be directly fused or joined by a nucleic acid linker to a portion of SEQ ID NO: 7, such as any one of SEQ ID NOs: 9-13 and 33, which is directly fused or joined by a nucleic acid linker to a different functional portion of SEQ ID NO: 8, such as SEQ ID NO: 15 or 35. In other embodiments, a functional portion or derivative of SEQ ID NO: 8, such as SEQ ID NO: 15 or 35 may be directly fused or joined by a nucleic acid linker to a portion of SEQ ID NO: 7, such as any one of SEQ ID NOs: 9-13 and 33, which is directly fused or joined by a nucleic acid linker to a different functional portion of SEQ ID NO: 8, such as SEQ ID NO: 14 or 34. For example, polynucleotides having at least 85% sequence identity (e.g., 85%, 90%, 95%, 96%, 97%, 98%, 99%, or more, sequence identity) to the nucleic acid sequence of SEQ ID NOs: 34, 33, and 35 can be fused to produce a polynucleotide having at least 85% sequence identity (e.g., 85%, 90%, 95%, 96%, 97%, 98%, 99%, or more, sequence identity) to the nucleic acid sequence of SEQ ID NO: 39. In some embodiments, polynucleotides having at least 85% sequence identity (e.g., 85%, 90%, 95%, 96%, 97%, 98%, 99%, or more, sequence identity) to the nucleic acid sequence of SEQ ID NOs: 35, 33, and 34 can be fused to produce a polynucleotide having at least 85% sequence identity (e.g., 85%, 90%, 95%, 96%, 97%, 98%, 99%, or more, sequence identity) to the nucleic acid sequence of SEQ ID NO: 40.

### Human Myosin 15 Promoters

The polynucleotides of the compositions and methods described herein may also include nucleic acid sequences from regions of the human Myo15 locus that are capable of expressing a transgene specifically in hair cells, or variants thereof, such as a nucleic acid sequences that have at least 85% sequence identity (e.g., 85%, 90%, 95%, 96%, 97%, 98%, 99%, or more, sequence identity) to regions of the human Myo15 locus that are capable of expressing a transgene specifically in hair cells. The polynucleotides of the compositions and methods described herein can optionally include a linker operably linking the regions of the human Myo15 locus that are capable of expressing a transgene specifically in hair cells, or the regions of the human Myo15 locus can be joined directly without an intervening linker.

In some embodiments, the human Myo 15 promoter for use in the compositions and methods described herein contains a first region having at least 85% sequence identity (e.g., 85%, 90%, 95%, 96%, 97%, 98%, 99%, or more, sequence identity) to the sequence set forth in SEQ ID NO: 23 or a functional portion or derivative thereof joined (e.g., operably linked) to a second region having at least 85% sequence identity (e.g., 85%, 90%, 95%, 96%, 97%, 98%, 99%, or more, sequence identity) to the sequence set forth in SEQ ID NO: 24 or a functional portion or derivative thereof. The functional portion of SEQ ID NO: 23 may have the sequence set forth in SEQ ID NO: 25. The functional portion of SEQ ID NO: 24 may have the sequence set forth in SEQ ID NO: 26 and/or the sequence set forth in SEQ ID NO: 27. The second region may contain the nucleic acid sequence of SEQ ID NO: 26 fused to the nucleic acid sequence of SEQ ID NO: 27 with no intervening nucleic acids, as set forth in SEQ ID NO: 28, or the second region may contain the nucleic acid sequence of SEQ ID NO: 27 fused to the nucleic acid sequence of SEQ ID NO: 26 with no intervening nucleic acids, as set forth in SEQ ID NO: 29. Alternatively, the second region may contain the sequences of SEQ ID NO: 26 and SEQ ID NO: 27 joined by the endogenous intervening nucleic acid sequence (as set forth in SEQ ID NO: 30) or a nucleic acid linker. In a human Myo15 promoter in which the second region contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26).

The first region and the second region of the human Myo15 promoter can be joined directly or can be joined by a nucleic acid linker. For example, the human Myo 15 promoter can contain the sequence of SEQ ID NO: 23 or a functional portion or derivative thereof (e.g., SEQ ID NO: 25) fused to the sequence of SEQ ID NO: 24 or a functional portion or derivative thereof (e.g., any one or more of SEQ ID NOs: 26-30, e.g., SEQ ID NOs: 26 and/or 27) with no intervening nucleic acids. Alternatively, a linker can be used to join the sequence of SEQ ID NO: 23 or a functional portion or derivative thereof (e.g., SEQ ID NO: 25) to the sequence of SEQ ID NO: 24 or a functional portion or derivative thereof (e.g., any one or more of SEQ ID NOs: 26-30, e.g., SEQ ID NOs: 26 and/or 27). Exemplary human Myo15 promoters containing functional portions of both SEQ ID NO: 23 and SEQ ID NO: 24 are provided in SEQ ID NOs: 31 and 32.

In some embodiments, the sequence of SEQ ID NO: 23 or a functional portion or derivative thereof (e.g., SEQ ID NO: 25) is joined (e.g., operably linked) to the sequence of SEQ ID NO: 24 or a functional portion or derivative thereof (e.g., any one or more of SEQ ID NOs: 26-30, e.g., SEQ ID NOS: 26 and 27), and, in some embodiments, the order of the regions is reversed (e.g., the sequence of SEQ ID NO: 24 or a functional portion or derivative thereof (e.g., any one or more of SEQ ID NOs: 26-30, e.g., SEQ ID NOs: 26 and/or 27) is joined (e.g., operably linked) to the sequence of SEQ ID NO: 23 or a functional portion or derivative thereof (e.g., SEQ ID NO: 25)). Regardless of order, the sequence of SEQ ID NO: 23 or a functional portion or derivative thereof and the sequence of SEQ ID NO: 24 or a functional portion or derivative thereof can be joined by direct fusion or a nucleic acid linker, as described above.

In some embodiments, the human Myo15 promoter for use in the compositions and methods described herein contain a region having at least 85% sequence identity (e.g., 85%, 90%, 95%, 96%, 97%, 98%, 99%, or more, sequence identity) to a region containing the sequence set forth in SEQ ID NO: 23 or a functional portion or derivative thereof. The functional portion of SEQ ID NO: 23 may have the sequence of nucleic acids set forth in SEQ ID NO: 25.

In some embodiments, the human Myo 15 promoter for use in the compositions and methods described herein contains a region having at least 85% sequence identity (e.g., 85%, 90%, 95%, 96%, 97%, 98%, 99%, or more, sequence identity) to the sequence set forth in SEQ ID NO: 18 or a functional portion or derivative thereof. The functional portion of SEQ ID NO: 24 may have the sequence set forth in SEQ ID NO: 26 and/or the sequence set forth in SEQ ID NO: 27. The human Myo 15 promoter may contain the nucleic acid sequence of SEQ ID NO: 26 fused to the nucleic acid sequence of SEQ ID NO: 27 with no intervening nucleic acids, as set forth in SEQ ID NO: 28, or the human Myo15 promoter may contain the nucleic acid sequence of SEQ ID NO: 27 fused to the nucleic acid sequence of SEQ ID NO: 26 with no intervening nucleic acids, as set forth in SEQ ID NO: 29. Alternatively, the human Myo15 promoter may contain the sequences of SEQ ID NO: 26 and SEQ ID NO: 27 joined by the endogenous intervening nucleic acid sequence (e.g., as set forth in SEQ ID NO: 30) or a nucleic acid linker. In a human Myo 15 promoter that contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26).

The length of a nucleic acid linker for use in a human Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the human Myo15 promoters described herein do not disrupt the ability of the Myo15 promoter of the invention to induce transgene expression in hair cells.

The foregoing Myo15 promoter sequences are summarized in Table 3, below.

TABLE 3
Exemplary nucleotide sequences for use
in the Myo15 promoter described herein

| | Description of | |
|---|---|---|
| SEQ | polynucleotide | |
| ID NO. | sequence | Polynucleotide Sequence |
| 7 | Region containing non- | CTGCAGCTCAGCCTACTACTTGCTTTCCAGGCTGTTCCTAGT |
| | coding exon 1 of | TCCCATGTCAGCTGCTTGTGCTTTCCAGAGACAAAACAGGAA |
| | murine Myo15 (−6755 to | TAATAGATGTCATTAAATATACATTGGGCCCCAGGCGGTCAAT |
| | −7209) | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTAAGCTTCTGC |
| | | CACTGGCTCCGGCATTGCAGAGAGAAGAGAAGGGGCGGCA |
| | | GAGCTGAACCTTAGCCTTGCCTTCCTGGGTACCCTTCTGAGC |
| | | CTCACTGTCTTCTGTGAGATGGGCAAAGTGCGGGTGTGACTC |
| | | CTTGGCAACGGTGTTACACCAGGGCAGGTAAAGTTGTAGTTA |
| | | TTTGTGGGGTACACCAGGACTGTTAAAGGTGTAACTAT |
| 8 | Region immediately | GGTCTCACCCAGCATTTTCACTTCTAATAAGTTCAAATGTGAT |
| | preceding the | ACGGCACCTTTCTAAAAATTAGTTTTCAGGGAAATAGGGTTCA |
| | translation start site of | AAACTGGTAGTGGTAGGGTCCATTCTCACGACCCCCAGGCCT |
| | murine Myo15 (−1 to | GCTAACCCTGACCAAGCTACCTATTACTTACCCTCCTCTTTCT |
| | −1157) | CCTCCTCCTCTTTCTCCTTCTCCTGCTTCCCCTCTTCCTTCTC |
| | | CCTCCCTTCCTCTCCCTCCTCCCCCTCCTTGGCTGTGATCAG |
| | | ATCCAGAGCCTGAATGAGCCTCCTGACCCCACACCCCCACTA |
| | | GCATGGGCCTGCAAGTGCCCAGAAGTCCCTCCTGCCTCCTA |
| | | AACTGCCCAGCCGATCCATTAGCTCTTCCTTCTTCCCAGTGA |
| | | AAGAAGCAGGCACAGCCTGTCCCTCCCGTTCTACAGAAAGG |
| | | AAGCTACAGCACAGGGAGGGCCAAAGGCCTTCCTGGGACTA |
| | | GACAGTTGATCAACAGCAGGACTGGAGAGCTGGGCTCCATTT |
| | | TTGTTCCTTGGTGCCCTGCCCCTCCCCATGACCTGCAGAGAC |
| | | ATTCAGCCTGCCAGGCTTTATGAGGTGGGAGCTGGGCTCTC |
| | | CCTGATGTATTATTCAGCTCCCTGGAGTTGGCCAGCTCCTGT |
| | | TACACTGGCCACAGCCCTGGGCATCCGCTTCTCACTTCTAGT |
| | | TTCCCCTCCAAGGTAATGTGGTGGGTCATGATCATTCTATCCT |
| | | GGCTTCAGGGACCTGACTCCACTTTGGGGCCATTCGAGGGG |
| | | TCTAGGGTAGATGATGTCCCCCTGTGGGGATTAATGTCCTGC |
| | | TCTGTAAAACTGAGCTAGCTGAGATCCAGGAGGGCTTGGCCA |
| | | GAGACAGCAAGTTGTTGCCATGGTGACTTTAAAGCCAGGTTG |
| | | CTGCCCCAGCACAGGCCTCCCAGTCTACCCTCACTAGAAAAC |
| | | AACACCCAGGCACTTTCCACCACCTCTCAAAGGTGAAACCCA |
| | | AGGCTGGTCTAGAGAATGAATTATGGATCCTCGCTGTCCGTG |
| | | CCACCCAGCTAGTCCCAGCGGCTCAGACACTGAGGAGAGAC |
| | | TGTAGGTTCAGCTACAAGCAAAAAGACCTAGCTGGTCTCCAA |
| | | GCAGTGTCTCCAAGTCCCTGAACCTGTGACACCTGCCCCAG |
| | | GCATCATCAGGCACAGAGGGCCACC |
| 9 | Portion of SEQ ID NO: | CCCATGTCAGCTGCTTGTGCTTTCCAGAGACAAAACAGGAAT |
| | 7 (−7166 to −7091) | AATAGATGTCATTAAATATACATTGGGCCCCAGG |
| 10 | Portion of SEQ ID NO: | AGCCTGAGCCTCCTTTCCATCTCTGTGGAGGCAGACATAGGA |
| | 7 (−7077 to −6983) | CCCCCAACAAACAGCATGCAGGTTGGGAGCCAGCCACAGGA |
| | | CCCAGGTAAGGG |
| 11 | Portion of SEQ ID NO: | CCCATGTCAGCTGCTTGTGCTTTCCAGAGACAAAACAGGAAT |
| | 7 (SEQ ID NO: 9 fused | AATAGATGTCATTAAATATACATTGGGCCCCAGGAGCCTGAG |
| | to SEQ ID NO: 10) | CCTCCTTTCCATCTCTGTGGAGGCAGACATAGGACCCCCAAC |
| | | AAACAGCATGCAGGTTGGGAGCCAGCCACAGGACCCAGGTA |
| | | AGGG |
| 12 | Portion of SEQ ID NO: | AGCCTGAGCCTCCTTTCCATCTCTGTGGAGGCAGACATAGGA |
| | 7 (SEQ ID NO: 10 | CCCCCAACAAACAGCATGCAGGTTGGGAGCCAGCCACAGGA |
| | fused to SEQ ID NO: 9) | CCCAGGTAAGGGCCCATGTCAGCTGCTTGTGCTTTCCAGAGA |
| | | CAAAACAGGAATAATAGATGTCATTAAATATACATTGGGCCCC |
| | | AGG |
| 13 | Portion of SEQ ID NO: | CCCATGTCAGCTGCTTGTGCTTTCCAGAGACAAAACAGGAAT |
| | 7 | AATAGATGTCATTAAATATACATTGGGCCCCAGGCGGTCAAT |
| | (−7166 to −6983) | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGG |
| 14 | Portion of SEQ ID NO: | TGAGGTGGGAGCTGGGCTCTCCCTGATGTATTATTCAGCTCC |
| | 8 (−590 to −509) | CTGGAGTTGGCCAGCTCCTGTTACACTGGCCACAGCCCTG |
| 15 | Portion of SEQ ID NO: | CACAGGCCTCCCAGTCTACCCTCACTAGAAAACAACACCCAG |
| | 8 (−266 to −161) | GCACTTTCCACCACCTCTCAAAGGTGAAACCCAAGGCTGGTC |
| | | TAGAGAATGAATTATGGATCCT |
| 16 | Portion of SEQ ID NO: | TGAGGTGGGAGCTGGGCTCTCCCTGATGTATTATTCAGCTCC |
| | 8 | CTGGAGTTGGCCAGCTCCTGTTACACTGGCCACAGCCCTG |
| | (SEQ ID NO: 14 fused | CACAGGCCTCCCAGTCTACCCTCACTAGAAAACAACACCCAG |
| | to SEQ ID NO: 15) | GCACTTTCCACCACCTCTCAAAGGTGAAACCCAAGGCTGGTC |
| | | TAGAGAATGAATTATGGATCCT |
| 17 | Portion of SEQ ID NO: | CACAGGCCTCCCAGTCTACCCTCACTAGAAAACAACACCCAG |
| | 8 | GCACTTTCCACCACCTCTCAAAGGTGAAACCCAAGGCTGGTC |
| | (SEQ ID NO: 15 fused | TAGAGAATGAATTATGGATCCTTGAGGTGGGAGCTGGGCTCT |
| | to SEQ ID NO: 14) | CCCTGATGTATTATTCAGCTCCCTGGAGTTGGCCAGCTCCTG |
| | | TTACACTGGCCACAGCCCTG |
| 18 | Portion of SEQ ID NO: | TGAGGTGGGAGCTGGGCTCTCCCTGATGTATTATTCAGCTCC |
| | 8 | CTGGAGTTGGCCAGCTCCTGTTACACTGGCCACAGCCCTGG |
| | (−590 to −161) | GCATCCGCTTCTCACTTCTAGTTTCCCCTCCAAGGTAATGTG |
| | | GTGGGTCATGATCATTCTATCCTGGCTTCAGGGACCTGACTC |
| | | CACTTTGGGGCCATTCGAGGGGTCTAGGGTAGATGATGTCC |
| | | CCCTGTGGGGATTAATGTCCTGCTCTGTAAAACTGAGCTAGC |
| | | TGAGATCCAGGAGGGCTTGGCCAGAGACAGCAAGTTGTTGC |
| | | CATGGTGACTTTAAAGCCAGGTTGCTGCCCCAGCACAGGCCT |
| | | CCCAGTCTACCCTCACTAGAAAACAACACCCAGGCACTTTCC |
| | | ACCACCTCTCAAAGGTGAAACCCAAGGCTGGTCTAGAGAATG |
| | | AATTATGGATCCT |
| 19 | SEQ ID NO: 7 fused to | CTGCAGCTCAGCCTACTACTTGCTTTCCAGGCTGTTCCTAGT |
| | SEQ ID NO: 8 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTAAGCTTCTGC |
| | | CACTGGCTCCGGCATTGCAGAGAGAAGAGAAGGGGCGGCA |
| | | GAGCTGAACCTTAGCCTTGCCTTCCTGGGTACCCTTCTGAGC |
| | | CTCACTGTCTTCTGTGAGATGGGCAAAGTGCGGGTGTGACTC |
| | | CTTGGCAACGGTGTTACACCAGGGCAGGTAAAGTTGTAGTTA |
| | | TTTGTGGGGTACACCAGGACTGTTAAAGGTGTAACTATGGTC |
| | | TCACCCAGCATTTTCACTTCTAATAAGTTCAAATGTGATACGG |
| | | CACCTTTCTAAAAATTAGTTTTCAGGGAAATAGGGTTCAAAAC |
| | | TGGTAGTGGTAGGGTCCATTCTCACGACCCCCAGGCCTGCT |
| | | AACCCTGACCAAGCTACCTATTACTTACCCTCCTCTTTCTCCT |
| | | CCTCCTCTTTCTCCTTCTCCTGCTTCCCCTCTTCCTTCTCCCT |
| | | CCCTTCCTCTCCCTCCTCCCCCTCCTTGGCTGTGATCAGATC |
| | | CAGAGCCTGAATGAGCCTCCTGACCCCACACCCCCACTAGC |
| | | ATGGGCCTGCAAGTGCCCAGAAGTCCCTCCTGCCTCCTAAAC |
| | | TGCCCAGCCGATCCATTAGCTCTTCCTTCTTCCCAGTGAAAG |
| | | AAGCAGGCACAGCCTGTCCCTCCCGTTCTACAGAAAGGAAG |
| | | CTACAGCACAGGGAGGGCCAAAGGCCTTCCTGGGACTAGAC |
| | | AGTTGATCAACAGCAGGACTGGAGAGCTGGGCTCCATTTTTG |
| | | TTCCTTGGTGCCCTGCCCCTCCCCATGACCTGCAGAGACATT |
| | | CAGCCTGCCAGGCTTTATGAGGTGGGAGCTGGGCTCTCCCT |
| | | GATGTATTATTCAGCTCCCTGGAGTTGGCCAGCTCCTGTTAC |
| | | ACTGGCCACAGCCCTGGGCATCCGCTTCTCACTTCTAGTTTC |
| | | CCCTCCAAGGTAATGTGGTGGGTCATGATCATTCTATCCTGG |
| | | CTTCAGGGACCTGACTCCACTTTGGGGCCATTCGAGGGGTC |
| | | TAGGGTAGATGATGTCCCCCTGTGGGGATTAATGTCCTGCTC |
| | | TGTAAAACTGAGCTAGCTGAGATCCAGGAGGGCTTGGCCAG |
| | | AGACAGCAAGTTGTTGCCATGGTGACTTTAAAGCCAGGTTGC |
| | | TGCCCCAGCACAGGCCTCCCAGTCTACCCTCACTAGAAAACA |
| | | ACACCCAGGCACTTTCCACCACCTCTCAAAGGTGAAACCCAA |
| | | GGCTGGTCTAGAGAATGAATTATGGATCCTCGCTGTCCGTGC |
| | | CACCCAGCTAGTCCCAGCGGCTCAGACACTGAGGAGAGACT |
| | | GTAGGTTCAGCTACAAGCAAAAAGACCTAGCTGGTCTCCAAG |
| | | CAGTGTCTCCAAGTCCCTGAACCTGTGACACCTGCCCCAGG |
| | | CATCATCAGGCACAGAGGGCCACC |
| 20 | SEQ ID NO: 8 fused to | GGTCTCACCCAGCATTTTCACTTCTAATAAGTTCAAATGTGAT |
| | SEQ ID NO: 7 | ACGGCACCTTTCTAAAAATTAGTTTTCAGGGAAATAGGGTTCA |
| | | AAACTGGTAGTGGTAGGGTCCATTCTCACGACCCCCAGGCCT |
| | | GCTAACCCTGACCAAGCTACCTATTACTTACCCTCCTCTTTCT |
| | | CCTCCTCCTCTTTCTCCTTCTCCTGCTTCCCCTCTTCCTTCTC |
| | | CCTCCCTTCCTCTCCCTCCTCCCCCTCCTTGGCTGTGATCAG |
| | | ATCCAGAGCCTGAATGAGCCTCCTGACCCCACACCCCCACTA |
| | | GCATGGGCCTGCAAGTGCCCAGAAGTCCCTCCTGCCTCCTA |
| | | AACTGCCCAGCCGATCCATTAGCTCTTCCTTCTTCCCAGTGA |
| | | AAGAAGCAGGCACAGCCTGTCCCTCCCGTTCTACAGAAAGG |
| | | AAGCTACAGCACAGGGAGGGCCAAAGGCCTTCCTGGGACTA |
| | | GACAGTTGATCAACAGCAGGACTGGAGAGCTGGGCTCCATTT |
| | | TTGTTCCTTGGTGCCCTGCCCCTCCCCATGACCTGCAGAGAC |
| | | ATTCAGCCTGCCAGGCTTTATGAGGTGGGAGCTGGGCTCTC |
| | | TACACTGGCCACAGCCCTGGGCATCCGCTTCTCACTTCTAGT |
| | | TTCCCCTCCAAGGTAATGTGGTGGGTCATGATCATTCTATCCT |
| | | GGCTTCAGGGACCTGACTCCACTTTGGGGCCATTCGAGGGG |
| | | TCTAGGGTAGATGATGTCCCCCTGTGGGGATTAATGTCCTGC |
| | | TCTGTAAAACTGAGCTAGCTGAGATCCAGGAGGGCTTGGCCA |
| | | GAGACAGCAAGTTGTTGCCATGGTGACTTTAAAGCCAGGTTG |
| | | CTGCCCCAGCACAGGCCTCCCAGTCTACCCTCACTAGAAAAC |
| | | AACACCCAGGCACTTTCCACCACCTCTCAAAGGTGAAACCCA |
| | | AGGCTGGTCTAGAGAATGAATTATGGATCCTCGCTGTCCGTG |
| | | CCACCCAGCTAGTCCCAGCGGCTCAGACACTGAGGAGAGAC |
| | | TGTAGGTTCAGCTACAAGCAAAAAGACCTAGCTGGTCTCCAA |
| | | GCAGTGTCTCCAAGTCCCTGAACCTGTGACACCTGCCCCAG |
| | | GCATCATCAGGCACAGAGGGCCACCCTGCAGCTCAGCCTAC |
| | | TACTTGCTTTCCAGGCTGTTCCTAGTTCCCATGTCAGCTGCTT |
| | | GTGCTTTCCAGAGACAAAACAGGAATAATAGATGTCATTAAAT |
| | | ATACATTGGGCCCCAGGCGGTCAATGTGGCAGCCTGAGCCT |
| | | CCTTTCCATCTCTGTGGAGGCAGACATAGGACCCCCAACAAA |
| | | CAGCATGCAGGTTGGGAGCCAGCCACAGGACCCAGGTAAGG |
| | | GGCCCTGGGTCCTTAAGCTTCTGCCACTGGCTCCGGCATTG |
| | | CAGAGAGAAGAGAAGGGGCGGCAGAGCTGAACCTTAGCCTT |
| | | GCCTTCCTGGGTACCCTTCTGAGCCTCACTGTCTTCTGTGAG |
| | | ATGGGCAAAGTGCGGGTGTGACTCCTTGGCAACGGTGTTAC |
| | | ACCAGGGCAGGTAAAGTTGTAGTTATTTGTGGGGTACACCAG |
| | | GACTGTTAAAGGTGTAACTAT |
| 21 | Portion of SEQ ID NO: | CTGCAGCTCAGCCTACTACTTGCTTTCCAGGCTGTTCCTAGT |
| | 7 that contains SEQ ID | TCCCATGTCAGCTGCTTGTGCTTTCCAGAGACAAAACAGGAA |
| | NO: 9 and SEQ ID NO: | TAATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTAAGCTTCTGC |
| | 14 and SEQ ID NO: 15 | CACTGGCTCCGGCATTGCAGAGAGAAGAGAAGGGGCGGCA |
| | | GACTGGAGAGCTGGGCTCCATTTTTGTTCCTTGGTGCCCTGC |
| | | CCCTCCCCATGACCTGCAGAGACATTCAGCCTGCCAGGCTTT |
| | | ATGAGGTGGGAGCTGGGCTCTCCCTGATGTATTATTCAGCTC |
| | | CCTGGAGTTGGCCAGCTCCTGTTACACTGGCCACAGCCCTG |
| | | GGCATCCGCTTCTCACTTCTAGTTTCCCCTCCAAGGTAATGT |
| | | GGTGGGTCATGATCATTCTATCCTGGCTTCAGGGACCTGACT |
| | | CCACTTTGGGGCCATTCGAGGGGTCTAGGGTAGATGATGTC |
| | | CCCCTGTGGGGATTAATGTCCTGCTCTGTAAAACTGAGCTAG |
| | | CTGAGATCCAGGAGGGCTTGGCCAGAGACAGCAAGTTGTTG |
| | | CCATGGTGACTTTAAAGCCAGGTTGCTGCCCCAGCACAGGC |
| | | CTCCCAGTCTACCCTCACTAGAAAACAACACCCAGGCACTTT |
| | | CCACCACCTCTCAAAGGTGAAACCCAAGGCTGGTCTAGAGAA |
| | | TGAATTATGGATCCTCGCTGTCCGTGCCACCCAGCTAGTCCC |
| | | AGCGGCTCAGACACTGAGGAGAGACTGTAGGTTCAGCTACA |
| | | AGCAAAAAGACCTAGCTGGTCTCCAAGCAGTGTCTCCAAGTC |
| | | CCTGAACCTGTGACACCTGCCCCAGGCATCATCAGGCACAG |
| | | AGGGCCACC |
| 22 | Portion of SEQ ID NO: | CTGCAGCTCAGCCTACTACTTGCTTTCCAGGCTGTTCCTAGT |
| | 7 that contains SEQ ID | TCCCATGTCAGCTGCTTGTGCTTTCCAGAGACAAAACAGGAA |
| | NO: 9 and SEQ ID NO: | TAATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTTTTATGAGGT |
| | 14 and SEQ ID NO: 15 | GGGAGCTGGGCTCTCCCTGATGTATTATTCAGCTCCCTGGAG |
| | | TTGGCCAGCTCCTGTTACACTGGCCACAGCCCTGGGCATCC |
| | | GCTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |
| 23 | Region 1 of the human | GTATGCCTTTTGAGATGGATGCAGCAGGTTCTGTGAGGCTGC |
| | Myo15 promoter | CAGGAGGGGTAGAGTTCCCGGGGGCCTCGGGCCCCGCTGG |
| | | AGTGTGGAGCAGGCCCATGCTCAGCTCTCCAGGCTGTTCGT |
| | | GGCTCCCCTGTCAGCTGCTCACTCCTTTCCAGAGACAAAACA |
| | | GGAATAATAGACATCATTAAATATACATAGGGCCCCAGGCGG |
| | | TCGGCGTGGTGGGCTGGGCCTCCCTTCC |
| 24 | Region 2 of human | TGCCCTGCCTTCTGAGCCGGCAGCCTGGCTCCCCACCCCAT |
| | Myo15 promoter | GTATTATTCAGCTCCTGAGAGCCAGCCAGCTCCTGTTACACT |
| | | GACCGCAGCCCAGCACCTGCTCTGCCCATTCCCCTCCTCCC |
| | | TTGCCTAGGACCTAGAGGGTTCAAAGTTCTCCTCCAAGATGA |
| | | CTTGGTGGGCTTTGGCCATCCCACCCTAGGCCCCACTTCTG |
| | | GCCCAGTGCAGGTGTGCTGGTGATTTAGGGCAGGTGGCATT |
| | | CCATCTCTGTGGCTCAATGTCTTCCTCTGTGAAGCCGAAGTG |
| | | ACCCAAGGGCTCCCTTCATGGGGTTGAGCCAGCTGTGGCCC |
| | | AGGGAGGGCCTAACCAGGATGAGCACTGATGTTGCCATGAC |
| | | GACTCCGAGGCCAGAATGTCTCCCCCAGCACAGGCCTCATA |
| | | GGCAGGCTTCCCCATCCTGGTAAACAACACCCACACACTTTC |
| | | TACTACTGCTCTAGGGTGAAACCCAAGGCGCTCTAGAGGAGA |
| | | TGAATTATGGATCCGCCCTCCCGGAATCCTGGCTCGGCCCTC |
| | | CCCACGCCACCCAGGGCCAGTCGGGTCTGCTCACAGCCCGA |
| | | GGAGGCCGCGTGTCCAGCCGCGGGCAAGAGACAGAGCAGG |
| | | TCCCTGTGTCTCCAAGTCCCTGAGCCCGTGACACCGGCCCC |
| | | AGGCCCTGTAGAGAGCAGGCAGCCACC |
| 25 | Portion of SEQ ID NO: | CCCCTGTCAGCTGCTCACTCCTTTCCAGAGACAAAACAGGAA |
| | 23 | TAATAGACATCATTAAATATACATAGGGCCCCAGG |
| 26 | Portion of SEQ ID NO: | TGAGCCGGCAGCCTGGCTCCCCACCCCATGTATTATTCAGCT |
| | 24 | CCTGAGAGCCAGCCAGCTCCTGTTACACTGACCGCAGCCC |
| 27 | Portion of SEQ ID NO: | CACAGGCCTCATAGGCAGGCTTCCCCATCCTGGTAAACAACA |
| | 24 | CCCACACACTTTCTACTACTGCTCTAGGGTGAAACCCAAGGC |
| | | GCTCTAGAGGAGATGAATTATGGATCC |
| 28 | Portion of SEQ ID NO: | TGAGCCGGCAGCCTGGCTCCCCACCCCATGTATTATTCAGCT |
| | 24 | CCTGAGAGCCAGCCAGCTCCTGTTACACTGACCGCAGCCCC |
| | (SEQ ID NO: 26 fused | ACAGGCCTCATAGGCAGGCTTCCCCATCCTGGTAAACAACAC |
| | to SEQ ID NO: 27) | CCACACACTTTCTACTACTGCTCTAGGGTGAAACCCAAGGCG |
| | | CTCTAGAGGAGATGAATTATGGATCC |
| 29 | Portion of SEQ ID NO: | CACAGGCCTCATAGGCAGGCTTCCCCATCCTGGTAAACAACA |
| | 24 | CCCACACACTTTCTACTACTGCTCTAGGGTGAAACCCAAGGC |
| | (SEQ ID NO: 27 fused | GCTCTAGAGGAGATGAATTATGGATCCTGAGCCGGCAGCCT |
| | to SEQ ID NO: 26) | GGCTCCCCACCCCATGTATTATTCAGCTCCTGAGAGCCAGCC |
| | | AGCTCCTGTTACACTGACCGCAGCCC |
| 30 | Portion of SEQ ID NO: | TGAGCCGGCAGCCTGGCTCCCCACCCCATGTATTATTCAGCT |
| | 24 (contiguous | CCTGAGAGCCAGCCAGCTCCTGTTACACTGACCGCAGCCCA |
| | sequence including | GCACCTGCTCTGCCCATTCCCCTCCTCCCTTGCCTAGGACCT |
| | SEQ ID NO: 26 and | AGAGGGTTCAAAGTTCTCCTCCAAGATGACTTGGTGGGCTTT |
| | SEQ ID NO: 27) | GGCCATCCCACCCTAGGCCCCACTTCTGGCCCAGTGCAGGT |
| | | GTGCTGGTGATTTAGGGCAGGTGGCATTCCATCTCTGTGGCT |
| | | CAATGTCTTCCTCTGTGAAGCCGAAGTGACCCAAGGGCTCCC |
| | | TTCATGGGGTTGAGCCAGCTGTGGCCCAGGGAGGGCCTAAC |
| | | CAGGATGAGCACTGATGTTGCCATGACGACTCCGAGGCCAG |
| | | AATGTCTCCCCCAGCACAGGCCTCATAGGCAGGCTTCCCCAT |
| | | CCTGGTAAACAACACCCACACACTTTCTACTACTGCTCTAGG |
| | | GTGAAACCCAAGGCGCTCTAGAGGAGATGAATTATGGATCC |
| 31 | Polynucleotide | GTATGCCTTTTGAGATGGATGCAGCAGGTTCTGTGAGGCTGC |
| | containing SEQ ID NO: | CAGGAGGGGTAGAGTTCCCGGGGGCCTCGGGCCCCGCTGG |
| | 23 and SEQ ID NO: 24 | AGTGTGGAGCAGGCCCATGCTCAGCTCTCCAGGCTGTTCGT |
| | | GGCTCCCCTGTCAGCTGCTCACTCCTTTCCAGAGACAAAACA |
| | | GGAATAATAGACATCATTAAATATACATAGGGCCCCAGGCGG |
| | | TCGGCGTGGTGGGCTGGGCCTCCCTTCCCCATAACACTGAG |
| | | CTGCTCTGCTGGGCCAACCGTGCTCCTGGGCCAGCCAGAGG |
| | | ACCCCCATGAGGCGGCATGCAGGCGGGGAGCAGGCCACAG |
| | | AACGCAGGTAAGGAGACCTTAGCCTAGAGTCCTTGGGGTCT |
| | | GTCACTGGCCACCCTCGCATCCCAGGCTGCAGGAAACTGAG |
| | | GCCCAGAGAGGACAAGGACTTTCCTGGACCCACACAGCCAG |
| | | TCAGTGACAGAGCCTAGGGTCTGAGCCAGGCCTGACCCAAC |
| | | CTCCATTTCTGCCTCTCTACCCCTGCCCCCGCCCCAACACAC |
| | | ACACACACACAAGTGGAGTTCCACTGAAACGCCCCTCCTTGC |
| | | CCTGCCTTCTGAGCCGGCAGCCTGGCTCCCCACCCCATGTA |
| | | TTATTCAGCTCCTGAGAGCCAGCCAGCTCCTGTTACACTGAC |
| | | CGCAGCCCAGCACCTGCTCTGCCCATTCCCCTCCTCCCTTGC |
| | | CTAGGACCTAGAGGGTTCAAAGTTCTCCTCCAAGATGACTTG |
| | | GTGGGCTTTGGCCATCCCACCCTAGGCCCCACTTCTGGCCC |
| | | AGTGCAGGTGTGCTGGTGATTTAGGGCAGGTGGCATTCCAT |
| | | CTCTGTGGCTCAATGTCTTCCTCTGTGAAGCCGAAGTGACCC |
| | | AAGGGCTCCCTTCATGGGGTTGAGCCAGCTGTGGCCCAGGG |
| | | AGGGCCTAACCAGGATGAGCACTGATGTTGCCATGACGACT |
| | | CCGAGGCCAGAATGTCTCCCCCAGCACAGGCCTCATAGGCA |
| | | GGCTTCCCCATCCTGGTAAACAACACCCACACACTTTCTACT |
| | | ACTGCTCTAGGGTGAAACCCAAGGCGCTCTAGAGGAGATGA |
| | | ATTATGGATCCGCCCTCCCGGAATCCTGGCTCGGCCCTCCC |
| | | CACGCCACCCAGGGCCAGTCGGGTCTGCTCACAGCCCGAG |
| | | GAGGCCGCGTGTCCAGCCGCGGGCAAGAGACAGAGCAGGT |
| | | CCCTGTGTCTCCAAGTCCCTGAGCCCGTGACACCGGCCCCA |
| | | GGCCCTGTAGAGAGCAGGCAGCCACC |
| 32 | Polynucleotide | GCAGGCCCATGCTCAGCTCTCCAGGCTGTTCGTGGCTCCCC |
| | containing SEQ ID NO: | TGTCAGCTGCTCACTCCTTTCCAGAGACAAAACAGGAATAAT |
| | 25, SEQ ID NO: 26, | AGACATCATTAAATATACATAGGGCCCCAGGCGGTCGGCGTG |
| | and SEQ ID NO: 27 | GTGGGCTGGGCCTCCCTTCCCCATAACACTGAGCTGCTCTG |
| | | CTGGGCCAACCGTGCTCCTGGGCCAGCCAGAGGACCCCCAT |
| | | GAGGCGGCATGCAGGCGGGGAGCAGGCCACAGAACGCAGG |
| | | TAAGGAGACCTTGCCTTCTGAGCCGGCAGCCTGGCTCCCCA |
| | | CCCCATGTATTATTCAGCTCCTGAGAGCCAGCCAGCTCCTGT |
| | | TACACTGACCGCAGCCCAGCACCTGCTCTGCCCATTCCCCTC |
| | | CTCCCTTGCCTAGGACCTAGAGGGTTCAAAGTTCTCCTCCAA |
| | | GATGACTTGGTGGGCTTTGGCCATCGGGCCTAACCAGGATG |
| | | AGCACTGATGTTGCCATGACGACTCCGAGGCCAGAATGTCTC |
| | | CCCCAGCACAGGCCTCATAGGCAGGCTTCCCCATCCTGGTA |
| | | AACAACACCCACACACTTTCTACTACTGCTCTAGGGTGAAAC |
| | | CCAAGGCGCTCTAGAGGAGATGAATTATGGATCCGCCCTCC |
| | | CGGAATCCTGGCTCGGCCCTCCCCACGC |
| 33 | Portion of SEQ ID NO: | CTGCAGCTCAGCCTACTACTTGCTTTCCAGGCTGTTCCTAGT |
| | 7 that contains SEQ ID | TCCCATGTCAGCTGCTTGTGCTTTCCAGAGACAAAACAGGAA |
| | NO: 9 and SEQ ID NO: | TAATAGATGTCATTAAATATACATTGGGCCCCAGGCGGTCAAT |
| | 10 | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTT |
| 34 | Portion of SEQ ID NO: | TTTATGAGGTGGGAGCTGGGCTCTCCCTGATGTATTATTCAG |
| | 8 that contains SEQ ID | CTCCCTGGAGTTGGCCAGCTCCTGTTACACTGGCCACAGCC |
| | NO: 14 | CTGGGCATCCGC |
| 35 | Portion of SEQ ID NO: | TGCCATGGTGACTTTAAAGCCAGGTTGCTGCCCCAGCACAG |
| | 8 that contains SEQ ID | GCCTCCCAGTCTACCCTCACTAGAAAACAACACCCAGGCACT |
| | NO: 15 | TTCCACCACCTCTCAAAGGTGAAACCCAAGGCTGGTCTAGAG |
| | | AATGAATTATGGATCCTCGCTGTCCGTGCCACCCAGCTAGTC |
| | | CCAGCGGCTCAGACACTG |
| 36 | SEQ ID NO: 33 fused | CTGCAGCTCAGCCTACTACTTGCTTTCCAGGCTGTTCCTAGT |
| | to SEQ ID NO: 34 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTTTTATGAGGT |
| | | GGGAGCTGGGCTCTCCCTGATGTATTATTCAGCTCCCTGGAG |
| | | TTGGCCAGCTCCTGTTACACTGGCCACAGCCCTGGGCATCC |
| | | GC |
| 37 | SEQ ID NO: 33 fused | CTGCAGCTCAGCCTACTACTTGCTTTCCAGGCTGTTCCTAGT |
| | to SEQ ID NO: 35 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTTGCCATGGTG |
| | | ACTTTAAAGCCAGGTTGCTGCCCCAGCACAGGCCTCCCAGTC |
| | | TACCCTCACTAGAAAACAACACCCAGGCACTTTCCACCACCT |
| | | CTCAAAGGTGAAACCCAAGGCTGGTCTAGAGAATGAATTATG |
| | | GATCCTCGCTGTCCGTGCCACCCAGCTAGTCCCAGCGGCTC |
| | | AGACACTG |
| 38 | SEQ ID NO: 34 fused | TTTATGAGGTGGGAGCTGGGCTCTCCCTGATGTATTATTCAG |
| | to SEQ ID NO: 35 | CTCCCTGGAGTTGGCCAGCTCCTGTTACACTGGCCACAGCC |
| | | CTGGGCATCCGCTGCCATGGTGACTTTAAAGCCAGGTTGCTG |
| | | CCCCAGCACAGGCCTCCCAGTCTACCCTCACTAGAAAACAAC |
| | | ACCCAGGCACTTTCCACCACCTCTCAAAGGTGAAACCCAAGG |
| | | CTGGTCTAGAGAATGAATTATGGATCCTCGCTGTCCGTGCCA |
| | | CCCAGCTAGTCCCAGCGGCTCAGACACTG |
| 39 | SEQ ID NO: 34 fused | TTTATGAGGTGGGAGCTGGGCTCTCCCTGATGTATTATTCAG |
| | to SEQ ID NO: 33, | CTCCCTGGAGTTGGCCAGCTCCTGTTACACTGGCCACAGCC |
| | which is fused to SEQ | CTGGGCATCCGCCTGCAGCTCAGCCTACTACTTGCTTTCCAG |
| | ID NO: 35 | GCTGTTCCTAGTTCCCATGTCAGCTGCTTGTGCTTTCCAGAG |
| | | ACAAAACAGGAATAATAGATGTCATTAAATATACATTGGGCCC |
| | | CAGGCGGTCAATGTGGCAGCCTGAGCCTCCTTTCCATCTCTG |
| | | TGGAGGCAGACATAGGACCCCCAACAAACAGCATGCAGGTT |
| | | GGGAGCCAGCCACAGGACCCAGGTAAGGGGCCCTGGGTCC |
| | | TTTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |
| 40 | SEQ ID NO: 35 fused | TGCCATGGTGACTTTAAAGCCAGGTTGCTGCCCCAGCACAG |
| | to SEQ ID NO: 33, | GCCTCCCAGTCTACCCTCACTAGAAAACAACACCCAGGCACT |
| | which is fused to SEQ | TTCCACCACCTCTCAAAGGTGAAACCCAAGGCTGGTCTAGAG |
| | ID NO: 34 | AATGAATTATGGATCCTCGCTGTCCGTGCCACCCAGCTAGTC |
| | | CCAGCGGCTCAGACACTGCTGCAGCTCAGCCTACTACTTGCT |
| | | TTCCAGGCTGTTCCTAGTTCCCATGTCAGCTGCTTGTGCTTTC |
| | | CAGAGACAAAACAGGAATAATAGATGTCATTAAATATACATTG |
| | | GGCCCCAGGCGGTCAATGTGGCAGCCTGAGCCTCCTTTCCA |
| | | TCTCTGTGGAGGCAGACATAGGACCCCCAACAAACAGCATG |
| | | CAGGTTGGGAGCCAGCCACAGGACCCAGGTAAGGGGCCCT |
| | | GGGTCCTTTTTATGAGGTGGGAGCTGGGCTCTCCCTGATGTA |
| | | TTATTCAGCTCCCTGGAGTTGGCCAGCTCCTGTTACACTGGC |
| | | CACAGCCCTGGGCATCCGC |
| 41 | SEQ ID NO: 34 fused | TTTATGAGGTGGGAGCTGGGCTCTCCCTGATGTATTATTCAG |
| | to SEQ ID NO: 35, | CTCCCTGGAGTTGGCCAGCTCCTGTTACACTGGCCACAGCC |
| | which is fused to SEQ | CTGGGCATCCGCTGCCATGGTGACTTTAAAGCCAGGTTGCTG |
| | ID NO: 33 | CCCCAGCACAGGCCTCCCAGTCTACCCTCACTAGAAAACAAC |
| | | ACCCAGGCACTTTCCACCACCTCTCAAAGGTGAAACCCAAGG |
| | | CTGGTCTAGAGAATGAATTATGGATCCTCGCTGTCCGTGCCA |
| | | CCCAGCTAGTCCCAGCGGCTCAGACACTGCTGCAGCTCAGC |
| | | CTACTACTTGCTTTCCAGGCTGTTCCTAGTTCCCATGTCAGCT |
| | | GCTTGTGCTTTCCAGAGACAAAACAGGAATAATAGATGTCATT |
| | | AAATATACATTGGGCCCCAGGCGGTCAATGTGGCAGCCTGA |
| | | GCCTCCTTTCCATCTCTGTGGAGGCAGACATAGGACCCCCAA |
| | | CAAACAGCATGCAGGTTGGGAGCCAGCCACAGGACCCAGGT |
| | | AAGGGGCCCTGGGTCCTT |
| 42 | Portion of SEQ ID NO: | TGCAGCTCAGCCTACTACTTGCTTTCCAGGCTGTTCCTAGTT |
| | 7 that contains SEQ ID | CCCATGTCAGCTGCTTGTGCTTTCCAGAGACAAAACAGGAAT |
| | NO: 9 and SEQ ID NO: | AATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTAAGCTTCTGC |
| | 14 and SEQ ID NO: 15 | CACTGGCTCCGGCATTGCAGAGAGAAGAGAAGGGGCGGCA |
| | | GACTGGAGAGCTGGGCTCCATTTTTGTTCCTTGGTGCCCTGC |
| | | CCCTCCCCATGACCTGCAGAGACATTCAGCCTGCCAGGCTTT |
| | | ATGAGGTGGGAGCTGGGCTCTCCCTGATGTATTATTCAGCTC |
| | | CCTGGAGTTGGCCAGCTCCTGTTACACTGGCCACAGCCCTG |
| | | GGCATCCGCTTCTCACTTCTAGTTTCCCCTCCAAGGTAATGT |
| | | GGTGGGTCATGATCATTCTATCCTGGCTTCAGGGACCTGACT |
| | | CCACTTTGGGGCCATTCGAGGGGTCTAGGGTAGATGATGTC |
| | | CCCCTGTGGGGATTAATGTCCTGCTCTGTAAAACTGAGCTAG |
| | | CTGAGATCCAGGAGGGCTTGGCCAGAGACAGCAAGTTGTTG |
| | | CCATGGTGACTTTAAAGCCAGGTTGCTGCCCCAGCACAGGC |
| | | CTCCCAGTCTACCCTCACTAGAAAACAACACCCAGGCACTTT |
| | | CCACCACCTCTCAAAGGTGAAACCCAAGGCTGGTCTAGAGAA |
| | | TGAATTATGGATCCTCGCTGTCCGTGCCACCCAGCTAGTCCC |
| | | AGCGGCTCAGACACTGAGGAGAGACTGTAGGTTCAGCTACA |
| | | AGCAAAAAGACCTAGCTGGTCTCCAAGCAGTGTCTCCAAGTC |
| | | CCTGAACCTGTGACACCTGCCCCAGGCATCATCAGGCACAG |
| | | AGGGCCACC |
| 43 | Portion of SEQ ID NO: | TGCAGCTCAGCCTACTACTTGCTTTCCAGGCTGTTCCTAGTT |
| | 7 that contains SEQ ID | CCCATGTCAGCTGCTTGTGCTTTCCAGAGACAAAACAGGAAT |
| | NO: 9 and SEQ ID NO: | AATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTTTTATGAGGT |
| | 14 and SEQ ID NO: 15 | GGGAGCTGGGCTCTCCCTGATGTATTATTCAGCTCCCTGGAG |
| | | TTGGCCAGCTCCTGTTACACTGGCCACAGCCCTGGGCATCC |
| | | GCTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |

Additional Myo15 promoters useful in conjunction with the compositions and methods described herein include nucleic acid molecules that have at least 85% sequence identity (e.g., 85%, 90%, 95%, 96%, 97%, 98%, 99%, or more, sequence identity) to the polynucleotide sequences set forth in Table 3, as well as functional portions or derivatives of the polynucleotide sequences set forth in Table 3. The Myo15 promoters listed in Table 3 are characterized in International Application Publication Nos. WO2019210181A1 and WO2020163761A1, which are incorporated herein by reference.

In embodiments in which an smCBA promoter is included in a dual vector system described herein (e.g., in the first vector in a dual vector system), the smCBA promoter may have the sequence of the smCBA promoter described in U.S. Pat. No. 8,298,818, which is incorporated herein by reference. In some embodiments, the smCBA promoter has the sequence of:

(SEQ ID NO: 44)
GGTACCTAGTTATTAATAGTAATCAATTACGGGGTCATTAGTTCATAG
CCCATATATGGAGTTCCGCGTTACATAACTTACGGTAAATGGCCCGCC
TGGCTGACCGCCCAACGACCCCCGCCCATTGACGTCAATAATGACGTA
TGTTCCCATAGTAACGCCAATAGGGACTTTCCATTGACGTCAATGGGT
GGAGTATTTACGGTAAACTGCCCACTTGGCAGTACATCAAGTGTATCA
TATGCCAAGTACGCCCCCTATTGACGTCAATGACGGTAAATGGCCCGC
CTGGCATTATGCCCAGTACATGACCTTATGGGACTTTCCTACTTGGCA
GTACATCTACGTATTAGTCATCGCTATTACCATGGTCGAGGTGAGCCC
CACGTTCTGCTTCACTCTCCCCATCTCCCCCCCCTCCCCACCCCCAAT
TTTGTATTTATTTATTTTTTAATTATTTTGTGCAGCGATGGGGGCGGG
GGGGGGGGGGGGGCGCGCGCCAGGCGGGGCGGGGCGGGGCGAGGGGCG
GGGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTA
TAAAAAGCGAAGCGCGCGGCGGGCGGGAGTCGCTGCGCGCTGCCTTCG
CCCCGTGCCCCGCTCCGCCGCCGCCTCGCGCCGCCCGCCCCGGCTCTG
ACTGACCGCGTTACTCCCACAGGTGAGCGGGCGGGACGGCCCTTCTCC
TCCGGGCTGTAATTAGCGCTTGGTTTAATGACGGCTTGTTTCTTTTCT
GTGGCTGCGTGAAAGCCTTGAGGGGCTCCGGGAGCTAGAGCCTCTGCT
AACCATGTTCATGCCTTCTTCTTTTTCCTACAGCTCCTGGGCAACGTG
CTGGTTATTGTGCTGTCTCATCATTTTGGCA.

Once a polynucleotide encoding OTOF has been incorporated into the nuclear DNA of a mammalian cell or stabilized in an episomal monomer or concatemer, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in the nucleic acid vectors for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode an OTOF protein and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv, et al., Nature 297:17 (1982). An enhancer may be spliced into a vector containing a polynucleotide encoding an OTOF protein, for example, at a position 5′ or 3′ to this gene. In a preferred orientation, the enhancer is positioned at the 5′ side of the promoter, which in turn is located 5′ relative to the polynucleotide encoding an OTOF protein.

The nucleic acid vectors described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. The WPRE can be located in the second nucleic acid vector between the polynucleotide encoding a C-terminal portion of an OTOF protein and the poly(A) sisequence. In some embodiments of the compositions and methods described herein, the WPRE has the sequence:

(SEQ ID NO: 45)
GATCCAATCAACCTCTGGATTACAAAATTTGTGAAAGATTGACTGGTATT
CTTAACTATGTTGCTCCTTTTACGCTATGTGGATACGCTGCTTTAATGCC
TTTGTATCATGCTATTGCTTCCCGTATGGCTTTCATTTTCTCCTCCTTGT
ATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTG
GGGCATTGCCACCACCTGTCAGCTCCTTTCCGGGACTTTCGCTTTCCCCC
TCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAA
ATCATCGTCCTTTCCTTGGCTGCTCGCCTGTGTTGCCACCTGGATTCTGC
GCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGA.

In other embodiments, the WPRE has the sequence:

(SEQ ID NO: 46)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTAGTTCTTGCCACGGCGGAACTCATCGCCGCCTGCCTTGCCCG
CTGCTGGACAGGGGCTCGGCTGTTGGGCACTGACAATTCCGTGGTGTTTA
TTTGTGAAATTTGTGATGCTATTGCTTTATTTGTAACCATCTAGCTTTAT
TTGTGAAATTTGTGATGCTATTGCTTTATTTGTAACCATTATAAGCTGCA
ATAAACAAGTTAACAACAACAATTGCATTCATTTTATGTTTCAGGTTCAG
GGGGAGATGTGGGAGGTTTTTTAAA

In some embodiments, the nucleic acid vectors for use in the compositions and methods described herein include a reporter sequence, which can be useful in verifying OTOF gene expression, for example, in specific cells and tissues (e.g., in cochlear hair cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Dual Hybrid Vectors for Expressing OTOF

An OTOF isoform 5 protein (e.g., an OTOF isoform 5 protein having the sequence of SEQ ID NO: 1) can be expressed in mammalian cells using a dual hybrid vector system. This approach uses two nucleic acid vectors (e.g., two adeno-associated virus vectors) to express a single, large protein. Each of the two nucleic acid vectors (e.g., two adeno-associated virus vectors) contains a portion of a polynucleotide that encodes the protein (e.g., one vector contains a polynucleotide encoding an N-terminal portion of the protein and the other vector contains a polynucleotide encoding a C-terminal portion of the protein, and the polynucleotide encoding the N-terminal portion of the protein and the polynucleotide encoding the C-terminal portion of the protein do not overlap). The dual hybrid vectors also feature an overlapping region at which homologous recombination can occur (e.g., a recombinogenic region that is contained within each vector) and splice donor and splice acceptor sequences (e.g., the first vector contains a splice donor sequence and the second vector contains a splice acceptor sequence). The recombinogenic region is 3′ of the splice donor sequence in the first nucleic acid vector and 5′ of the splice acceptor sequence in the second nucleic acid vector. The first and second polynucleotide sequences can then join to form a single sequence based on one of two mechanisms: 1) recombination at the overlapping region, or 2) concatemerization of the ITRs. The remaining recombinogenic region(s) and/or the concatemerized ITRs can be removed by splicing, leading to the formation of a contiguous polynucleotide sequence that encodes the full-length protein of interest.

Recombinogenic regions that can be used in the compositions and methods described herein include the F1 phage AK gene having a sequence of: GGGATTTTGCCGATTTCGGCCTATTGGTTAA AAAATGAGCTGATTTAACAAAAATTTAACGCGAATTTTAACAAAAT (SEQ ID NO: 47) and alkaline phosphatase (AP) gene fragments as described in U.S. Pat. No. 8,236,557, which are incorporated herein by reference. In some embodiments, the AP gene fragment has the sequence of:

(SEQ ID NO: 48)
CCCCGGGTGCGCGGCGTCGGTGGTGCCGGCGGGGGGCGCCAGGTCGCAGG
CGGTGTAGGGCTCCAGGCAGGCGGCGAAGGCCATGACGTGCGCTATGAAG
GTCTGCTCCTGCACGCCGTGAACCAGGTGCGCCTGCGGGCCGCGCGCGAA
CACCGCCACGTCCTCGCCTGCGTGGGTCTCTTCGTCCAGGGGCACTGCTG
ACTGCTGCCGATACTCGGGGCTCCCGCTCTCGCTCTCGGTAACATCCGGC
CGGGCGCCGTCCTTGAGCACATAGCCTGGACCGTTTCCGTATAGGAGGAC
CGTGTAGGCCTTCCTGTCCCGGGCCTTGCCAGCGGCCAGCCCGATGAAGG
AGCTCCCTCGCAGGGGGTAGCCTCCGAAGGAGAAGACGTGGGAGTGGTCG
GCAGTGACGAGGCTCAGCGTGTCCTCCTCGCTGGTGAGCTGGCCCGCCCT
CTCAATGGCGTCGTCGAACATGATCGTCTCAGTCAGTGCCCGGTAAGCCC
TGCTTTCATGATGACCATGGTCGATGCGACCACCCTCCACGAAGAGGAAG
AAGCCGCGGGGGTGTCTGCTCAGCAGGCGCAGGGCAGCCTCTGTCATCTC
CATCAGGGAGGGGTCCAGTGTGGAGTCTCGGTGGATCTCGTATTTCATGT
CTCCAGGCTCAAAGAGACCCATGAGATGGGTCACAGACGGGTCCAGGGAA
GCCTGCATGAGCTCAGTGCGGTTCCACACGTACCGGGCACCCTGGCGTTC
GCCGAGCCATTCCTGCACCAGATTCTTCCCGTCCAGCCTGGTCCCACCTT
GGCTGTAGTCATCTGGGTACTCAGGGTCTGGGGTTCCCATGCGAAACATG
TACTTTCGGCCTCCA.

In some embodiments, the AP gene fragment has the sequence of:

(SEQ ID NO: 49)
CCCCGGGTGCGCGGCGTCGGTGGTGCCGGCGGGGGGCGCCAGGTCGCAGG
CGGTGTAGGGCTCCAGGCAGGCGGCGAAGGCCATGACGTGCGCTATGAAG
GTCTGCTCCTGCACGCCGTGAACCAGGTGCGCCTGCGGGCCGCGCGCGAA
CACCGCCACGTCCTCGCCTGCGTGGGTCTCTTCGTCCAGGGGCACTGCTG
ACTGCTGCCGATACTCGGGGCTCCCGCTCTCGCTCTCGGTAACATCCGGC
CGGGCGCCGTCCTTGAGCACATAGCCTGGACCGTTTCCGTATAGGAGGAC
CGTGTAGGCCTTCCTGTCCCGGGCCTTGCCAGCGGCCAGCCCGATGAAGG
AGCTCCCTCGCAGGGGGTAGCCTCCGAAGGAGAAGACGTGGGAGTGGTCG
GCAGTGACGAGGCTCAGCGTGTCCTCCTCG CTGGTGA.

In some embodiments, the AP gene fragment has the sequence of:

(SEQ ID NO: 50)
GCTGGCCCGCCCTCTCAATGGCGTCGTCGAACATGATCGTCTCAGTCAGT
GCCCGGTAAGCCCTGCTTTCATGATGACCATGGTCGATGCGACCACCCTC
CACGAAGAGGAAGAAGCCGCGGGGGTGTCTGCTCAGCAGGCGCAGGGCAG
CCTCTGTCATCTCCATCAGGGAGGGGTCCAGTGTGGAGTCTCGGTGGATC
TCGTATTTCATGTCTCCAGGCTCAAAGAGACCCATGAGATGGGTCACAGA
CGGGTCCAGGGAAGCCTGCATGAGCTCAGTGCGGTTCCACACGTACCGGG
CACCCTGGCGTTCGCCGAGCCATTCCTGCACCAGATTCTTCCCGTCCAGC
CTGGTCCCACCTTGGCTGTAGTCATCTGGGTACTCAGGGTCTGGGGTTCC
CATGCGAAACATGTACTTTCGGCCTCCA.

In some embodiments, the AP gene fragment has the sequence of:

(SEQ ID NO: 51)
CCCCGGGTGCGCGGCGTCGGTGGTGCCGGCGGGGGGCGCCAGGTCGCAGG
CGGTGTAGGGCTCCAGGCAGGCGGCGAAGGCCATGACGTGCGCTATGAAG
GTCTGCTCCTGCACGCCGTGAACCAGGTGCGCCTGCGGGCCGCGCGCGAA
CACCGCCACGTCCTCGCCTGCGTGGGTCTCTTCGTCCAGGGGCACTGCTG
ACTGCTGCCGATACTCGGGGCTCCCGCTCTCGCTCTCGGTAACATCCGGC
CGGGCGCCGTCCTTGAGCACATAGCCTGGACCGTTTC

In some embodiments, the AP gene fragment has the sequence of:

(SEQ ID NO: 52)
CGTATAGGAGGACCGTGTAGGCCTTCCTGTCCCGGGCCTTGCCAGCGGCC
AGCCCGATGAAGGAGCTCCCTCGCAGGGGGTAGCCTCCGAAGGAGAAGAC
GTGGGAGTGGTCGGCAGTGACGAGGCTCAGCGTGTCCTCCTCGCTGGTGA
GCTGGCCCGCCCTCTCAATGGCGTCGTCGAACATGATCGTCTCAGTCAGT
GCCCGGTAAGCCCTGCTTTCATGATGACCATGGTCGATGCGACCACCCTC
CACGAAGAGGAAGAAGCCGCGGGGGTGTCTGCTCAGCAGG.

In some embodiments, the AP gene fragment has the sequence of:

(SEQ ID NO: 53)
CGCAGGGCAGCCTCTGTCATCTCCATCAGGGAGGGGTCCAGTGTGGAGTC
TCGGTGGATCTCGTATTTCATGTCTCCAGGCTCAAAGAGACCCATGAGAT
GGGTCACAGACGGGTCCAGGGAAGCCTGCATGAGCTCAGTGCGGTTCCAC
ACGTACCGGGCACCCTGGCGTTCGCCGAGCCATTCCTGCACCAGATTCTT
CCCGTCCAGCCTGGTCCCACCTTGGCTGTAGTCATCTGGGTACTCAGGGT
CTGGGGTTCCCATGCGAAACATGTACTTTCGGCCTCCA.

An exemplary splice donor sequence for use in the methods and compositions described herein can include the sequence:

(SEQ ID NO: 54)
GTAAGTATCAAGGTTACAAGACAGGTTTAAGGAGACCAATAGAAACTGGG
CTTGTCGAGACAGAGAAGACTCTTGCGTTTCTGA.

An exemplary splice acceptor sequence for use in the methods and compositions described herein can include the sequence:

(SEQ ID NO: 55)
TAGGCACCTATTGGTCTTACTGACATCCACTTTGCCTTTCTCTCCACAG.

Additional examples of splice donor and splice acceptor sequences are known in the art.

Dual hybrid vectors for use in the methods and compositions described herein are designed such that approximately half of the OTOF gene is contained within each vector (e.g., each vector contains a polynucleotide that encodes approximately half of the OTOF isoform 5 protein). The determination of how to split the polynucleotide sequence between the two nucleic acid vectors can be made based on the size of the promoter and the locations of the portions of the polynucleotide that encode the OTOF C2 domains. When a short promoter is used in the dual hybrid vector system (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter), such as CAG, CMV, smCBA, or a Myo15 promoter having a sequence that is 1 kb or shorter (e.g., a Myo15 promoter described hereinabove, e.g., a Myo15 promoter having the sequence of SEQ ID NO: 21 or SEQ ID NO: 42), the OTOF polynucleotide sequence can be divided between the two nucleic acid vectors at an exon boundary that occurs after the portion of the polynucleotide that encodes the C2D domain and before the portion of the polynucleotide that encodes C2E domain, for example, the exon 26/27 boundary. The nucleic acid vectors containing promoters of this size can optionally contain OTOF UTRs (e.g., full-length 5′ and 3′ UTRs). When a long promoter is used in the dual hybrid vector system (e.g., a promoter that is longer than 1 kb, e.g., 1.1 kb, 1.25 kb, 1.5 kb, 1.75 kb, 2 kb, 2.5 kb, 3 kb or longer), such as a Myo15 promoter that is longer than 1 kb (e.g., SEQ ID NO: 19), the OTOF polynucleotide sequence can be divided between the two nucleic acid vectors at an exon boundary that occurs after the portion of the polynucleotide that encodes the C2C domain, and either before the portion of the polynucleotide that encodes the C2D domain, such as the exon 19/20 boundary or the exon 20/21 boundary, or within the portion of the polynucleotide that encodes the C2D domain, such as the exon 25/26 boundary. A short promoter (e.g., a CMV promoter, CAG promoter, smCBA promoter, or a Myo15 promoter having a sequence that is 1 kb or shorter, e.g., a Myo15 promoter having the sequence of SEQ ID NO: 21 or SEQ ID NO: 42) can also be used in the dual vector systems designed for large promoters, in which case additional elements (e.g., OTOF UTR sequences) may be included in the first vector (e.g., the vector containing the portion of the polynucleotide the encodes the C2C domain).

One exemplary dual hybrid vector system that uses a short promoter includes a first nucleic acid vector containing a CAG promoter operably linked to exons 1-26 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 27-45 and 47 of a polynucleotide encoding an OTOF isoform 5 protein (e.g., human OTOF isoform 5, e.g., SEQ ID NO: 1), and a poly(A) sequence (e.g., a bGH poly(A) signal sequence). The first and second nucleic acid vectors can also contain full-length 5′ and 3′ OTOF UTRs, respectively (e.g., the 127 bp human OTOF 5′ UTR can be included in the first nucleic acid vector, and the 1035 bp human OTOF 3′ UTR can be included in the second nucleic acid vector). Another exemplary dual hybrid vector system that uses a short promoter includes a first nucleic acid vector containing a smCBA promoter or a Myo15 promoter that is 1 kb or shorter (e.g., a Myo15 promoter having the sequence of SEQ ID NO: 21 or SEQ ID NO: 42) operably linked to exons 1-20 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 21-45 and 47 of a polynucleotide encoding an OTOF isoform 5 protein (e.g., human OTOF, e.g., SEQ ID NO: 1), and a poly(A) sequence (e.g., a bGH poly(A) signal sequence). The first nucleic acid vector can also contain the full-length 5′ OTOF UTRs (e.g., the 127 bp human OTOF 5′ UTR can be included in the first nucleic acid vector). The CMV promoter can be used in place of the CAG, smCBA, or Myo15 promoter in either of the foregoing dual vector systems.

An exemplary dual hybrid vector system that uses a long promoter includes a first nucleic acid vector containing a Myo 15 promoter that is longer than 1 kb (e.g., SEQ ID NO: 19) operably linked to exons 1-19 or exons 1-20 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 20-45 and 47 (when the first nucleic acid vector contains exons 1-19 of the polynucleotide) or exons 21-45 and 47 (when the first nucleic acid vector contains exons 1-20 of the polynucleotide) of a polynucleotide encoding an OTOF isoform 5 protein (e.g., human OTOF isoform 5, e.g., SEQ ID NO: 1), and a poly(A) sequence (e.g., a bGH poly(A) signal sequence). Neither the first nor the second nucleic acid vector in the foregoing Myo15 promoter dual hybrid vector system contains an OTOF UTR. A short promoter (e.g., a CMV promoter, CAG promoter, smCBA promoter, or a Myo 15 promoter having a sequence that is 1 kb or shorter, e.g., a Myo15 promoter having the sequence of SEQ ID NO: 21 or SEQ ID NO: 42) can also be used in the foregoing dual vector systems designed for large promoters. If these dual vector systems contain a short promoter, they may also include a 5′ OTOF UTR in the first vector.

To accommodate an OTOF UTR, the OTOF coding sequence can be divided in a different position. For example, in a dual hybrid vector system in which the first nucleic acid vector contains a Myo15 promoter that is longer than 1 kb (e.g., SEQ ID NO: 19) operably linked to exons 1-25 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and in which the second nucleic acid vector contains a recombinogenic region, a splice acceptor sequence 3′ of the recombinogenic region, a polynucleotide 3′ of the splice acceptor sequence that contains exons 26-45 and 47 of a polynucleotide encoding an OTOF protein (e.g., human OTOF isoform 5, e.g., SEQ ID NO: 1), and a poly(A) sequence (e.g., a bGH poly(A) signal sequence), the second nucleic acid can also contain a full-length OTOF 3′ UTR (e.g., the 1035 bp human OTOF UTR). A short promoter (e.g., a CMV promoter, CAG promoter, smCBA promoter, or a Myo15 promoter having a sequence that is 1 kb or shorter, e.g., a Myo15 promoter having the sequence of SEQ ID NO: 21 or SEQ ID NO: 42) can also be used in the foregoing dual vector system designed for large promoters. If these dual vector systems contain a short promoter, they may also include a 5′ OTOF UTR in the first vector.

The polynucleotide sequence encoding an OTOF isoform 5 protein can be a cDNA sequence (e.g., a sequence that does not include introns). In some embodiments, the first and/or the second nucleic acid vector in the dual vector system can include intronic sequence. The intronic sequence may be included between one or more exons in the OTOF coding sequence, or the intronic sequence can be included between an exon of the coding sequence and another component of the nucleic acid vector (e.g., between an exon of the OTOF coding sequence and the splice donor sequence in the first nucleic acid vector or between an exon of the OTOF coding sequence and the splice acceptor sequence in the second nucleic acid vector).

In some embodiments, the polynucleotide encoding OTOF isoform 5 is divided between the first and second nucleic acid vectors (e.g., AAV vectors) in the dual vector system at the exon 20/21 boundary. When the polynucleotide encoding OTOF isoform 5 is divided between the first and second nucleic acid vectors (e.g., AAV vectors) at the exon 20/21 boundary, the polynucleotide sequence encoding the N-terminal portion of OTOF has the sequence of:

(SEQ ID NO: 56)
ATGGCCTTGCTCATCCACCTCAAGACAGTCTCGGAGCTGCGGGGCAGGGGCGACC
GGATCGCCAAAGTGACTTTCCGAGGGCAATCCTTCTACTCTCGGGTCCTGGAGAAC
TGTGAGGATGTGGCTGACTTTGATGAGACATTTCGGTGGCCGGTGGCCAGCAGCAT
CGACAGAAATGAGATGCTGGAGATTCAGGTTTTCAACTACAGCAAAGTCTTCAGCAA
CAAGCTCATCGGGACCTTCCGCATGGTGCTGCAGAAGGTGGTAGAGGAGAGCCAT
GTGGAGGTGACTGACACGCTGATTGATGACAACAATGCTATCATCAAGACCAGCCT
GTGCGTGGAGGTCCGGTATCAGGCCACTGACGGCACAGTGGGCTCCTGGGACGAT
GGGGACTTCCTGGGAGATGAGTCTCTTCAAGAGGAAGAGAAGGACAGCCAAGAGA
CGGATGGACTGCTCCCAGGCTCCCGGCCCAGCTCCCGGCCCCCAGGAGAGAAGA
GCTTCCGGAGAGCCGGGAGGAGCGTGTTCTCCGCCATGAAGCTCGGCAAAAACCG
GTCTCACAAGGAGGAGCCCCAAAGACCAGATGAACCGGCGGTGCTGGAGATGGAA
GACCTTGACCATCTGGCCATTCGGCTAGGAGATGGACTGGATCCCGACTCGGTGTC
TCTAGCCTCAGTCACAGCTCTCACCACTAATGTCTCCAACAAGCGATCTAAGCCAGA
CATTAAGATGGAGCCAAGTGCTGGGCGGCCCATGGATTACCAGGTCAGCATCACGG
TGATCGAGGCCCGGCAGCTGGTGGGCTTGAACATGGACCCTGTGGTGTGCGTGGA
GGTGGGTGACGACAAGAAGTACACATCCATGAAGGAGTCCACTAACTGCCCCTATT
ACAACGAGTACTTCGTCTTCGACTTCCATGTCTCTCCGGATGTCATGTTTGACAAGA
TCATCAAGATTTCGGTGATTCACTCCAAGAACCTGCTGCGCAGTGGCACCCTGGTG
GGCTCCTTCAAAATGGACGTGGGAACCGTGTACTCGCAGCCAGAGCACCAGTTCCA
TCACAAGTGGGCCATCCTGTCTGACCCCGATGACATCTCCTCGGGGCTGAAGGGCT
ACGTGAAGTGTGACGTTGCCGTGGTGGGCAAAGGGGACAACATCAAGACGCCCCA
CAAGGCCAATGAGACCGACGAAGATGACATTGAGGGGAACTTGCTGCTCCCCGAG
GGGGTGCCCCCCGAACGCCAGTGGGCCCGGTTCTATGTGAAAATTTACCGAGCAG
AGGGGCTGCCCCGTATGAACACAAGCCTCATGGCCAATGTAAAGAAGGCTTTCATC
GGTGAAAACAAGGACCTCGTGGACCCCTACGTGCAAGTCTTCTTTGCTGGCCAGAA
GGGCAAGACTTCAGTGCAGAAGAGCAGCTATGAGCCCCTGTGGAATGAGCAGGTC
GTCTTTACAGACCTCTTCCCCCCACTCTGCAAACGCATGAAGGTGCAGATCCGAGA
CTCGGACAAGGTCAACGACGTGGCCATCGGCACCCACTTCATTGACCTGCGCAAGA
TTTCTAATGACGGAGACAAAGGCTTCCTGCCCACACTGGGCCCAGCCTGGGTGAAC
ATGTACGGCTCCACACGTAACTACACGCTGCTGGATGAGCATCAGGACCTGAACGA
GGGCCTGGGGGAGGGTGTGTCCTTCCGGGCCCGGCTCCTGCTGGGCCTGGCTGT
GGAGATCGTAGACACCTCCAACCCTGAGCTCACCAGCTCCACAGAGGTGCAGGTG
GAGCAGGCCACGCCCATCTCGGAGAGCTGTGCAGGTAAAATGGAAGAATTCTTTCT
CTTTGGAGCCTTCCTGGAGGCCTCAATGATCGACCGGAGAAACGGAGACAAGCCCA
TCACCTTTGAGGTCACCATAGGCAACTATGGGAACGAAGTTGATGGCCTGTCCCGG
CCCCAGCGGCCTCGGCCCCGGAAGGAGCCGGGGGATGAGGAAGAAGTAGACCTG
ATTCAGAACGCAAGTGATGACGAGGCCGGTGATGCCGGGGACCTGGCCTCAGTCT
CCTCCACTCCACCAATGCGGCCCCAGGTCACCGACAGGAACTACTTCCATCTGCCC
TACCTGGAGCGAAAGCCCTGCATCTACATCAAGAGCTGGTGGCCGGACCAGCGCC
GCCGCCTCTACAATGCCAACATCATGGACCACATTGCCGACAAGCTGGAAGAAGGC
CTGAACGACATACAGGAGATGATCAAAACGGAGAAGTCCTACCCTGAGCGTCGCCT
GCGGGGCGTCCTGGAGGAGCTGAGCTGTGGCTGCTGCCGCTTCCTCTCCCTCGCT
GACAAGGACCAGGGCCACTCATCCCGCACCAGGCTTGACCGGGAGCGCCTCAAGT
CCTGCATGAGGGAGCTG.

When the polynucleotide encoding OTOF isoform 5 is divided between the first and second nucleic acid vectors (e.g., AAV vectors) at the exon 20/21 boundary, the polynucleotide sequence encoding the C-terminal portion of OTOF has the sequence of:

(SEQ ID NO: 57)
GAAAACATGGGGCAGCAGGCCAGGATGCTGCGGGCCCAGGTGAAGCGGCACACG
GTGCGGGACAAGCTGAGGCTGTGCCAGAACTTCCTGCAGAAGCTGCGCTTCCTGG
CGGACGAGCCCCAGCACAGCATTCCCGACATCTTCATCTGGATGATGAGCAACAAC
AAGCGTGTCGCCTATGCCCGTGTGCCCTCCAAGGACCTGCTCTTCTCCATCGTGGA
GGAGGAGACTGGCAAGGACTGCGCCAAGGTCAAGACGCTCTTCCTTAAGCTGCCA
GGGAAGCGGGGCTTCGGCTCGGCAGGCTGGACAGTGCAGGCCAAGGTGGAGCTG
TACCTGTGGCTGGGCCTCAGCAAACAGCGCAAGGAGTTCCTGTGCGGCCTGCCCT
GTGGCTTCCAGGAGGTCAAGGCAGCCCAGGGCCTGGGCCTGCATGCCTTCCCACC
CGTCAGCCTGGTCTACACCAAGAAGCAGGCGTTCCAGCTCCGAGCGCACATGTACC
AGGCCCGCAGCCTCTTTGCCGCCGACAGCAGCGGACTCTCAGACCCCTTTGCCCG
CGTCTTCTTCATCAATCAGAGTCAGTGCACAGAGGTGCTGAATGAGACCCTGTGTCC
CACCTGGGACCAGATGCTGGTGTTCGACAACCTGGAGCTCTATGGTGAAGCTCATG
AGCTGAGGGACGATCCGCCCATCATTGTCATTGAAATCTATGACCAGGATTCCATGG
GCAAAGCTGACTTCATGGGCCGGACCTTCGCCAAACCCCTGGTGAAGATGGCAGAC
GAGGCGTACTGCCCACCCCGCTTCCCACCTCAGCTCGAGTACTACCAGATCTACCG
TGGCAACGCCACAGCTGGAGACCTGCTGGCGGCCTTCGAGCTGCTGCAGATTGGA
CCAGCAGGGAAGGCTGACCTGCCCCCCATCAATGGCCCGGTGGACGTGGACCGAG
GTCCCATCATGCCCGTGCCCATGGGCATCCGGCCCGTGCTCAGCAAGTACCGAGT
GGAGGTGCTGTTCTGGGGCCTACGGGACCTAAAGCGGGTGAACCTGGCCCAGGTG
GACCGGCCACGGGTGGACATCGAGTGTGCAGGGAAGGGGGTGCAGTCGTCCCTG
ATCCACAATTATAAGAAGAACCCCAACTTCAACACCCTCGTCAAGTGGTTTGAAGTG
GACCTCCCAGAGAACGAGCTGCTGCACCCGCCCTTGAACATCCGTGTGGTGGACT
GCCGGGCCTTCGGTCGCTACACACTGGTGGGCTCCCATGCCGTCAGCTCCCTGCG
ACGCTTCATCTACCGGCCCCCAGACCGCTCGGCCCCCAGCTGGAACACCACGGTC
AGGCTTCTCCGGCGCTGCCGTGTGCTGTGCAATGGGGGCTCCTCCTCTCACTCCAC
AGGGGAGGTTGTGGTGACTATGGAGCCAGAGGTACCCATCAAGAAACTGGAGACC
ATGGTGAAGCTGGACGCGACTTCTGAAGCTGTTGTCAAGGTGGATGTGGCTGAGGA
GGAGAAGGAGAAGAAGAAGAAGAAGAAGGGCACTGCGGAGGAGCCAGAGGAGGA
GGAGCCAGACGAGAGCATGCTGGACTGGTGGTCCAAGTACTTTGCCTCCATTGACA
CCATGAAGGAGCAACTTCGACAACAAGAGCCCTCTGGAATTGACTTGGAGGAGAAG
GAGGAAGTGGACAATACCGAGGGCCTGAAGGGGTCAATGAAGGGCAAGGAGAAGG
CAAGGGCTGCCAAAGAGGAGAAGAAGAAGAAAACTCAGAGCTCTGGCTCTGGCCA
GGGGTCCGAGGCCCCCGAGAAGAAGAAACCCAAGATTGATGAGCTTAAGGTATACC
CCAAAGAGCTGGAGTCCGAGTTTGATAACTTTGAGGACTGGCTGCACACTTTCAACT
TGCTTCGGGGCAAGACCGGGGATGATGAGGATGGCTCCACCGAGGAGGAGCGCAT
TGTGGGACGCTTCAAGGGCTCCCTCTGCGTGTACAAAGTGCCACTCCCAGAGGACG
TGTCCCGGGAAGCCGGCTACGACTCCACCTACGGCATGTTCCAGGGCATCCCGAG
CAATGACCCCATCAATGTGCTGGTCCGAGTCTATGTGGTCCGGGCCACGGACCTGC
ACCCTGCTGACATCAACGGCAAAGCTGACCCCTACATCGCCATCCGGCTAGGCAAG
ACTGACATCCGCGACAAGGAGAACTACATCTCCAAGCAGCTCAACCCTGTCTTTGG
GAAGTCCTTTGACATCGAGGCCTCCTTCCCCATGGAATCCATGCTGACGGTGGCTG
TGTATGACTGGGACCTGGTGGGCACTGATGACCTCATTGGGGAAACCAAGATCGAC
CTGGAGAACCGCTTCTACAGCAAGCACCGCGCCACCTGCGGCATCGCCCAGACCT
ACTCCACACATGGCTACAATATCTGGCGGGACCCCATGAAGCCCAGCCAGATCCTG
ACCCGCCTCTGCAAAGACGGCAAAGTGGACGGCCCCCACTTTGGGCCCCCTGGGA
GAGTGAAGGTGGCCAACCGCGTCTTCACTGGGCCCTCTGAGATTGAGGACGAGAA
CGGTCAGAGGAAGCCCACAGACGAGCATGTGGCGCTGTTGGCCCTGAGGCACTGG
GAGGACATCCCCCGCGCAGGCTGCCGCCTGGTGCCAGAGCATGTGGAGACGAGG
CCGCTGCTCAACCCCGACAAGCCGGGCATCGAGCAGGGCCGCCTGGAGCTGTGG
GTGGACATGTTCCCCATGGACATGCCAGCCCCTGGGACGCCTCTGGACATCTCACC
TCGGAAGCCCAAGAAGTACGAGCTGCGGGTCATCATCTGGAACACAGATGAGGTG
GTCTTGGAGGACGACGACTTCTTCACAGGGGAGAAGTCCAGTGACATCTTCGTGAG
GGGGTGGCTGAAGGGCCAGCAGGAGGACAAGCAGGACACAGACGTCCACTACCAC
TCCCTCACTGGCGAGGGCAACTTCAACTGGCGCTACCTGTTCCCCTTCGACTACCT
GGCGGCGGAGGAGAAGATCGTCATCTCCAAGAAGGAGTCCATGTTCTCCTGGGAC
GAGACCGAGTACAAGATCCCCGCGCGGCTCACCCTGCAGATCTGGGATGCGGACC
ACTTCTCCGCTGACGACTTCCTGGGGGCCATCGAGCTGGACCTGAACCGGTTCCCG
CGGGGCGCAAAGACAGCCAAGCAGTGCACCATGGAGATGGCCACCGGGGAGGTG
GACGTGCCCCTCGTGTCCATCTTCAAGCAAAAGCGCGTCAAAGGCTGGTGGCCCCT
CCTGGCCCGCAATGAGAACGATGAGTTTGAGCTCACGGGCAAGGTGGAGGCTGAG
CTGCATTTACTGACAGCAGAGGAGGCAGAGAAGAACCCAGTGGGCCTGGCCCGCA
ATGAACCTGACCCCCTAGAGAAACCCAACCGGCCCGACACGGCCTTCGTCTGGTTC
CTCAACCCTCTCAAGTCCATCAAGTACCTCATCTGCACCCGGTACAAGTGGCTCATC
ATCAAGATCGTGCTGGCGCTGTTGGGGCTGCTCATGTTGGGGCTCTTCCTCTACAG
CCTCCCTGGCTACATGGTCAAAAAGCTCCTTGGGGCATGA.

In embodiments in which the polynucleotide encoding OTOF isoform 5 is divided between the first and second nucleic acid vectors (e.g., AAV vectors) at the exon 20/21 boundary, the N-terminal portion of the OTOF polypeptide has the sequence of:

(SEQ ID NO: 58)
MALLIHLKTVSELRGRGDRIAKVTFRGQSFYSRVLENCEDVADFDETFRW
PVASSIDRNEMLEIQVFNYSKVFSNKLIGTFRMVLQKVVEESHVEVTDTL
IDDNNAIIKTSLCVEVRYQATDGTVGSWDDGDFLGDESLQEEEKDSQETD
GLLPGSRPSSRPPGEKSFRRAGRSVFSAMKLGKNRSHKEEPQRPDEPAVL
EMEDLDHLAIRLGDGLDPDSVSLASVTALTTNVSNKRSKPDIKMEPSAGR
PMDYQVSITVIEARQLVGLNMDPVVCVEVGDDKKYTSMKESTNCPYYNEY
FVFDFHVSPDVMFDKIIKISVIHSKNLLRSGTLVGSFKMDVGTVYSQPEH
QFHHKWAILSDPDDISSGLKGYVKCDVAVVGKGDNIKTPHKANETDEDDI
EGNLLLPEGVPPERQWARFYVKIYRAEGLPRMNTSLMANVKKAFIGENKD
LVDPYVQVFFAGQKGKTSVQKSSYEPLWNEQVVFTDLFPPLCKRMKVQIR
DSDKVNDVAIGTHFIDLRKISNDGDKGFLPTLGPAWVNMYGSTRNYTLLD
EHQDLNEGLGEGVSFRARLLLGLAVEIVDTSNPELTSSTEVQVEQATPIS
ESCAGKMEEFFLFGAFLEASMIDRRNGDKPITFEVTIGNYGNEVDGLSRP
QRPRPRKEPGDEEEVDLIQNASDDEAGDAGDLASVSSTPPMRPQVTDRNY
FHLPYLERKPCIYIKSWWPDQRRRLYNANIMDHIADKLEEGLNDIQEMIK
TEKSYPERRLRGVLEELSCGCCRFLSLADKDQGHSSRTRLDRERLKSCMR
EL.

In embodiments in which the polynucleotide encoding OTOF isoform 5 is divided between the first and second nucleic acid vectors (e.g., AAV vectors) at the exon 20/21 boundary, the C-terminal portion of the OTOF polypeptide has the sequence of:

(SEQ ID NO: 59)
ENMGQQARMLRAQVKRHTVRDKLRLCQNFLQKLRFLADEPQHSIPDIFIW
MMSNNKRVAYARVPSKDLLFSIVEEETGKDCAKVKTLFLKLPGKRGFGSA
GWTVQAKVELYLWLGLSKQRKEFLCGLPCGFQEVKAAQGLGLHAFPPVSL
VYTKKQAFQLRAHMYQARSLFAADSSGLSDPFARVFFINQSQCTEVLNET
LCPTWDQMLVFDNLELYGEAHELRDDPPIIVIEIYDQDSMGKADFMGRTF
AKPLVKMADEAYCPPRFPPQLEYYQIYRGNATAGDLLAAFELLQIGPAGK
ADLPPINGPVDVDRGPIMPVPMGIRPVLSKYRVEVLFWGLRDLKRVNLAQ
VDRPRVDIECAGKGVQSSLIHNYKKNPNFNTLVKWFEVDLPENELLHPPL
NIRVVDCRAFGRYTLVGSHAVSSLRRFIYRPPDRSAPSWNTTVRLLRRCR
VLCNGGSSSHSTGEVVVTMEPEVPIKKLETMVKLDATSEAVVKVDVAEEE
KEKKKKKKGTAEEPEEEEPDESMLDWWSKYFASIDTMKEQLRQQEPSGID
LEEKEEVDNTEGLKGSMKGKEKARAAKEEKKKKTQSSGSGQGSEAPEKKK
PKIDELKVYPKELESEFDNFEDWLHTFNLLRGKTGDDEDGSTEEERIVGR
FKGSLCVYKVPLPEDVSREAGYDSTYGMFQGIPSNDPINVLVRVYVVRAT
DLHPADINGKADPYIAIRLGKTDIRDKENYISKQLNPVFGKSFDIEASFP
MESMLTVAVYDWDLVGTDDLIGETKIDLENRFYSKHRATCGIAQTYSTHG
YNIWRDPMKPSQILTRLCKDGKVDGPHFGPPGRVKVANRVFTGPSEIEDE
NGQRKPTDEHVALLALRHWEDIPRAGCRLVPEHVETRPLLNPDKPGIEQG
RLELWVDMFPMDMPAPGTPLDISPRKPKKYELRVIIWNTDEVVLEDDDFF
TGEKSSDIFVRGWLKGQQEDKQDTDVHYHSLTGEGNFNWRYLFPFDYLAA
EEKIVISKKESMFSWDETEYKIPARLTLQIWDADHFSADDFLGAIELDLN
RFPRGAKTAKQCTMEMATGEVDVPLVSIFKQKRVKGWWPLLARNENDEFE
LTGKVEAELHLLTAEEAEKNPVGLARNEPDPLEKPNRPDTAFVWFLNPLK
SIKYLICTRYKWLIIKIVLALLGLLMLGLFLYSLPGYMVKKLLGA.

Transfer plasmids that may be used to produce the nucleic acid vectors for use in the compositions and methods described herein are provided in Table 4. A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. Nucleic acid vectors (e.g., a nucleic acid vector (e.g., an AAV vector) containing a polynucleotide encoding an N-terminal portion of OTOF isoform 5 and a nucleic acid vector (e.g., an AAV vector) containing a polynucleotide encoding a C-terminal portion of OTOF isoform 5) can be combined (e.g., in a single formulation) prior to administration. The following transfer plasmids are designed to produce nucleic acid vectors (e.g., AAV vectors) for co-formulation or co-administration (e.g., administration simultaneously or sequentially) in a dual hybrid vector system: SEQ ID NO: 60 and SEQ ID NO: 61; SEQ ID NO: 62 and SEQ ID NO: 63; SEQ ID NO: 64 and SEQ ID NO: 61; SEQ ID NO: 65 and SEQ ID NO: 63; SEQ ID NO: 66 and SEQ ID NO: 67; and SEQ ID NO: 68 and SEQ ID NO: 67.

TABLE 4
Transfer plasmids for the production of dual hybrid vector systems

| SEQ | | |
|---|---|---|
| ID | | |
| NO. | Description | Plasmid Sequence |
| 60 | 5′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda (Biologically inert and | TTTCCATCATTCCAGTCGAACTCACACACAACACCAAA |
| | inactivated DNA derived from | TGCATTTAAGTCGCTTGAAATTGCTATAAGCAGAGCAT |
| | bacteriophage lambda to | GTTGCGCCAGCATGATTAATACAGCATTTAATACAGAG |
| | reduce off-target DNA | CCGTGTTTATTGAGTCGGTATTCAGAGTCTGACCAGAA |
| | encapsidation) at positions 53- | ATTATTAATCTGGTGAAGTTATTCCTCTGTCATTACGTC |
| | 2027 | ATGGTCGATTTCAATTTCTATTGATGCTTTCCAGTCGTA |
| | ITR at positions 2049-2178 | ATCAATGATGTATTTTTTGATGTTTGACCTCTGTTCATA |
| | Myo15 promoter at positions | TCCTCACAGATAAAAAATCGCCCTCACACTGGAGGGC |
| | 2272-3236 | AAAGAAGATTTCCAATAATCAGAACAAGTCGGCTCCTG |
| | Kozak sequence (Site to initiate | TTTAGTTACGAGCGACATTGCTCCGTGTATTCACTCGT |
| | protein translation) at positions | TGGAATGAATACACAGTGCAGTGTTTATTCTGTTATTTA |
| | 3253-3262 | TGCCAAAAATTAAGGCCACTATCAGGCAGCTTTGTTGT |
| | N-terminal portion of human | TCTGTTTACCAAGTTCTCTGGCAATCATTGCCGTCGTT |
| | OTOF isoform 5 at positions | CGTATTGCCCATTTATCGACATATTTCCCATCTTCCTAT |
| | 3259-5664 | ACAGGAAACATTTCTTCAGGCTTAACCATGCATTCCGA |
| | Splice donor (APSD) sequence | TTGCAGCTTGCATCCATTGCATCGCTTGAATTGTCCAC |
| | at positions 5665-5748 | ACCATTGATTTTTATCAATAGTCGTAGTTTAACGGATAG |
| | AP head sequence | TCCTGGTATTGTTCCATCACATCCTGAGGATGCCCTTC |
| | (recombinogenic region) at | GAACTCTTCAAATTCTTCTTCCTAATATCACCTTAAATA |
| | positions 5755-6041 | GTGGATTGCGGTAGTAAAGATTGTGCCTGTCTTTTAAC |
| | ITR at positions 6135-6264 | CACATCAGGCTCGGTGGTTCTCGTGTACCCCTACAGC |
| | Lambda at positions 6275-8287 | GAGAAATCGGATAAACTATTACAACCCCTACAGTTTGT |
| | Ori (origin of replication) at | AGAGTATAGAAAATGATCCACTCGTTATTCTCGGACGA |
| | positions 8344-8932 | GTGTTCAGTAATGAACCTCTGGAGAGAACCATCTATAT |
| | KanR (antibiotic resistance | GATCGTTATCTGGGTTTGACTTCTGCTTTTAAGCCCAG |
| | gene) at positions 9110-9919 | ATAACTTGCCTGAATATGTTAATGAGAGAATCGGTATT |
| | Transgene to be transferred | CCTCATGTGTGGCATGTTTTCGTCTTTGCTCTTGCATTT |
| | into vector in dual vector | TCACTAGCAATTAATGTGCATCGATTATCAGCTATTGC |
| | system at positions 2049-6264 | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCCAATTC |
| | | TGCAGCTCAGCCTACTACTTGCTTTCCAGGCTGTTCCT |
| | | AGTTCCCATGTCAGCTGCTTGTGCTTTCCAGAGACAAA |
| | | ACAGGAATAATAGATGTCATTAAATATACATTGGGCCC |
| | | CAGGCGGTCAATGTGGCAGCCTGAGCCTCCTTTCCAT |
| | | CTCTGTGGAGGCAGACATAGGACCCCCAACAAACAGC |
| | | ATGCAGGTTGGGAGCCAGCCACAGGACCCAGGTAAG |
| | | GGGCCCTGGGTCCTTAAGCTTCTGCCACTGGCTCCGG |
| | | CATTGCAGAGAGAAGAGAAGGGGCGGCAGACTGGAG |
| | | AGCTGGGCTCCATTTTTGTTCCTTGGTGCCCTGCCCCT |
| | | CCCCATGACCTGCAGAGACATTCAGCCTGCCAGGCTT |
| | | TATGAGGTGGGAGCTGGGCTCTCCCTGATGTATTATTC |
| | | AGCTCCCTGGAGTTGGCCAGCTCCTGTTACACTGGCC |
| | | ACAGCCCTGGGCATCCGCTTCTCACTTCTAGTTTCCCC |
| | | TCCAAGGTAATGTGGTGGGTCATGATCATTCTATCCTG |
| | | GCTTCAGGGACCTGACTCCACTTTGGGGCCATTCGAG |
| | | GGGTCTAGGGTAGATGATGTCCCCCTGTGGGGATTAA |
| | | TGTCCTGCTCTGTAAAACTGAGCTAGCTGAGATCCAG |
| | | GAGGGCTTGGCCAGAGACAGCAAGTTGTTGCCATGGT |
| | | GACTTTAAAGCCAGGTTGCTGCCCCAGCACAGGCCTC |
| | | CCAGTCTACCCTCACTAGAAAACAACACCCAGGCACTT |
| | | TCCACCACCTCTCAAAGGTGAAACCCAAGGCTGGTCT |
| | | AGAGAATGAATTATGGATCCTCGCTGTCCGTGCCACC |
| | | CAGCTAGTCCCAGCGGCTCAGACACTGAGGAGAGACT |
| | | GTAGGTTCAGCTACAAGCAAAAAGACCTAGCTGGTCT |
| | | CCAAGCAGTGTCTCCAAGTCCCTGAACCTGTGACACC |
| | | TGCCCCAGGCATCATCAGGCACAGAGGGCCACCAAGA |
| | | ATTCTAGCGGCCGCCACCATGGCCTTGCTCATCCACC |
| | | TCAAGACAGTCTCGGAGCTGCGGGGCAGGGGCGACC |
| | | GGATCGCCAAAGTGACTTTCCGAGGGCAATCCTTCTA |
| | | CTCTCGGGTCCTGGAGAACTGTGAGGATGTGGCTGAC |
| | | TTTGATGAGACATTTCGGTGGCCGGTGGCCAGCAGCA |
| | | TCGACAGAAATGAGATGCTGGAGATTCAGGTTTTCAAC |
| | | TACAGCAAAGTCTTCAGCAACAAGCTCATCGGGACCTT |
| | | CCGCATGGTGCTGCAGAAGGTGGTAGAGGAGAGCCA |
| | | TGTGGAGGTGACTGACACGCTGATTGATGACAACAAT |
| | | GCTATCATCAAGACCAGCCTGTGCGTGGAGGTCCGGT |
| | | ATCAGGCCACTGACGGCACAGTGGGCTCCTGGGACG |
| | | ATGGGGACTTCCTGGGAGATGAGTCTCTTCAAGAGGA |
| | | AGAGAAGGACAGCCAAGAGACGGATGGACTGCTCCCA |
| | | GGCTCCCGGCCCAGCTCCCGGCCCCCAGGAGAGAAG |
| | | AGCTTCCGGAGAGCCGGGAGGAGCGTGTTCTCCGCC |
| | | ATGAAGCTCGGCAAAAACCGGTCTCACAAGGAGGAGC |
| | | CCCAAAGACCAGATGAACCGGCGGTGCTGGAGATGG |
| | | AAGACCTTGACCATCTGGCCATTCGGCTAGGAGATGG |
| | | ACTGGATCCCGACTCGGTGTCTCTAGCCTCAGTCACA |
| | | GCTCTCACCACTAATGTCTCCAACAAGCGATCTAAGCC |
| | | AGACATTAAGATGGAGCCAAGTGCTGGGCGGCCCATG |
| | | GATTACCAGGTCAGCATCACGGTGATCGAGGCCCGGC |
| | | AGCTGGTGGGCTTGAACATGGACCCTGTGGTGTGCGT |
| | | GGAGGTGGGTGACGACAAGAAGTACACATCCATGAAG |
| | | GAGTCCACTAACTGCCCCTATTACAACGAGTACTTCGT |
| | | CTTCGACTTCCATGTCTCTCCGGATGTCATGTTTGACA |
| | | AGATCATCAAGATTTCGGTGATTCACTCCAAGAACCTG |
| | | CTGCGCAGTGGCACCCTGGTGGGCTCCTTCAAAATGG |
| | | ACGTGGGAACCGTGTACTCGCAGCCAGAGCACCAGTT |
| | | CCATCACAAGTGGGCCATCCTGTCTGACCCCGATGAC |
| | | ATCTCCTCGGGGCTGAAGGGCTACGTGAAGTGTGACG |
| | | TTGCCGTGGTGGGCAAAGGGGACAACATCAAGACGCC |
| | | CCACAAGGCCAATGAGACCGACGAAGATGACATTGAG |
| | | GGGAACTTGCTGCTCCCCGAGGGGGTGCCCCCCGAA |
| | | CGCCAGTGGGCCCGGTTCTATGTGAAAATTTACCGAG |
| | | CAGAGGGGCTGCCCCGTATGAACACAAGCCTCATGGC |
| | | CAATGTAAAGAAGGCTTTCATCGGTGAAAACAAGGACC |
| | | TCGTGGACCCCTACGTGCAAGTCTTCTTTGCTGGCCA |
| | | GAAGGGCAAGACTTCAGTGCAGAAGAGCAGCTATGAG |
| | | CCCCTGTGGAATGAGCAGGTCGTCTTTACAGACCTCTT |
| | | CCCCCCACTCTGCAAACGCATGAAGGTGCAGATCCGA |
| | | GACTCGGACAAGGTCAACGACGTGGCCATCGGCACC |
| | | CACTTCATTGACCTGCGCAAGATTTCTAATGACGGAGA |
| | | CAAAGGCTTCCTGCCCACACTGGGCCCAGCCTGGGTG |
| | | AACATGTACGGCTCCACACGTAACTACACGCTGCTGG |
| | | ATGAGCATCAGGACCTGAACGAGGGCCTGGGGGAGG |
| | | GTGTGTCCTTCCGGGCCCGGCTCCTGCTGGGCCTGG |
| | | CTGTGGAGATCGTAGACACCTCCAACCCTGAGCTCAC |
| | | CAGCTCCACAGAGGTGCAGGTGGAGCAGGCCACGCC |
| | | CATCTCGGAGAGCTGTGCAGGTAAAATGGAAGAATTC |
| | | TTTCTCTTTGGAGCCTTCCTGGAGGCCTCAATGATCGA |
| | | CCGGAGAAACGGAGACAAGCCCATCACCTTTGAGGTC |
| | | ACCATAGGCAACTATGGGAACGAAGTTGATGGCCTGT |
| | | CCCGGCCCCAGCGGCCTCGGCCCCGGAAGGAGCCG |
| | | GGGGATGAGGAAGAAGTAGACCTGATTCAGAACGCAA |
| | | GTGATGACGAGGCCGGTGATGCCGGGGACCTGGCCT |
| | | CAGTCTCCTCCACTCCACCAATGCGGCCCCAGGTCAC |
| | | CGACAGGAACTACTTCCATCTGCCCTACCTGGAGCGA |
| | | AAGCCCTGCATCTACATCAAGAGCTGGTGGCCGGACC |
| | | AGCGCCGCCGCCTCTACAATGCCAACATCATGGACCA |
| | | CATTGCCGACAAGCTGGAAGAAGGCCTGAACGACATA |
| | | CAGGAGATGATCAAAACGGAGAAGTCCTACCCTGAGC |
| | | GTCGCCTGCGGGGCGTCCTGGAGGAGCTGAGCTGTG |
| | | GCTGCTGCCGCTTCCTCTCCCTCGCTGACAAGGACCA |
| | | GGGCCACTCATCCCGCACCAGGCTTGACCGGGAGCG |
| | | CCTCAAGTCCTGCATGAGGGAGCTGGTAAGTATCAAG |
| | | GTTACAAGACAGGTTTAAGGAGACCAATAGAAACTGG |
| | | GCTTGTCGAGACAGAGAAGACTCTTGCGTTTCTGAGC |
| | | TAGCCCCCGGGTGCGCGGCGTCGGTGGTGCCGGCG |
| | | GGGGGCGCCAGGTCGCAGGCGGTGTAGGGCTCCAG |
| | | GCAGGCGGCGAAGGCCATGACGTGCGCTATGAAGGT |
| | | CTGCTCCTGCACGCCGTGAACCAGGTGCGCCTGCGG |
| | | GCCGCGCGCGAACACCGCCACGTCCTCGCCTGCGTG |
| | | GGTCTCTTCGTCCAGGGGCACTGCTGACTGCTGCCGA |
| | | TACTCGGGGCTCCCGCTCTCGCTCTCGGTAACATCCG |
| | | GCCGGGCGCCGTCCTTGAGCACATAGCCTGGACCGTT |
| | | TCGTCGACCTCGAGTTAAGGGCGAATTCCCGATAAGG |
| | | ATCTTCCTAGAGCATGGCTACGTAGATAAGTAGCATGG |
| | | CGGGTTAATCATTAACTACAAGGAACCCCTAGTGATGG |
| | | AGTTGGCCACTCCCTCTCTGCGCGCTCGCTCGCTCAC |
| | | TGAGGCCGGGCGACCAAAGGTCGCCCGACGCCCGGG |
| | | CTTTGCCCGGGCGGCCTCAGTGAGCGAGCGAGCGCG |
| | | CAGCCTTAATTAAATCCACATCTGTATGTTTTTTATATT |
| | | AATTTATTTTTTGCAGGGGGGCATTGTTTGGTAGGTGA |
| | | GAGTTCTGAATTGCTATGTTTAGTGAGTTGTATCTATTT |
| | | ATTTTTCAATAAATACAATTAGTTATGTGTTTTGGGGGC |
| | | GATCGTGAGGCAAAGAAAACCCGGCGCTGAGGCCGG |
| | | GTTATTCTTGTTCTCTGGTCAAATTATATAGTTGGAAAA |
| | | CAAGGATGCATATATGAATGAACGATGCAGAGGCAAT |
| | | GCCGATGGCGATAGTGGGTATCAGGTAGCCGCTTATG |
| | | CTGGAAAGAAGCAATAACCCGCAGAAAAACAAAGCTC |
| | | CAAGCTCAACAAAACTAAGGGCATAGACAATAACTACC |
| | | TATGTCATATACCCATACTCTCTAATCTTGGCCAGTCG |
| | | GCGCGTTCTGCTTCCGATTAGAAACGTCAAGGCAGCA |
| | | ATCAGGATTGCAATCTTGGTTCCTGCATAGGATGACAA |
| | | TGTCGCCCCAAGACCATCTCTATGAGCTGAAAAAGAAA |
| | | CACAAGGAATGTAGTGGCGGAAAAGGAGATAGCAAAT |
| | | GCTTACGATAACGTAAGGAATTATTACTATGTAAACAC |
| | | CAGGCAAGATTCTGTTCCGTATAATTACTCCTGATAATT |
| | | AATCCTTAACTTTGCCCACCTGCCTTTTAAAACATTCCA |
| | | GTATATCACTTTTCATTCTTGCGTAGCAATATGCCCTCT |
| | | CTTCAGCTATCTCAGCATTGGTGACCTTGTTCAGAGGC |
| | | GCTGAGAGATGGCCTTTTTCTGATAGATAATGTTCTGT |
| | | TAAAATATCTCCGGCCTCATCTTTTGCCCGCAGGCTAA |
| | | TGTCTGAAAATTGAGGTGACGGGTTAAAAATAATATCC |
| | | TTGGCAACCTTTTTTATATCCCTTTTAAATTTTGGCTTA |
| | | ATGACTATATCCAATGAGTCAAAAAGCTCCCCTTCAAT |
| | | ATCTGTTGCCCCTAAGACCTTTAATATATCGCCAAATA |
| | | CAGGTAGCTTGGCTTCTACCTTCACCGTTGTTCTGCCG |
| | | ATGAAATGCTAATGCATAACATCGTCTTTGGTGGTTCC |
| | | CCTCATCAGTGGCTCTATCTGAACGCGCTCTCCACTG |
| | | CTTAATGACATTCCTTTCCCGATTAAAAAATCTGTCAGA |
| | | TCGGATGTGGTCGGCCCGAAAACAGTTCTGGCAAAAC |
| | | CAATGGTGTCGCCTTCAACAAACAAAAAAGATGGGAAT |
| | | CCCAATGATTCGTCATCTGCGAGGCTGTTCTTAATATC |
| | | TTCAACTGTAGCTTTAGAGCGATTTATCTTCTGAACCA |
| | | GACTCTTGTCATTTGTTTTGGTAAAGAGAAAAGTTTTTC |
| | | CATCGATTTTATGAATATACAAATAATTGGAGCCAACCT |
| | | TCAGGTGATGATTATCAGCCAGCAGAGAATTAAGGAAA |
| | | ACAGACAGGTTTATTGAGCACTTATCTTTCCCTTTATTT |
| | | TTGCTGCGGTAAGTCGCATAAAAACCATTCTTCACAAT |
| | | TCAATCCATTTACTATGTTATGTTCTGAGGGGAGTGAA |
| | | AATTCCCCTAATTCGATGAAGATTCTTGCTAAATTGTTA |
| | | TCAGCTATGCGCCGACCAGAACACCTTGCCGATCAGC |
| | | CAAACGTCTAATCAGGCCACTGACTAGCGATAACTTTC |
| | | CCCACAACGGAACAACTCTCATTGCATGGGATAATTGG |
| | | GTACTGTGGGTTTAGTGGTTGTAAAAACACCTGACCGC |
| | | TATCCCTGATCAGTTTCTTGAAGGTAAACTCATCACCC |
| | | CCAAGTCTGGCTATACAGAAATCACCTGGCTCAACAG |
| | | CCTGCTCAGGGTCAACGAGAATTTACATTCCGTCAGG |
| | | ATAGCTTGGCTTGGAGCCTGTTGGTGCGGTCACGGAA |
| | | TTACCTTCAACCTCAAGCCAGAATGCAGAATCACTGGC |
| | | TTTTTTGGTTGTGCTTACCCATCTCTCCGCATCACCTTT |
| | | GGTAAAGGTTCTAAGCTAAGGTGAGAACATCCCTGCC |
| | | TGAACATGAGAAAAAACAGGGTACTCATACTCACTTAT |
| | | TAGTGACGGCTATGAGCAAAAGGCCAGCAAAAGGCCA |
| | | GGAACCGTAAAAAGGCCGCGTTGCTGGCGTTTTTCCA |
| | | TAGGCTCCGCCCCCCTGACGAGCATCACAAAAATCGA |
| | | CGCTCAAGTCAGAGGTGGCGAAACCCGACAGGACTAT |
| | | AAAGATACCAGGCGTTTCCCCCTGGAAGCTCCCTCGT |
| | | GCGCTCTCCTGTTCCGACCCTGCCGCTTACCGGATAC |
| | | CTGTCCGCCTTTCTCCCTTCGGGAAGCGTGGCGCTTT |
| | | CTCATAGCTCACGCTGTAGGTATCTCAGTTCGGTGTAG |
| | | GTCGTTCGCTCCAAGCTGGGCTGTGTGCACGAACCCC |
| | | CCGTTCAGCCCGACCGCTGCGCCTTATCCGGTAACTA |
| | | TCGTCTTGAGTCCAACCCGGTAAGACACGACTTATCG |
| | | CCACTGGCAGCAGCCACTGGTAACAGGATTAGCAGAG |
| | | CGAGGTATGTAGGCGGTGCTACAGAGTTCTTGAAGTG |
| | | GTGGCCTAACTACGGCTACACTAGAAGAACAGTATTTG |
| | | GTATCTGCGCTCTGCTGAAGCCAGTTACCTTCGGAAAA |
| | | AGAGTTGGTAGCTCTTGATCCGGCAAACAAACCACCG |
| | | CTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAAC |
| | | TCACGTTAAGGGATTTTGGTCATGAGATTATCAAAAAG |
| | | GATCTTCACCTAGATCCTTTTAAATTAAAAATGAAGTTT |
| | | TAAATCAAGCCCAATCTGAATAATGTTACAACCAATTAA |
| | | CCAATTCTGATTAGAAAAACTCATCGAGCATCAAATGA |
| | | AACTGCAATTTATTCATATCAGGATTATCAATACCATAT |
| | | TTTTGAAAAAGCCGTTTCTGTAATGAAGGAGAAAACTC |
| | | ACCGAGGCAGTTCCATAGGATGGCAAGATCCTGGTAT |
| | | CGGTCTGCGATTCCGACTCGTCCAACATCAATACAACC |
| | | TATTAATTTCCCCTCGTCAAAAATAAGGTTATCAAGTGA |
| | | GAAATCACCATGAGTGACGACTGAATCCGGTGAGAAT |
| | | GGCAAAAGTTTATGCATTTCTTTCCAGACTTGTTCAACA |
| | | GGCCAGCCATTACGCTCGTCATCAAAATCACTCGCATC |
| | | AACCAAACCGTTATTCATTCGTGATTGCGCCTGAGCAA |
| | | GACGAAATACGCGATCGCTGTTAAAAGGACAATTACAA |
| | | ACAGGAATCGAATGCAACCGGCGCAGGAACACTGCCA |
| | | GCGCATCAACAATATTTTCACCTGAATCAGGATATTCTT |
| | | CTAATACCTGGAATGCTGTTTTTCCGGGGATCGCAGTG |
| | | GTGAGTAACCATGCATCATCAGGAGTACGGATAAAATG |
| | | CTTGATGGTCGGAAGAGGCATAAATTCCGTCAGCCAG |
| | | TTTAGTCTGACCATCTCATCTGTAACATCATTGGCAAC |
| | | GCTACCTTTGCCATGTTTCAGAAACAACTCTGGCGCAT |
| | | CGGGCTTCCCATACAAGCGATAGATTGTCGCACCTGA |
| | | TTGCCCGACATTATCGCGAGCCCATTTATACCCATATA |
| | | AATCAGCATCCATGTTGGAATTTAATCGCGGCCTCGAC |
| | | GTTTCCCGTTGAATATGGCTCATAACACCCCTTGTATT |
| | | ACTGTTTATGTAAGCAGACAGTTTTATTGTTCATGATGA |
| | | TATATTTTTATCTTGTGCAATGTAACATCAGAGATTTTG |
| | | AGACACGGGCCAGAGCTGCA |
| 61 | 3′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda at positions 53-2027 | TTTCCATCATTCCAGTCGAACTCACACACAACACCAAA |
| | ITR at positions 2049-2178 | TGCATTTAAGTCGCTTGAAATTGCTATAAGCAGAGCAT |
| | AP head sequence at positions | GTTGCGCCAGCATGATTAATACAGCATTTAATACAGAG |
| | 2267-2553 | CCGTGTTTATTGAGTCGGTATTCAGAGTCTGACCAGAA |
| | Splice acceptor sequence at | ATTATTAATCTGGTGAAGTTATTCCTCTGTCATTACGTC |
| | positions 2576-2624 | ATGGTCGATTTCAATTTCTATTGATGCTTTCCAGTCGTA |
| | C-terminal portion of human | ATCAATGATGTATTTTTTGATGTTTGACCTCTGTTCATA |
| | OTOF isoform 5 at positions | TCCTCACAGATAAAAAATCGCCCTCACACTGGAGGGC |
| | 2625-6212 | AAAGAAGATTTCCAATAATCAGAACAAGTCGGCTCCTG |
| | bGH poly(A) sequence at | TTTAGTTACGAGCGACATTGCTCCGTGTATTCACTCGT |
| | positions 6255-6476 | TGGAATGAATACACAGTGCAGTGTTTATTCTGTTATTTA |
| | ITR at positions 6564-6693 | TGCCAAAAATTAAGGCCACTATCAGGCAGCTTTGTTGT |
| | Lambda at positions 6704-8716 | TCTGTTTACCAAGTTCTCTGGCAATCATTGCCGTCGTT |
| | Ori at positions 8773-9361 | CGTATTGCCCATTTATCGACATATTTCCCATCTTCCTAT |
| | KanR at positions 9539-10,348 | ACAGGAAACATTTCTTCAGGCTTAACCATGCATTCCGA |
| | Transgene to be transferred | TTGCAGCTTGCATCCATTGCATCGCTTGAATTGTCCAC |
| | into vector in dual vector | ACCATTGATTTTTATCAATAGTCGTAGTTTAACGGATAG |
| | system at positions 2049-6693 | TCCTGGTATTGTTCCATCACATCCTGAGGATGCCCTTC |
| | | GAACTCTTCAAATTCTTCTTCCTAATATCACCTTAAATA |
| | | GTGGATTGCGGTAGTAAAGATTGTGCCTGTCTTTTAAC |
| | | CACATCAGGCTCGGTGGTTCTCGTGTACCCCTACAGC |
| | | GAGAAATCGGATAAACTATTACAACCCCTACAGTTTGT |
| | | AGAGTATAGAAAATGATCCACTCGTTATTCTCGGACGA |
| | | GTGTTCAGTAATGAACCTCTGGAGAGAACCATCTATAT |
| | | GATCGTTATCTGGGTTTGACTTCTGCTTTTAAGCCCAG |
| | | ATAACTTGCCTGAATATGTTAATGAGAGAATCGGTATT |
| | | CCTCATGTGTGGCATGTTTTCGTCTTTGCTCTTGCATTT |
| | | TCACTAGCAATTAATGTGCATCGATTATCAGCTATTGC |
| | | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCCCCCG |
| | | GGTGCGCGGCGTCGGTGGTGCCGGCGGGGGGCGCC |
| | | AGGTCGCAGGCGGTGTAGGGCTCCAGGCAGGCGGCG |
| | | AAGGCCATGACGTGCGCTATGAAGGTCTGCTCCTGCA |
| | | CGCCGTGAACCAGGTGCGCCTGCGGGCCGCGCGCGA |
| | | ACACCGCCACGTCCTCGCCTGCGTGGGTCTCTTCGTC |
| | | CAGGGGCACTGCTGACTGCTGCCGATACTCGGGGCT |
| | | CCCGCTCTCGCTCTCGGTAACATCCGGCCGGGCGCC |
| | | GTCCTTGAGCACATAGCCTGGACCGTTTCCTTAAGCG |
| | | ACGCATGCTCGCGATAGGCACCTATTGGTCTTACTGA |
| | | CATCCACTTTGCCTTTCTCTCCACAGGAAAACATGGGG |
| | | CAGCAGGCCAGGATGCTGCGGGCCCAGGTGAAGCGG |
| | | CACACGGTGCGGGACAAGCTGAGGCTGTGCCAGAAC |
| | | TTCCTGCAGAAGCTGCGCTTCCTGGCGGACGAGCCCC |
| | | AGCACAGCATTCCCGACATCTTCATCTGGATGATGAGC |
| | | AACAACAAGCGTGTCGCCTATGCCCGTGTGCCCTCCA |
| | | AGGACCTGCTCTTCTCCATCGTGGAGGAGGAGACTGG |
| | | CAAGGACTGCGCCAAGGTCAAGACGCTCTTCCTTAAG |
| | | CTGCCAGGGAAGCGGGGCTTCGGCTCGGCAGGCTGG |
| | | ACAGTGCAGGCCAAGGTGGAGCTGTACCTGTGGCTG |
| | | GGCCTCAGCAAACAGCGCAAGGAGTTCCTGTGCGGC |
| | | CTGCCCTGTGGCTTCCAGGAGGTCAAGGCAGCCCAG |
| | | GGCCTGGGCCTGCATGCCTTCCCACCCGTCAGCCTG |
| | | GTCTACACCAAGAAGCAGGCGTTCCAGCTCCGAGCGC |
| | | ACATGTACCAGGCCCGCAGCCTCTTTGCCGCCGACAG |
| | | CAGCGGACTCTCAGACCCCTTTGCCCGCGTCTTCTTC |
| | | ATCAATCAGAGTCAGTGCACAGAGGTGCTGAATGAGA |
| | | CCCTGTGTCCCACCTGGGACCAGATGCTGGTGTTCGA |
| | | CAACCTGGAGCTCTATGGTGAAGCTCATGAGCTGAGG |
| | | GACGATCCGCCCATCATTGTCATTGAAATCTATGACCA |
| | | GGATTCCATGGGCAAAGCTGACTTCATGGGCCGGACC |
| | | TTCGCCAAACCCCTGGTGAAGATGGCAGACGAGGCGT |
| | | ACTGCCCACCCCGCTTCCCACCTCAGCTCGAGTACTA |
| | | CCAGATCTACCGTGGCAACGCCACAGCTGGAGACCTG |
| | | CTGGCGGCCTTCGAGCTGCTGCAGATTGGACCAGCAG |
| | | GGAAGGCTGACCTGCCCCCCATCAATGGCCCGGTGG |
| | | ACGTGGACCGAGGTCCCATCATGCCCGTGCCCATGG |
| | | GCATCCGGCCCGTGCTCAGCAAGTACCGAGTGGAGG |
| | | TGCTGTTCTGGGGCCTACGGGACCTAAAGCGGGTGAA |
| | | CCTGGCCCAGGTGGACCGGCCACGGGTGGACATCGA |
| | | GTGTGCAGGGAAGGGGGTGCAGTCGTCCCTGATCCA |
| | | CAATTATAAGAAGAACCCCAACTTCAACACCCTCGTCA |
| | | AGTGGTTTGAAGTGGACCTCCCAGAGAACGAGCTGCT |
| | | GCACCCGCCCTTGAACATCCGTGTGGTGGACTGCCGG |
| | | GCCTTCGGTCGCTACACACTGGTGGGCTCCCATGCCG |
| | | TCAGCTCCCTGCGACGCTTCATCTACCGGCCCCCAGA |
| | | CCGCTCGGCCCCCAGCTGGAACACCACGGTCAGGCT |
| | | TCTCCGGCGCTGCCGTGTGCTGTGCAATGGGGGCTC |
| | | CTCCTCTCACTCCACAGGGGAGGTTGTGGTGACTATG |
| | | GAGCCAGAGGTACCCATCAAGAAACTGGAGACCATGG |
| | | TGAAGCTGGACGCGACTTCTGAAGCTGTTGTCAAGGT |
| | | GGATGTGGCTGAGGAGGAGAAGGAGAAGAAGAAGAA |
| | | GAAGAAGGGCACTGCGGAGGAGCCAGAGGAGGAGGA |
| | | GCCAGACGAGAGCATGCTGGACTGGTGGTCCAAGTAC |
| | | TTTGCCTCCATTGACACCATGAAGGAGCAACTTCGACA |
| | | ACAAGAGCCCTCTGGAATTGACTTGGAGGAGAAGGAG |
| | | GAAGTGGACAATACCGAGGGCCTGAAGGGGTCAATGA |
| | | AGGGCAAGGAGAAGGCAAGGGCTGCCAAAGAGGAGA |
| | | AGAAGAAGAAAACTCAGAGCTCTGGCTCTGGCCAGGG |
| | | GTCCGAGGCCCCCGAGAAGAAGAAACCCAAGATTGAT |
| | | GAGCTTAAGGTATACCCCAAAGAGCTGGAGTCCGAGT |
| | | TTGATAACTTTGAGGACTGGCTGCACACTTTCAACTTG |
| | | CTTCGGGGCAAGACCGGGGATGATGAGGATGGCTCC |
| | | ACCGAGGAGGAGCGCATTGTGGGACGCTTCAAGGGC |
| | | TCCCTCTGCGTGTACAAAGTGCCACTCCCAGAGGACG |
| | | TGTCCCGGGAAGCCGGCTACGACTCCACCTACGGCAT |
| | | GTTCCAGGGCATCCCGAGCAATGACCCCATCAATGTG |
| | | CTGGTCCGAGTCTATGTGGTCCGGGCCACGGACCTGC |
| | | ACCCTGCTGACATCAACGGCAAAGCTGACCCCTACAT |
| | | CGCCATCCGGCTAGGCAAGACTGACATCCGCGACAAG |
| | | GAGAACTACATCTCCAAGCAGCTCAACCCTGTCTTTGG |
| | | GAAGTCCTTTGACATCGAGGCCTCCTTCCCCATGGAAT |
| | | CCATGCTGACGGTGGCTGTGTATGACTGGGACCTGGT |
| | | GGGCACTGATGACCTCATTGGGGAAACCAAGATCGAC |
| | | CTGGAGAACCGCTTCTACAGCAAGCACCGCGCCACCT |
| | | GCGGCATCGCCCAGACCTACTCCACACATGGCTACAA |
| | | TATCTGGCGGGACCCCATGAAGCCCAGCCAGATCCTG |
| | | ACCCGCCTCTGCAAAGACGGCAAAGTGGACGGCCCC |
| | | CACTTTGGGCCCCCTGGGAGAGTGAAGGTGGCCAAC |
| | | CGCGTCTTCACTGGGCCCTCTGAGATTGAGGACGAGA |
| | | ACGGTCAGAGGAAGCCCACAGACGAGCATGTGGCGC |
| | | TGTTGGCCCTGAGGCACTGGGAGGACATCCCCCGCG |
| | | CAGGCTGCCGCCTGGTGCCAGAGCATGTGGAGACGA |
| | | GGCCGCTGCTCAACCCCGACAAGCCGGGCATCGAGC |
| | | AGGGCCGCCTGGAGCTGTGGGTGGACATGTTCCCCAT |
| | | GGACATGCCAGCCCCTGGGACGCCTCTGGACATCTCA |
| | | CCTCGGAAGCCCAAGAAGTACGAGCTGCGGGTCATCA |
| | | TCTGGAACACAGATGAGGTGGTCTTGGAGGACGACGA |
| | | CTTCTTCACAGGGGAGAAGTCCAGTGACATCTTCGTG |
| | | AGGGGGTGGCTGAAGGGCCAGCAGGAGGACAAGCAG |
| | | GACACAGACGTCCACTACCACTCCCTCACTGGCGAGG |
| | | GCAACTTCAACTGGCGCTACCTGTTCCCCTTCGACTAC |
| | | CTGGCGGCGGAGGAGAAGATCGTCATCTCCAAGAAG |
| | | GAGTCCATGTTCTCCTGGGACGAGACCGAGTACAAGA |
| | | TCCCCGCGCGGCTCACCCTGCAGATCTGGGATGCGG |
| | | ACCACTTCTCCGCTGACGACTTCCTGGGGGCCATCGA |
| | | GCTGGACCTGAACCGGTTCCCGCGGGGCGCAAAGAC |
| | | AGCCAAGCAGTGCACCATGGAGATGGCCACCGGGGA |
| | | GGTGGACGTGCCCCTCGTGTCCATCTTCAAGCAAAAG |
| | | CGCGTCAAAGGCTGGTGGCCCCTCCTGGCCCGCAAT |
| | | GAGAACGATGAGTTTGAGCTCACGGGCAAGGTGGAG |
| | | GCTGAGCTGCATTTACTGACAGCAGAGGAGGCAGAGA |
| | | AGAACCCAGTGGGCCTGGCCCGCAATGAACCTGACCC |
| | | CCTAGAGAAACCCAACCGGCCCGACACGGCCTTCGTC |
| | | TGGTTCCTCAACCCTCTCAAGTCCATCAAGTACCTCAT |
| | | CTGCACCCGGTACAAGTGGCTCATCATCAAGATCGTG |
| | | CTGGCGCTGTTGGGGCTGCTCATGTTGGGGCTCTTCC |
| | | TCTACAGCCTCCCTGGCTACATGGTCAAAAAGCTCCTT |
| | | GGGGCATGAACGGCCGCTATGCTAGCTTGGTACCAAG |
| | | GGCGGATCCTGCATAGAGCTCGCTGATCAGCCTCGAC |
| | | TGTGCCTTCTAGTTGCCAGCCATCTGTTGTTTGCCCCT |
| | | CCCCCGTGCCTTCCTTGACCCTGGAAGGTGCCACTCC |
| | | CACTGTCCTTTCCTAATAAAATGAGGAAATTGCATCGC |
| | | ATTGTCTGAGTAGGTGTCATTCTATTCTGGGGGGTGG |
| | | GGTGGGGCAGGACAGCAAGGGGGAGGATTGGGAAGA |
| | | CAATAGCAGGCATCTCGAGTTAAGGGCGAATTCCCGA |
| | | TAAGGATCTTCCTAGAGCATGGCTACGTAGATAAGTAG |
| | | CATGGCGGGTTAATCATTAACTACAAGGAACCCCTAGT |
| | | GATGGAGTTGGCCACTCCCTCTCTGCGCGCTCGCTCG |
| | | CTCACTGAGGCCGGGCGACCAAAGGTCGCCCGACGC |
| | | CCGGGCTTTGCCCGGGCGGCCTCAGTGAGCGAGCGA |
| | | GCGCGCAGCCTTAATTAAATCCACATCTGTATGTTTTTT |
| | | ATATTAATTTATTTTTTGCAGGGGGGCATTGTTTGGTAG |
| | | GTGAGAGTTCTGAATTGCTATGTTTAGTGAGTTGTATC |
| | | TATTTATTTTTCAATAAATACAATTAGTTATGTGTTTTGG |
| | | GGGCGATCGTGAGGCAAAGAAAACCCGGCGCTGAGG |
| | | CCGGGTTATTCTTGTTCTCTGGTCAAATTATATAGTTG |
| | | GAAAACAAGGATGCATATATGAATGAACGATGCAGAG |
| | | GCAATGCCGATGGCGATAGTGGGTATCAGGTAGCCGC |
| | | TTATGCTGGAAAGAAGCAATAACCCGCAGAAAAACAAA |
| | | GCTCCAAGCTCAACAAAACTAAGGGCATAGACAATAAC |
| | | TACCTATGTCATATACCCATACTCTCTAATCTTGGCCA |
| | | GTCGGCGCGTTCTGCTTCCGATTAGAAACGTCAAGGC |
| | | AGCAATCAGGATTGCAATCTTGGTTCCTGCATAGGATG |
| | | ACAATGTCGCCCCAAGACCATCTCTATGAGCTGAAAAA |
| | | GAAACACAAGGAATGTAGTGGCGGAAAAGGAGATAGC |
| | | AAATGCTTACGATAACGTAAGGAATTATTACTATGTAAA |
| | | CACCAGGCAAGATTCTGTTCCGTATAATTACTCCTGAT |
| | | AATTAATCCTTAACTTTGCCCACCTGCCTTTTAAAACAT |
| | | TCCAGTATATCACTTTTCATTCTTGCGTAGCAATATGCC |
| | | CTCTCTTCAGCTATCTCAGCATTGGTGACCTTGTTCAG |
| | | AGGCGCTGAGAGATGGCCTTTTTCTGATAGATAATGTT |
| | | CTGTTAAAATATCTCCGGCCTCATCTTTTGCCCGCAGG |
| | | CTAATGTCTGAAAATTGAGGTGACGGGTTAAAAATAAT |
| | | ATCCTTGGCAACCTTTTTTATATCCCTTTTAAATTTTGG |
| | | CTTAATGACTATATCCAATGAGTCAAAAAGCTCCCCTT |
| | | CAATATCTGTTGCCCCTAAGACCTTTAATATATCGCCA |
| | | AATACAGGTAGCTTGGCTTCTACCTTCACCGTTGTTCT |
| | | GCCGATGAAATGCTAATGCATAACATCGTCTTTGGTGG |
| | | TTCCCCTCATCAGTGGCTCTATCTGAACGCGCTCTCCA |
| | | CTGCTTAATGACATTCCTTTCCCGATTAAAAAATCTGTC |
| | | AGATCGGATGTGGTCGGCCCGAAAACAGTTCTGGCAA |
| | | AACCAATGGTGTCGCCTTCAACAAACAAAAAAGATGGG |
| | | AATCCCAATGATTCGTCATCTGCGAGGCTGTTCTTAAT |
| | | ATCTTCAACTGTAGCTTTAGAGCGATTTATCTTCTGAAC |
| | | CAGACTCTTGTCATTTGTTTTGGTAAAGAGAAAAGTTTT |
| | | TCCATCGATTTTATGAATATACAAATAATTGGAGCCAAC |
| | | CTTCAGGTGATGATTATCAGCCAGCAGAGAATTAAGGA |
| | | AAACAGACAGGTTTATTGAGCACTTATCTTTCCCTTTAT |
| | | TTTTGCTGCGGTAAGTCGCATAAAAACCATTCTTCACA |
| | | ATTCAATCCATTTACTATGTTATGTTCTGAGGGGAGTG |
| | | AAAATTCCCCTAATTCGATGAAGATTCTTGCTAAATTGT |
| | | TATCAGCTATGCGCCGACCAGAACACCTTGCCGATCA |
| | | GCCAAACGTCTAATCAGGCCACTGACTAGCGATAACTT |
| | | TCCCCACAACGGAACAACTCTCATTGCATGGGATAATT |
| | | GGGTACTGTGGGTTTAGTGGTTGTAAAAACACCTGAC |
| | | CGCTATCCCTGATCAGTTTCTTGAAGGTAAACTCATCA |
| | | CCCCCAAGTCTGGCTATACAGAAATCACCTGGCTCAA |
| | | CAGCCTGCTCAGGGTCAACGAGAATTTACATTCCGTCA |
| | | GGATAGCTTGGCTTGGAGCCTGTTGGTGCGGTCACGG |
| | | AATTACCTTCAACCTCAAGCCAGAATGCAGAATCACTG |
| | | GCTTTTTTGGTTGTGCTTACCCATCTCTCCGCATCACC |
| | | TTTGGTAAAGGTTCTAAGCTAAGGTGAGAACATCCCTG |
| | | CCTGAACATGAGAAAAAACAGGGTACTCATACTCACTT |
| | | ATTAGTGACGGCTATGAGCAAAAGGCCAGCAAAAGGC |
| | | CAGGAACCGTAAAAAGGCCGCGTTGCTGGCGTTTTTC |
| | | CATAGGCTCCGCCCCCCTGACGAGCATCACAAAAATC |
| | | GACGCTCAAGTCAGAGGTGGCGAAACCCGACAGGACT |
| | | ATAAAGATACCAGGCGTTTCCCCCTGGAAGCTCCCTC |
| | | GTGCGCTCTCCTGTTCCGACCCTGCCGCTTACCGGAT |
| | | ACCTGTCCGCCTTTCTCCCTTCGGGAAGCGTGGCGCT |
| | | TTCTCATAGCTCACGCTGTAGGTATCTCAGTTCGGTGT |
| | | AGGTCGTTCGCTCCAAGCTGGGCTGTGTGCACGAACC |
| | | CCCCGTTCAGCCCGACCGCTGCGCCTTATCCGGTAAC |
| | | TATCGTCTTGAGTCCAACCCGGTAAGACACGACTTATC |
| | | GCCACTGGCAGCAGCCACTGGTAACAGGATTAGCAGA |
| | | GCGAGGTATGTAGGCGGTGCTACAGAGTTCTTGAAGT |
| | | GGTGGCCTAACTACGGCTACACTAGAAGAACAGTATTT |
| | | GGTATCTGCGCTCTGCTGAAGCCAGTTACCTTCGGAA |
| | | AAAGAGTTGGTAGCTCTTGATCCGGCAAACAAACCAC |
| | | CGCTGGTAGCGGTGGTTTTTTTGTTTGCAAGCAGCAG |
| | | ATTACGCGCAGAAAAAAAGGATCTCAAGAAGATCCTTT |
| | | GATCTTTTCTACGGGGTCTGACGCTCAGTGGAACGAA |
| | | AACTCACGTTAAGGGATTTTGGTCATGAGATTATCAAA |
| | | AAGGATCTTCACCTAGATCCTTTTAAATTAAAAATGAAG |
| | | TTTTAAATCAAGCCCAATCTGAATAATGTTACAACCAAT |
| | | TAACCAATTCTGATTAGAAAAACTCATCGAGCATCAAAT |
| | | GAAACTGCAATTTATTCATATCAGGATTATCAATACCAT |
| | | ATTTTTGAAAAAGCCGTTTCTGTAATGAAGGAGAAAAC |
| | | TCACCGAGGCAGTTCCATAGGATGGCAAGATCCTGGT |
| | | ATCGGTCTGCGATTCCGACTCGTCCAACATCAATACAA |
| | | CCTATTAATTTCCCCTCGTCAAAAATAAGGTTATCAAGT |
| | | GAGAAATCACCATGAGTGACGACTGAATCCGGTGAGA |
| | | ATGGCAAAAGTTTATGCATTTCTTTCCAGACTTGTTCAA |
| | | CAGGCCAGCCATTACGCTCGTCATCAAAATCACTCGC |
| | | ATCAACCAAACCGTTATTCATTCGTGATTGCGCCTGAG |
| | | CAAGACGAAATACGCGATCGCTGTTAAAAGGACAATTA |
| | | CAAACAGGAATCGAATGCAACCGGCGCAGGAACACTG |
| | | CCAGCGCATCAACAATATTTTCACCTGAATCAGGATAT |
| | | TCTTCTAATACCTGGAATGCTGTTTTTCCGGGGATCGC |
| | | AGTGGTGAGTAACCATGCATCATCAGGAGTACGGATA |
| | | AAATGCTTGATGGTCGGAAGAGGCATAAATTCCGTCA |
| | | GCCAGTTTAGTCTGACCATCTCATCTGTAACATCATTG |
| | | GCAACGCTACCTTTGCCATGTTTCAGAAACAACTCTGG |
| | | CGCATCGGGCTTCCCATACAAGCGATAGATTGTCGCA |
| | | CCTGATTGCCCGACATTATCGCGAGCCCATTTATACCC |
| | | ATATAAATCAGCATCCATGTTGGAATTTAATCGCGGCC |
| | | TCGACGTTTCCCGTTGAATATGGCTCATAACACCCCTT |
| | | GTATTACTGTTTATGTAAGCAGACAGTTTTATTGTTCAT |
| | | GATGATATATTTTTATCTTGTGCAATGTAACATCAGAGA |
| | | TTTTGAGACACGGGCCAGAGCTGCA |
| 62 | 5′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | Myo15 promoter at positions | AGTGAGCGAGCGAGCGCGCAGAGAGGGAGTGGCCAA |
| | 182-1146 | CTCCATCACTAGGGGTTCCTCAGATCTGAATTCGGTAC |
| | N-terminal portion of human | CTGCAGCTCAGCCTACTACTTGCTTTCCAGGCTGTTCC |
| | OTOF isoform 5 at positions | TAGTTCCCATGTCAGCTGCTTGTGCTTTCCAGAGACAA |
| | 1167-3572 | AACAGGAATAATAGATGTCATTAAATATACATTGGGCC |
| | Splice donor sequence at | CCAGGCGGTCAATGTGGCAGCCTGAGCCTCCTTTCCA |
| | positions 3573-3656 | TCTCTGTGGAGGCAGACATAGGACCCCCAACAAACAG |
| | AP head sequence at positions | CATGCAGGTTGGGAGCCAGCCACAGGACCCAGGTAA |
| | 3663-3949 | GGGGCCCTGGGTCCTTAAGCTTCTGCCACTGGCTCCG |
| | ITR at positions 3973-4115 | GCATTGCAGAGAGAAGAGAAGGGGCGGCAGACTGGA |
| | KanR at positions 4641-5435 | GAGCTGGGCTCCATTTTTGTTCCTTGGTGCCCTGCCC |
| | pUC ori at positions 5821-6491 | CTCCCCATGACCTGCAGAGACATTCAGCCTGCCAGGC |
| | Transgene to be transferred | TTTATGAGGTGGGAGCTGGGCTCTCCCTGATGTATTAT |
| | into vector in dual vector | TCAGCTCCCTGGAGTTGGCCAGCTCCTGTTACACTGG |
| | system at positions 19-4115 | CCACAGCCCTGGGCATCCGCTTCTCACTTCTAGTTTCC |
| | | CCTCCAAGGTAATGTGGTGGGTCATGATCATTCTATCC |
| | | TGGCTTCAGGGACCTGACTCCACTTTGGGGCCATTCG |
| | | AGGGGTCTAGGGTAGATGATGTCCCCCTGTGGGGATT |
| | | AATGTCCTGCTCTGTAAAACTGAGCTAGCTGAGATCCA |
| | | GGAGGGCTTGGCCAGAGACAGCAAGTTGTTGCCATGG |
| | | TGACTTTAAAGCCAGGTTGCTGCCCCAGCACAGGCCT |
| | | CCCAGTCTACCCTCACTAGAAAACAACACCCAGGCAC |
| | | TTTCCACCACCTCTCAAAGGTGAAACCCAAGGCTGGT |
| | | CTAGAGAATGAATTATGGATCCTCGCTGTCCGTGCCAC |
| | | CCAGCTAGTCCCAGCGGCTCAGACACTGAGGAGAGAC |
| | | TGTAGGTTCAGCTACAAGCAAAAAGACCTAGCTGGTCT |
| | | CCAAGCAGTGTCTCCAAGTCCCTGAACCTGTGACACC |
| | | TGCCCCAGGCATCATCAGGCACAGAGGGCCACCGAAT |
| | | TCTAGCGGCCGCCACCATGGCCTTGCTCATCCACCTC |
| | | AAGACAGTCTCGGAGCTGCGGGGCAGGGGCGACCGG |
| | | ATCGCCAAAGTGACTTTCCGAGGGCAATCCTTCTACTC |
| | | TCGGGTCCTGGAGAACTGTGAGGATGTGGCTGACTTT |
| | | GATGAGACATTTCGGTGGCCGGTGGCCAGCAGCATCG |
| | | ACAGAAATGAGATGCTGGAGATTCAGGTTTTCAACTAC |
| | | AGCAAAGTCTTCAGCAACAAGCTCATCGGGACCTTCC |
| | | GCATGGTGCTGCAGAAGGTGGTAGAGGAGAGCCATGT |
| | | GGAGGTGACTGACACGCTGATTGATGACAACAATGCT |
| | | ATCATCAAGACCAGCCTGTGCGTGGAGGTCCGGTATC |
| | | AGGCCACTGACGGCACAGTGGGCTCCTGGGACGATG |
| | | GGGACTTCCTGGGAGATGAGTCTCTTCAAGAGGAAGA |
| | | GAAGGACAGCCAAGAGACGGATGGACTGCTCCCAGG |
| | | CTCCCGGCCCAGCTCCCGGCCCCCAGGAGAGAAGAG |
| | | CTTCCGGAGAGCCGGGAGGAGCGTGTTCTCCGCCAT |
| | | GAAGCTCGGCAAAAACCGGTCTCACAAGGAGGAGCCC |
| | | CAAAGACCAGATGAACCGGCGGTGCTGGAGATGGAA |
| | | GACCTTGACCATCTGGCCATTCGGCTAGGAGATGGAC |
| | | TGGATCCCGACTCGGTGTCTCTAGCCTCAGTCACAGC |
| | | TCTCACCACTAATGTCTCCAACAAGCGATCTAAGCCAG |
| | | ACATTAAGATGGAGCCAAGTGCTGGGCGGCCCATGGA |
| | | TTACCAGGTCAGCATCACGGTGATCGAGGCCCGGCAG |
| | | CTGGTGGGCTTGAACATGGACCCTGTGGTGTGCGTGG |
| | | AGGTGGGTGACGACAAGAAGTACACATCCATGAAGGA |
| | | GTCCACTAACTGCCCCTATTACAACGAGTACTTCGTCT |
| | | TCGACTTCCATGTCTCTCCGGATGTCATGTTTGACAAG |
| | | ATCATCAAGATTTCGGTGATTCACTCCAAGAACCTGCT |
| | | GCGCAGTGGCACCCTGGTGGGCTCCTTCAAAATGGAC |
| | | GTGGGAACCGTGTACTCGCAGCCAGAGCACCAGTTCC |
| | | ATCACAAGTGGGCCATCCTGTCTGACCCCGATGACAT |
| | | CTCCTCGGGGCTGAAGGGCTACGTGAAGTGTGACGTT |
| | | GCCGTGGTGGGCAAAGGGGACAACATCAAGACGCCC |
| | | CACAAGGCCAATGAGACCGACGAAGATGACATTGAGG |
| | | GGAACTTGCTGCTCCCCGAGGGGGTGCCCCCCGAAC |
| | | GCCAGTGGGCCCGGTTCTATGTGAAAATTTACCGAGC |
| | | AGAGGGGCTGCCCCGTATGAACACAAGCCTCATGGCC |
| | | AATGTAAAGAAGGCTTTCATCGGTGAAAACAAGGACCT |
| | | CGTGGACCCCTACGTGCAAGTCTTCTTTGCTGGCCAG |
| | | AAGGGCAAGACTTCAGTGCAGAAGAGCAGCTATGAGC |
| | | CCCTGTGGAATGAGCAGGTCGTCTTTACAGACCTCTTC |
| | | CCCCCACTCTGCAAACGCATGAAGGTGCAGATCCGAG |
| | | ACTCGGACAAGGTCAACGACGTGGCCATCGGCACCCA |
| | | CTTCATTGACCTGCGCAAGATTTCTAATGACGGAGACA |
| | | AAGGCTTCCTGCCCACACTGGGCCCAGCCTGGGTGAA |
| | | CATGTACGGCTCCACACGTAACTACACGCTGCTGGAT |
| | | GAGCATCAGGACCTGAACGAGGGCCTGGGGGAGGGT |
| | | GTGTCCTTCCGGGCCCGGCTCCTGCTGGGCCTGGCT |
| | | GTGGAGATCGTAGACACCTCCAACCCTGAGCTCACCA |
| | | GCTCCACAGAGGTGCAGGTGGAGCAGGCCACGCCCA |
| | | TCTCGGAGAGCTGTGCAGGTAAAATGGAAGAATTCTTT |
| | | CTCTTTGGAGCCTTCCTGGAGGCCTCAATGATCGACC |
| | | GGAGAAACGGAGACAAGCCCATCACCTTTGAGGTCAC |
| | | CATAGGCAACTATGGGAACGAAGTTGATGGCCTGTCC |
| | | CGGCCCCAGCGGCCTCGGCCCCGGAAGGAGCCGGG |
| | | GGATGAGGAAGAAGTAGACCTGATTCAGAACGCAAGT |
| | | GATGACGAGGCCGGTGATGCCGGGGACCTGGCCTCA |
| | | GTCTCCTCCACTCCACCAATGCGGCCCCAGGTCACCG |
| | | ACAGGAACTACTTCCATCTGCCCTACCTGGAGCGAAA |
| | | GCCCTGCATCTACATCAAGAGCTGGTGGCCGGACCAG |
| | | CGCCGCCGCCTCTACAATGCCAACATCATGGACCACA |
| | | TTGCCGACAAGCTGGAAGAAGGCCTGAACGACATACA |
| | | GGAGATGATCAAAACGGAGAAGTCCTACCCTGAGCGT |
| | | CGCCTGCGGGGCGTCCTGGAGGAGCTGAGCTGTGGC |
| | | TGCTGCCGCTTCCTCTCCCTCGCTGACAAGGACCAGG |
| | | GCCACTCATCCCGCACCAGGCTTGACCGGGAGCGCC |
| | | TCAAGTCCTGCATGAGGGAGCTGGTAAGTATCAAGGT |
| | | TACAAGACAGGTTTAAGGAGACCAATAGAAACTGGGC |
| | | TTGTCGAGACAGAGAAGACTCTTGCGTTTCTGAGCTAG |
| | | CCCCCGGGTGCGCGGCGTCGGTGGTGCCGGCGGGG |
| | | GGCGCCAGGTCGCAGGCGGTGTAGGGCTCCAGGCAG |
| | | GCGGCGAAGGCCATGACGTGCGCTATGAAGGTCTGCT |
| | | CCTGCACGCCGTGAACCAGGTGCGCCTGCGGGCCGC |
| | | GCGCGAACACCGCCACGTCCTCGCCTGCGTGGGTCT |
| | | CTTCGTCCAGGGGCACTGCTGACTGCTGCCGATACTC |
| | | GGGGCTCCCGCTCTCGCTCTCGGTAACATCCGGCCG |
| | | GGCGCCGTCCTTGAGCACATAGCCTGGACCGTTTCGT |
| | | CGACTGGGGAGAGATCTGAGGAACCCCTAGTGATGGA |
| | | GTTGGCCACTCCCTCTCTGCGCGCTCGCTCGCTCACT |
| | | GAGGCCGCCCGGGCAAAGCCCGGGCGTCGGGCGAC |
| | | CTTTGGTCGCCCGGCCTCAGTGAGCGAGCGAGCGCG |
| | | CAGAGAGGGAGTGGCCAACCCCCCCCCCCCCCCCCC |
| | | TGCAGCCTGGCGTAATAGCGAAGAGGCCCGCACCGAT |
| | | CGCCCTTCCCAACAGTTGCGTAGCCTGAATGGCGAAT |
| | | GGCGCGACGCGCCCTGTAGCGGCGCATTAAGCGCGG |
| | | CGGGTGTGGTGGTTACGCGCAGCGTGACCGCTACACT |
| | | TGCCAGCGCCCTAGCGCCCGCTCCTTTCGCTTTCTTC |
| | | CCTTCCTTTCTCGCCACGTTCGCCGGCTTTCCCCGTCA |
| | | AGCTCTAAATCGGGGGCTCCCTTTAGGGTTCCGATTTA |
| | | GTGCTTTACGGCACCTCGACCCCAAAAAACTTGATTAG |
| | | GGTGATGGTTCACGTAGTGGGCCATCGCCCTGATAGA |
| | | CGGTTTTTCGCCCTTTGACGTTGGAGTCCACGTTCTTT |
| | | AATAGTGGACTCTTGTTCCAAACTGGAACAACACTCAA |
| | | CCCTATCGCGGTCTATTCTTTTGATTTATAAGGGATGTT |
| | | GCCGATTTCGGCCTATTGGTTAAAAAATGAGCTGATTT |
| | | AACAAAAATTTTAACAAAATTCAGAAGAACTCGTCAAGA |
| | | AGGCGATAGAAGGCGATGCGCTGCGAATCGGGAGCG |
| | | GCGATACCGTAAAGCACGAGGAAGCGGTCAGCCCATT |
| | | CGCCGCCAAGCTCTTCAGCAATATCACGGGTAGCCAA |
| | | CGCTATGTCCTGATAGCGGTCCGCCACACCCAGCCGG |
| | | CCACAGTCGATGAATCCAGAAAAGCGGCCATTTTCCA |
| | | CCATGATATTCGGCAAGCAGGCATCGCCATGGGTCAC |
| | | GACGAGATCCTCGCCGTCGGGCATGCTCGCCTTGAGC |
| | | CTGGCGAACAGTTCGGCTGGCGCGAGCCCCTGATGC |
| | | TCTTCGTCCAGATCATCCTGATCGACAAGACCGGCTTC |
| | | CATCCGAGTACGTGCTCGCTCGATGCGATGTTTCGCT |
| | | TGGTGGTCGAATGGGCAGGTAGCCGGATCAAGCGTAT |
| | | GCAGCCGCCGCATTGCATCAGCCATGATGGATACTTT |
| | | CTCGGCAGGAGCAAGGTGAGATGACAGGAGATCCTG |
| | | CCCCGGCACTTCGCCCAATAGCAGCCAGTCCCTTCCC |
| | | GCTTCAGTGACAACGTCGAGCACAGCTGCGCAAGGAA |
| | | CGCCCGTCGTGGCCAGCCACGATAGCCGCGCTGCCT |
| | | CGTCTTGCAGTTCATTCAGGGCACCGGACAGGTCGGT |
| | | CTTGACAAAAAGAACCGGGCGCCCCTGCGCTGACAGC |
| | | CGGAACACGGCGGCATCAGAGCAGCCGATTGTCTGTT |
| | | GTGCCCAGTCATAGCCGAATAGCCTCTCCACCCAAGC |
| | | GGCCGGAGAACCTGCGTGCAATCCATCTTGTTCAATC |
| | | ATGCGAAACGATCCTCATCCTGTCTCTTGATCAGATCT |
| | | TGATCCCCTGCGCCATCAGATCCTTGGCGGCGAGAAA |
| | | GCCATCCAGTTTACTTTGCAGGGCTTCCCAACCTTACC |
| | | AGAGGGCGCCCCAGCTGGCAATTCCGGTTCGCTTGCT |
| | | GTCCATAAAACCGCCCAGTCTAGCTATCGCCATGTAAG |
| | | CCCACTGCAAGCTACCTGCTTTCTCTTTGCGCTTGCGT |
| | | TTTCCCTTGTCCAGATAGCCCAGTAGCTGACATTCATC |
| | | CGGGGTCAGCACCGTTTCTGCGGACTGGCTTTCTACG |
| | | TGAAAAGGATCTAGGTGAAGATCCTTTTTGATAATCTC |
| | | ATGACCAAAATCCCTTAACGTGAGTTTTCGTTCCACTG |
| | | AGCGTCAGACCCCGTAGAAAAGATCAAAGGATCTTCTT |
| | | GAGATCCTTTTTTTCTGCGCGTAATCTGCTGCTTGCAA |
| | | ACAAAAAAACCACCGCTACCAGCGGTGGTTTGTTTGC |
| | | CGGATCAAGAGCTACCAACTCTTTTTCCGAAGGTAACT |
| | | GGCTTCAGCAGAGCGCAGATACCAAATACTGTCCTTCT |
| | | AGTGTAGCCGTAGTTAGGCCACCACTTCAAGAACTCT |
| | | GTAGCACCGCCTACATACCTCGCTCTGCTAATCCTGTT |
| | | ACCAGTGGCTGCTGCCAGTGGCGATAAGTCGTGTCTT |
| | | ACCGGGTTGGACTCAAGACGATAGTTACCGGATAAGG |
| | | CGCAGCGGTCGGGCTGAACGGGGGGTTCGTGCACAC |
| | | AGCCCAGCTTGGAGCGAACGACCTACACCGAACTGAG |
| | | ATACCTACAGCGTGAGCTATGAGAAAGCGCCACGCTT |
| | | CCCGAAGGGAGAAAGGCGGACAGGTATCCGGTAAGC |
| | | GGCAGGGTCGGAACAGGAGAGCGCACGAGGGAGCTT |
| | | CCAGGGGGAAACGCCTGGTATCTTTATAGTCCTGTCG |
| | | GGTTTCGCCACCTCTGACTTGAGCGTCGATTTTTGTGA |
| | | TGCTCGTCAGGGGGGCGGAGCCTATGGAAAAACGCC |
| | | AGCAACGCGGCCTTTTTACGGTTCCTGGGCTTTTGCT |
| | | GGCCTTTTGCTCACATGTTCTTTCCTGCGTTATCCCCT |
| | | GATTCTGTGGATAACCGTATTACCGCCTTTGAGTGAGC |
| | | TGATACCGCTCGCCGCAGCCGAACGACCGAGCGCAG |
| | | CGAGTCAGTGAGCGAGGAAGCGGAAGAGCGCCCAAT |
| | | ACGCAAACCGCCTCTCCCCGCGCGTTGGCCGATTCAT |
| | | TAATGCAGGGCTGCA |
| 63 | 3′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | AP head sequence at positions | AGTGAGCGAGCGAGCGCGCAGAGAGGGAGTGGCCAA |
| | 187-473 | CTCCATCACTAGGGGTTCCTCAGATCTGAATTCTAGCG |
| | Splice acceptor sequence at | GCCGCCCCCGGGTGCGCGGCGTCGGTGGTGCCGGC |
| | positions 496-544 | GGGGGGCGCCAGGTCGCAGGCGGTGTAGGGCTCCA |
| | C-terminal portion of human | GGCAGGCGGCGAAGGCCATGACGTGCGCTATGAAGG |
| | OTOF isoform 5 at positions | TCTGCTCCTGCACGCCGTGAACCAGGTGCGCCTGCG |
| | 545-4132 | GGCCGCGCGCGAACACCGCCACGTCCTCGCCTGCGT |
| | bGH poly(A) sequence at | GGGTCTCTTCGTCCAGGGGCACTGCTGACTGCTGCCG |
| | positions 4175-4396 | ATACTCGGGGCTCCCGCTCTCGCTCTCGGTAACATCC |
| | ITR at positions 4447-4589 | GGCCGGGCGCCGTCCTTGAGCACATAGCCTGGACCG |
| | KanR at positions 5115-5909 | TTTCCTTAAGCGACGCATGCTCGCGATAGGCACCTATT |
| | pUC ori at positions 6295-6965 | GGTCTTACTGACATCCACTTTGCCTTTCTCTCCACAGG |
| | Transgene to be transferred | AAAACATGGGGCAGCAGGCCAGGATGCTGCGGGCCC |
| | into vector in dual vector | AGGTGAAGCGGCACACGGTGCGGGACAAGCTGAGGC |
| | system at positions 19-4589 | TGTGCCAGAACTTCCTGCAGAAGCTGCGCTTCCTGGC |
| | | GGACGAGCCCCAGCACAGCATTCCCGACATCTTCATC |
| | | TGGATGATGAGCAACAACAAGCGTGTCGCCTATGCCC |
| | | GTGTGCCCTCCAAGGACCTGCTCTTCTCCATCGTGGA |
| | | GGAGGAGACTGGCAAGGACTGCGCCAAGGTCAAGAC |
| | | GCTCTTCCTTAAGCTGCCAGGGAAGCGGGGCTTCGGC |
| | | TCGGCAGGCTGGACAGTGCAGGCCAAGGTGGAGCTG |
| | | TACCTGTGGCTGGGCCTCAGCAAACAGCGCAAGGAGT |
| | | TCCTGTGCGGCCTGCCCTGTGGCTTCCAGGAGGTCAA |
| | | GGCAGCCCAGGGCCTGGGCCTGCATGCCTTCCCACC |
| | | CGTCAGCCTGGTCTACACCAAGAAGCAGGCGTTCCAG |
| | | CTCCGAGCGCACATGTACCAGGCCCGCAGCCTCTTTG |
| | | CCGCCGACAGCAGCGGACTCTCAGACCCCTTTGCCCG |
| | | CGTCTTCTTCATCAATCAGAGTCAGTGCACAGAGGTGC |
| | | TGAATGAGACCCTGTGTCCCACCTGGGACCAGATGCT |
| | | GGTGTTCGACAACCTGGAGCTCTATGGTGAAGCTCAT |
| | | GAGCTGAGGGACGATCCGCCCATCATTGTCATTGAAA |
| | | TCTATGACCAGGATTCCATGGGCAAAGCTGACTTCATG |
| | | GGCCGGACCTTCGCCAAACCCCTGGTGAAGATGGCA |
| | | GACGAGGCGTACTGCCCACCCCGCTTCCCACCTCAGC |
| | | TCGAGTACTACCAGATCTACCGTGGCAACGCCACAGC |
| | | TGGAGACCTGCTGGCGGCCTTCGAGCTGCTGCAGATT |
| | | GGACCAGCAGGGAAGGCTGACCTGCCCCCCATCAAT |
| | | GGCCCGGTGGACGTGGACCGAGGTCCCATCATGCCC |
| | | GTGCCCATGGGCATCCGGCCCGTGCTCAGCAAGTACC |
| | | GAGTGGAGGTGCTGTTCTGGGGCCTACGGGACCTAAA |
| | | GCGGGTGAACCTGGCCCAGGTGGACCGGCCACGGGT |
| | | GGACATCGAGTGTGCAGGGAAGGGGGTGCAGTCGTC |
| | | CCTGATCCACAATTATAAGAAGAACCCCAACTTCAACA |
| | | CCCTCGTCAAGTGGTTTGAAGTGGACCTCCCAGAGAA |
| | | CGAGCTGCTGCACCCGCCCTTGAACATCCGTGTGGTG |
| | | GACTGCCGGGCCTTCGGTCGCTACACACTGGTGGGCT |
| | | CCCATGCCGTCAGCTCCCTGCGACGCTTCATCTACCG |
| | | GCCCCCAGACCGCTCGGCCCCCAGCTGGAACACCAC |
| | | GGTCAGGCTTCTCCGGCGCTGCCGTGTGCTGTGCAAT |
| | | GGGGGCTCCTCCTCTCACTCCACAGGGGAGGTTGTG |
| | | GTGACTATGGAGCCAGAGGTACCCATCAAGAAACTGG |
| | | AGACCATGGTGAAGCTGGACGCGACTTCTGAAGCTGT |
| | | TGTCAAGGTGGATGTGGCTGAGGAGGAGAAGGAGAA |
| | | GAAGAAGAAGAAGAAGGGCACTGCGGAGGAGCCAGA |
| | | GGAGGAGGAGCCAGACGAGAGCATGCTGGACTGGTG |
| | | GTCCAAGTACTTTGCCTCCATTGACACCATGAAGGAGC |
| | | AACTTCGACAACAAGAGCCCTCTGGAATTGACTTGGA |
| | | GGAGAAGGAGGAAGTGGACAATACCGAGGGCCTGAA |
| | | GGGGTCAATGAAGGGCAAGGAGAAGGCAAGGGCTGC |
| | | CAAAGAGGAGAAGAAGAAGAAAACTCAGAGCTCTGGC |
| | | TCTGGCCAGGGGTCCGAGGCCCCCGAGAAGAAGAAA |
| | | CCCAAGATTGATGAGCTTAAGGTATACCCCAAAGAGCT |
| | | GGAGTCCGAGTTTGATAACTTTGAGGACTGGCTGCAC |
| | | ACTTTCAACTTGCTTCGGGGCAAGACCGGGGATGATG |
| | | AGGATGGCTCCACCGAGGAGGAGCGCATTGTGGGAC |
| | | GCTTCAAGGGCTCCCTCTGCGTGTACAAAGTGCCACT |
| | | CCCAGAGGACGTGTCCCGGGAAGCCGGCTACGACTC |
| | | CACCTACGGCATGTTCCAGGGCATCCCGAGCAATGAC |
| | | CCCATCAATGTGCTGGTCCGAGTCTATGTGGTCCGGG |
| | | CCACGGACCTGCACCCTGCTGACATCAACGGCAAAGC |
| | | TGACCCCTACATCGCCATCCGGCTAGGCAAGACTGAC |
| | | ATCCGCGACAAGGAGAACTACATCTCCAAGCAGCTCA |
| | | ACCCTGTCTTTGGGAAGTCCTTTGACATCGAGGCCTC |
| | | CTTCCCCATGGAATCCATGCTGACGGTGGCTGTGTAT |
| | | GACTGGGACCTGGTGGGCACTGATGACCTCATTGGGG |
| | | AAACCAAGATCGACCTGGAGAACCGCTTCTACAGCAA |
| | | GCACCGCGCCACCTGCGGCATCGCCCAGACCTACTC |
| | | CACACATGGCTACAATATCTGGCGGGACCCCATGAAG |
| | | CCCAGCCAGATCCTGACCCGCCTCTGCAAAGACGGCA |
| | | AAGTGGACGGCCCCCACTTTGGGCCCCCTGGGAGAG |
| | | TGAAGGTGGCCAACCGCGTCTTCACTGGGCCCTCTGA |
| | | GATTGAGGACGAGAACGGTCAGAGGAAGCCCACAGA |
| | | CGAGCATGTGGCGCTGTTGGCCCTGAGGCACTGGGA |
| | | GGACATCCCCCGCGCAGGCTGCCGCCTGGTGCCAGA |
| | | GCATGTGGAGACGAGGCCGCTGCTCAACCCCGACAA |
| | | GCCGGGCATCGAGCAGGGCCGCCTGGAGCTGTGGGT |
| | | GGACATGTTCCCCATGGACATGCCAGCCCCTGGGACG |
| | | CCTCTGGACATCTCACCTCGGAAGCCCAAGAAGTACG |
| | | AGCTGCGGGTCATCATCTGGAACACAGATGAGGTGGT |
| | | CTTGGAGGACGACGACTTCTTCACAGGGGAGAAGTCC |
| | | AGTGACATCTTCGTGAGGGGGTGGCTGAAGGGCCAG |
| | | CAGGAGGACAAGCAGGACACAGACGTCCACTACCACT |
| | | CCCTCACTGGCGAGGGCAACTTCAACTGGCGCTACCT |
| | | GTTCCCCTTCGACTACCTGGCGGCGGAGGAGAAGATC |
| | | GTCATCTCCAAGAAGGAGTCCATGTTCTCCTGGGACG |
| | | AGACCGAGTACAAGATCCCCGCGCGGCTCACCCTGCA |
| | | GATCTGGGATGCGGACCACTTCTCCGCTGACGACTTC |
| | | CTGGGGGCCATCGAGCTGGACCTGAACCGGTTCCCG |
| | | CGGGGCGCAAAGACAGCCAAGCAGTGCACCATGGAG |
| | | ATGGCCACCGGGGAGGTGGACGTGCCCCTCGTGTCC |
| | | ATCTTCAAGCAAAAGCGCGTCAAAGGCTGGTGGCCCC |
| | | TCCTGGCCCGCAATGAGAACGATGAGTTTGAGCTCAC |
| | | GGGCAAGGTGGAGGCTGAGCTGCATTTACTGACAGCA |
| | | GAGGAGGCAGAGAAGAACCCAGTGGGCCTGGCCCGC |
| | | AATGAACCTGACCCCCTAGAGAAACCCAACCGGCCCG |
| | | ACACGGCCTTCGTCTGGTTCCTCAACCCTCTCAAGTCC |
| | | ATCAAGTACCTCATCTGCACCCGGTACAAGTGGCTCAT |
| | | CATCAAGATCGTGCTGGCGCTGTTGGGGCTGCTCATG |
| | | TTGGGGCTCTTCCTCTACAGCCTCCCTGGCTACATGG |
| | | TCAAAAAGCTCCTTGGGGCATGAACGGCCGCTATGCT |
| | | AGCTTGGTACCAAGGGCGGATCCTGCATAGAGCTCGC |
| | | TGATCAGCCTCGACTGTGCCTTCTAGTTGCCAGCCATC |
| | | TGTTGTTTGCCCCTCCCCCGTGCCTTCCTTGACCCTG |
| | | GAAGGTGCCACTCCCACTGTCCTTTCCTAATAAAATGA |
| | | GGAAATTGCATCGCATTGTCTGAGTAGGTGTCATTCTA |
| | | TTCTGGGGGGTGGGGTGGGGCAGGACAGCAAGGGG |
| | | GAGGATTGGGAAGACAATAGCAGGCATGCTGGGGAG |
| | | AGATCTGAGGACTAGTCCGTCGACTGGGGAGAGATCT |
| | | GAGGAACCCCTAGTGATGGAGTTGGCCACTCCCTCTC |
| | | TGCGCGCTCGCTCGCTCACTGAGGCCGCCCGGGCAA |
| | | AGCCCGGGCGTCGGGCGACCTTTGGTCGCCCGGCCT |
| | | CAGTGAGCGAGCGAGCGCGCAGAGAGGGAGTGGCCA |
| | | ACCCCCCCCCCCCCCCCCCTGCAGCCTGGCGTAATAG |
| | | CGAAGAGGCCCGCACCGATCGCCCTTCCCAACAGTTG |
| | | CGTAGCCTGAATGGCGAATGGCGCGACGCGCCCTGT |
| | | AGCGGCGCATTAAGCGCGGCGGGTGTGGTGGTTACG |
| | | CGCAGCGTGACCGCTACACTTGCCAGCGCCCTAGCG |
| | | CCCGCTCCTTTCGCTTTCTTCCCTTCCTTTCTCGCCAC |
| | | GTTCGCCGGCTTTCCCCGTCAAGCTCTAAATCGGGGG |
| | | CTCCCTTTAGGGTTCCGATTTAGTGCTTTACGGCACCT |
| | | CGACCCCAAAAAACTTGATTAGGGTGATGGTTCACGTA |
| | | GTGGGCCATCGCCCTGATAGACGGTTTTTCGCCCTTT |
| | | GACGTTGGAGTCCACGTTCTTTAATAGTGGACTCTTGT |
| | | TCCAAACTGGAACAACACTCAACCCTATCGCGGTCTAT |
| | | TCTTTTGATTTATAAGGGATGTTGCCGATTTCGGCCTA |
| | | TTGGTTAAAAAATGAGCTGATTTAACAAAAATTTTAACA |
| | | AAATTCAGAAGAACTCGTCAAGAAGGCGATAGAAGGC |
| | | GATGCGCTGCGAATCGGGAGCGGCGATACCGTAAAG |
| | | CACGAGGAAGCGGTCAGCCCATTCGCCGCCAAGCTCT |
| | | TCAGCAATATCACGGGTAGCCAACGCTATGTCCTGATA |
| | | GCGGTCCGCCACACCCAGCCGGCCACAGTCGATGAA |
| | | TCCAGAAAAGCGGCCATTTTCCACCATGATATTCGGCA |
| | | AGCAGGCATCGCCATGGGTCACGACGAGATCCTCGCC |
| | | GTCGGGCATGCTCGCCTTGAGCCTGGCGAACAGTTCG |
| | | GCTGGCGCGAGCCCCTGATGCTCTTCGTCCAGATCAT |
| | | CCTGATCGACAAGACCGGCTTCCATCCGAGTACGTGC |
| | | TCGCTCGATGCGATGTTTCGCTTGGTGGTCGAATGGG |
| | | CAGGTAGCCGGATCAAGCGTATGCAGCCGCCGCATTG |
| | | CATCAGCCATGATGGATACTTTCTCGGCAGGAGCAAG |
| | | GTGAGATGACAGGAGATCCTGCCCCGGCACTTCGCCC |
| | | AATAGCAGCCAGTCCCTTCCCGCTTCAGTGACAACGT |
| | | CGAGCACAGCTGCGCAAGGAACGCCCGTCGTGGCCA |
| | | GCCACGATAGCCGCGCTGCCTCGTCTTGCAGTTCATT |
| | | CAGGGCACCGGACAGGTCGGTCTTGACAAAAAGAACC |
| | | GGGCGCCCCTGCGCTGACAGCCGGAACACGGCGGCA |
| | | TCAGAGCAGCCGATTGTCTGTTGTGCCCAGTCATAGC |
| | | CGAATAGCCTCTCCACCCAAGCGGCCGGAGAACCTGC |
| | | GTGCAATCCATCTTGTTCAATCATGCGAAACGATCCTC |
| | | ATCCTGTCTCTTGATCAGATCTTGATCCCCTGCGCCAT |
| | | CAGATCCTTGGCGGCGAGAAAGCCATCCAGTTTACTTT |
| | | GCAGGGCTTCCCAACCTTACCAGAGGGCGCCCCAGCT |
| | | GGCAATTCCGGTTCGCTTGCTGTCCATAAAACCGCCC |
| | | AGTCTAGCTATCGCCATGTAAGCCCACTGCAAGCTAC |
| | | CTGCTTTCTCTTTGCGCTTGCGTTTTCCCTTGTCCAGA |
| | | TAGCCCAGTAGCTGACATTCATCCGGGGTCAGCACCG |
| | | TTTCTGCGGACTGGCTTTCTACGTGAAAAGGATCTAGG |
| | | TGAAGATCCTTTTTGATAATCTCATGACCAAAATCCCTT |
| | | AACGTGAGTTTTCGTTCCACTGAGCGTCAGACCCCGT |
| | | AGAAAAGATCAAAGGATCTTCTTGAGATCCTTTTTTTCT |
| | | GCGCGTAATCTGCTGCTTGCAAACAAAAAAACCACCG |
| | | CTACCAGCGGTGGTTTGTTTGCCGGATCAAGAGCTAC |
| | | CAACTCTTTTTCCGAAGGTAACTGGCTTCAGCAGAGCG |
| | | CAGATACCAAATACTGTCCTTCTAGTGTAGCCGTAGTT |
| | | AGGCCACCACTTCAAGAACTCTGTAGCACCGCCTACA |
| | | TACCTCGCTCTGCTAATCCTGTTACCAGTGGCTGCTGC |
| | | CAGTGGCGATAAGTCGTGTCTTACCGGGTTGGACTCA |
| | | AGACGATAGTTACCGGATAAGGCGCAGCGGTCGGGCT |
| | | GAACGGGGGGTTCGTGCACACAGCCCAGCTTGGAGC |
| | | GAACGACCTACACCGAACTGAGATACCTACAGCGTGA |
| | | GCTATGAGAAAGCGCCACGCTTCCCGAAGGGAGAAAG |
| | | GCGGACAGGTATCCGGTAAGCGGCAGGGTCGGAACA |
| | | GGAGAGCGCACGAGGGAGCTTCCAGGGGGAAACGCC |
| | | TGGTATCTTTATAGTCCTGTCGGGTTTCGCCACCTCTG |
| | | ACTTGAGCGTCGATTTTTGTGATGCTCGTCAGGGGGG |
| | | CGGAGCCTATGGAAAAACGCCAGCAACGCGGCCTTTT |
| | | TACGGTTCCTGGGCTTTTGCTGGCCTTTTGCTCACATG |
| | | TTCTTTCCTGCGTTATCCCCTGATTCTGTGGATAACCG |
| | | TATTACCGCCTTTGAGTGAGCTGATACCGCTCGCCGC |
| | | AGCCGAACGACCGAGCGCAGCGAGTCAGTGAGCGAG |
| | | GAAGCGGAAGAGCGCCCAATACGCAAACCGCCTCTCC |
| | | CCGCGCGTTGGCCGATTCATTAATGCAGGGCTGCA |
| 64 | 5′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda at positions 53-2027 | TTTCCATCATTCCAGTCGAACTCACACACAACACCAAA |
| | ITR at positions 2049-2178 | TGCATTTAAGTCGCTTGAAATTGCTATAAGCAGAGCAT |
| | CMV i.e enhancer at positions | GTTGCGCCAGCATGATTAATACAGCATTTAATACAGAG |
| | 2267-2636 (part of smCBA | CCGTGTTTATTGAGTCGGTATTCAGAGTCTGACCAGAA |
| | promoter) | ATTATTAATCTGGTGAAGTTATTCCTCTGTCATTACGTC |
| | Chicken β-actin promoter at | ATGGTCGATTTCAATTTCTATTGATGCTTTCCAGTCGTA |
| | positions 2633-2915 (part of | ATCAATGATGTATTTTTTGATGTTTGACCTCTGTTCATA |
| | smCBA promoter) | TCCTCACAGATAAAAAATCGCCCTCACACTGGAGGGC |
| | Exon1 at positions 2916-3008 | AAAGAAGATTTCCAATAATCAGAACAAGTCGGCTCCTG |
| | (part of smCBA promoter) | TTTAGTTACGAGCGACATTGCTCCGTGTATTCACTCGT |
| | Chimeric intron at positions | TGGAATGAATACACAGTGCAGTGTTTATTCTGTTATTTA |
| | 3008-3209 (part of smCBA | TGCCAAAAATTAAGGCCACTATCAGGCAGCTTTGTTGT |
| | promoter) | TCTGTTTACCAAGTTCTCTGGCAATCATTGCCGTCGTT |
| | Kozak sequence at positions | CGTATTGCCCATTTATCGACATATTTCCCATCTTCCTAT |
| | 3226-3235 | ACAGGAAACATTTCTTCAGGCTTAACCATGCATTCCGA |
| | N-terminal portion of human | TTGCAGCTTGCATCCATTGCATCGCTTGAATTGTCCAC |
| | OTOF isoform 5 at positions | ACCATTGATTTTTATCAATAGTCGTAGTTTAACGGATAG |
| | 3232-5637 | TCCTGGTATTGTTCCATCACATCCTGAGGATGCCCTTC |
| | Splice donor sequence at | GAACTCTTCAAATTCTTCTTCCTAATATCACCTTAAATA |
| | positions 5638-5721 | GTGGATTGCGGTAGTAAAGATTGTGCCTGTCTTTTAAC |
| | AP head sequence at positions | CACATCAGGCTCGGTGGTTCTCGTGTACCCCTACAGC |
| | 5728-6014 | GAGAAATCGGATAAACTATTACAACCCCTACAGTTTGT |
| | ITR at positions 6108-6237 | AGAGTATAGAAAATGATCCACTCGTTATTCTCGGACGA |
| | Lambda at positions 6248-8260 | GTGTTCAGTAATGAACCTCTGGAGAGAACCATCTATAT |
| | Ori at positions 8317-8905 | GATCGTTATCTGGGTTTGACTTCTGCTTTTAAGCCCAG |
| | KanR at positions 9083-9892 | ATAACTTGCCTGAATATGTTAATGAGAGAATCGGTATT |
| | Transgene to be transferred | CCTCATGTGTGGCATGTTTTCGTCTTTGCTCTTGCATTT |
| | into vector in dual vector | TCACTAGCAATTAATGTGCATCGATTATCAGCTATTGC |
| | system at positions 2049-6237 | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCGGTACC |
| | | TAGTTATTAATAGTAATCAATTACGGGGTCATTAGTTCA |
| | | TAGCCCATATATGGAGTTCCGCGTTACATAACTTACGG |
| | | TAAATGGCCCGCCTGGCTGACCGCCCAACGACCCCC |
| | | GCCCATTGACGTCAATAATGACGTATGTTCCCATAGTA |
| | | ACGCCAATAGGGACTTTCCATTGACGTCAATGGGTGG |
| | | AGTATTTACGGTAAACTGCCCACTTGGCAGTACATCAA |
| | | GTGTATCATATGCCAAGTACGCCCCCTATTGACGTCAA |
| | | TGACGGTAAATGGCCCGCCTGGCATTATGCCCAGTAC |
| | | ATGACCTTATGGGACTTTCCTACTTGGCAGTACATCTA |
| | | CGTATTAGTCATCGCTATTACCATGGTCGAGGTGAGCC |
| | | CCACGTTCTGCTTCACTCTCCCCATCTCCCCCCCCTCC |
| | | CCACCCCCAATTTTGTATTTATTTATTTTTTAATTATTTT |
| | | GTGCAGCGATGGGGGCGGGGGGGGGGGGGGGGCG |
| | | CGCGCCAGGCGGGGCGGGGCGGGGCGAGGGGCGG |
| | | GGCGGGGCGAGGCGGAGAGGTGCGGCGGCAGCCAA |
| | | TCAGAGCGGCGCGCTCCGAAAGTTTCCTTTTATGGCG |
| | | AGGCGGCGGCGGCGGCGGCCCTATAAAAAGCGAAGC |
| | | GCGCGGCGGGCGGGAGTCGCTGCGCGCTGCCTTCGC |
| | | CCCGTGCCCCGCTCCGCCGCCGCCTCGCGCCGCCCG |
| | | CCCCGGCTCTGACTGACCGCGTTACTCCCACAGGTGA |
| | | GCGGGCGGGACGGCCCTTCTCCTCCGGGCTGTAATTA |
| | | GCGCTTGGTTTAATGACGGCTTGTTTCTTTTCTGTGGC |
| | | TGCGTGAAAGCCTTGAGGGGCTCCGGGAGCTAGAGC |
| | | CTCTGCTAACCATGTTCATGCCTTCTTCTTTTTCCTACA |
| | | GCTCCTGGGCAACGTGCTGGTTATTGTGCTGTCTCAT |
| | | CATTTTGGCAAAGAATTCTAGCGGCCGCCACCATGGC |
| | | CTTGCTCATCCACCTCAAGACAGTCTCGGAGCTGCGG |
| | | GGCAGGGGCGACCGGATCGCCAAAGTGACTTTCCGA |
| | | GGGCAATCCTTCTACTCTCGGGTCCTGGAGAACTGTG |
| | | AGGATGTGGCTGACTTTGATGAGACATTTCGGTGGCC |
| | | GGTGGCCAGCAGCATCGACAGAAATGAGATGCTGGAG |
| | | ATTCAGGTTTTCAACTACAGCAAAGTCTTCAGCAACAA |
| | | GCTCATCGGGACCTTCCGCATGGTGCTGCAGAAGGTG |
| | | GTAGAGGAGAGCCATGTGGAGGTGACTGACACGCTGA |
| | | TTGATGACAACAATGCTATCATCAAGACCAGCCTGTGC |
| | | GTGGAGGTCCGGTATCAGGCCACTGACGGCACAGTG |
| | | GGCTCCTGGGACGATGGGGACTTCCTGGGAGATGAG |
| | | TCTCTTCAAGAGGAAGAGAAGGACAGCCAAGAGACGG |
| | | ATGGACTGCTCCCAGGCTCCCGGCCCAGCTCCCGGC |
| | | CCCCAGGAGAGAAGAGCTTCCGGAGAGCCGGGAGGA |
| | | GCGTGTTCTCCGCCATGAAGCTCGGCAAAAACCGGTC |
| | | TCACAAGGAGGAGCCCCAAAGACCAGATGAACCGGC |
| | | GGTGCTGGAGATGGAAGACCTTGACCATCTGGCCATT |
| | | CGGCTAGGAGATGGACTGGATCCCGACTCGGTGTCTC |
| | | TAGCCTCAGTCACAGCTCTCACCACTAATGTCTCCAAC |
| | | AAGCGATCTAAGCCAGACATTAAGATGGAGCCAAGTG |
| | | CTGGGCGGCCCATGGATTACCAGGTCAGCATCACGGT |
| | | GATCGAGGCCCGGCAGCTGGTGGGCTTGAACATGGA |
| | | CCCTGTGGTGTGCGTGGAGGTGGGTGACGACAAGAA |
| | | GTACACATCCATGAAGGAGTCCACTAACTGCCCCTATT |
| | | ACAACGAGTACTTCGTCTTCGACTTCCATGTCTCTCCG |
| | | GATGTCATGTTTGACAAGATCATCAAGATTTCGGTGAT |
| | | TCACTCCAAGAACCTGCTGCGCAGTGGCACCCTGGTG |
| | | GGCTCCTTCAAAATGGACGTGGGAACCGTGTACTCGC |
| | | AGCCAGAGCACCAGTTCCATCACAAGTGGGCCATCCT |
| | | GTCTGACCCCGATGACATCTCCTCGGGGCTGAAGGGC |
| | | TACGTGAAGTGTGACGTTGCCGTGGTGGGCAAAGGG |
| | | GACAACATCAAGACGCCCCACAAGGCCAATGAGACCG |
| | | ACGAAGATGACATTGAGGGGAACTTGCTGCTCCCCGA |
| | | GGGGGTGCCCCCCGAACGCCAGTGGGCCCGGTTCTA |
| | | TGTGAAAATTTACCGAGCAGAGGGGCTGCCCCGTATG |
| | | AACACAAGCCTCATGGCCAATGTAAAGAAGGCTTTCAT |
| | | CGGTGAAAACAAGGACCTCGTGGACCCCTACGTGCAA |
| | | GTCTTCTTTGCTGGCCAGAAGGGCAAGACTTCAGTGC |
| | | AGAAGAGCAGCTATGAGCCCCTGTGGAATGAGCAGGT |
| | | CGTCTTTACAGACCTCTTCCCCCCACTCTGCAAACGCA |
| | | TGAAGGTGCAGATCCGAGACTCGGACAAGGTCAACGA |
| | | CGTGGCCATCGGCACCCACTTCATTGACCTGCGCAAG |
| | | ATTTCTAATGACGGAGACAAAGGCTTCCTGCCCACACT |
| | | GGGCCCAGCCTGGGTGAACATGTACGGCTCCACACGT |
| | | AACTACACGCTGCTGGATGAGCATCAGGACCTGAACG |
| | | AGGGCCTGGGGGAGGGTGTGTCCTTCCGGGCCCGGC |
| | | TCCTGCTGGGCCTGGCTGTGGAGATCGTAGACACCTC |
| | | CAACCCTGAGCTCACCAGCTCCACAGAGGTGCAGGTG |
| | | GAGCAGGCCACGCCCATCTCGGAGAGCTGTGCAGGT |
| | | AAAATGGAAGAATTCTTTCTCTTTGGAGCCTTCCTGGA |
| | | GGCCTCAATGATCGACCGGAGAAACGGAGACAAGCCC |
| | | ATCACCTTTGAGGTCACCATAGGCAACTATGGGAACG |
| | | AAGTTGATGGCCTGTCCCGGCCCCAGCGGCCTCGGC |
| | | CCCGGAAGGAGCCGGGGGATGAGGAAGAAGTAGACC |
| | | TGATTCAGAACGCAAGTGATGACGAGGCCGGTGATGC |
| | | CGGGGACCTGGCCTCAGTCTCCTCCACTCCACCAATG |
| | | CGGCCCCAGGTCACCGACAGGAACTACTTCCATCTGC |
| | | CCTACCTGGAGCGAAAGCCCTGCATCTACATCAAGAG |
| | | CTGGTGGCCGGACCAGCGCCGCCGCCTCTACAATGC |
| | | CAACATCATGGACCACATTGCCGACAAGCTGGAAGAA |
| | | GGCCTGAACGACATACAGGAGATGATCAAAACGGAGA |
| | | AGTCCTACCCTGAGCGTCGCCTGCGGGGCGTCCTGG |
| | | AGGAGCTGAGCTGTGGCTGCTGCCGCTTCCTCTCCCT |
| | | CGCTGACAAGGACCAGGGCCACTCATCCCGCACCAG |
| | | GCTTGACCGGGAGCGCCTCAAGTCCTGCATGAGGGA |
| | | GCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACCTCGAGTTAAGGGCGA |
| | | ATTCCCGATAAGGATCTTCCTAGAGCATGGCTACGTAG |
| | | ATAAGTAGCATGGCGGGTTAATCATTAACTACAAGGAA |
| | | CCCCTAGTGATGGAGTTGGCCACTCCCTCTCTGCGCG |
| | | CTCGCTCGCTCACTGAGGCCGGGCGACCAAAGGTCG |
| | | CCCGACGCCCGGGCTTTGCCCGGGCGGCCTCAGTGA |
| | | GCGAGCGAGCGCGCAGCCTTAATTAAATCCACATCTG |
| | | TATGTTTTTTATATTAATTTATTTTTTGCAGGGGGGCAT |
| | | TGTTTGGTAGGTGAGAGTTCTGAATTGCTATGTTTAGT |
| | | GAGTTGTATCTATTTATTTTTCAATAAATACAATTAGTTA |
| | | TGTGTTTTGGGGGCGATCGTGAGGCAAAGAAAACCCG |
| | | GCGCTGAGGCCGGGTTATTCTTGTTCTCTGGTCAAATT |
| | | ATATAGTTGGAAAACAAGGATGCATATATGAATGAACG |
| | | ATGCAGAGGCAATGCCGATGGCGATAGTGGGTATCAG |
| | | GTAGCCGCTTATGCTGGAAAGAAGCAATAACCCGCAG |
| | | AAAAACAAAGCTCCAAGCTCAACAAAACTAAGGGCATA |
| | | GACAATAACTACCTATGTCATATACCCATACTCTCTAAT |
| | | CTTGGCCAGTCGGCGCGTTCTGCTTCCGATTAGAAAC |
| | | GTCAAGGCAGCAATCAGGATTGCAATCTTGGTTCCTG |
| | | CATAGGATGACAATGTCGCCCCAAGACCATCTCTATGA |
| | | GCTGAAAAAGAAACACAAGGAATGTAGTGGCGGAAAA |
| | | GGAGATAGCAAATGCTTACGATAACGTAAGGAATTATT |
| | | ACTATGTAAACACCAGGCAAGATTCTGTTCCGTATAAT |
| | | TACTCCTGATAATTAATCCTTAACTTTGCCCACCTGCCT |
| | | TTTAAAACATTCCAGTATATCACTTTTCATTCTTGCGTA |
| | | GCAATATGCCCTCTCTTCAGCTATCTCAGCATTGGTGA |
| | | CCTTGTTCAGAGGCGCTGAGAGATGGCCTTTTTCTGAT |
| | | AGATAATGTTCTGTTAAAATATCTCCGGCCTCATCTTTT |
| | | GCCCGCAGGCTAATGTCTGAAAATTGAGGTGACGGGT |
| | | TAAAAATAATATCCTTGGCAACCTTTTTTATATCCCTTTT |
| | | AAATTTTGGCTTAATGACTATATCCAATGAGTCAAAAAG |
| | | CTCCCCTTCAATATCTGTTGCCCCTAAGACCTTTAATAT |
| | | ATCGCCAAATACAGGTAGCTTGGCTTCTACCTTCACCG |
| | | TTGTTCTGCCGATGAAATGCTAATGCATAACATCGTCT |
| | | TTGGTGGTTCCCCTCATCAGTGGCTCTATCTGAACGC |
| | | GCTCTCCACTGCTTAATGACATTCCTTTCCCGATTAAA |
| | | AAATCTGTCAGATCGGATGTGGTCGGCCCGAAAACAG |
| | | TTCTGGCAAAACCAATGGTGTCGCCTTCAACAAACAAA |
| | | AAAGATGGGAATCCCAATGATTCGTCATCTGCGAGGC |
| | | TGTTCTTAATATCTTCAACTGTAGCTTTAGAGCGATTTA |
| | | TCTTCTGAACCAGACTCTTGTCATTTGTTTTGGTAAAGA |
| | | GAAAAGTTTTTCCATCGATTTTATGAATATACAAATAAT |
| | | TGGAGCCAACCTTCAGGTGATGATTATCAGCCAGCAG |
| | | AGAATTAAGGAAAACAGACAGGTTTATTGAGCACTTAT |
| | | CTTTCCCTTTATTTTTGCTGCGGTAAGTCGCATAAAAAC |
| | | CATTCTTCACAATTCAATCCATTTACTATGTTATGTTCT |
| | | GAGGGGAGTGAAAATTCCCCTAATTCGATGAAGATTCT |
| | | TGCTAAATTGTTATCAGCTATGCGCCGACCAGAACACC |
| | | TTGCCGATCAGCCAAACGTCTAATCAGGCCACTGACTA |
| | | GCGATAACTTTCCCCACAACGGAACAACTCTCATTGCA |
| | | TGGGATAATTGGGTACTGTGGGTTTAGTGGTTGTAAAA |
| | | ACACCTGACCGCTATCCCTGATCAGTTTCTTGAAGGTA |
| | | AACTCATCACCCCCAAGTCTGGCTATACAGAAATCACC |
| | | TGGCTCAACAGCCTGCTCAGGGTCAACGAGAATTTAC |
| | | ATTCCGTCAGGATAGCTTGGCTTGGAGCCTGTTGGTG |
| | | CGGTCACGGAATTACCTTCAACCTCAAGCCAGAATGC |
| | | AGAATCACTGGCTTTTTTGGTTGTGCTTACCCATCTCT |
| | | CCGCATCACCTTTGGTAAAGGTTCTAAGCTAAGGTGAG |
| | | AACATCCCTGCCTGAACATGAGAAAAAACAGGGTACTC |
| | | ATACTCACTTATTAGTGACGGCTATGAGCAAAAGGCCA |
| | | GCAAAAGGCCAGGAACCGTAAAAAGGCCGCGTTGCTG |
| | | GCGTTTTTCCATAGGCTCCGCCCCCCTGACGAGCATC |
| | | ACAAAAATCGACGCTCAAGTCAGAGGTGGCGAAACCC |
| | | GACAGGACTATAAAGATACCAGGCGTTTCCCCCTGGA |
| | | AGCTCCCTCGTGCGCTCTCCTGTTCCGACCCTGCCGC |
| | | TTACCGGATACCTGTCCGCCTTTCTCCCTTCGGGAAG |
| | | CGTGGCGCTTTCTCATAGCTCACGCTGTAGGTATCTCA |
| | | GTTCGGTGTAGGTCGTTCGCTCCAAGCTGGGCTGTGT |
| | | GCACGAACCCCCCGTTCAGCCCGACCGCTGCGCCTTA |
| | | TCCGGTAACTATCGTCTTGAGTCCAACCCGGTAAGACA |
| | | CGACTTATCGCCACTGGCAGCAGCCACTGGTAACAGG |
| | | ATTAGCAGAGCGAGGTATGTAGGCGGTGCTACAGAGT |
| | | TCTTGAAGTGGTGGCCTAACTACGGCTACACTAGAAG |
| | | AACAGTATTTGGTATCTGCGCTCTGCTGAAGCCAGTTA |
| | | CCTTCGGAAAAAGAGTTGGTAGCTCTTGATCCGGCAA |
| | | ACAAACCACCGCTGGTAGCGGTGGTTTTTTTGTTTGCA |
| | | AGCAGCAGATTACGCGCAGAAAAAAAGGATCTCAAGA |
| | | AGATCCTTTGATCTTTTCTACGGGGTCTGACGCTCAGT |
| | | GGAACGAAAACTCACGTTAAGGGATTTTGGTCATGAGA |
| | | TTATCAAAAAGGATCTTCACCTAGATCCTTTTAAATTAA |
| | | AAATGAAGTTTTAAATCAAGCCCAATCTGAATAATGTTA |
| | | CAACCAATTAACCAATTCTGATTAGAAAAACTCATCGA |
| | | GCATCAAATGAAACTGCAATTTATTCATATCAGGATTAT |
| | | CAATACCATATTTTTGAAAAAGCCGTTTCTGTAATGAAG |
| | | GAGAAAACTCACCGAGGCAGTTCCATAGGATGGCAAG |
| | | ATCCTGGTATCGGTCTGCGATTCCGACTCGTCCAACAT |
| | | CAATACAACCTATTAATTTCCCCTCGTCAAAAATAAGGT |
| | | TATCAAGTGAGAAATCACCATGAGTGACGACTGAATCC |
| | | GGTGAGAATGGCAAAAGTTTATGCATTTCTTTCCAGAC |
| | | TTGTTCAACAGGCCAGCCATTACGCTCGTCATCAAAAT |
| | | CACTCGCATCAACCAAACCGTTATTCATTCGTGATTGC |
| | | GCCTGAGCAAGACGAAATACGCGATCGCTGTTAAAAG |
| | | GACAATTACAAACAGGAATCGAATGCAACCGGCGCAG |
| | | GAACACTGCCAGCGCATCAACAATATTTTCACCTGAAT |
| | | CAGGATATTCTTCTAATACCTGGAATGCTGTTTTTCCG |
| | | GGGATCGCAGTGGTGAGTAACCATGCATCATCAGGAG |
| | | TACGGATAAAATGCTTGATGGTCGGAAGAGGCATAAAT |
| | | TCCGTCAGCCAGTTTAGTCTGACCATCTCATCTGTAAC |
| | | ATCATTGGCAACGCTACCTTTGCCATGTTTCAGAAACA |
| | | ACTCTGGCGCATCGGGCTTCCCATACAAGCGATAGAT |
| | | TGTCGCACCTGATTGCCCGACATTATCGCGAGCCCAT |
| | | TTATACCCATATAAATCAGCATCCATGTTGGAATTTAAT |
| | | CGCGGCCTCGACGTTTCCCGTTGAATATGGCTCATAA |
| | | CACCCCTTGTATTACTGTTTATGTAAGCAGACAGTTTTA |
| | | TTGTTCATGATGATATATTTTTATCTTGTGCAATGTAAC |
| | | ATCAGAGATTTTGAGACACGGGCCAGAGCTGCA |
| 65 | 5′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | CMV enhancer at positions | AGTGAGCGAGCGAGCGCGCAGAGAGGGAGTGGCCAA |
| | 177-546 (part of smCBA | CTCCATCACTAGGGGTTCCTCAGATCTGAATTCGGTAC |
| | promoter) | CTAGTTATTAATAGTAATCAATTACGGGGTCATTAGTTC |
| | Chicken β-actin promoter at | ATAGCCCATATATGGAGTTCCGCGTTACATAACTTACG |
| | positions 548-825 (part of | GTAAATGGCCCGCCTGGCTGACCGCCCAACGACCCC |
| | smCBA promoter) | CGCCCATTGACGTCAATAATGACGTATGTTCCCATAGT |
| | Exon1 at positions 826-918 | AACGCCAATAGGGACTTTCCATTGACGTCAATGGGTG |
| | (part of smCBA promoter) | GAGTATTTACGGTAAACTGCCCACTTGGCAGTACATCA |
| | Chimeric intron at positions | AGTGTATCATATGCCAAGTACGCCCCCTATTGACGTCA |
| | 918-1119 (part of smCBA | ATGACGGTAAATGGCCCGCCTGGCATTATGCCCAGTA |
| | promoter) | CATGACCTTATGGGACTTTCCTACTTGGCAGTACATCT |
| | N-terminal portion of human | ACGTATTAGTCATCGCTATTACCATGGTCGAGGTGAGC |
| | OTOF isoform 5 at positions | CCCACGTTCTGCTTCACTCTCCCCATCTCCCCCCCCTC |
| | 1142-3547 | CCCACCCCCAATTTTGTATTTATTTATTTTTTAATTATTT |
| | Splice donor sequence at | TGTGCAGCGATGGGGGCGGGGGGGGGGGGGGGGCG |
| | positions 3548-3631 | CGCGCCAGGCGGGGCGGGGCGGGGCGAGGGGCGG |
| | AP head sequence at positions | GGCGGGGCGAGGCGGAGAGGTGCGGCGGCAGCCAA |
| | 3638-3924 | TCAGAGCGGCGCGCTCCGAAAGTTTCCTTTTATGGCG |
| | ITR at positions 3948-4090 | AGGCGGCGGCGGCGGCGGCCCTATAAAAAGCGAAGC |
| | KanR at positions 4616-5410 | GCGCGGCGGGCGGGAGTCGCTGCGCGCTGCCTTCGC |
| | pUC ori at positions 5796-6466 | CCCGTGCCCCGCTCCGCCGCCGCCTCGCGCCGCCCG |
| | Transgene to be transferred | CCCCGGCTCTGACTGACCGCGTTACTCCCACAGGTGA |
| | into vector in dual vector | GCGGGCGGGACGGCCCTTCTCCTCCGGGCTGTAATTA |
| | system at positions 19-4090 | GCGCTTGGTTTAATGACGGCTTGTTTCTTTTCTGTGGC |
| | | TGCGTGAAAGCCTTGAGGGGCTCCGGGAGCTAGAGC |
| | | CTCTGCTAACCATGTTCATGCCTTCTTCTTTTTCCTACA |
| | | GCTCCTGGGCAACGTGCTGGTTATTGTGCTGTCTCAT |
| | | CATTTTGGCAAAGAATTCTAGCGGCCGCCACCATGGC |
| | | CTTGCTCATCCACCTCAAGACAGTCTCGGAGCTGCGG |
| | | GGCAGGGGCGACCGGATCGCCAAAGTGACTTTCCGA |
| | | GGGCAATCCTTCTACTCTCGGGTCCTGGAGAACTGTG |
| | | AGGATGTGGCTGACTTTGATGAGACATTTCGGTGGCC |
| | | GGTGGCCAGCAGCATCGACAGAAATGAGATGCTGGAG |
| | | ATTCAGGTTTTCAACTACAGCAAAGTCTTCAGCAACAA |
| | | GCTCATCGGGACCTTCCGCATGGTGCTGCAGAAGGTG |
| | | GTAGAGGAGAGCCATGTGGAGGTGACTGACACGCTGA |
| | | TTGATGACAACAATGCTATCATCAAGACCAGCCTGTGC |
| | | GTGGAGGTCCGGTATCAGGCCACTGACGGCACAGTG |
| | | GGCTCCTGGGACGATGGGGACTTCCTGGGAGATGAG |
| | | TCTCTTCAAGAGGAAGAGAAGGACAGCCAAGAGACGG |
| | | ATGGACTGCTCCCAGGCTCCCGGCCCAGCTCCCGGC |
| | | CCCCAGGAGAGAAGAGCTTCCGGAGAGCCGGGAGGA |
| | | GCGTGTTCTCCGCCATGAAGCTCGGCAAAAACCGGTC |
| | | TCACAAGGAGGAGCCCCAAAGACCAGATGAACCGGC |
| | | GGTGCTGGAGATGGAAGACCTTGACCATCTGGCCATT |
| | | CGGCTAGGAGATGGACTGGATCCCGACTCGGTGTCTC |
| | | TAGCCTCAGTCACAGCTCTCACCACTAATGTCTCCAAC |
| | | AAGCGATCTAAGCCAGACATTAAGATGGAGCCAAGTG |
| | | CTGGGCGGCCCATGGATTACCAGGTCAGCATCACGGT |
| | | GATCGAGGCCCGGCAGCTGGTGGGCTTGAACATGGA |
| | | CCCTGTGGTGTGCGTGGAGGTGGGTGACGACAAGAA |
| | | GTACACATCCATGAAGGAGTCCACTAACTGCCCCTATT |
| | | ACAACGAGTACTTCGTCTTCGACTTCCATGTCTCTCCG |
| | | GATGTCATGTTTGACAAGATCATCAAGATTTCGGTGAT |
| | | TCACTCCAAGAACCTGCTGCGCAGTGGCACCCTGGTG |
| | | GGCTCCTTCAAAATGGACGTGGGAACCGTGTACTCGC |
| | | AGCCAGAGCACCAGTTCCATCACAAGTGGGCCATCCT |
| | | GTCTGACCCCGATGACATCTCCTCGGGGCTGAAGGGC |
| | | TACGTGAAGTGTGACGTTGCCGTGGTGGGCAAAGGG |
| | | GACAACATCAAGACGCCCCACAAGGCCAATGAGACCG |
| | | ACGAAGATGACATTGAGGGGAACTTGCTGCTCCCCGA |
| | | GGGGGTGCCCCCCGAACGCCAGTGGGCCCGGTTCTA |
| | | TGTGAAAATTTACCGAGCAGAGGGGCTGCCCCGTATG |
| | | AACACAAGCCTCATGGCCAATGTAAAGAAGGCTTTCAT |
| | | CGGTGAAAACAAGGACCTCGTGGACCCCTACGTGCAA |
| | | GTCTTCTTTGCTGGCCAGAAGGGCAAGACTTCAGTGC |
| | | AGAAGAGCAGCTATGAGCCCCTGTGGAATGAGCAGGT |
| | | CGTCTTTACAGACCTCTTCCCCCCACTCTGCAAACGCA |
| | | TGAAGGTGCAGATCCGAGACTCGGACAAGGTCAACGA |
| | | CGTGGCCATCGGCACCCACTTCATTGACCTGCGCAAG |
| | | ATTTCTAATGACGGAGACAAAGGCTTCCTGCCCACACT |
| | | GGGCCCAGCCTGGGTGAACATGTACGGCTCCACACGT |
| | | AACTACACGCTGCTGGATGAGCATCAGGACCTGAACG |
| | | AGGGCCTGGGGGAGGGTGTGTCCTTCCGGGCCCGGC |
| | | TCCTGCTGGGCCTGGCTGTGGAGATCGTAGACACCTC |
| | | CAACCCTGAGCTCACCAGCTCCACAGAGGTGCAGGTG |
| | | GAGCAGGCCACGCCCATCTCGGAGAGCTGTGCAGGT |
| | | AAAATGGAAGAATTCTTTCTCTTTGGAGCCTTCCTGGA |
| | | GGCCTCAATGATCGACCGGAGAAACGGAGACAAGCCC |
| | | ATCACCTTTGAGGTCACCATAGGCAACTATGGGAACG |
| | | AAGTTGATGGCCTGTCCCGGCCCCAGCGGCCTCGGC |
| | | CCCGGAAGGAGCCGGGGGATGAGGAAGAAGTAGACC |
| | | TGATTCAGAACGCAAGTGATGACGAGGCCGGTGATGC |
| | | CGGGGACCTGGCCTCAGTCTCCTCCACTCCACCAATG |
| | | CGGCCCCAGGTCACCGACAGGAACTACTTCCATCTGC |
| | | CCTACCTGGAGCGAAAGCCCTGCATCTACATCAAGAG |
| | | CTGGTGGCCGGACCAGCGCCGCCGCCTCTACAATGC |
| | | CAACATCATGGACCACATTGCCGACAAGCTGGAAGAA |
| | | GGCCTGAACGACATACAGGAGATGATCAAAACGGAGA |
| | | AGTCCTACCCTGAGCGTCGCCTGCGGGGCGTCCTGG |
| | | AGGAGCTGAGCTGTGGCTGCTGCCGCTTCCTCTCCCT |
| | | CGCTGACAAGGACCAGGGCCACTCATCCCGCACCAG |
| | | GCTTGACCGGGAGCGCCTCAAGTCCTGCATGAGGGA |
| | | GCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACTGGGGAGAGATCTGAG |
| | | GAACCCCTAGTGATGGAGTTGGCCACTCCCTCTCTGC |
| | | GCGCTCGCTCGCTCACTGAGGCCGCCCGGGCAAAGC |
| | | CCGGGCGTCGGGCGACCTTTGGTCGCCCGGCCTCAG |
| | | TGAGCGAGCGAGCGCGCAGAGAGGGAGTGGCCAACC |
| | | CCCCCCCCCCCCCCCCTGCAGCCTGGCGTAATAGCG |
| | | AAGAGGCCCGCACCGATCGCCCTTCCCAACAGTTGCG |
| | | TAGCCTGAATGGCGAATGGCGCGACGCGCCCTGTAG |
| | | CGGCGCATTAAGCGCGGCGGGTGTGGTGGTTACGCG |
| | | CAGCGTGACCGCTACACTTGCCAGCGCCCTAGCGCCC |
| | | GCTCCTTTCGCTTTCTTCCCTTCCTTTCTCGCCACGTT |
| | | CGCCGGCTTTCCCCGTCAAGCTCTAAATCGGGGGCTC |
| | | CCTTTAGGGTTCCGATTTAGTGCTTTACGGCACCTCGA |
| | | CCCCAAAAAACTTGATTAGGGTGATGGTTCACGTAGTG |
| | | GGCCATCGCCCTGATAGACGGTTTTTCGCCCTTTGAC |
| | | GTTGGAGTCCACGTTCTTTAATAGTGGACTCTTGTTCC |
| | | AAACTGGAACAACACTCAACCCTATCGCGGTCTATTCT |
| | | TTTGATTTATAAGGGATGTTGCCGATTTCGGCCTATTG |
| | | GTTAAAAAATGAGCTGATTTAACAAAAATTTTAACAAAA |
| | | TTCAGAAGAACTCGTCAAGAAGGCGATAGAAGGCGAT |
| | | GCGCTGCGAATCGGGAGCGGCGATACCGTAAAGCAC |
| | | GAGGAAGCGGTCAGCCCATTCGCCGCCAAGCTCTTCA |
| | | GCAATATCACGGGTAGCCAACGCTATGTCCTGATAGC |
| | | GGTCCGCCACACCCAGCCGGCCACAGTCGATGAATCC |
| | | AGAAAAGCGGCCATTTTCCACCATGATATTCGGCAAGC |
| | | AGGCATCGCCATGGGTCACGACGAGATCCTCGCCGTC |
| | | GGGCATGCTCGCCTTGAGCCTGGCGAACAGTTCGGCT |
| | | GGCGCGAGCCCCTGATGCTCTTCGTCCAGATCATCCT |
| | | GATCGACAAGACCGGCTTCCATCCGAGTACGTGCTCG |
| | | CTCGATGCGATGTTTCGCTTGGTGGTCGAATGGGCAG |
| | | GTAGCCGGATCAAGCGTATGCAGCCGCCGCATTGCAT |
| | | CAGCCATGATGGATACTTTCTCGGCAGGAGCAAGGTG |
| | | AGATGACAGGAGATCCTGCCCCGGCACTTCGCCCAAT |
| | | AGCAGCCAGTCCCTTCCCGCTTCAGTGACAACGTCGA |
| | | GCACAGCTGCGCAAGGAACGCCCGTCGTGGCCAGCC |
| | | ACGATAGCCGCGCTGCCTCGTCTTGCAGTTCATTCAG |
| | | GGCACCGGACAGGTCGGTCTTGACAAAAAGAACCGG |
| | | GCGCCCCTGCGCTGACAGCCGGAACACGGCGGCATC |
| | | AGAGCAGCCGATTGTCTGTTGTGCCCAGTCATAGCCG |
| | | AATAGCCTCTCCACCCAAGCGGCCGGAGAACCTGCGT |
| | | GCAATCCATCTTGTTCAATCATGCGAAACGATCCTCAT |
| | | CCTGTCTCTTGATCAGATCTTGATCCCCTGCGCCATCA |
| | | GATCCTTGGCGGCGAGAAAGCCATCCAGTTTACTTTG |
| | | CAGGGCTTCCCAACCTTACCAGAGGGCGCCCCAGCTG |
| | | GCAATTCCGGTTCGCTTGCTGTCCATAAAACCGCCCA |
| | | GTCTAGCTATCGCCATGTAAGCCCACTGCAAGCTACCT |
| | | GCTTTCTCTTTGCGCTTGCGTTTTCCCTTGTCCAGATA |
| | | GCCCAGTAGCTGACATTCATCCGGGGTCAGCACCGTT |
| | | TCTGCGGACTGGCTTTCTACGTGAAAAGGATCTAGGT |
| | | GAAGATCCTTTTTGATAATCTCATGACCAAAATCCCTTA |
| | | ACGTGAGTTTTCGTTCCACTGAGCGTCAGACCCCGTA |
| | | GAAAAGATCAAAGGATCTTCTTGAGATCCTTTTTTTCTG |
| | | CGCGTAATCTGCTGCTTGCAAACAAAAAAACCACCGCT |
| | | ACCAGCGGTGGTTTGTTTGCCGGATCAAGAGCTACCA |
| | | ACTCTTTTTCCGAAGGTAACTGGCTTCAGCAGAGCGCA |
| | | GATACCAAATACTGTCCTTCTAGTGTAGCCGTAGTTAG |
| | | GCCACCACTTCAAGAACTCTGTAGCACCGCCTACATAC |
| | | CTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGA |
| | | ACGACCTACACCGAACTGAGATACCTACAGCGTGAGC |
| | | TATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGGCTTTTGCTGGCCTTTTGCTCACATGTT |
| | | CTTTCCTGCGTTATCCCCTGATTCTGTGGATAACCGTA |
| | | TTACCGCCTTTGAGTGAGCTGATACCGCTCGCCGCAG |
| | | CCGAACGACCGAGCGCAGCGAGTCAGTGAGCGAGGA |
| | | AGCGGAAGAGCGCCCAATACGCAAACCGCCTCTCCCC |
| | | GCGCGTTGGCCGATTCATTAATGCAGGGCTGCA |
| 66 | 5′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | Myo15 promoter at positions | GGGAGTGGCCAACTCCATCACTAGGGGTTCCTTGTAG |
| | 235-1199 | TTAATGATTAACCCGCCATGCTACTTATCTACGTAGCC |
| | Kozak sequence at positions | ATGCTCTAGGAAGATCGGAATTCGCCCTTAAGCTAGC |
| | 1216-1225 | GGCGCGCCCAATTCTGCAGCTCAGCCTACTACTTGCT |
| | N-terminal portion of human | TTCCAGGCTGTTCCTAGTTCCCATGTCAGCTGCTTGTG |
| | OTOF isoform 5 at positions | CTTTCCAGAGACAAAACAGGAATAATAGATGTCATTAA |
| | 1222-3627 | ATATACATTGGGCCCCAGGCGGTCAATGTGGCAGCCT |
| | Splice donor sequence at | GAGCCTCCTTTCCATCTCTGTGGAGGCAGACATAGGA |
| | positions 3628-3711 | CCCCCAACAAACAGCATGCAGGTTGGGAGCCAGCCAC |
| | AP head sequence at positions | AGGACCCAGGTAAGGGGCCCTGGGTCCTTAAGCTTCT |
| | 3718-4004 | GCCACTGGCTCCGGCATTGCAGAGAGAAGAGAAGGG |
| | ITR at positions 4098-4227 | GCGGCAGACTGGAGAGCTGGGCTCCATTTTTGTTCCT |
| | M13 fwd at positions 4246-4262 | TGGTGCCCTGCCCCTCCCCATGACCTGCAGAGACATT |
| | f1 ori at positions 4404-4859 | CAGCCTGCCAGGCTTTATGAGGTGGGAGCTGGGCTCT |
| | AmpR promoter at positions | CCCTGATGTATTATTCAGCTCCCTGGAGTTGGCCAGCT |
| | 4885-4989 | CCTGTTACACTGGCCACAGCCCTGGGCATCCGCTTCT |
| | KanR at positions 4990-5799 | CACTTCTAGTTTCCCCTCCAAGGTAATGTGGTGGGTCA |
| | mutBsmBI at positions 5430- | TGATCATTCTATCCTGGCTTCAGGGACCTGACTCCACT |
| | 5430 | TTGGGGCCATTCGAGGGGTCTAGGGTAGATGATGTCC |
| | ori at positions 5970-6558 | CCCTGTGGGGATTAATGTCCTGCTCTGTAAAACTGAGC |
| | CAP binding site at positions | TAGCTGAGATCCAGGAGGGCTTGGCCAGAGACAGCAA |
| | 6846-6867 | GTTGTTGCCATGGTGACTTTAAAGCCAGGTTGCTGCC |
| | lac promoter at positions 6882- | CCAGCACAGGCCTCCCAGTCTACCCTCACTAGAAAAC |
| | 6912 | AACACCCAGGCACTTTCCACCACCTCTCAAAGGTGAAA |
| | lac operator at positions 6920- | CCCAAGGCTGGTCTAGAGAATGAATTATGGATCCTCG |
| | 6936 | CTGTCCGTGCCACCCAGCTAGTCCCAGCGGCTCAGAC |
| | M13 rev at positions 6944-6960 | ACTGAGGAGAGACTGTAGGTTCAGCTACAAGCAAAAA |
| | Transgene to be transferred | GACCTAGCTGGTCTCCAAGCAGTGTCTCCAAGTCCCT |
| | into vector in dual vector | GAACCTGTGACACCTGCCCCAGGCATCATCAGGCACA |
| | system at positions 12-4227 | GAGGGCCACCAAGAATTCTAGCGGCCGCCACCATGG |
| | | CCTTGCTCATCCACCTCAAGACAGTCTCGGAGCTGCG |
| | | GGGCAGGGGCGACCGGATCGCCAAAGTGACTTTCCG |
| | | AGGGCAATCCTTCTACTCTCGGGTCCTGGAGAACTGT |
| | | GAGGATGTGGCTGACTTTGATGAGACATTTCGGTGGC |
| | | CGGTGGCCAGCAGCATCGACAGAAATGAGATGCTGGA |
| | | GATTCAGGTTTTCAACTACAGCAAAGTCTTCAGCAACA |
| | | AGCTCATCGGGACCTTCCGCATGGTGCTGCAGAAGGT |
| | | GGTAGAGGAGAGCCATGTGGAGGTGACTGACACGCT |
| | | GATTGATGACAACAATGCTATCATCAAGACCAGCCTGT |
| | | GCGTGGAGGTCCGGTATCAGGCCACTGACGGCACAG |
| | | TGGGCTCCTGGGACGATGGGGACTTCCTGGGAGATG |
| | | AGTCTCTTCAAGAGGAAGAGAAGGACAGCCAAGAGAC |
| | | GGATGGACTGCTCCCAGGCTCCCGGCCCAGCTCCCG |
| | | GCCCCCAGGAGAGAAGAGCTTCCGGAGAGCCGGGAG |
| | | GAGCGTGTTCTCCGCCATGAAGCTCGGCAAAAACCGG |
| | | TCTCACAAGGAGGAGCCCCAAAGACCAGATGAACCGG |
| | | CGGTGCTGGAGATGGAAGACCTTGACCATCTGGCCAT |
| | | TCGGCTAGGAGATGGACTGGATCCCGACTCGGTGTCT |
| | | CTAGCCTCAGTCACAGCTCTCACCACTAATGTCTCCAA |
| | | CAAGCGATCTAAGCCAGACATTAAGATGGAGCCAAGT |
| | | GCTGGGCGGCCCATGGATTACCAGGTCAGCATCACG |
| | | GTGATCGAGGCCCGGCAGCTGGTGGGCTTGAACATG |
| | | GACCCTGTGGTGTGCGTGGAGGTGGGTGACGACAAG |
| | | AAGTACACATCCATGAAGGAGTCCACTAACTGCCCCTA |
| | | TTACAACGAGTACTTCGTCTTCGACTTCCATGTCTCTC |
| | | CGGATGTCATGTTTGACAAGATCATCAAGATTTCGGTG |
| | | ATTCACTCCAAGAACCTGCTGCGCAGTGGCACCCTGG |
| | | TGGGCTCCTTCAAAATGGACGTGGGAACCGTGTACTC |
| | | GCAGCCAGAGCACCAGTTCCATCACAAGTGGGCCATC |
| | | CTGTCTGACCCCGATGACATCTCCTCGGGGCTGAAGG |
| | | GCTACGTGAAGTGTGACGTTGCCGTGGTGGGCAAAGG |
| | | GGACAACATCAAGACGCCCCACAAGGCCAATGAGACC |
| | | GACGAAGATGACATTGAGGGGAACTTGCTGCTCCCCG |
| | | AGGGGGTGCCCCCCGAACGCCAGTGGGCCCGGTTCT |
| | | ATGTGAAAATTTACCGAGCAGAGGGGCTGCCCCGTAT |
| | | GAACACAAGCCTCATGGCCAATGTAAAGAAGGCTTTCA |
| | | TCGGTGAAAACAAGGACCTCGTGGACCCCTACGTGCA |
| | | AGTCTTCTTTGCTGGCCAGAAGGGCAAGACTTCAGTG |
| | | CAGAAGAGCAGCTATGAGCCCCTGTGGAATGAGCAGG |
| | | TCGTCTTTACAGACCTCTTCCCCCCACTCTGCAAACGC |
| | | ATGAAGGTGCAGATCCGAGACTCGGACAAGGTCAACG |
| | | ACGTGGCCATCGGCACCCACTTCATTGACCTGCGCAA |
| | | GATTTCTAATGACGGAGACAAAGGCTTCCTGCCCACA |
| | | CTGGGCCCAGCCTGGGTGAACATGTACGGCTCCACAC |
| | | GTAACTACACGCTGCTGGATGAGCATCAGGACCTGAA |
| | | CGAGGGCCTGGGGGAGGGTGTGTCCTTCCGGGCCCG |
| | | GCTCCTGCTGGGCCTGGCTGTGGAGATCGTAGACACC |
| | | TCCAACCCTGAGCTCACCAGCTCCACAGAGGTGCAGG |
| | | TGGAGCAGGCCACGCCCATCTCGGAGAGCTGTGCAG |
| | | GTAAAATGGAAGAATTCTTTCTCTTTGGAGCCTTCCTG |
| | | GAGGCCTCAATGATCGACCGGAGAAACGGAGACAAGC |
| | | CCATCACCTTTGAGGTCACCATAGGCAACTATGGGAA |
| | | CGAAGTTGATGGCCTGTCCCGGCCCCAGCGGCCTCG |
| | | GCCCCGGAAGGAGCCGGGGGATGAGGAAGAAGTAGA |
| | | CCTGATTCAGAACGCAAGTGATGACGAGGCCGGTGAT |
| | | GCCGGGGACCTGGCCTCAGTCTCCTCCACTCCACCAA |
| | | TGCGGCCCCAGGTCACCGACAGGAACTACTTCCATCT |
| | | GCCCTACCTGGAGCGAAAGCCCTGCATCTACATCAAG |
| | | AGCTGGTGGCCGGACCAGCGCCGCCGCCTCTACAAT |
| | | GCCAACATCATGGACCACATTGCCGACAAGCTGGAAG |
| | | AAGGCCTGAACGACATACAGGAGATGATCAAAACGGA |
| | | GAAGTCCTACCCTGAGCGTCGCCTGCGGGGCGTCCT |
| | | GGAGGAGCTGAGCTGTGGCTGCTGCCGCTTCCTCTCC |
| | | CTCGCTGACAAGGACCAGGGCCACTCATCCCGCACCA |
| | | GGCTTGACCGGGAGCGCCTCAAGTCCTGCATGAGGG |
| | | AGCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACCTCGAGTTAAGGGCGA |
| | | ATTCCCGATAAGGATCTTCCTAGAGCATGGCTACGTAG |
| | | ATAAGTAGCATGGCGGGTTAATCATTAACTACAAGGAA |
| | | CCCCTAGTGATGGAGTTGGCCACTCCCTCTCTGCGCG |
| | | CTCGCTCGCTCACTGAGGCCGGGCGACCAAAGGTCG |
| | | CCCGACGCCCGGGCTTTGCCCGGGCGGCCTCAGTGA |
| | | GCGAGCGAGCGCGCAGCCTTAATTAACCTAATTCACT |
| | | GGCCGTCGTTTTACAACGTCGTGACTGGGAAAACCCT |
| | | GGCGTTACCCAACTTAATCGCCTTGCAGCACATCCCC |
| | | CTTTCGCCAGCTGGCGTAATAGCGAAGAGGCCCGCAC |
| | | CGATCGCCCTTCCCAACAGTTGCGCAGCCTGAATGGC |
| | | GAATGGGACGCGCCCTGTAGCGGCGCATTAAGCGCG |
| | | GCGGGTGTGGTGGTTACGCGCAGCGTGACCGCTACA |
| | | CTTGCCAGCGCCCTAGCGCCCGCTCCTTTCGCTTTCT |
| | | TCCCTTCCTTTCTCGCCACGTTCGCCGGCTTTCCCCGT |
| | | CAAGCTCTAAATCGGGGGCTCCCTTTAGGGTTCCGAT |
| | | TTAGTGCTTTACGGCACCTCGACCCCAAAAAACTTGAT |
| | | TAGGGTGATGGTTCACGTAGTGGGCCATCGCCCTGAT |
| | | AGACGGTTTTTCGCCCTTTGACGTTGGAGTCCACGTTC |
| | | TTTAATAGTGGACTCTTGTTCCAAACTGGAACAACACT |
| | | CAACCCTATCTCGGTCTATTCTTTTGATTTATAAGGGAT |
| | | TTTGCCGATTTCGGCCTATTGGTTAAAAAATGAGCTGA |
| | | TTTAACAAAAATTTAACGCGAATTTTAACAAAATATTAA |
| | | CGCTTACAATTTAGGTGGCACTTTTCGGGGAAATGTGC |
| | | GCGGAACCCCTATTTGTTTATTTTTCTAAATACATTCAA |
| | | ATATGTATCCGCTCATGAGACAATAACCCTGATAAATG |
| | | CTTCAATAATATTGAAAAAGGAAGAGTATGAGCCATAT |
| | | TCAACGGGAAACGTCGAGGCCGCGATTAAATTCCAAC |
| | | ATGGATGCTGATTTATATGGGTATAAATGGGCTCGCGA |
| | | TAATGTCGGGCAATCAGGTGCGACAATCTATCGCTTGT |
| | | ATGGGAAGCCCGATGCGCCAGAGTTGTTTCTGAAACA |
| | | TGGCAAAGGTAGCGTTGCCAATGATGTTACAGATGAG |
| | | ATGGTCAGACTAAACTGGCTGACGGAATTTATGCCTCT |
| | | TCCGACCATCAAGCATTTTATCCGTACTCCTGATGATG |
| | | CATGGTTACTCACCACTGCGATCCCCGGAAAAACAGC |
| | | ATTCCAGGTATTAGAAGAATATCCTGATTCAGGTGAAA |
| | | ATATTGTTGATGCGCTGGCAGTGTTCCTGCGCCGGTT |
| | | GCATTCGATTCCTGTTTGTAATTGTCCTTTTAACAGCGA |
| | | TCGCGTATTTCGTCTTGCTCAGGCGCAATCACGAATGA |
| | | ATAACGGTTTGGTTGATGCGAGTGATTTTGATGACGAG |
| | | CGTAATGGCTGGCCTGTTGAACAAGTCTGGAAAGAAA |
| | | TGCATAAACTTTTGCCATTCTCACCGGATTCAGTCGTC |
| | | ACTCATGGTGATTTCTCACTTGATAACCTTATTTTTGAC |
| | | GAGGGGAAATTAATAGGTTGTATTGATGTTGGACGAGT |
| | | CGGAATCGCAGACCGATACCAGGATCTTGCCATCCTA |
| | | TGGAACTGCCTCGGTGAGTTTTCTCCTTCATTACAGAA |
| | | ACGGCTTTTTCAAAAATATGGTATTGATAATCCTGATAT |
| | | GAATAAATTGCAGTTTCATTTGATGCTCGATGAGTTTTT |
| | | CTAACTGTCAGACCAAGTTTACTCATATATACTTTAGAT |
| | | TGATTTAAAACTTCATTTTTAATTTAAAAGGATCTAGGT |
| | | GAAGATCCTTTTTGATAATCTCATGACCAAAATCCCTTA |
| | | ACGTGAGTTTTCGTTCCACTGAGCGTCAGACCCCGTA |
| | | GAAAAGATCAAAGGATCTTCTTGAGATCCTTTTTTTCTG |
| | | CGCGTAATCTGCTGCTTGCAAACAAAAAAACCACCGCT |
| | | ACCAGCGGTGGTTTGTTTGCCGGATCAAGAGCTACCA |
| | | ACTCTTTTTCCGAAGGTAACTGGCTTCAGCAGAGCGCA |
| | | GATACCAAATACTGTTCTTCTAGTGTAGCCGTAGTTAG |
| | | GCCACCACTTCAAGAACTCTGTAGCACCGCCTACATAC |
| | | CTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGA |
| | | ACGACCTACACCGAACTGAGATACCTACAGCGTGAGC |
| | | TATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGCCTTTTGCTGGCCTTTTGCTCACATGTT |
| | | CTTTCCTGCGTTATCCCCTGATTCTGTGGATAACCGTA |
| | | TTACCGCCTTTGAGTGAGCTGATACCGCTCGCCGCAG |
| | | CCGAACGACCGAGCGCAGCGAGTCAGTGAGCGAGGA |
| | | AGCGGAAGAGCGCCCAATACGCAAACCGCCTCTCCCC |
| | | GCGCGTTGGCCGATTCATTAATGCAGCTGGCACGACA |
| | | GGTTTCCCGACTGGAAAGCGGGCAGTGAGCGCAACG |
| | | CAATTAATGTGAGTTAGCTCACTCATTAGGCACCCCAG |
| | | GCTTTACACTTTATGCTTCCGGCTCGTATGTTGTGTGG |
| | | AATTGTGAGCGGATAACAATTTCACACAGGAAACAGCT |
| | | ATGACCATGATTACGCCAGATTTAATTAAGG |
| 67 | 3′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | AP head sequence at positions | GGGAGTGGCCAACTCCATCACTAGGGGTTCCTTGTAG |
| | 229-515 | TTAATGATTAACCCGCCATGCTACTTATCTACGTAGCC |
| | Splice acceptor sequence at | ATGCTCTAGGAAGATCGGAATTCGCCCTTAAGCTAGC |
| | positions 538-586 | GGCGCGCCCCCGGGTGCGCGGCGTCGGTGGTGCCG |
| | C-terminal portion of human | GCGGGGGGCGCCAGGTCGCAGGCGGTGTAGGGCTC |
| | OTOF isoform 5 at positions | CAGGCAGGCGGCGAAGGCCATGACGTGCGCTATGAA |
| | 587-4174 | GGTCTGCTCCTGCACGCCGTGAACCAGGTGCGCCTG |
| | bGH poly(A) sequence at | CGGGCCGCGCGCGAACACCGCCACGTCCTCGCCTGC |
| | positions 4217-4438 | GTGGGTCTCTTCGTCCAGGGGCACTGCTGACTGCTGC |
| | ITR at positions 4526-4655 | CGATACTCGGGGCTCCCGCTCTCGCTCTCGGTAACAT |
| | M13 fwd at positions 4674-4690 | CCGGCCGGGCGCCGTCCTTGAGCACATAGCCTGGAC |
| | f1 ori at positions 4832-5287 | CGTTTCCTTAAGCGACGCATGCTCGCGATAGGCACCT |
| | AmpR promoter at positions | ATTGGTCTTACTGACATCCACTTTGCCTTTCTCTCCACA |
| | 5313-5417 | GGAAAACATGGGGCAGCAGGCCAGGATGCTGCGGGC |
| | KanR at positions 5418-6227 | CCAGGTGAAGCGGCACACGGTGCGGGACAAGCTGAG |
| | mutBsmBI at positions 5858- | GCTGTGCCAGAACTTCCTGCAGAAGCTGCGCTTCCTG |
| | 5858 | GCGGACGAGCCCCAGCACAGCATTCCCGACATCTTCA |
| | ori at positions 6398-6986 | TCTGGATGATGAGCAACAACAAGCGTGTCGCCTATGC |
| | CAP binding site at positions | CCGTGTGCCCTCCAAGGACCTGCTCTTCTCCATCGTG |
| | 7274-7295 | GAGGAGGAGACTGGCAAGGACTGCGCCAAGGTCAAG |
| | lac promoter at positions 7310- | ACGCTCTTCCTTAAGCTGCCAGGGAAGCGGGGCTTCG |
| | 7340 | GCTCGGCAGGCTGGACAGTGCAGGCCAAGGTGGAGC |
| | lac operator at positions 7348- | TGTACCTGTGGCTGGGCCTCAGCAAACAGCGCAAGGA |
| | 7364 | GTTCCTGTGCGGCCTGCCCTGTGGCTTCCAGGAGGTC |
| | M13 rev at positions 7372-7388 | AAGGCAGCCCAGGGCCTGGGCCTGCATGCCTTCCCA |
| | Transgene to be transferred | CCCGTCAGCCTGGTCTACACCAAGAAGCAGGCGTTCC |
| | into vector in dual vector | AGCTCCGAGCGCACATGTACCAGGCCCGCAGCCTCTT |
| | system at positions 12-4655 | TGCCGCCGACAGCAGCGGACTCTCAGACCCCTTTGCC |
| | | CGCGTCTTCTTCATCAATCAGAGTCAGTGCACAGAGGT |
| | | GCTGAATGAGACCCTGTGTCCCACCTGGGACCAGATG |
| | | CTGGTGTTCGACAACCTGGAGCTCTATGGTGAAGCTC |
| | | ATGAGCTGAGGGACGATCCGCCCATCATTGTCATTGA |
| | | AATCTATGACCAGGATTCCATGGGCAAAGCTGACTTCA |
| | | TGGGCCGGACCTTCGCCAAACCCCTGGTGAAGATGGC |
| | | AGACGAGGCGTACTGCCCACCCCGCTTCCCACCTCAG |
| | | CTCGAGTACTACCAGATCTACCGTGGCAACGCCACAG |
| | | CTGGAGACCTGCTGGCGGCCTTCGAGCTGCTGCAGAT |
| | | TGGACCAGCAGGGAAGGCTGACCTGCCCCCCATCAAT |
| | | GGCCCGGTGGACGTGGACCGAGGTCCCATCATGCCC |
| | | GTGCCCATGGGCATCCGGCCCGTGCTCAGCAAGTACC |
| | | GAGTGGAGGTGCTGTTCTGGGGCCTACGGGACCTAAA |
| | | GCGGGTGAACCTGGCCCAGGTGGACCGGCCACGGGT |
| | | GGACATCGAGTGTGCAGGGAAGGGGGTGCAGTCGTC |
| | | CCTGATCCACAATTATAAGAAGAACCCCAACTTCAACA |
| | | CCCTCGTCAAGTGGTTTGAAGTGGACCTCCCAGAGAA |
| | | CGAGCTGCTGCACCCGCCCTTGAACATCCGTGTGGTG |
| | | GACTGCCGGGCCTTCGGTCGCTACACACTGGTGGGCT |
| | | CCCATGCCGTCAGCTCCCTGCGACGCTTCATCTACCG |
| | | GCCCCCAGACCGCTCGGCCCCCAGCTGGAACACCAC |
| | | GGTCAGGCTTCTCCGGCGCTGCCGTGTGCTGTGCAAT |
| | | GGGGGCTCCTCCTCTCACTCCACAGGGGAGGTTGTG |
| | | GTGACTATGGAGCCAGAGGTACCCATCAAGAAACTGG |
| | | AGACCATGGTGAAGCTGGACGCGACTTCTGAAGCTGT |
| | | TGTCAAGGTGGATGTGGCTGAGGAGGAGAAGGAGAA |
| | | GAAGAAGAAGAAGAAGGGCACTGCGGAGGAGCCAGA |
| | | GGAGGAGGAGCCAGACGAGAGCATGCTGGACTGGTG |
| | | GTCCAAGTACTTTGCCTCCATTGACACCATGAAGGAGC |
| | | AACTTCGACAACAAGAGCCCTCTGGAATTGACTTGGA |
| | | GGAGAAGGAGGAAGTGGACAATACCGAGGGCCTGAA |
| | | GGGGTCAATGAAGGGCAAGGAGAAGGCAAGGGCTGC |
| | | CAAAGAGGAGAAGAAGAAGAAAACTCAGAGCTCTGGC |
| | | TCTGGCCAGGGGTCCGAGGCCCCCGAGAAGAAGAAA |
| | | CCCAAGATTGATGAGCTTAAGGTATACCCCAAAGAGCT |
| | | GGAGTCCGAGTTTGATAACTTTGAGGACTGGCTGCAC |
| | | ACTTTCAACTTGCTTCGGGGCAAGACCGGGGATGATG |
| | | AGGATGGCTCCACCGAGGAGGAGCGCATTGTGGGAC |
| | | GCTTCAAGGGCTCCCTCTGCGTGTACAAAGTGCCACT |
| | | CCCAGAGGACGTGTCCCGGGAAGCCGGCTACGACTC |
| | | CACCTACGGCATGTTCCAGGGCATCCCGAGCAATGAC |
| | | CCCATCAATGTGCTGGTCCGAGTCTATGTGGTCCGGG |
| | | CCACGGACCTGCACCCTGCTGACATCAACGGCAAAGC |
| | | TGACCCCTACATCGCCATCCGGCTAGGCAAGACTGAC |
| | | ATCCGCGACAAGGAGAACTACATCTCCAAGCAGCTCA |
| | | ACCCTGTCTTTGGGAAGTCCTTTGACATCGAGGCCTC |
| | | CTTCCCCATGGAATCCATGCTGACGGTGGCTGTGTAT |
| | | GACTGGGACCTGGTGGGCACTGATGACCTCATTGGGG |
| | | AAACCAAGATCGACCTGGAGAACCGCTTCTACAGCAA |
| | | GCACCGCGCCACCTGCGGCATCGCCCAGACCTACTC |
| | | CACACATGGCTACAATATCTGGCGGGACCCCATGAAG |
| | | CCCAGCCAGATCCTGACCCGCCTCTGCAAAGACGGCA |
| | | AAGTGGACGGCCCCCACTTTGGGCCCCCTGGGAGAG |
| | | TGAAGGTGGCCAACCGCGTCTTCACTGGGCCCTCTGA |
| | | GATTGAGGACGAGAACGGTCAGAGGAAGCCCACAGA |
| | | CGAGCATGTGGCGCTGTTGGCCCTGAGGCACTGGGA |
| | | GGACATCCCCCGCGCAGGCTGCCGCCTGGTGCCAGA |
| | | GCATGTGGAGACGAGGCCGCTGCTCAACCCCGACAA |
| | | GCCGGGCATCGAGCAGGGCCGCCTGGAGCTGTGGGT |
| | | GGACATGTTCCCCATGGACATGCCAGCCCCTGGGACG |
| | | CCTCTGGACATCTCACCTCGGAAGCCCAAGAAGTACG |
| | | AGCTGCGGGTCATCATCTGGAACACAGATGAGGTGGT |
| | | CTTGGAGGACGACGACTTCTTCACAGGGGAGAAGTCC |
| | | AGTGACATCTTCGTGAGGGGGTGGCTGAAGGGCCAG |
| | | CAGGAGGACAAGCAGGACACAGACGTCCACTACCACT |
| | | CCCTCACTGGCGAGGGCAACTTCAACTGGCGCTACCT |
| | | GTTCCCCTTCGACTACCTGGCGGCGGAGGAGAAGATC |
| | | GTCATCTCCAAGAAGGAGTCCATGTTCTCCTGGGACG |
| | | AGACCGAGTACAAGATCCCCGCGCGGCTCACCCTGCA |
| | | GATCTGGGATGCGGACCACTTCTCCGCTGACGACTTC |
| | | CTGGGGGCCATCGAGCTGGACCTGAACCGGTTCCCG |
| | | CGGGGCGCAAAGACAGCCAAGCAGTGCACCATGGAG |
| | | ATGGCCACCGGGGAGGTGGACGTGCCCCTCGTGTCC |
| | | ATCTTCAAGCAAAAGCGCGTCAAAGGCTGGTGGCCCC |
| | | TCCTGGCCCGCAATGAGAACGATGAGTTTGAGCTCAC |
| | | GGGCAAGGTGGAGGCTGAGCTGCATTTACTGACAGCA |
| | | GAGGAGGCAGAGAAGAACCCAGTGGGCCTGGCCCGC |
| | | AATGAACCTGACCCCCTAGAGAAACCCAACCGGCCCG |
| | | ACACGGCCTTCGTCTGGTTCCTCAACCCTCTCAAGTCC |
| | | ATCAAGTACCTCATCTGCACCCGGTACAAGTGGCTCAT |
| | | CATCAAGATCGTGCTGGCGCTGTTGGGGCTGCTCATG |
| | | TTGGGGCTCTTCCTCTACAGCCTCCCTGGCTACATGG |
| | | TCAAAAAGCTCCTTGGGGCATGAACGGCCGCTATGCT |
| | | AGCTTGGTACCAAGGGCGGATCCTGCATAGAGCTCGC |
| | | TGATCAGCCTCGACTGTGCCTTCTAGTTGCCAGCCATC |
| | | TGTTGTTTGCCCCTCCCCCGTGCCTTCCTTGACCCTG |
| | | GAAGGTGCCACTCCCACTGTCCTTTCCTAATAAAATGA |
| | | GGAAATTGCATCGCATTGTCTGAGTAGGTGTCATTCTA |
| | | TTCTGGGGGGTGGGGTGGGGCAGGACAGCAAGGGG |
| | | GAGGATTGGGAAGACAATAGCAGGCATCTCGAGTTAA |
| | | GGGCGAATTCCCGATAAGGATCTTCCTAGAGCATGGC |
| | | TACGTAGATAAGTAGCATGGCGGGTTAATCATTAACTA |
| | | CAAGGAACCCCTAGTGATGGAGTTGGCCACTCCCTCT |
| | | CTGCGCGCTCGCTCGCTCACTGAGGCCGGGCGACCA |
| | | AAGGTCGCCCGACGCCCGGGCTTTGCCCGGGCGGCC |
| | | TCAGTGAGCGAGCGAGCGCGCAGCCTTAATTAACCTA |
| | | ATTCACTGGCCGTCGTTTTACAACGTCGTGACTGGGAA |
| | | AACCCTGGCGTTACCCAACTTAATCGCCTTGCAGCACA |
| | | TCCCCCTTTCGCCAGCTGGCGTAATAGCGAAGAGGCC |
| | | CGCACCGATCGCCCTTCCCAACAGTTGCGCAGCCTGA |
| | | ATGGCGAATGGGACGCGCCCTGTAGCGGCGCATTAA |
| | | GCGCGGCGGGTGTGGTGGTTACGCGCAGCGTGACCG |
| | | CTACACTTGCCAGCGCCCTAGCGCCCGCTCCTTTCGC |
| | | TTTCTTCCCTTCCTTTCTCGCCACGTTCGCCGGCTTTC |
| | | CCCGTCAAGCTCTAAATCGGGGGCTCCCTTTAGGGTT |
| | | CCGATTTAGTGCTTTACGGCACCTCGACCCCAAAAAAC |
| | | TTGATTAGGGTGATGGTTCACGTAGTGGGCCATCGCC |
| | | CTGATAGACGGTTTTTCGCCCTTTGACGTTGGAGTCCA |
| | | CGTTCTTTAATAGTGGACTCTTGTTCCAAACTGGAACA |
| | | ACACTCAACCCTATCTCGGTCTATTCTTTTGATTTATAA |
| | | GGGATTTTGCCGATTTCGGCCTATTGGTTAAAAAATGA |
| | | GCTGATTTAACAAAAATTTAACGCGAATTTTAACAAAAT |
| | | ATTAACGCTTACAATTTAGGTGGCACTTTTCGGGGAAA |
| | | TGTGCGCGGAACCCCTATTTGTTTATTTTTCTAAATACA |
| | | TTCAAATATGTATCCGCTCATGAGACAATAACCCTGAT |
| | | AAATGCTTCAATAATATTGAAAAAGGAAGAGTATGAGC |
| | | CATATTCAACGGGAAACGTCGAGGCCGCGATTAAATT |
| | | CCAACATGGATGCTGATTTATATGGGTATAAATGGGCT |
| | | CGCGATAATGTCGGGCAATCAGGTGCGACAATCTATC |
| | | GCTTGTATGGGAAGCCCGATGCGCCAGAGTTGTTTCT |
| | | GAAACATGGCAAAGGTAGCGTTGCCAATGATGTTACA |
| | | GATGAGATGGTCAGACTAAACTGGCTGACGGAATTTAT |
| | | GCCTCTTCCGACCATCAAGCATTTTATCCGTACTCCTG |
| | | ATGATGCATGGTTACTCACCACTGCGATCCCCGGAAA |
| | | AACAGCATTCCAGGTATTAGAAGAATATCCTGATTCAG |
| | | GTGAAAATATTGTTGATGCGCTGGCAGTGTTCCTGCG |
| | | CCGGTTGCATTCGATTCCTGTTTGTAATTGTCCTTTTAA |
| | | CAGCGATCGCGTATTTCGTCTTGCTCAGGCGCAATCA |
| | | CGAATGAATAACGGTTTGGTTGATGCGAGTGATTTTGA |
| | | TGACGAGCGTAATGGCTGGCCTGTTGAACAAGTCTGG |
| | | AAAGAAATGCATAAACTTTTGCCATTCTCACCGGATTC |
| | | AGTCGTCACTCATGGTGATTTCTCACTTGATAACCTTAT |
| | | TTTTGACGAGGGGAAATTAATAGGTTGTATTGATGTTG |
| | | GACGAGTCGGAATCGCAGACCGATACCAGGATCTTGC |
| | | CATCCTATGGAACTGCCTCGGTGAGTTTTCTCCTTCAT |
| | | TACAGAAACGGCTTTTTCAAAAATATGGTATTGATAATC |
| | | CTGATATGAATAAATTGCAGTTTCATTTGATGCTCGATG |
| | | AGTTTTTCTAACTGTCAGACCAAGTTTACTCATATATAC |
| | | TTTAGATTGATTTAAAACTTCATTTTTAATTTAAAAGGAT |
| | | CTAGGTGAAGATCCTTTTTGATAATCTCATGACCAAAAT |
| | | CCCTTAACGTGAGTTTTCGTTCCACTGAGCGTCAGACC |
| | | CCGTAGAAAAGATCAAAGGATCTTCTTGAGATCCTTTT |
| | | TTTCTGCGCGTAATCTGCTGCTTGCAAACAAAAAAACC |
| | | ACCGCTACCAGCGGTGGTTTGTTTGCCGGATCAAGAG |
| | | CTACCAACTCTTTTTCCGAAGGTAACTGGCTTCAGCAG |
| | | AGCGCAGATACCAAATACTGTTCTTCTAGTGTAGCCGT |
| | | AGTTAGGCCACCACTTCAAGAACTCTGTAGCACCGCC |
| | | TACATACCTCGCTCTGCTAATCCTGTTACCAGTGGCTG |
| | | CTGCCAGTGGCGATAAGTCGTGTCTTACCGGGTTGGA |
| | | CTCAAGACGATAGTTACCGGATAAGGCGCAGCGGTCG |
| | | GGCTGAACGGGGGGTTCGTGCACACAGCCCAGCTTG |
| | | GAGCGAACGACCTACACCGAACTGAGATACCTACAGC |
| | | GTGAGCTATGAGAAAGCGCCACGCTTCCCGAAGGGAG |
| | | AAAGGCGGACAGGTATCCGGTAAGCGGCAGGGTCGG |
| | | AACAGGAGAGCGCACGAGGGAGCTTCCAGGGGGAAA |
| | | CGCCTGGTATCTTTATAGTCCTGTCGGGTTTCGCCACC |
| | | TCTGACTTGAGCGTCGATTTTTGTGATGCTCGTCAGGG |
| | | GGGCGGAGCCTATGGAAAAACGCCAGCAACGCGGCC |
| | | TTTTTACGGTTCCTGGCCTTTTGCTGGCCTTTTGCTCA |
| | | CATGTTCTTTCCTGCGTTATCCCCTGATTCTGTGGATA |
| | | ACCGTATTACCGCCTTTGAGTGAGCTGATACCGCTCG |
| | | CCGCAGCCGAACGACCGAGCGCAGCGAGTCAGTGAG |
| | | CGAGGAAGCGGAAGAGCGCCCAATACGCAAACCGCC |
| | | TCTCCCCGCGCGTTGGCCGATTCATTAATGCAGCTGG |
| | | CACGACAGGTTTCCCGACTGGAAAGCGGGCAGTGAG |
| | | CGCAACGCAATTAATGTGAGTTAGCTCACTCATTAGGC |
| | | ACCCCAGGCTTTACACTTTATGCTTCCGGCTCGTATGT |
| | | TGTGTGGAATTGTGAGCGGATAACAATTTCACACAGGA |
| | | AACAGCTATGACCATGATTACGCCAGATTTAATTAAGG |
| 68 | 5′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | CMV i.e enhancer at positions | GGGAGTGGCCAACTCCATCACTAGGGGTTCCTTGTAG |
| | 230-594 | TTAATGATTAACCCGCCATGCTACTTATCTACGTAGCC |
| | CMV enhancer at positions | ATGCTCTAGGAAGATCGGAATTCGCCCTTAAGCTAGC |
| | 296-599 | GGCGCGCCGGTACCTAGTTATTAATAGTAATCAATTAC |
| | Chicken β-actin promoter at | GGGGTCATTAGTTCATAGCCCATATATGGAGTTCCGC |
| | positions 596-878 | GTTACATAACTTACGGTAAATGGCCCGCCTGGCTGAC |
| | Exon 1 at positions 879-971 | CGCCCAACGACCCCCGCCCATTGACGTCAATAATGAC |
| | Chimeric intron at positions | GTATGTTCCCATAGTAACGCCAATAGGGACTTTCCATT |
| | 971-1172 | GACGTCAATGGGTGGAGTATTTACGGTAAACTGCCCA |
| | Kozak sequence at positions | CTTGGCAGTACATCAAGTGTATCATATGCCAAGTACGC |
| | 1189-1198 | CCCCTATTGACGTCAATGACGGTAAATGGCCCGCCTG |
| | N-terminal portion of human | GCATTATGCCCAGTACATGACCTTATGGGACTTTCCTA |
| | OTOF isoform 5 at positions | CTTGGCAGTACATCTACGTATTAGTCATCGCTATTACC |
| | 1195-3600 | ATGGTCGAGGTGAGCCCCACGTTCTGCTTCACTCTCC |
| | Splice donor sequence at | CCATCTCCCCCCCCTCCCCACCCCCAATTTTGTATTTA |
| | positions 3601-3684 | TTTATTTTTTAATTATTTTGTGCAGCGATGGGGGCGGG |
| | AP head sequence at positions | GGGGGGGGGGGGGCGCGCGCCAGGCGGGGCGGGG |
| | 3691-3977 | CGGGGCGAGGGGCGGGGCGGGGCGAGGCGGAGAG |
| | ITR at positions 4071-4200 | GTGCGGCGGCAGCCAATCAGAGCGGCGCGCTCCGAA |
| | M13 fwd at positions 4219-4235 | AGTTTCCTTTTATGGCGAGGCGGCGGCGGCGGCGGC |
| | f1 ori at positions 4377-4832 | CCTATAAAAAGCGAAGCGCGCGGCGGGCGGGAGTCG |
| | AmpR promoter at positions | CTGCGCGCTGCCTTCGCCCCGTGCCCCGCTCCGCCG |
| | 4858-4962 | CCGCCTCGCGCCGCCCGCCCCGGCTCTGACTGACCG |
| | KanR at positions 4963-5772 | CGTTACTCCCACAGGTGAGCGGGCGGGACGGCCCTT |
| | mutBsmBI at positions 4963- | CTCCTCCGGGCTGTAATTAGCGCTTGGTTTAATGACG |
| | 5772 | GCTTGTTTCTTTTCTGTGGCTGCGTGAAAGCCTTGAGG |
| | ori at positions 5943-6531 | GGCTCCGGGAGCTAGAGCCTCTGCTAACCATGTTCAT |
| | CAP binding site at positions | GCCTTCTTCTTTTTCCTACAGCTCCTGGGCAACGTGCT |
| | 6819-6840 | GGTTATTGTGCTGTCTCATCATTTTGGCAAAGAATTCTA |
| | lac promoter at positions 6855- | GCGGCCGCCACCATGGCCTTGCTCATCCACCTCAAGA |
| | 6885 | CAGTCTCGGAGCTGCGGGGCAGGGGCGACCGGATCG |
| | lac operator at positions 6835- | CCAAAGTGACTTTCCGAGGGCAATCCTTCTACTCTCGG |
| | 6909 | GTCCTGGAGAACTGTGAGGATGTGGCTGACTTTGATG |
| | M13 rev at positions 6917-6933 | AGACATTTCGGTGGCCGGTGGCCAGCAGCATCGACAG |
| | Transgene to be transferred | AAATGAGATGCTGGAGATTCAGGTTTTCAACTACAGCA |
| | into vector in dual vector | AAGTCTTCAGCAACAAGCTCATCGGGACCTTCCGCAT |
| | system at positions 12-4200 | GGTGCTGCAGAAGGTGGTAGAGGAGAGCCATGTGGA |
| | | GGTGACTGACACGCTGATTGATGACAACAATGCTATCA |
| | | TCAAGACCAGCCTGTGCGTGGAGGTCCGGTATCAGGC |
| | | CACTGACGGCACAGTGGGCTCCTGGGACGATGGGGA |
| | | CTTCCTGGGAGATGAGTCTCTTCAAGAGGAAGAGAAG |
| | | GACAGCCAAGAGACGGATGGACTGCTCCCAGGCTCC |
| | | CGGCCCAGCTCCCGGCCCCCAGGAGAGAAGAGCTTC |
| | | CGGAGAGCCGGGAGGAGCGTGTTCTCCGCCATGAAG |
| | | CTCGGCAAAAACCGGTCTCACAAGGAGGAGCCCCAAA |
| | | GACCAGATGAACCGGCGGTGCTGGAGATGGAAGACC |
| | | TTGACCATCTGGCCATTCGGCTAGGAGATGGACTGGA |
| | | TCCCGACTCGGTGTCTCTAGCCTCAGTCACAGCTCTC |
| | | ACCACTAATGTCTCCAACAAGCGATCTAAGCCAGACAT |
| | | TAAGATGGAGCCAAGTGCTGGGCGGCCCATGGATTAC |
| | | CAGGTCAGCATCACGGTGATCGAGGCCCGGCAGCTG |
| | | GTGGGCTTGAACATGGACCCTGTGGTGTGCGTGGAG |
| | | GTGGGTGACGACAAGAAGTACACATCCATGAAGGAGT |
| | | CCACTAACTGCCCCTATTACAACGAGTACTTCGTCTTC |
| | | GACTTCCATGTCTCTCCGGATGTCATGTTTGACAAGAT |
| | | CATCAAGATTTCGGTGATTCACTCCAAGAACCTGCTGC |
| | | GCAGTGGCACCCTGGTGGGCTCCTTCAAAATGGACGT |
| | | GGGAACCGTGTACTCGCAGCCAGAGCACCAGTTCCAT |
| | | CACAAGTGGGCCATCCTGTCTGACCCCGATGACATCT |
| | | CCTCGGGGCTGAAGGGCTACGTGAAGTGTGACGTTGC |
| | | CGTGGTGGGCAAAGGGGACAACATCAAGACGCCCCA |
| | | CAAGGCCAATGAGACCGACGAAGATGACATTGAGGGG |
| | | AACTTGCTGCTCCCCGAGGGGGTGCCCCCCGAACGC |
| | | CAGTGGGCCCGGTTCTATGTGAAAATTTACCGAGCAG |
| | | AGGGGCTGCCCCGTATGAACACAAGCCTCATGGCCAA |
| | | TGTAAAGAAGGCTTTCATCGGTGAAAACAAGGACCTC |
| | | GTGGACCCCTACGTGCAAGTCTTCTTTGCTGGCCAGA |
| | | AGGGCAAGACTTCAGTGCAGAAGAGCAGCTATGAGCC |
| | | CCTGTGGAATGAGCAGGTCGTCTTTACAGACCTCTTCC |
| | | CCCCACTCTGCAAACGCATGAAGGTGCAGATCCGAGA |
| | | CTCGGACAAGGTCAACGACGTGGCCATCGGCACCCAC |
| | | TTCATTGACCTGCGCAAGATTTCTAATGACGGAGACAA |
| | | AGGCTTCCTGCCCACACTGGGCCCAGCCTGGGTGAAC |
| | | ATGTACGGCTCCACACGTAACTACACGCTGCTGGATG |
| | | AGCATCAGGACCTGAACGAGGGCCTGGGGGAGGGTG |
| | | TGTCCTTCCGGGCCCGGCTCCTGCTGGGCCTGGCTGT |
| | | GGAGATCGTAGACACCTCCAACCCTGAGCTCACCAGC |
| | | TCCACAGAGGTGCAGGTGGAGCAGGCCACGCCCATC |
| | | TCGGAGAGCTGTGCAGGTAAAATGGAAGAATTCTTTCT |
| | | CTTTGGAGCCTTCCTGGAGGCCTCAATGATCGACCGG |
| | | AGAAACGGAGACAAGCCCATCACCTTTGAGGTCACCA |
| | | TAGGCAACTATGGGAACGAAGTTGATGGCCTGTCCCG |
| | | GCCCCAGCGGCCTCGGCCCCGGAAGGAGCCGGGGG |
| | | ATGAGGAAGAAGTAGACCTGATTCAGAACGCAAGTGA |
| | | TGACGAGGCCGGTGATGCCGGGGACCTGGCCTCAGT |
| | | CTCCTCCACTCCACCAATGCGGCCCCAGGTCACCGAC |
| | | AGGAACTACTTCCATCTGCCCTACCTGGAGCGAAAGC |
| | | CCTGCATCTACATCAAGAGCTGGTGGCCGGACCAGCG |
| | | CCGCCGCCTCTACAATGCCAACATCATGGACCACATT |
| | | GCCGACAAGCTGGAAGAAGGCCTGAACGACATACAGG |
| | | AGATGATCAAAACGGAGAAGTCCTACCCTGAGCGTCG |
| | | CCTGCGGGGCGTCCTGGAGGAGCTGAGCTGTGGCTG |
| | | CTGCCGCTTCCTCTCCCTCGCTGACAAGGACCAGGGC |
| | | CACTCATCCCGCACCAGGCTTGACCGGGAGCGCCTCA |
| | | AGTCCTGCATGAGGGAGCTGGTAAGTATCAAGGTTAC |
| | | AAGACAGGTTTAAGGAGACCAATAGAAACTGGGCTTG |
| | | TCGAGACAGAGAAGACTCTTGCGTTTCTGAGCTAGCC |
| | | CCCGGGTGCGCGGCGTCGGTGGTGCCGGCGGGGGG |
| | | CGCCAGGTCGCAGGCGGTGTAGGGCTCCAGGCAGGC |
| | | GGCGAAGGCCATGACGTGCGCTATGAAGGTCTGCTCC |
| | | TGCACGCCGTGAACCAGGTGCGCCTGCGGGCCGCGC |
| | | GCGAACACCGCCACGTCCTCGCCTGCGTGGGTCTCTT |
| | | CGTCCAGGGGCACTGCTGACTGCTGCCGATACTCGG |
| | | GGCTCCCGCTCTCGCTCTCGGTAACATCCGGCCGGG |
| | | CGCCGTCCTTGAGCACATAGCCTGGACCGTTTCGTCG |
| | | ACCTCGAGTTAAGGGCGAATTCCCGATAAGGATCTTC |
| | | CTAGAGCATGGCTACGTAGATAAGTAGCATGGCGGGT |
| | | TAATCATTAACTACAAGGAACCCCTAGTGATGGAGTTG |
| | | GCCACTCCCTCTCTGCGCGCTCGCTCGCTCACTGAGG |
| | | CCGGGCGACCAAAGGTCGCCCGACGCCCGGGCTTTG |
| | | CCCGGGCGGCCTCAGTGAGCGAGCGAGCGCGCAGCC |
| | | TTAATTAACCTAATTCACTGGCCGTCGTTTTACAACGTC |
| | | GTGACTGGGAAAACCCTGGCGTTACCCAACTTAATCG |
| | | CCTTGCAGCACATCCCCCTTTCGCCAGCTGGCGTAAT |
| | | AGCGAAGAGGCCCGCACCGATCGCCCTTCCCAACAGT |
| | | TGCGCAGCCTGAATGGCGAATGGGACGCGCCCTGTA |
| | | GCGGCGCATTAAGCGCGGCGGGTGTGGTGGTTACGC |
| | | GCAGCGTGACCGCTACACTTGCCAGCGCCCTAGCGC |
| | | CCGCTCCTTTCGCTTTCTTCCCTTCCTTTCTCGCCACG |
| | | TTCGCCGGCTTTCCCCGTCAAGCTCTAAATCGGGGGC |
| | | TCCCTTTAGGGTTCCGATTTAGTGCTTTACGGCACCTC |
| | | GACCCCAAAAAACTTGATTAGGGTGATGGTTCACGTAG |
| | | TGGGCCATCGCCCTGATAGACGGTTTTTCGCCCTTTG |
| | | ACGTTGGAGTCCACGTTCTTTAATAGTGGACTCTTGTT |
| | | CCAAACTGGAACAACACTCAACCCTATCTCGGTCTATT |
| | | CTTTTGATTTATAAGGGATTTTGCCGATTTCGGCCTATT |
| | | GGTTAAAAAATGAGCTGATTTAACAAAAATTTAACGCG |
| | | AATTTTAACAAAATATTAACGCTTACAATTTAGGTGGCA |
| | | CTTTTCGGGGAAATGTGCGCGGAACCCCTATTTGTTTA |
| | | TTTTTCTAAATACATTCAAATATGTATCCGCTCATGAGA |
| | | CAATAACCCTGATAAATGCTTCAATAATATTGAAAAAGG |
| | | AAGAGTATGAGCCATATTCAACGGGAAACGTCGAGGC |
| | | CGCGATTAAATTCCAACATGGATGCTGATTTATATGGG |
| | | TATAAATGGGCTCGCGATAATGTCGGGCAATCAGGTG |
| | | CGACAATCTATCGCTTGTATGGGAAGCCCGATGCGCC |
| | | AGAGTTGTTTCTGAAACATGGCAAAGGTAGCGTTGCCA |
| | | ATGATGTTACAGATGAGATGGTCAGACTAAACTGGCTG |
| | | ACGGAATTTATGCCTCTTCCGACCATCAAGCATTTTAT |
| | | CCGTACTCCTGATGATGCATGGTTACTCACCACTGCGA |
| | | TCCCCGGAAAAACAGCATTCCAGGTATTAGAAGAATAT |
| | | CCTGATTCAGGTGAAAATATTGTTGATGCGCTGGCAGT |
| | | GTTCCTGCGCCGGTTGCATTCGATTCCTGTTTGTAATT |
| | | GTCCTTTTAACAGCGATCGCGTATTTCGTCTTGCTCAG |
| | | GCGCAATCACGAATGAATAACGGTTTGGTTGATGCGA |
| | | GTGATTTTGATGACGAGCGTAATGGCTGGCCTGTTGA |
| | | ACAAGTCTGGAAAGAAATGCATAAACTTTTGCCATTCT |
| | | CACCGGATTCAGTCGTCACTCATGGTGATTTCTCACTT |
| | | GATAACCTTATTTTTGACGAGGGGAAATTAATAGGTTG |
| | | TATTGATGTTGGACGAGTCGGAATCGCAGACCGATAC |
| | | CAGGATCTTGCCATCCTATGGAACTGCCTCGGTGAGT |
| | | TTTCTCCTTCATTACAGAAACGGCTTTTTCAAAAATATG |
| | | GTATTGATAATCCTGATATGAATAAATTGCAGTTTCATT |
| | | TGATGCTCGATGAGTTTTTCTAACTGTCAGACCAAGTT |
| | | TACTCATATATACTTTAGATTGATTTAAAACTTCATTTTT |
| | | AATTTAAAAGGATCTAGGTGAAGATCCTTTTTGATAATC |
| | | TCATGACCAAAATCCCTTAACGTGAGTTTTCGTTCCAC |
| | | TGAGCGTCAGACCCCGTAGAAAAGATCAAAGGATCTT |
| | | CTTGAGATCCTTTTTTTCTGCGCGTAATCTGCTGCTTG |
| | | CAAACAAAAAAACCACCGCTACCAGCGGTGGTTTGTTT |
| | | GCCGGATCAAGAGCTACCAACTCTTTTTCCGAAGGTAA |
| | | CTGGCTTCAGCAGAGCGCAGATACCAAATACTGTTCTT |
| | | CTAGTGTAGCCGTAGTTAGGCCACCACTTCAAGAACTC |
| | | TGTAGCACCGCCTACATACCTCGCTCTGCTAATCCTGT |
| | | TACCAGTGGCTGCTGCCAGTGGCGATAAGTCGTGTCT |
| | | TACCGGGTTGGACTCAAGACGATAGTTACCGGATAAG |
| | | GCGCAGCGGTCGGGCTGAACGGGGGGTTCGTGCACA |
| | | CAGCCCAGCTTGGAGCGAACGACCTACACCGAACTGA |
| | | GATACCTACAGCGTGAGCTATGAGAAAGCGCCACGCT |
| | | TCCCGAAGGGAGAAAGGCGGACAGGTATCCGGTAAG |
| | | CGGCAGGGTCGGAACAGGAGAGCGCACGAGGGAGCT |
| | | TCCAGGGGGAAACGCCTGGTATCTTTATAGTCCTGTC |
| | | GGGTTTCGCCACCTCTGACTTGAGCGTCGATTTTTGTG |
| | | ATGCTCGTCAGGGGGGCGGAGCCTATGGAAAAACGC |
| | | CAGCAACGCGGCCTTTTTACGGTTCCTGGCCTTTTGCT |
| | | GGCCTTTTGCTCACATGTTCTTTCCTGCGTTATCCCCT |
| | | GATTCTGTGGATAACCGTATTACCGCCTTTGAGTGAGC |
| | | TGATACCGCTCGCCGCAGCCGAACGACCGAGCGCAG |
| | | CGAGTCAGTGAGCGAGGAAGCGGAAGAGCGCCCAAT |
| | | ACGCAAACCGCCTCTCCCCGCGCGTTGGCCGATTCAT |
| | | TAATGCAGCTGGCACGACAGGTTTCCCGACTGGAAAG |
| | | CGGGCAGTGAGCGCAACGCAATTAATGTGAGTTAGCT |
| | | CACTCATTAGGCACCCCAGGCTTTACACTTTATGCTTC |
| | | CGGCTCGTATGTTGTGTGGAATTGTGAGCGGATAACA |
| | | ATTTCACACAGGAAACAGCTATGACCATGATTACGCCA |
| | | GATTTAATTAAGG |

### Vectors for the Expression of OTOF

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes a portion of OTOF, as well as, e.g., additional sequence elements used for the expression of these agents and/or the integration of these polynucleotide sequences into the genome of a mammalian cell. Certain vectors that can be used for the expression of OTOF include plasmids that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the compositions and methods described herein are recombinant nucleic acid constructs that include (1) a heterologous sequence to be expressed (e.g., a polynucleotide encoding an N-terminal or C-terminal portion of an OTOF protein) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV1, AAV2, AAV2quad (Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. For targeting cochlear hair cells, AAV1, AAV2, AAV6, AAV9, Anc80, Anc80L65, DJ/9, 7m8, and PHP.B may be particularly useful. Serotypes evolved for transduction of the retina may also be used in the methods and compositions described herein. The first and second nucleic acid vectors (e.g., AAV vectors) in the compositions and methods described herein may have the same serotype or different serotypes. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the use of AAV vectors for delivering a functional OTOF isoform 5 protein requires the use of a dual vector system, in in which the first member of the dual vector system encodes an N-terminal portion of an OTOF isoform 5 protein and the second member encodes a C-terminal portion of an OTOF isoform 5 protein such that, upon administration of the dual vector system to a cell, the polynucleotide sequences contained within the two vectors can join to form a single sequence that results in the production of a full-length OTOF isoform 5 protein.

In some embodiments, the first member of the dual vector system will also include, in 5′ to 3′ order, a first inverted terminal repeat (“ITR”); a promoter (e.g., a Myo15 promoter); a Kozak sequence; an N-terminal portion of an OTOF isoform 5 coding sequence; a splice donor sequence; an AP gene fragment (e.g., an AP head sequence); and a second ITR; and the second member of the dual vector system will include, in 5′ to 3′ order, a first ITR; an AP gene fragment (e.g., an AP head sequence); a splice acceptor sequence; a C-terminal portion of an OTOF isoform 5 coding sequence; a polyA sequence; and a second ITR. In some embodiments, the N-terminal portion of the OTOF isoform 5 coding sequence and the C-terminal portion of the OTOF isoform 5 coding sequence do not overlap and are joined in a cell (e.g., by recombination at the overlapping region (the AP gene fragment), or by concatemerization of the ITRs) to produce the full-length OTOF isoform 5 amino sequence as set forth in SEQ ID NO:1. In particular embodiments, the N-terminal portion of the OTOF isoform 5 coding sequence encodes amino acids 1-802 of SEQ ID NO:1 (SEQ ID NO: 58) and the C-terminal portion of the OTOF isoform 5 coding sequence encodes amino acids 803-1997 of SEQ ID NO:1 (SEQ ID NO: 59).

In some embodiments, the first member of the dual vector system includes the Myo15 promoter of SEQ ID NO:21 (also represented by nucleotides 235-1199 of SEQ ID NO:66) operably linked to nucleotides that encode the N-terminal 802 amino acids of the OTOF isoform 5 protein (amino acids 1-802 of SEQ ID NO:1), which are encoded by exons 1-20 of the native polynucleotide sequence encoding that protein. In certain embodiments, the nucleotide sequence that encodes the N-terminal amino acids of the OTOF isoform 5 protein is nucleotides 1222-3627 of SEQ ID NO:66. In some embodiments, the nucleotide sequence that encodes the N-terminal amino acids of the OTOF isoform 5 protein is any nucleotide sequence that, by redundancy of the genetic code, encodes amino acids 1-802 of SEQ ID NO: 1. The nucleotide sequences that encode the OTOF isoform 5 protein can be partially or fully codon-optimized for expression. In some embodiments, the first member of the dual vector system includes the Kozak sequence corresponding to nucleotides 1216-1225 of SEQ ID NO:66. In some embodiments, the first member of the dual vector system includes the splice donor sequence corresponding to nucleotides 3628-3711 of SEQ ID NO:66. In some embodiments, the first member of the dual vector system includes the AP head sequence corresponding to nucleotides 3718-4004 of SEQ ID NO:66. In particular embodiments, the first member of the dual vector system includes nucleotides 235-4004 of SEQ ID NO: 66 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO:66 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4098-4227 of SEQ ID NO: 66 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., any of SEQ ID NOs: 60, 62, 64, 65, 66, or 68), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-4227 of SEQ ID NO:66.

In some embodiments, the second member of the dual vector system includes nucleotides that encode the C-terminal 1195 amino acids of the OTOF isoform 5 protein (amino acids 803-1997 of SEQ ID NO: 1) immediately followed by a stop codon. In certain embodiments, the nucleotide sequence that encodes the C-terminal amino acids of the OTOF isoform 5 protein is nucleotides 587-4174 of SEQ ID NO: 67. In some embodiments, the nucleotide sequence that encodes the C-terminal amino acids of the OTOF isoform 5 protein is any nucleotide sequence that, by redundancy of the genetic code, encodes amino acids 803-1997 of SEQ ID NO:1. The nucleotide sequences that encode the OTOF isoform 5 protein can be partially or fully codon-optimized for expression. In some embodiments, the second member of the dual vector system includes the splice acceptor sequence corresponding to nucleotides 538-586 of SEQ ID NO:67. In some embodiments, the second member of the dual vector system includes the AP head sequence corresponding to nucleotides 229-515 of SEQ ID NO:67. In some embodiments, the second member of the dual vector system includes the poly(A) sisequence corresponding to nucleotides 4217-4438 of SEQ ID NO:67. In particular embodiments, the second member of the dual vector system includes nucleotides 229-4438 of SEQ ID NO:67 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO:67 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4526-4655 of SEQ ID NO:67 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., any of SEQ ID NOs: 61, 63, or 67), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-4655 of SEQ ID NO:67.

In some embodiments, the dual vector system is an AAV1 dual vector system.

In some embodiments, the dual vector system is an AAV9 dual vector system.

### Pharmaceutical Compositions

The nucleic acid vectors (e.g., AAV vectors) described herein may be incorporated into a vehicle for administration into a patient, such as a human patient suffering from sensorineural hearing loss or auditory neuropathy, as described herein. Pharmaceutical compositions containing vectors, such as viral vectors, that contain a polynucleotide encoding a portion of an OTOF isoform 5 protein can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acid vectors (e.g., AAV vectors) described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject with sensorineural hearing loss or auditory neuropathy by a variety of routes, such as local administration to the inner ear (e.g., administration into the perilymph or endolymph, e.g., by injection or catheter insertion through the round window membrane, injection into a semicircular canal, by canalostomy, or by intratympanic or transtympanic injection, e.g., administration to a cochlear hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. If the compositions are administered by direct delivery to the inner ear, a second fenestration or vent hole may be added elsewhere in the inner ear. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly). In some embodiments, the first and second nucleic acid vectors (e.g., AAV vectors) are administered simultaneously (e.g., in one composition). In some embodiments, the first and second nucleic acid vectors (e.g., AAV vectors) are administered sequentially (e.g., the second nucleic acid vector is administered immediately after the first nucleic acid vector, or 5 minutes, 10 minutes, 15 minutes, 20 minutes, 25 minutes, 30 minutes, 45 minutes, 1 hour, 2 hours, 8 hours, 12 hours, 1 day, 2 days, 7 days, two weeks, 1 month or more after the first nucleic acid vector). The first and second nucleic acid vector can have the same serotype or different serotypes (e.g., AAV serotypes).

Subjects that may be treated as described herein are subjects having or at risk of developing sensorineural hearing loss or auditory neuropathy. The compositions and methods described herein can be used to treat subjects having a mutation in OTOF (e.g., a mutation that reduces OTOF function or expression, or an OTOF mutation associated with sensorineural hearing loss), subjects having a family history of autosomal recessive sensorineural hearing loss or deafness (e.g., a family history of OTOF-related hearing loss), or subjects whose OTOF mutational status and/or OTOF activity level is unknown. The methods described herein may include a step of screening a subject for a mutation in OTOF prior to treatment with or administration of the compositions described herein. A subject can be screened for an OTOF mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, ABR, electrocochleography (ECOG), and otoacoustic emissions. The compositions and methods described herein may also be administered as a preventative treatment to patients at risk of developing hearing loss or auditory neuropathy, e.g., patients who have a family history of inherited hearing loss or patients carrying an OTOF mutation who do not yet exhibit hearing loss or impairment.

Treatment may include administration of a composition containing the nucleic acid vectors (e.g., AAV vectors) described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the cochlea. In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the AAV vectors may have a titer of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve hearing, increase WT OTOF expression (e.g., expression of OTOF isoform 5 in a cochlear hair cell, e.g., an inner hair cell), or increase OTOF function. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss or auditory neuropathy (e.g., in subjects who carry a mutation in OTOF or have a family history of autosomal recessive hearing loss but do not exhibit hearing impairment, or in subjects exhibiting mild to moderate hearing loss). OTOF expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100% or more) compared to OTOF expression prior to administration of the compositions described herein. OTOF function may be evaluated directly (e.g., using electrophysiological methods or imaging methods to assess exocytosis) or indirectly based on hearing tests, and may be increased by 5% or more (e.g., 5%, 10%, 15%, 20%, 30%, 40%, 50%, 60%, 70%, 80%, 90%, 100% or more) compared to OTOF function prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating sensorineural hearing loss or auditory neuropathy (e.g., hearing loss associated with a mutation in OTOF). Compositions may include nucleic acid vectors (e.g., AAV vectors) described herein (e.g., a first nucleic acid vector containing a polynucleotide that encodes and N-terminal portion of an OTOF isoform 5 protein and a second nucleic acid vector containing a polynucleotide that encodes a C-terminal portion of an OTOF isoform 5 protein), optionally packaged in an AAV virus capsid (e.g., an AAV1 capsid). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Human OTOF Isoform 5, but not Human OTOF Isoform 1, Rescued Function in OTOF^{Q828X/Q828X} Mice

There are multiple long and short isoforms of the Otoferlin (OTOF) gene. Studies of human genetic deafness have suggested that long isoforms are important for inner ear function. However, the role of these individual long isoforms and other protein variants in inner ear function is not understood. To develop effective gene transfer therapies for patients who experience deafness secondary to genetically driven OTOF deficiency, a cDNA sequence that encodes functional OTOF isoforms in the ear must be identified.

In human, seven OTOF isoforms have been identified in extra-aural tissues. Two of these isoforms (isoform 1 (V1) and isoform 5 (V5)) are long (both 1997 amino acids in length).

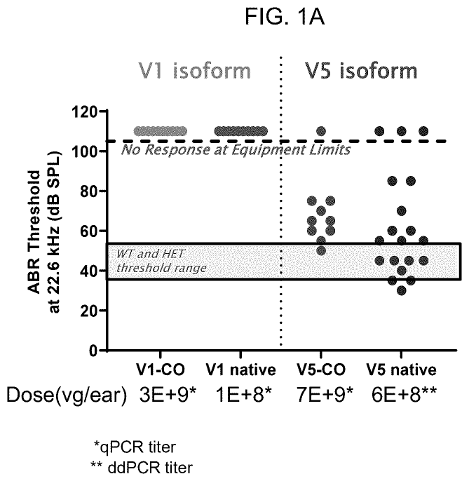

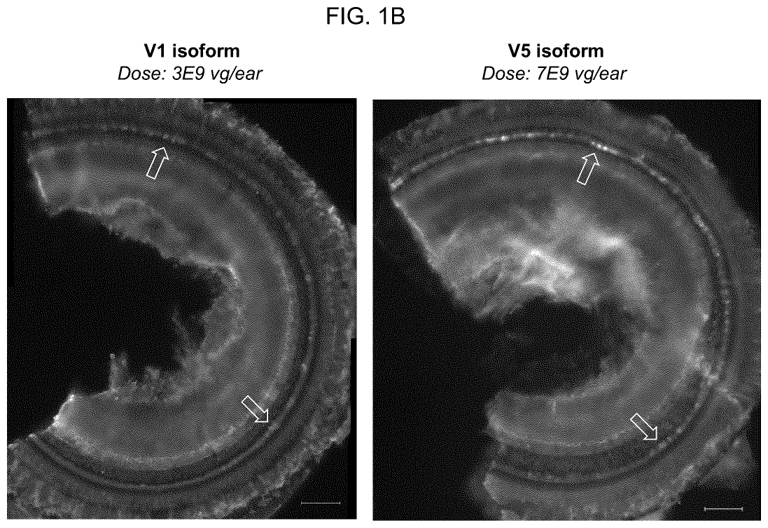

We used dual hybrid AAV technology to locally deliver the human V1 and V5 isoforms to the inner ears of genetically engineered, congenitally deaf mice with Otoferlin deficiency. One month after delivery of the human V5 isoform in the congenitally deaf mice, we observed rescue of hearing (FIG. 1A). By contrast, we saw no hearing recovery in mice that were injected with the V1 human long isoform. These studies establish the OTOF V5 sequence as being capable for restoring hearing in the context of a gene transfer therapeutic.

The experiments performed to compare functional recovery and OTOF expression using dual hybrid vector systems encoding OTOF isoform 1 or OTOF isoform 5 are described in detail below.

### AAV Delivery to Inner Ear

Vector (AAV2quad (Y-F)-smCBA-OTOF isoform 1 (native or codon-optimized) or AAV2quad (Y-F)-smCBA-OTOF isoform 5 (native or codon-optimized)) was delivered to P30-50 OTOF^{Q828X/Q828X} mutant mice (a mouse model of human OTOF mutation p.Gln828Ter) by injection through the round window membrane. The polynucleotide encoding the N-terminal portion of the OTOF protein and the polynucleotide encoding the C-terminal portion of the OTOF protein were divided between the two vectors in the dual hybrid vector system using the exon 20/21 boundary and an AP gene fragment (SEQ ID NO: 51) was used as the recombinogenic region in both vectors of the dual hybrid vector system. Vector was delivered as follows. Animals were anesthetized with isoflurane. Ophthalmic ointment (Puralube Vet) was applied to both eyes, and Meloxicam (Putney)/Rimadyl was administered at 0.3-2.0 mg/kg, SC as an analgesic. The surgical site was shaved and disinfected by scrubbing with Betadine (Avrio Health L.P.) and 70% alcohol before post-auricular incision to expose the bulla. A small hole was created in the bulla using a sterile 26-30 G needle or diameter 0.004-0.008 mm drill bits (Performance Micro Tool) and expanded using sterile sharp forceps to visualize the round window niche. The round window was then punctured with the tip of a pulled glass micropipette or a 35 G polyimide tubing (WPI Instrument) and any leaking fluid was absorbed using sterile gauze. Two microliters of vector were then injected using a micropipette (approximately 0.5 mm depth) or a polyimide tubing. Injection lasted approximately 30 seconds. When delivery was complete, a small piece of the cleidomastoideus muscle was cut and pushed through the bulla to cover the round window. Fat tissue was moved back to its original area. The skin incision was then closed using three to four drops of GLUture (Zoetis Inc.), allowing 1-2 minutes for it to dry. Animals were then moved to a clean and warm recovery cage. Animals were checked post-operatively for five days for signs of pain, infection, or other signs of distress. Analgesic was given once daily for the first three days after surgery. Animals were also given saline, nutritional support, and additional analgesia as appropriate.

### Auditory Brainstem Response

Mice were evaluated for peripheral auditory function by auditory brainstem response (ABR) at four weeks post-injection. Following administration of anesthesia (ketamine 100 mg/kg, i.p., xylazine 10 mg/kg, i.p.; supplemental ketamine as needed) and verifying appropriate anesthetic depth, subdermal needle electrodes were placed at vertex and ventral pinna (differential recording) and at base of tail (ground). A custom acoustic system (Eaton-Peabody Laboratories, MEE) containing two speakers and a probe-tube microphone was placed in the ear canal and in-ear calibration was performed before each test session. Probe-tube microphone calibration was performed using a reference ¼-in. microphone and preamplifier (PCB Piezotronics models 2530, 426B31; Larson-Davis model 2221); in-ear calibration was performed with the probe-tube microphone and custom microphone amplifier (Eaton-Peabody Laboratories, MEE). All ABR stimuli and responses were generated with and acquired by an RZ6 Multi-I/O signal processor and BioSigRZ software (Tucker-Davis Technologies). Responses from needle electrodes were amplified and digitized with a RA4PA Medusa preamp connected to a RA4LI low-impedance headstage (Tucker-Davis Technologies). ABR stimuli were 5 ms tone pips (0.5 ms cos² ramp) at 22.6 kHz, presented at a rate of 40/s with alternating polarity, from 5 to 105 dB SPL in 5 dB steps. For each sound level, a total of 1024 trials were averaged (after artifact rejection), digitally filtered (0.1-5 kHz), then additionally high-pass filtered offline (0.3 kHz). ABR threshold was determined by visual inspection of stacked waveforms (“waterfall plot”) as the lowest sound level at which a reproducible peak or trough was detected. If no response was detected up to equipment limit (105 dB SPL), a ceiling value of 110 db SPL was assigned. The results of ABR threshold at 22.6 kHz are shown in FIG. 1A.

### Immunohistochemistry

After physiology testing was completed, mice were euthanized via carbon dioxide inhalation overdose. Immediately after euthanasia, mice were perfused via the vascular system with 10% neutral buffered formalin (NBF). The inner ear temporal bone was collected and submerged in 10% NBF for 2-16 hours and then decalcified in 8% EDTA for 3 days. The cochlea was dissected from the de-calcified temporal bone and washed 3 times in PBS. After incubating with a blocking solution of 5% horse serum in PBST (0.5% triton X-100 in 1×PBS) at room temperature for one hour, the dissected cochlear pieces were incubated with a mouse anti-Otoferlin antibody (abcam ab53233, 1:200 diluted in PBST) at 4 °C for 12-16 hours, followed by 3 washes in PBS and incubation in Alexa 568 donkey anti-mouse secondary antibody (ThermoFisher Scientific A10037) at room temperature for 1-2 hours. After counter staining with DAPI and final washes with PBS, the cochlear pieces were mounted on a glass slide using Vectashield mounting medium (Vector Laboratories H-1000) and covered with a glass coverslip. The tissue was imaged using a Zeiss Axio Imager M2 microscope. Images are shown in FIG. 1B.

### Example 2. Administration of Higher Doses of OTOF Isoform 5 Improves Functional Recovery in OTOF^{Q828X/Q828X} Mice

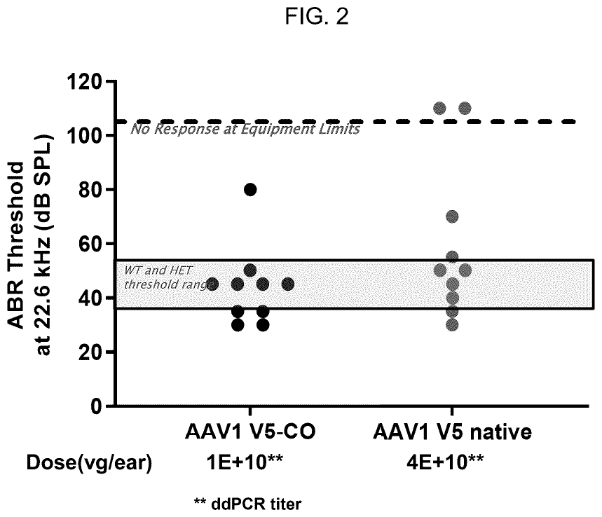

To evaluate the effect of administration of a higher dose of OTOF isoform 5 on functional recovery in OTOF^{Q828X/Q828X} mutant mice, vector (AAV1-smCBA-OTOF isoform 5 (native or codon-optimized)) was injected through the round window membrane of 4-7-week-old OTOF^{Q828X/Q828X} mice as described in Example 1, above. Similar to the vectors used in Example 1, the polynucleotide encoding the N-terminal portion of the OTOF protein and the polynucleotide encoding the C-terminal portion of the OTOF protein were divided between the two vectors in the dual hybrid vector system using the exon 20/21 boundary and an AP gene fragment (SEQ ID NO: 51) was used as the recombinogenic region in both vectors of the dual hybrid vector system. ABR responses were measured approximately four weeks after injection of the viruses, as described in Example 1, above. Relative to animals dosed with 7E9 or 6E9 vector genomes/ear (FIG. 1A), these animals showed even stronger ABR responses irrespective of codon-optimization (FIG. 2).

### Example 3. Administration of a Composition Containing Dual Hybrid Vectors that Express OTOF to Mice Restores Electrophysiological Signatures of Hearing Function

Homozygous (HOM) OTOF-Q828X mice (7 weeks old) were either left untreated or treated (by injection through the round window membrane) with 4E10 (4×10¹⁰) vector genomes (vg)/ear of an AAV1-Myo15 (SEQ ID NO: 21)-hOTOF (isoform 5, SEQ ID NO: 1) dual hybrid vector system in which exons 1-20 of the polynucleotide encoding the N-terminal portion of the OTOF protein (SEQ ID NO: 56) and exons 21-45 and 47 of the polynucleotide encoding the C-terminal portion of the OTOF protein (SEQ ID NO: 57) were delivered in separate vectors (FIG. 3A). An AP recombinogenic region (SEQ ID NO: 51) was included in both vectors of the dual hybrid vector system. Auditory brainstem response (ABR) thresholds were used to assess hearing function. Untreated animals (untreated Otof HOM) showed no detectable recovery in hearing function, whereas treated animals exhibited a robust recovery, which was consistent from four weeks post-treatment (Otof HOM at 4 weeks after treatment) to eight weeks post-treatment (Otof HOM at 8-11 weeks after treatment). ABR thresholds in heterozygous animals (Otof HET) were also tested.

In a separate set of experiments, homozygous OTOF-Q828X mice were either left untreated or treated (by injection through the round window membrane) with 4E10 (4×10¹⁰) vg/ear of an AAV1-truncated chimeric CMV-chicken β-actin (smCBA, SEQ ID NO: 44)-hOTOF (isoform 5, SEQ ID NO: 1) dual hybrid vector system as described above (FIG. 3B). The first vector contained exons 1-20 of the polynucleotide encoding the N-terminal portion of the OTOF protein (SEQ ID NO: 56) and exons 21-45 and 47 of the polynucleotide encoding the C-terminal portion of the OTOF protein (SEQ ID NO: 57) and both vectors contained an AP recombinogenic region (SEQ ID NO: 51). Untreated animals had no detectable recovery in hearing function, whereas treated animals exhibited a robust recovery at 4 weeks post-treatment (Otof HOM at 4 weeks after treatment). When these same animals were evaluated at 8 weeks post-treatment (Otof HOM at 8 weeks after treatment), ABR thresholds increased, suggesting less durable recovery with the smCBA promoter. ABR thresholds in heterozygous animals were also tested.

In yet another set of experiments, homozygous OTOF-Q828X mice were either left untreated or treated (by injection through the round window membrane) with an AAV1-smCBA (SEQ ID NO: 44)-hOTOF (isoform 5, SEQ ID NO: 1) dual hybrid vector, as described above, at either 8E9 (8×10⁹) vg/ear (low dose), 1.6E10 (1.6×10¹⁰) vg vg/ear (mid dose), or 6.4E10 (6.4×10¹⁰) vg/ear (high dose). The first vector contained exons 1-20 of the polynucleotide encoding the N-terminal portion of the OTOF protein (SEQ ID NO: 56) and exons 21-45 and 47 of the polynucleotide encoding the C-terminal portion of the OTOF protein (SEQ ID NO: 57) and both vectors contained an AP recombinogenic region (SEQ ID NO: 51). ABR thresholds were used to assess hearing function at four weeks and eight weeks post-treatment (FIG. 3C). A dose-dependent recovery in ABR was observed at both timepoints. When comparing the eight weeks versus the four weeks timepoint, recovery of hearing function was steady for the low and mid doses but decreased for the high dose animals. ABR thresholds in heterozygous animals were also tested.

### Example 4. Administration of a Composition Containing Dual Hybrid Vectors that Express Murine OTOF to Mice Restores Electrophysiological Signatures of Hearing Function

Homozygous OTOF-Q828X mice (6-7 weeks old) were treated with AAV2quad (Y-F)-Myo15 (SEQ ID NO: 21)-murine OTOF (mOTOF, transcript variant 1, RefSeq NM_001100395) and AAV2quad (Y-F)-Myo15 (SEQ ID NO: 31)-mOTOF (transcript variant 1, RefSeq NM_001100395) dual hybrid vector systems by injection through the round window membrane as described above (2 μL total volume injected, 1 μL of each vector) and ABR thresholds were used to assess hearing function. Exons 1-20 of the polynucleotide encoding the N-terminal portion of the mOTOF protein were included in the 5′ vector and the remaining C-terminal portion of the mOTOF polynucleotide was included in the 3′ vector. An AP recombinogenic region (SEQ ID NO: 51) was included in both vectors of the dual hybrid vector system. The titer of the 5′ AAV2quad (Y-F)-Myo15 (SEQ ID NO: 21)-mOTOF vector was 1.49E12 vg/mL, the titer of the 5′ AAV2quad (Y-F)-Myo15 (SEQ ID NO: 31)-mOTOF vector was 2.68E12 vg/mL, and the titer of the corresponding 3′ vectors was 1.05E12 vg/mL and 1.58E12 vg/mL. Untreated animals showed no detectable recovery in hearing function, whereas treated animals exhibited robust recovery, which was consistent from four weeks post-treatment to seventeen weeks post-treatment (FIG. 4). FIG. 4 depicts mean hearing thresholds at 22.6 kHz+/−standard deviation.

### Example 5. Dual Hybrid Vectors can be Used to Express Full Length, Functional GFP in Hair Cells of a Non-Human Primate

A non-human primate of 2.6 years of age received a local injection to the round window of the inner ear at a flow rate of 6 μL/min with an AAV1-Myo15 (SEQ ID NO: 21)-GFP (viral titer of 3.18E13 vg/mL for the 5′ vector and 3.42E13 vg/mL for the 3′ vector) dual hybrid vector system (60 μL total volume injected, 30 μL of each vector). Four weeks post-injection, inner ears were removed, and a surface preparation of the basilar membranes was made. Dual hybrid vectors resulted in GFP expression in hair cells across the entire baso-apical axis of the cochlea. High magnification images at 4 kHz showed GFP expression was observed within inner hair cells (IHCs) (FIG. 5B). Immunohistochemistry for Myo7A was used to visualize hair cells (FIG. 5A) and nuclei were stained with DAPI (FIG. 5C).

### Example 6. Construction of Dual Hybrid Vector Systems that Encode Human OTOF

HEK293T cells (obtained from ATCC, Manassas, VA) were seeded into cell culture-treated dishes until they reached 70-80% confluence in the vessel. Transfection was carried out using the AAV-1 Packaging System from Cell Biolabs, Inc. (San Diego, CA). Per 175 cm² of culture surface area, 16 μg pHelper, 8 μg pAAV-RC1, and 8 μg of transfer plasmid SEQ ID NO:66 were mixed with PEI (PElpro, polyplus) at a 1:1 weight ratio. The DNA/PEI mixture was subsequently added into the cell culture medium. Three days after transfection, the cell culture medium and the cells were collected to extract and purify the AAV. AAV from the cell culture medium was concentrated through tangential flow filtration. AAV from the cells was released from cells through three cycles of freeze thaw. AAV from either fraction was finally purified through iodixanol density gradient purification and the buffer was exchanged by passing the purified AAV and sterile DPBS (Mg+, Ca+) with 0.01% pluronic F68 across a 100 kDa MWCO centrifugal filter to produce purified AAV, which is the first member of the dual vector system. A similar procedure was carried out using transfer plasmid SEQ ID NO:67 to create the second member of the dual vector system.

The same procedure was used to create a dual vector system that encodes OTOF in AAV9, except that pAAV-RC1 was replaced with a comparable plasmid that contained the AAV9 cap gene instead of the AAV1 cap gene during the transfection step.

### Example 7. Dual Hybrid Vector Systems that Encode Human OTOF are Expressed in Non-Human Primates

In non-human primates (NHP) native Otoferlin protein is expressed in the sensory cells of the inner ear. We used the BaseScope™ system to detect expression of virally transduced human Otoferlin in NHPs.

A first cohort of six naïve NHP (1.5-4 years old) were injected through the round window membrane with the Myo15-Otoferlin dual hybrid vectors packaged into the AAV1 serotype as described in Example 6. Each ear received a dose of 1.1×10¹² copies of each of the two vectors together in a total volume of 60 μl. The ears were vented at the lateral semicircular canals to allow an outflow of perilymph during viral delivery. A second cohort of naïve NHP (1.5-4 years old) was injected in the same manner and dosage as the first cohort with a Myo15-Otoferlin dual hybrid system packaged into the AAV9 serotype, also as described in Example 6.

All animals were sacrificed by cardiac perfusion of 10% neutral buffered formalin (NBF) four weeks after viral vector injections and their temporal bones were harvested. After decalcification for five days in Immunocal, the temporal bones were embedded in paraffin, sectioned in 5 μm intervals and stained for BaseScope™ (Advanced Cell Diagnostics, Newark, CA) using a probe specific to the spliced mRNA at the junction of the two vectors between Exons 20 and 21 of human Otoferlin. Thus, detection required that the dual vectors properly hybridized in vivo and expressed full-length human OTOF isoform 5.

The BaseScope™ assay was performed on the Leica Bond RX automatic staining platform. Briefly, paraffin sections were baked for 30 min at 60 °C and deparaffinized. A target retrieval step for 5 min at 95 °C was followed by a proteinase step for 10 min at 40 °C and finally a standard BaseScope™ protocol including 8 amplification steps was run on the E20/21-probe. The probe-signal was detected using the Fast Red dye, which could be observed as a red staining in bright field microscopy. Initially, images where scanned and digitized at 40× magnification and screened for positivity. Afterward, the positive signal was confirmed using the fluorescent signal of the fast red dye using confocal imaging (63×, 1.4 NA, excitation: 568 nm at 1% laser power; emission BP 578-730 nm at 642V detector). FIG. 6 shows sections from representative NHPs treated with the Myo15-Otoferlin AAV1 dual vector system. In the AAV1-injected animals, clear positivity could be observed in sensory cells in all injected animals with a bias toward the apical turns of the cochlea and the utricle. In the AAV9-injected animals, three out of the six animals showed clear positivity with a similar trend toward cochlear apical turn and utricular expression (figure not shown). In each case, the OTOF transcript was localized to inner hair cells, outer hair cells and hair cells in the vestibular organs.

### Example 8. Quantification and Localization of Surrogate Myo15 Promoter Dual Hybrid Vector Systems Encoding eGFP in NHP

To ascertain the specificity of the hair cell-specific Myo15 promoter and transduction efficiency in NHPs, dual vector systems having the same recombinatory regions and promoter as described in Example 6 were used, except that the 5′ and 3′ portions of human otoferlin in the two respective vectors were replaced with the first 393 nucleotides encoding a 5′ portion of eGFP in the 5′ vector and the last 908 nucleotides encoding a 3′ portion of eGFP in the 3′ vector, respectively. eGFP is easily detectable even in an otoferlin wildtype background.

All animals received injections of 60 μl the dual viral vectors through the round window membrane and their ears were vented at the lateral semicircular canal. The animals used in this study were 1.5-4 years old. Six NHP ears were injected with a surrogate vector expressing eGFP under the Myo15 promoter in the AAV1 serotype described above at a titer of 1.6×10¹² vector genomes per ear. Another six NHP ears were injected with a similar surrogate dual viral vector system expressing eGFP under the Myo15 promoter in the AAV9 serotype at a titer of 1.9×10¹² vector genomes per ear. The dose for the NHP study was scaled by correlating mouse expression data from prior mid and high dose experiments and comparing it to a smaller scale NHP mid dose experiment (Table 5, below). We estimated the potential outcome in NHP as a percentage of Inner hair cells (IHCs) expressing eGFP from a high dose dual vector approach (Table 5).

TABLE 5
Dose scaling from mouse to NHP in dual vector eGFP delivery

| | Predicted Minimum | | | | | |
|---|---|---|---|---|---|---|
| | Efficacious Dose | | | Predicted Dose Range for | | |
| | (MED) Range | | | Saturation of Efficacy | | |
| | Dose | Total | % IHC | Dose | Total | % IHC |
| Species | Concentration | Dose* | expression** | Concentration | Dose* | expression** |
| Mouse | 3.9e10 vg/μL | 7.4e10 | 57% | 6.2e10 vg/μL | 1.2e11 | 82% |
| | | vg/ear | | | vg/ear | |
| Cynomolgus | 3.1e10 vg/μL | 1.2e12 | 40-60% | 5.2e10 vg/μL | 2e12 | 75-100% |
| | | vg/ear | | | vg/ear | |

vg = viral genomes
*Toal Dose calculated as 5′ + 3′
**% IHC expression of hOTOF in mouse averaged across 8, 11.3, 16, 22.6, and 32 kHz
**% IHC expression of dualGFP in cynomolgus monkey averaged across 1, 2, 4, and 8 kHz

We confirmed expression in NHPs throughout the organ of Corti using whole mount confocal imaging (40×, 0.95 NA, excitation: 488 nm at 14% laser power; emission BP 495-543 nm at 600V detector gain) using native eGFP signal as a readout (FIG. 7A). The quantification of the number of inner hair cells showing expression correlated well to the prediction from dose scaling (FIG. 7B) for the AAV1 serotype, while the AAV9 serotype showed a lower expression percentage.

### Example 9. Administration of a Composition Containing Dual Hybrid Vectors that Express OTOF Isoform 5 to a Subject with Sensorineural Hearing Loss

According to the methods disclosed herein, a physician of skill in the art can treat a patient, such as a human patient, with sensorineural hearing loss (e.g., sensorineural hearing loss associated with a mutation in OTOF) so as to improve or restore hearing. To this end, a physician of skill in the art can administer to the human patient a composition containing a first AAV vector (e.g., AAV1) containing a Myo15 promoter (e.g., SEQ ID NO: 19, 21, 22, 31, or 32) operably linked to exons 1-20 of a polynucleotide encoding an OTOF isoform 5 protein (e.g., human OTOF isoform 5, e.g., SEQ ID NO: 1, e.g., a polynucleotide having the sequence of SEQ ID NO: 56), a splice donor sequence 3′ of the polynucleotide sequence, and an AP recombinogenic region (e.g., an AP gene fragment, any one of SEQ ID NOs: 48-53, e.g., SEQ ID NO: 51) 3′ of the splice donor sequence, and a second AAV vector (e.g., AAV1) containing an AP recombinogenic region (an AP gene fragment, any one of SEQ ID NOs: 48-53, e.g., SEQ ID NO: 51), a splice acceptor sequence 3′ of the recombinogenic region, a polynucleotide 3′ of the splice acceptor sequence that contains exons 21-45 and 47 of a polynucleotide encoding an OTOF isoform 5 protein (e.g., human OTOF isoform 5, e.g., SEQ ID NO: 1, e.g., a polynucleotide having the sequence of SEQ ID NO: 57), and a bGH poly(A) sequence. The composition containing the dual hybrid AAV vectors may be administered to the patient, for example, by local administration to the inner ear (e.g., injection through the round window membrane, injection into a semicircular canal, or by canalostomy), to treat sensorineural hearing loss.

Following administration of the composition to a patient, a practitioner of skill in the art can monitor the expression of OTOF, and the patient's improvement in response to the therapy, by a variety of methods. For example, a physician can monitor the patient's hearing by performing standard tests, such as audiometry, ABR, electrocochleography (ECOG), and measuring otoacoustic emissions following administration of the composition. A finding that the patient exhibits improved hearing (e.g., improved ABR) in one or more of the tests following administration of the composition compared to hearing test results prior to administration of the composition indicates that the patient is responding favorably to the treatment. Subsequent doses can be determined and administered as needed.

Exemplary embodiments of the invention are described in the enumerated paragraphs below.
E1. A dual vector system comprising:
   a first adeno-associated virus (AAV) vector comprising a Myo15 promoter operably linked to a first coding polynucleotide that encodes an N-terminal portion of an Otoferlin (OTOF) isoform 5 protein, a splice donor signal sequence positioned 3′ of the first coding polynucleotide, and a first recombinogenic region positioned 3′ of the splice donor signal sequence; and
   a second AAV vector comprising a second recombinogenic region, a splice acceptor signal sequence positioned 3′ of the second recombinogenic region, a second coding polynucleotide that encodes a C-terminal portion of the OTOF isoform 5 protein positioned 3′ of the splice acceptor signal sequence, and a poly(A) sequence positioned 3′ of the second coding polynucleotide;
   wherein the first coding polynucleotide and the second coding polynucleotide that encode the OTOF isoform 5 protein do not overlap, and wherein neither the first nor second AAV vector encodes the full-length OTOF isoform 5 protein.
E2. The dual vector system of E1, wherein the first AAV vector and the second AAV vector comprise an AAV1 capsid.
E3. The dual vector system of E1 or E2, wherein the Myo 15 promoter comprises a first region having at least 85% sequence identity to SEQ ID NO: 7 or a functional portion or derivative thereof comprising the sequence of SEQ ID NO: 9 and/or SEQ ID NO: 10 operably linked to a second region having at least 85% sequence identity to SEQ ID NO: 8 or a functional portion or derivative thereof comprising the sequence of SEQ ID NO: 14 and/or SEQ ID NO: 15, optionally comprising a linker comprising one to one hundred nucleotides between the first region and the second region.
E4 The dual vector system of E3, wherein the first region comprises or consists of the sequence of SEQ ID NO: 7.
E5. The dual vector system of E3, wherein the functional portion of SEQ ID NO: 7 comprises the sequence of SEQ ID NO: 9.
E6. The dual vector system of E3, wherein the functional portion of SEQ ID NO: 7 comprises the sequence of SEQ ID NO: 10.
E7. The dual vector system of E3, wherein the functional portion of SEQ ID NO: 7 comprises the sequence of SEQ ID NO: 9 and the sequence of SEQ ID NO: 10.
E8. The dual vector system of E7, wherein the functional portion of SEQ ID NO: 7 comprises the sequence of SEQ ID NO: 11.
E9. The dual vector system of E7, wherein the functional portion of SEQ ID NO: 7 comprises the sequence of SEQ ID NO: 12.
E10. The dual vector system of E7, wherein the functional portion of SEQ ID NO: 7 comprises the sequence of SEQ ID NO: 13.
E11. The dual vector system of E7, wherein the functional portion of SEQ ID NO: 7 comprises the sequence of SEQ ID NO: 33.
E12. The dual vector system of any one of E3-E11, wherein the second region comprises or consists of the sequence of SEQ ID NO: 8.
E13. The dual vector system of any one of E3-E11, wherein the functional portion of SEQ ID NO: 8 comprises the sequence of SEQ ID NO: 14.
E14. The dual vector system of any one of E3-E11, wherein the functional portion of SEQ ID NO: 8 comprises the sequence of SEQ ID NO: 15.
E15. The dual vector system of any one of E3-E11, wherein the functional portion of SEQ ID NO: 8 comprises the sequence of SEQ ID NO: 14 and the sequence of SEQ ID NO: 15.
E16. The dual vector system of E15, wherein the functional portion of SEQ ID NO: 8 comprises the sequence of SEQ ID NO: 16.
E17. The dual vector system of E15, wherein the functional portion of SEQ ID NO: 8 comprises the sequence of SEQ ID NO: 17.
E18. The dual vector system of E15, wherein the functional portion of SEQ ID NO: 8 comprises the sequence of SEQ ID NO: 18.
E19. The dual vector system of any one of E3-E11, wherein the functional portion of SEQ ID NO: 8 comprises the sequence of SEQ ID NO: 34.
E20. The dual vector system of any one of E3-E11, wherein the functional portion of SEQ ID NO: 8 comprises the sequence of SEQ ID NO: 35.
E21. The dual vector system of any one of E3-E11, wherein the functional portion of SEQ ID NO: 8 comprises the sequence of SEQ ID NO: 34 and the sequence of SEQ ID NO: 35.
E22. The dual vector system of E21, wherein the functional portion of SEQ ID NO: 8 comprises the sequence of SEQ ID NO: 38.
E23. The dual vector system of E3, wherein the Myo15 promoter comprises or consists of the sequence of SEQ ID NO: 19.
E24. The dual vector system of E3, wherein the Myo15 promoter comprises or consists of the sequence of SEQ ID NO: 21.
E25. The dual vector system of E3, wherein the Myo15 promoter comprises or consists of the sequence of SEQ ID NO: 22.
E26. The dual vector system of E3, wherein the Myo15 promoter comprises or consists of the sequence of SEQ ID NO: 42.
E27. The dual vector system of E3, wherein the Myo15 promoter comprises or consists of the sequence of SEQ ID NO: 43.
E28. The dual vector system of E1 or E2, wherein the Myo15 promoter comprises a first region having at least 85% sequence identity to SEQ ID NO: 23 or a functional portion or derivative thereof comprising the sequence of SEQ ID NO: 25 operably linked to a second region having at least 85% sequence identity to SEQ ID NO: 24 or a functional portion or derivative thereof comprising the sequence of SEQ ID NO: 26 and/or SEQ ID NO: 27, optionally comprising a linker comprising one to four hundred nucleotides between the first region and the second region.
E29. The dual vector system of E28, wherein the first region comprises or consists of the sequence of SEQ ID NO: 23.
E30. The dual vector system of E28, wherein the functional portion of SEQ ID NO: 23 comprises the sequence of SEQ ID NO: 25.
E31. The dual vector system of any one of E28-E30, wherein the second region comprises or consists of the sequence of SEQ ID NO: 24.
E32. The dual vector system of any one of E28-E30, wherein the functional portion of SEQ ID NO: 24 comprises the sequence of SEQ ID NO: 26.
E33. The dual vector system of any one of E28-E30, wherein the functional portion of SEQ ID NO: 24 comprises the sequence of SEQ ID NO: 27.
E34. The dual vector system of any one of E28-E30, wherein the functional portion of SEQ ID NO: 24 comprises the sequence of SEQ ID NO: 26 and the sequence of SEQ ID NO: 27.
E35. The dual vector system of E34, wherein the functional portion of SEQ ID NO: 24 comprises the sequence of SEQ ID NO: 28.
E36. The dual vector system of E34, wherein the functional portion of SEQ ID NO: 24 comprises the sequence of SEQ ID NO: 29.
E37. The dual vector system of E34, wherein the functional portion of SEQ ID NO: 24 comprises the sequence of SEQ ID NO: 30.
E38. The dual vector system of E28, wherein the Myo15 promoter comprises or consists of the sequence of SEQ ID NO: 31.
E39. The dual vector system of E28, wherein the Myo15 promoter comprises or consists of the sequence of SEQ ID NO: 32.
E40. The dual vector system of E1 or E2, wherein the Myo 15 promoter comprises a sequence having at least 85% sequence identity to SEQ ID NO: 34.
E41. The dual vector system of E1 or E2, wherein the Myo15 promoter comprises a sequence having at least 85% sequence identity to SEQ ID NO: 38.
E42. The dual vector system of E1 or E2, wherein the Myo15 promoter comprises a sequence having at least 85% sequence identity to SEQ ID NO: 39.
E43. The dual vector system of E1 or E2, wherein the Myo 15 promoter comprises a sequence having at least 85% sequence identity to SEQ ID NO: 40.
E44. A dual vector system comprising:
   a first AAV1 vector comprising a ubiquitous promoter operably linked to a first coding polynucleotide that encodes an N-terminal portion of an OTOF isoform 5 protein, a splice donor signal sequence positioned 3′ of the first coding polynucleotide, and a first recombinogenic region positioned 3′ of the splice donor signal sequence; and
   a second AAV1 vector comprising a second recombinogenic region, a splice acceptor signal sequence positioned 3′ of the second recombinogenic region, a second coding polynucleotide that encodes a C-terminal portion of the OTOF isoform 5 protein positioned 3′ of the splice acceptor signal sequence, and a poly(A) sequence positioned 3′ of the second coding polynucleotide;
   wherein the first coding polynucleotide and the second coding polynucleotide do not overlap, and
   wherein neither the first nor second AAV1 vector encodes the full-length OTOF isoform 5 protein.
E45. The dual vector system of E44, wherein the ubiquitous promoter is selected from the group consisting of a CAG promoter, a cytomegalovirus (CMV) promoter, and a truncated CMV-chicken β-actin promoter (smCBA promoter).
E46. The dual vector system of E45, wherein the ubiquitous promoter is the smCBA promoter.
E47. The dual vector system of E46, wherein the smCBA promoter comprises or consists of the sequence of SEQ ID NO: 44.
E48. The dual vector system of any one of E1-E47, wherein the first and second recombinogenic regions are the same.
E49. The dual vector system of any one of E1-E48, wherein the first recombinogenic region and/or the second recombinogenic region is an AK recombinogenic region.
E50. The dual vector system of E49, wherein the AK recombinogenic region comprises or consists of the sequence of SEQ ID NO: 47.
E51. The dual vector system of any one of E1-E48, wherein the first recombinogenic region and/or the second recombinogenic region is an AP gene fragment.
E52. The dual vector system of E51, wherein the AP gene fragment comprises or consists of the sequence of any one of SEQ ID NOs: 48-53.
E53. The dual vector system of E52, wherein the AP gene fragment comprises or consists of the sequence of SEQ ID NO: 51.
E54. The dual vector system of any one of E1-E53, wherein each of the first and second coding polynucleotides encode about half of the OTOF isoform 5 protein sequence.
E55. The dual vector system of any one of E1-E54, wherein the first and second coding polynucleotides are divided at an OTOF exon boundary.
E56. The dual vector system of E55, wherein the first and second coding polynucleotides are divided at the OTOF exon 20/exon 21 boundary.
E57. The dual vector system of any one of E1-E55, wherein the first coding polynucleotide consists of exons 1-20 of a polynucleotide encoding the OTOF isoform 5 protein and the second coding polynucleotide consists of exons 21-45 and 47 of a polynucleotide encoding the OTOF isoform 5 protein.
E58. The dual vector system of any one of E1-E57, wherein the first and second coding polynucleotides that encode the OTOF isoform 5 protein do not comprise introns.
E59. The dual vector system of any one of E1-E58, wherein the OTOF isoform 5 protein is a human OTOF isoform 5 protein.
E60. The dual vector system of any one of E1-E59, wherein the OTOF isoform 5 protein comprises the sequence of SEQ ID NO: 1 or a variant thereof having one or more conservative amino acid substitutions.
E61. The dual vector system of E60, wherein no more than 10% of the amino acids in the OTOF isoform 5 protein variant are conservative amino acid substitutions.
E62. The dual vector system of E60, wherein the OTOF isoform 5 protein consists of the sequence of SEQ ID NO: 1.
E63. The dual vector system of any one of E1-E60 and E62, wherein the OTOF isoform 5 protein is encoded by the sequence of SEQ ID NO: 2.
E64. The dual vector system of any one of E1-E60 and E62, wherein the OTOF isoform 5 protein is encoded by the sequence of SEQ ID NO: 3.
E65. The dual vector system of any one of E1-E64, wherein the first coding polynucleotide encodes amino acids 1-802 of SEQ ID NO: 1 and the second coding polynucleotide encodes amino acids 803-1997 of SEQ ID NO: 1.
E66. The dual vector system of any one of E1-E65, wherein the N-terminal portion of the OTOF isoform 5 protein consists of the sequence of SEQ ID NO: 58 or a variant thereof having one or more conservative amino acid substitutions.
E67. The dual vector system of E66, wherein no more than 10% (e.g., 10%, 9%, 8%, 7%, 6%, 5%, 4%, 3%, 2%, 1% or fewer) of the amino acids in the N-terminal portion of the OTOF isoform 5 protein variant are conservative amino acid substitutions.
E68. The dual vector system of E66, wherein the N-terminal portion of the OTOF isoform 5 protein consists of the sequence of SEQ ID NO: 58.
E69. The dual vector system of any one of E1-E66 and E68, wherein the N-terminal portion of the OTOF isoform 5 protein is encoded by the sequence of SEQ ID NO: 56.
E70. The dual vector system of any one of E1-E69, wherein the C-terminal portion of the OTOF isoform 5 protein consists of the sequence of SEQ ID NO: 59 or a variant thereof having one or more conservative amino acid substitutions.
E71. The dual vector system of E70, wherein no more than 10% (e.g., 10%, 9%, 8%, 7%, 6%, 5%, 4%, 3%, 2%, 1% or fewer) of the amino acids in the C-terminal portion of the OTOF isoform 5 protein variant are conservative amino acid substitutions.
E72. The dual vector system of E70, wherein the C-terminal portion of the OTOF isoform 5 protein consists of the sequence of SEQ ID NO: 59.
E73. The dual vector system of any one of E1-E70 and E72, wherein the C-terminal portion of the OTOF isoform 5 protein is encoded by the sequence of SEQ ID NO: 57.
E74. The dual vector system of any one of E1-E73, wherein the first vector comprises a first inverted terminal repeat (ITR) sequence 5′ of the promoter and a second ITR sequence 3′ of the recombinogenic region, and the second vector comprises a first ITR sequence 5′ of the recombinogenic region and a second ITR sequence 3′ of the poly(A) sisequence.
E75. The dual vector system of E74, wherein the ITRs in the first vector and second vector are AAV2 ITRs or have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.
E76. The dual vector system of any one of E1-E75, wherein the poly(A) sisequence is a bovine growth hormone (bGH) poly(A) signal sequence.
E77. The dual vector system of any one of E1-E76, wherein the splice donor sequence in the first vector comprises or consists of the sequence of SEQ ID NO: 54.
E78. The dual vector system of any one of E1-E77, wherein the splice acceptor sequence in the second vector comprises or consists of the sequence of SEQ ID NO: 55.
E79. The dual vector system of any one of E1-E78, wherein the first AAV vector comprises a Kozak sequence 3′ of the promoter and 5′ of the first coding polynucleotide that encodes the N-terminal portion of the OTOF isoform 5 protein.
E80. The dual vector system of E1, wherein the first AAV vector comprises a polynucleotide sequence comprising the sequence of nucleotides 2272 to 6041 of SEQ ID NO: 60.
E81. The dual vector system of E1 or E80, wherein the first AAV vector comprises a polynucleotide sequence comprising or consisting of the sequence of nucleotides 2049 to 6264 of SEQ ID NO: 60.
E82. The dual vector system of E1, wherein the first AAV vector comprises a polynucleotide sequence comprising the sequence of nucleotides 182 to 3949 of SEQ ID NO: 62.
E83. The dual vector system of E1 or E82, wherein the first AAV vector comprises a polynucleotide sequence comprising or consisting of the sequence of nucleotides 19 to 4115 of SEQ ID NO: 62.
E84. The dual vector system of E44, wherein the first AAV vector comprises a polynucleotide sequence comprising the sequence of positions 2267 to 6014 of SEQ ID NO: 64.
E85. The dual vector system of E44 or E84, wherein the first AAV vector comprises a polynucleotide sequence comprising or consisting of the sequence of positions 2049 to 6237 of SEQ ID NO: 64.
E86. The dual vector system of E44, wherein the first AAV vector comprises a polynucleotide sequence comprising the sequence of positions 177 to 3924 of SEQ ID NO: 65.
E87. The dual vector system of E44 or E86, wherein the first AAV vector comprises a polynucleotide sequence comprising or consisting of the sequence of positions 19 to 4090 of SEQ ID NO: 65.
E88. The dual vector system of any one of E1, E44, E80, E81, E84, and E85, wherein the second AAV vector comprises a polynucleotide sequence comprising the sequence of nucleotides 2267 to 6476 of SEQ ID NO: 61.
E89. The dual vector system of any one of E1, E44, E80, E81, E84, E85, and E88, wherein the second AAV vector comprises a polynucleotide sequence comprising or consisting of the sequence of nucleotides 2049 to 6693 of SEQ ID NO: 61.
E90. The dual vector system of any one of E1, E44, E82, E83, E86, and E87, wherein the second AAV vector comprises a polynucleotide sequence comprising the sequence of nucleotides 187 to 4396 of SEQ ID NO: 63.
E91. The dual vector system of any one of E1, E44, E82, E83, E86, E87, and E90, wherein the second AAV vector comprises a polynucleotide sequence comprising or consisting of the sequence of nucleotides 19 to 4589 of SEQ ID NO: 63.
E92. The dual vector system of E1, wherein the first AAV vector comprises a polynucleotide sequence comprising the sequence of nucleotides 235 to 4004 of SEQ ID NO: 66.
E93. The dual vector system of E1 or E92, wherein the first AAV vector comprises a polynucleotide sequence comprising or consisting of the sequence of nucleotides 12 to 4227 of SEQ ID NO: 66.
E94. The dual vector system of E44, wherein the first AAV vector comprises a polynucleotide sequence comprising the sequence of nucleotides 230 to 3977 of SEQ ID NO: 68.
E95. The dual vector system of E44 or E94, wherein the first AAV vector comprises a polynucleotide sequence comprising or consisting of the sequence of nucleotides 12 to 4200 of SEQ ID NO: 68.
E96. The dual vector system of any one of E1, E44, and E92-E95, wherein the second AAV vector comprises a polynucleotide sequence comprising the sequence of nucleotides 229 to 4438 of SEQ ID NO: 67.
E97. The dual vector system of any one of E1, E44, and E92-E96, wherein the second AAV vector comprises a polynucleotide sequence comprising or consisting of the sequence of nucleotides 12 to 4655 of SEQ ID NO: 67.
E98. A method of increasing OTOF expression in a subject in need thereof, the method comprising administering to the subject a therapeutically effective amount of the dual vector system of any one of E1-E97.
E99. A method of treating a subject having or at risk of developing sensorineural hearing loss, the method comprising administering to the subject a therapeutically effective amount of the dual vector system of any one of E1-E97.
E100. A method of treating a subject having or at risk of developing auditory neuropathy, the method comprising administering to the subject a therapeutically effective amount of the dual vector system of any one of E1-E97.
E101. The method of any one of E98-E100, wherein the subject has a mutation in OTOF.
E102. The method of any one of E98-E100, wherein the subject has been identified as having a mutation in OTOF.
E103. The method of any one of E98-E100, wherein the method further comprises identifying the subject as having a mutation in OTOF prior to administering the dual vector system.
E104. The method of any one of E98-E103, wherein the subject has or is identified as having Deafness, Autosomal Recessive 9 (DFNB9).
E105. The method of any one of E98-E104, wherein the method further comprises evaluating the hearing of the subject prior to administering the dual vector system.
E106. The method of any one of E98-E105, wherein the dual vector system is administered locally to the middle or inner ear.
E107. The method of E106, wherein the dual vector system is administered by injection through the round window membrane, injection into a semicircular canal, canalostomy, insertion of a catheter through the round window membrane, transtympanic injection, or intratympanic injection.
E108. The method of any one of E98-107, wherein the method increases OTOF expression in a cochlear hair cell.
E109. The method of E108, wherein the cochlear hair cell is an inner hair cell.
E110. The method of any one of E98-E109, wherein the subject is a mammal.
E111. The method of E110, wherein the subject is a human.
E112. The method of any one of E98-E111, wherein the method further comprises evaluating the hearing of the subject after administering the dual vector system.
E113. The method of any one of E98-E112, wherein the dual vector system prevents or reduces hearing loss, delays the development of hearing loss, slows the progression of hearing loss, improves hearing, improves speech discrimination, or improves hair cell function.
E114. The method of any one of E98-E113, wherein the dual vector system is administered in an amount sufficient to increase OTOF expression in a cochlear hair cell, prevent or reduce hearing loss, delay the development of hearing loss, slow the progression of hearing loss, improve hearing, improve speech discrimination, or improve hair cell function.
E115. A method of increasing OTOF expression in a cell, the method comprising introducing the dual vector system of any one of E1-E97 into the cell.
E116. The method of E115, wherein the cell is a cochlear hair cell.
E117. The method of E116, wherein the cell is an inner hair cell.
E118. The method of any one of E115-117, wherein the cell is a mammalian cell.
E119. The method of E118, wherein the cell is a human cell.
E120. The method of any one of E98-E119, wherein the first vector and the second vector are administered concurrently.
E121. The method of any one of E98-E120, wherein the first vector and the second vector are administered sequentially.
E122. The method of any one of E98-E121, wherein the first vector and the second vector are administered at a concentration of about 1×10⁷ vector genomes (VG)/ear to about 2×10¹⁵ VG/ear.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A method of treating a subject having or at risk of developing sensorineural hearing loss or auditory neuropathy, the method comprising administering to the subject a dual vector system, wherein the dual vector system comprises:
(i) a first adeno-associated virus (AAV) vector comprising a Myo15 promoter operably linked to a first coding polynucleotide that encodes an N-terminal portion of an Otoferlin (OTOF) isoform 5 protein, a splice donor signal sequence positioned 3′ of the first coding polynucleotide, and a first recombinogenic region positioned 3′ of the splice donor signal sequence; and
(ii) a second AAV vector comprising a second recombinogenic region, a splice acceptor signal sequence positioned 3′ of the second recombinogenic region, a second coding polynucleotide that encodes a C-terminal portion of the OTOF isoform 5 protein positioned 3′ of the splice acceptor signal sequence, and a poly(A) sequence positioned 3′ of the second coding polynucleotide,
wherein the first coding polynucleotide and the second coding polynucleotide that encode the OTOF isoform 5 protein do not overlap,
wherein neither the first nor second AAV vector encodes the full-length OTOF isoform 5 protein,
and wherein:
(a) the first AAV vector comprises a polynucleotide sequence comprising the sequence of nucleotides 235 to 4004 of SEQ ID NO: 66 and the second AAV vector comprises a polynucleotide sequence comprising the sequence of nucleotides 229 to 4438 of SEQ ID NO: 67; or
(b) the first AAV vector comprises a polynucleotide sequence comprising the sequence of nucleotides 182 to 3949 of SEQ ID NO: 62 and the second AAV vector comprises a polynucleotide sequence comprising the sequence of nucleotides 187 to 4396 of SEQ ID NO: 63.

2. The method of claim 1, wherein the first AAV vector and the second AAV vector comprise an AAV1 capsid.

3. The method of claim 1, wherein:
(i) the first AAV vector comprises a first inverted terminal repeat (ITR) sequence 5′ of the promoter and a second ITR sequence 3′ of the recombinogenic region; and
(ii) the second AAV vector comprises a first ITR sequence 5′ of the recombinogenic region and a second ITR sequence 3′ of the poly(A) sisequence.

4. The method of claim 3, wherein the ITRs in the first AAV vector and the second AAV vector have at least 80% sequence identity to AAV2 ITRs.

5. The method of claim 1, wherein the subject has a mutation in OTOF.

6. The method of claim 1, wherein the subject has Deafness, Autosomal Recessive 9 (DFNB9).

7. The method of claim 1, wherein the dual vector system is administered locally to the ear of the subject.

8. The method of claim 1, wherein the dual vector system is administered by injection through the round window membrane, injection into a semicircular canal, canalostomy, insertion of a catheter through the round window membrane, transtympanic injection, or intratympanic injection.

9. The method of claim 1, wherein the method increases OTOF expression in a cochlear hair cell of the subject.

10. The method of claim 9, wherein the cochlear hair cell is an inner hair cell.

11. The method of claim 1, wherein the subject is a mammal.

12. The method of claim 1, wherein the subject is a human.

13. The method of claim 1, wherein the method improves hearing, reduces hearing loss, delays the development of hearing loss, slows the progression of hearing loss, improves speech discrimination, or improves hair cell function in the subject.

14. The method of claim 1, wherein the first AAV vector and the second AAV vector are administered concurrently.

15. The method of claim 1, wherein the first AAV vector and the second AAV vector are administered sequentially.

16. The method of claim 1, wherein the first AAV vector and the second AAV vector are administered to the subject at a concentration of about 1×10⁷ vector genomes (VG)/ear to about 2×10¹⁵ VG/ear.

17. A method of increasing OTOF expression in a subject in need thereof, the method comprising administering to the subject a dual vector system, wherein the dual vector system comprises:
(i) a first AAV vector comprising a Myo15 promoter operably linked to a first coding polynucleotide that encodes an N-terminal portion of an OTOF isoform 5 protein, a splice donor signal sequence positioned 3′ of the first coding polynucleotide, and a first recombinogenic region positioned 3′ of the splice donor signal sequence; and
(ii) a second AAV vector comprising a second recombinogenic region, a splice acceptor signal sequence positioned 3′ of the second recombinogenic region, a second coding polynucleotide that encodes a C-terminal portion of the OTOF isoform 5 protein positioned 3′ of the splice acceptor signal sequence, and a poly(A) sequence positioned 3′ of the second coding polynucleotide,
wherein the first coding polynucleotide and the second coding polynucleotide that encode the OTOF isoform 5 protein do not overlap,
wherein neither the first nor second AAV vector encodes the full-length OTOF isoform 5 protein,
and wherein:
(a) the first AAV vector comprises a polynucleotide sequence comprising the sequence of nucleotides 235 to 4004 of SEQ ID NO: 66 and the second AAV vector comprises a polynucleotide sequence comprising the sequence of nucleotides 229 to 4438 of SEQ ID NO: 67; or
(b) the first AAV vector comprises a polynucleotide sequence comprising the sequence of nucleotides 182 to 3949 of SEQ ID NO: 62 and the second AAV vector comprises a polynucleotide sequence comprising the sequence of nucleotides 187 to 4396 of SEQ ID NO: 63.

18. The method of claim 17, wherein the first AAV vector and the second AAV vector comprise an AAV1 capsid.

19. The method of claim 17, wherein:
(i) the first AAV vector comprises a first inverted terminal repeat (ITR) sequence 5′ of the promoter and a second ITR sequence 3′ of the recombinogenic region; and
(ii) the second AAV vector comprises a first ITR sequence 5′ of the recombinogenic region and a second ITR sequence 3′ of the poly(A) sisequence.

20. The method of claim 19, wherein the ITRs in the first AAV vector and the second AAV vector have at least 80% sequence identity to AAV2 ITRs.


SEQUENCE LISTING (68 sequences)

SEQ ID NO: 1 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 2 - DNA, 5994 bp, Homo sapiens
  source 1..5994: mol_type = other DNA
atggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatga

SEQ ID NO: 3 - DNA, 5994 bp, synthetic construct
  source 1..5994: mol_type = other DNA
atggcactgctgatccacctgaaaaccgtctccgaactgagaggcagaggggacagaatcgctaaagtcaccttccggggacagagcttttacagcagggtgctggagaactgcgaggacgtggccgactttgacgagacattcaggtggcccgtggccagctccatcgatcgcaatgagatgctggagatccaggtgtttaactatagcaaggtgttctccaataagctgatcggcaccttccggatggtgctgcagaaggtggtggaggagtcccacgtggaggtgaccgacacactgatcgacgataacaatgccatcatcaagacatccctgtgcgtggaggtgcgctaccaggccaccgatggcacagtgggctcttgggacgatggcgacttcctgggcgatgagtccctgcaggaggaggagaaggactctcaggagacagatggcctgctgcctggctcccggccatctagccgcccccctggcgagaagtcttttaggagagccggcaggtccgtgttctctgccatgaagctgggcaagaacaggagccacaaggaggagcctcagaggcccgacgagccagccgtgctggagatggaggacctggatcacctggccatcagactgggcgatggcctggaccctgatagcgtgtccctggcctccgtgaccgccctgaccacaaacgtgtctaataagcggagcaagccagacatcaagatggagccatctgccggcaggcccatggattaccaggtgagcatcacagtgatcgaggccagacagctggtgggcctgaacatggaccccgtggtgtgcgtggaagtgggcgacgataagaagtacacctccatgaaggagtctacaaactgtccatactacaacgagtacttcgtgtttgatttccacgtgagccccgacgtgatgttcgataagatcatcaagatcagcgtgatccactccaagaatctgctgcggtctggcaccctggtgggaagctttaagatggacgtgggcacagtgtactctcagcctgagcaccagttccaccacaagtgggccatcctgagcgatccagacgatatctcctctggcctgaagggctatgtgaagtgcgacgtggcagtggtgggcaagggcgataacatcaagaccccacacaaggccaatgagacagacgaggacgatatcgagggaaacctgctgctgccagagggagtgccacccgagaggcagtgggccaggttctacgtgaagatctatagggcagagggcctgcctaggatgaacaccagcctgatggccaatgtgaagaaggccttcatcggcgagaacaaggacctggtggatccctacgtgcaggtgttctttgccggccagaagggcaagacctccgtgcagaagagctcctatgagcctctgtggaatgagcaggtggtgtttacagacctgttccctccactgtgcaagaggatgaaggtgcagatcagagactctgataaggtgaacgacgtggccatcggcacccactttatcgatctgaggaagatcagcaatgacggcgataagggcttcctgcccaccctgggccccgcctgggtgaacatgtacggcagcaccagaaattatacactgctggacgagcaccaggatctgaacgagggcctgggcgagggcgtgagctttagagccaggctgctgctgggcctggccgtggagatcgtggacacctccaatcccgagctgacctctagcacagaggtgcaggtggagcaggccacacctatctctgagagctgtgccggcaagatggaggagttctttctgtttggcgccttcctggaggcctccatgatcgaccggcgcaacggcgataagcctatcaccttcgaggtgacaatcggcaactacggcaatgaggtggacggcctgtctcggccccagcgcccaaggcccagaaaggagcctggcgacgaggaggaggtggatctgatccagaacgccagcgacgatgaggcaggcgacgcaggcgatctggcctccgtgtcctctaccccccctatgcggccacaggtgacagaccgcaattactttcacctgccttatctggagcgcaagccatgcatctacatcaagtcttggtggcccgatcagaggagacggctgtataacgccaatatcatggaccacatcgccgataagctggaggagggcctgaatgacatccaggagatgatcaagaccgagaagtcctatccagagcgcaggctgaggggcgtgctggaggagctgagctgtggctgctgtagattcctgtccctggccgacaaggatcaggggcactcatcacggacacggctggaccgggagcggctgaaatcatgtatgcgggagctggaaaatatgggacagcaggcaaggatgctgcgcgcccaggtgaagaggcacaccgtgagagacaagctgcggctgtgccagaacttcctgcagaagctgcgctttctggccgatgagccacagcacagcatccccgacatcttcatctggatgatgtccaacaataagagagtggcctacgcccgggtgccctctaaggatctgctgtttagcatcgtggaggaggagacaggcaaggactgtgccaaggtgaagaccctgttcctgaagctgcctggcaagagaggctttggcagcgccggatggaccgtgcaggcaaaggtggagctgtatctgtggctgggcctgtctaagcagcggaaggagttcctgtgcggcctgccctgtggctttcaggaggtgaaggcagcacagggactgggactgcacgccttcccccccgtgagcctggtgtacaccaagaagcaggcctttcagctgagggcccatatgtaccaggccaggtctctgttcgccgccgatagctccggactgagcgacccttttgccagggtgttctttatcaatcagagccagtgcacagaggtgctgaacgagaccctgtgcccaacatgggatcagatgctggtgttcgacaacctggagctgtacggagaggcacacgagctgagggacgatccacccatcatcgtgatcgagatctatgatcaggactccatgggcaaggccgatttcatgggcaggacctttgccaagcccctggtgaagatggccgacgaggcctactgccctccaagattcccccctcagctcgagtactatcagatctataggggaaatgcaaccgccggagacctgctggccgcctttgagctgctgcagatcggccccgccggaaaggcagacctgccacccatcaacggcccagtggatgtggacagaggccccatcatgcctgtgccaatgggcatcagaccagtgctgtccaagtacagggtggaggtgctgttctggggactgcgcgacctgaagagggtgaatctggcccaggtggataggcccagagtggacatcgagtgcgccggaaagggcgtgcagtctagcctgatccacaactataagaagaacccaaatttcaacaccctggtgaagtggtttgaggtggatctgcccgagaatgagctgctgcaccctccactgaacatccgggtggtggactgtagagccttcggcaggtacaccctggtgggcagccacgccgtgagcagcctgaggaggttcatctacaggccccctgacaggtccgccccttcttggaataccacagtgagactgctgcggcgctgcagggtgctgtgcaacggaggcagctcctctcactctaccggcgaggtggtggtgacaatggagcctgaggtacccatcaagaagctggagaccatggtgaagctggatgccacaagcgaggcagtggtgaaggtggacgtggcagaggaggagaaggagaagaagaagaagaagaagggaaccgccgaggagcctgaggaagaggagccagatgagagcatgctggactggtggtccaagtacttcgcctctatcgacacaatgaaggagcagctgagacagcaggagcctagcggcatcgatctggaggagaaggaggaggtggacaataccgagggcctgaagggctccatgaagggcaaggagaaggcaagggcagcaaaggaagagaagaagaagaagacccagagcagcggctctggacagggcagcgaggcaccagagaagaagaagcctaagatcgatgagctgaaggtgtacccaaaggagctggagtccgagttcgataattttgaggactggctgcacaccttcaacctgctgcgcggcaagacaggcgacgatgaggacggcagcaccgaggaggagagaatcgtgggccggtttaagggctccctgtgcgtgtacaaggtgccactgcctgaggacgtgagcagggaggccggatacgactctacctatggcatgttccagggcatcccctctaatgatcctatcaacgtgctggtgcgcgtgtatgtggtgagggccacagatctgcaccccgccgacatcaacggcaaggccgacccttacatcgccatccgcctgggcaagaccgatatcagggacaaggagaattatatctccaagcagctgaaccccgtgttcggcaagtcttttgacatcgaggccagcttccctatggagtccatgctgaccgtggccgtgtacgattgggacctggtgggcaccgacgatctgatcggcgagacaaagatcgatctggagaatcgcttttattctaagcacagggcaacctgcggaatcgcacagacctacagcacacacggctataacatctggcgcgaccccatgaagcctagccagatcctgacaaggctgtgcaaggatggcaaggtggacggaccacacttcggaccacccggcagagtgaaggtggccaatcgggtgtttacaggcccttccgagatcgaggatgagaacggccagcgcaagccaaccgacgagcacgtggccctgctggccctgaggcactgggaggatatcccaagggccggatgtaggctggtgcctgagcacgtggagaccagaccactgctgaatccagacaagccaggaatcgagcagggcaggctggagctgtgggtggatatgttcccaatggacatgccagccccaggaacacccctggatatctcccctagaaagccaaagaagtacgagctgagagtgatcatctggaacacagacgaggtggtgctggaggacgatgacttctttaccggcgagaagtctagcgatatctttgtgcgcggatggctgaagggacagcaggaggacaagcaggatacagacgtgcactaccactccctgaccggcgagggcaatttcaactggagatacctgttcccttttgattatctggccgccgaggagaagatcgtgatctctaagaaggagagcatgttttcctgggacgagacagagtataagatcccagccagactgaccctgcagatctgggatgccgaccacttcagcgccgatgactttctgggcgccatcgagctggacctgaaccggttcccaagaggcgccaagaccgccaagcagtgcacaatggagatggcaaccggagaggtggacgtgcctctggtgtctatcttcaagcagaagcgggtgaagggatggtggccactgctggccaggaacgagaatgatgagtttgagctgacaggcaaggtggaggcagagctgcacctgctgaccgccgaggaggcagagaagaacccagtgggcctggccaggaatgagcccgaccctctggagaagccaaacaggcccgatacagccttcgtgtggtttctgaatcctctgaagagcatcaagtacctgatctgtaccaggtataagtggctgatcatcaagatcgtgctggccctgctgggactgctgatgctgggcctgtttctgtactccctgcccggctatatggtgaagaagctgctgggcgcctga

SEQ ID NO: 4 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 5 - DNA, 5979 bp, Homo sapiens
  source 1..5979: mol_type = other DNA
atggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagcaaaggcagagagaagaccaagggaggcagagatggcgagcacaaagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctga

SEQ ID NO: 6 - DNA, 5994 bp, synthetic construct
  source 1..5994: mol_type = other DNA
atggcactgctgatccacctgaaaaccgtctccgaactgagaggcagaggggacagaatcgctaaagtcaccttccggggacagagcttttacagcagggtgctggagaactgcgaggacgtggccgactttgacgagacattcaggtggcccgtggccagctccatcgatcgcaatgagatgctggagatccaggtgtttaactatagcaaggtgttctccaataagctgatcggcaccttccggatggtgctgcagaaggtggtggaggagtcccacgtggaggtgaccgacacactgatcgacgataacaatgccatcatcaagacatccctgtgcgtggaggtgcgctaccaggccaccgatggcacagtgggctcttgggacgatggcgacttcctgggcgatgagtccctgcaggaggaggagaaggactctcaggagacagatggcctgctgcctggctcccggccatctagccgcccccctggcgagaagtcttttaggagagccggcaggtccgtgttctctgccatgaagctgggcaagaacaggagccacaaggaggagcctcagaggcccgacgagccagccgtgctggagatggaggacctggatcacctggccatcagactgggcgatggcctggaccctgatagcgtgtccctggcctccgtgaccgccctgaccacaaacgtgtctaataagcggagcaagccagacatcaagatggagccatctgccggcaggcccatggattaccaggtgagcatcacagtgatcgaggccagacagctggtgggcctgaacatggaccccgtggtgtgcgtggaagtgggcgacgataagaagtacacctccatgaaggagtctacaaactgtccatactacaacgagtacttcgtgtttgatttccacgtgagccccgacgtgatgttcgataagatcatcaagatcagcgtgatccactccaagaatctgctgcggtctggcaccctggtgggaagctttaagatggacgtgggcacagtgtactctcagcctgagcaccagttccaccacaagtgggccatcctgagcgatccagacgatatctcctctggcctgaagggctatgtgaagtgcgacgtggcagtggtgggcaagggcgataacatcaagaccccacacaaggccaatgagacagacgaggacgatatcgagggaaacctgctgctgccagagggagtgccacccgagaggcagtgggccaggttctacgtgaagatctatagggcagagggcctgcctaggatgaacaccagcctgatggccaatgtgaagaaggccttcatcggcgagaacaaggacctggtggatccctacgtgcaggtgttctttgccggccagaagggcaagacctccgtgcagaagagctcctatgagcctctgtggaatgagcaggtggtgtttacagacctgttccctccactgtgcaagaggatgaaggtgcagatcagagactctgataaggtgaacgacgtggccatcggcacccactttatcgatctgaggaagatcagcaatgacggcgataagggcttcctgcccaccctgggccccgcctgggtgaacatgtacggcagcaccagaaattatacactgctggacgagcaccaggatctgaacgagggcctgggcgagggcgtgagctttagagccaggctgctgctgggcctggccgtggagatcgtggacacctccaatcccgagctgacctctagcacagaggtgcaggtggagcaggccacacctatctctgagagctgtgccggcaagatggaggagttctttctgtttggcgccttcctggaggcctccatgatcgaccggcgcaacggcgataagcctatcaccttcgaggtgacaatcggcaactacggcaatgaggtggacggcctgtctcggccccagcgcccaaggcccagaaaggagcctggcgacgaggaggaggtggatctgatccagaacgccagcgacgatgaggcaggcgacgcaggcgatctggcctccgtgtcctctaccccccctatgcggccacaggtgacagaccgcaattactttcacctgccttatctggagcgcaagccatgcatctacatcaagtcttggtggcccgatcagaggagacggctgtataacgccaatatcatggaccacatcgccgataagctggaggagggcctgaatgacatccaggagatgatcaagaccgagaagtcctatccagagcgcaggctgaggggcgtgctggaggagctgagctgtggctgctgtagattcctgtccctggccgacaaggatcaggggcactcatcacggacacggctggaccgggagcggctgaaatcatgtatgcgggagctggaaaatatgggacagcaggcaaggatgctgcgcgcccaggtgaagaggcacaccgtgagagacaagctgcggctgtgccagaacttcctgcagaagctgcgctttctggccgatgagccacagcacagcatccccgacatcttcatctggatgatgtccaacaataagagagtggcctacgcccgggtgccctctaaggatctgctgtttagcatcgtggaggaggagacaggcaaggactgtgccaaggtgaagaccctgttcctgaagctgcctggcaagagaggctttggcagcgccggatggaccgtgcaggcaaaggtggagctgtatctgtggctgggcctgtctaagcagcggaaggagttcctgtgcggcctgccctgtggctttcaggaggtgaaggcagcacagggactgggactgcacgccttcccccccgtgagcctggtgtacaccaagaagcaggcctttcagctgagggcccatatgtaccaggccaggtctctgttcgccgccgatagctccggactgagcgacccttttgccagggtgttctttatcaatcagagccagtgcacagaggtgctgaacgagaccctgtgcccaacatgggatcagatgctggtgttcgacaacctggagctgtacggagaggcacacgagctgagggacgatccacccatcatcgtgatcgagatctatgatcaggactccatgggcaaggccgatttcatgggcaggacctttgccaagcccctggtgaagatggccgacgaggcctactgccctccaagattcccccctcagctcgagtactatcagatctataggggaaatgcaaccgccggagacctgctggccgcctttgagctgctgcagatcggccccgccggaaaggcagacctgccacccatcaacggcccagtggatgtggacagaggccccatcatgcctgtgccaatgggcatcagaccagtgctgtccaagtacagggtggaggtgctgttctggggactgcgcgacctgaagagggtgaatctggcccaggtggataggcccagagtggacatcgagtgcgccggaaagggcgtgcagtctagcctgatccacaactataagaagaacccaaatttcaacaccctggtgaagtggtttgaggtggatctgcccgagaatgagctgctgcaccctccactgaacatccgggtggtggactgtagagccttcggcaggtacaccctggtgggcagccacgccgtgagcagcctgaggaggttcatctacaggccccctgacaggtccgccccttcttggaataccacagtgagactgctgcggcgctgcagggtgctgtgcaacggaggcagctcctctcactctaccggcgaggtggtggtgacaatggagcctgaggtacccatcaagaagctggagaccatggtgaagctggatgccacaagcgaggcagtggtgaaggtggacgtggcagaggaggagaaggagaagaagaagaagaagaagggaaccgccgaggagcctgaggaagaggagccagatgagagcatgctggactggtggtccaagtacttcgcctctatcgacacaatgaaggagcagctgagacagcaggagcctagcggcatcgatctggaggagaaggaggaggtggacaataccgagggcctgaagggctccatgaagggcaaggagaaggcaagggcagcaaaggaagagaagaagaagaagacccagagcagcggctctggacagggcagcgaggcaccagagaagaagaagcctaagatcgatgagctgaaggtgtacccaaaggagctggagtccgagttcgataattttgaggactggctgcacaccttcaacctgctgcgcggcaagacaggcgacgatgaggacggcagcaccgaggaggagagaatcgtgggccggtttaagggctccctgtgcgtgtacaaggtgccactgcctgaggacgtgagcagggaggccggatacgactctacctatggcatgttccagggcatcccctctaatgatcctatcaacgtgctggtgcgcgtgtatgtggtgagggccacagatctgcaccccgccgacatcaacggcaaggccgacccttacatcgccatccgcctgggcaagaccgatatcagggacaaggagaattatatctccaagcagctgaaccccgtgttcggcaagtcttttgacatcgaggccagcttccctatggagtccatgctgaccgtggccgtgtacgattgggacctggtgggcaccgacgatctgatcggcgagacaaagatcgatctggagaatcgcttttattctaagcacagggcaacctgcggaatcgcacagacctacagcacacacggctataacatctggcgcgaccccatgaagcctagccagatcctgacaaggctgtgcaaggatggcaaggtggacggaccacacttcggaccacccggcagagtgaaggtggccaatcgggtgtttacaggcccttccgagatcgaggatgagaacggccagcgcaagccaaccgacgagcacgtggccctgctggccctgaggcactgggaggatatcccaagggccggatgtaggctggtgcctgagcacgtggagaccagaccactgctgaatccagacaagccaggaatcgagcagggcaggctggagctgtgggtggatatgttcccaatggacatgccagccccaggaacacccctggatatctcccctagaaagccaaagaagtacgagctgagagtgatcatctggaacacagacgaggtggtgctggaggacgatgacttctttaccggcgagaagtctagcgatatctttgtgcgcggatggctgaagggacagcaggaggacaagcaggatacagacgtgcactaccactccctgaccggcgagggcaatttcaactggagatacctgttcccttttgattatctggccgccgaggagaagatcgtgatctctaagaaggagagcatgttttcctgggacgagacagagtataagatcccagccagactgaccctgcagatctgggatgccgaccacttcagcgccgatgactttctgggcgccatcgagctggacctgaaccggttcccaagaggcgccaagaccgccaagcagtgcacaatggagatggcaaccggagaggtggacgtgcctctggtgtctatcttcaagcagaagagggtgaagggctggtggccactgctggccagaaacgagaatgatgagtttgagctgacaggcaaggtggaggcagagctgcacctgctgaccgccgaggaggcagagaagaacccagtgggcctggccaggaatgagcccgaccctctggagaagccaaacaggcccgacaccagcttcatctggtttctgaatcctctgaagtccgcccggtacttcctgtggcacacctatcgctggctgctgctgaagctgttattactgttattactgctgctgctgctggccctgtttctgtacagcgtgcccggctatctggtgaagaagatcctgggcgcctga

SEQ ID NO: 7 - DNA, 454 bp, Mus musculus
  source 1..454: mol_type = genomic DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 8 - DNA, 1157 bp, Mus musculus
  source 1..1157: mol_type = genomic DNA
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 9 - DNA, 76 bp, Mus musculus
  source 1..76: mol_type = genomic DNA
cccatgtcagctgcttgtgctttccagagacaaaacaggaataatagatgtcattaaatatacattgggccccagg

SEQ ID NO: 10 - DNA, 95 bp, Mus musculus
  source 1..95: mol_type = genomic DNA
agcctgagcctcctttccatctctgtggaggcagacataggacccccaacaaacagcatgcaggttgggagccagccacaggacccaggtaaggg

SEQ ID NO: 11 - DNA, 171 bp, synthetic construct
  source 1..171: mol_type = other DNA
cccatgtcagctgcttgtgctttccagagacaaaacaggaataatagatgtcattaaatatacattgggccccaggagcctgagcctcctttccatctctgtggaggcagacataggacccccaacaaacagcatgcaggttgggagccagccacaggacccaggtaaggg

SEQ ID NO: 12 - DNA, 171 bp, synthetic construct
  source 1..171: mol_type = other DNA
agcctgagcctcctttccatctctgtggaggcagacataggacccccaacaaacagcatgcaggttgggagccagccacaggacccaggtaagggcccatgtcagctgcttgtgctttccagagacaaaacaggaataatagatgtcattaaatatacattgggccccagg

SEQ ID NO: 13 - DNA, 184 bp, Mus musculus
  source 1..184: mol_type = genomic DNA
cccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggg

SEQ ID NO: 14 - DNA, 82 bp, Mus musculus
  source 1..82: mol_type = genomic DNA
tgaggtgggagctgggctctccctgatgtattattcagctccctggagttggccagctcctgttacactggccacagccctg

SEQ ID NO: 15 - DNA, 106 bp, Mus musculus
  source 1..106: mol_type = genomic DNA
cacaggcctcccagtctaccctcactagaaaacaacacccaggcactttccaccacctctcaaaggtgaaacccaaggctggtctagagaatgaattatggatcct

SEQ ID NO: 16 - DNA, 188 bp, synthetic construct
  source 1..188: mol_type = other DNA
tgaggtgggagctgggctctccctgatgtattattcagctccctggagttggccagctcctgttacactggccacagccctgcacaggcctcccagtctaccctcactagaaaacaacacccaggcactttccaccacctctcaaaggtgaaacccaaggctggtctagagaatgaattatggatcct

SEQ ID NO: 17 - DNA, 188 bp, synthetic construct
  source 1..188: mol_type = other DNA
cacaggcctcccagtctaccctcactagaaaacaacacccaggcactttccaccacctctcaaaggtgaaacccaaggctggtctagagaatgaattatggatccttgaggtgggagctgggctctccctgatgtattattcagctccctggagttggccagctcctgttacactggccacagccctg

SEQ ID NO: 18 - DNA, 430 bp, Mus musculus
  source 1..430: mol_type = genomic DNA
tgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcct

SEQ ID NO: 19 - DNA, 1611 bp, synthetic construct
  source 1..1611: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactatggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 20 - DNA, 1611 bp, synthetic construct
  source 1..1611: mol_type = other DNA
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 21 - DNA, 965 bp, synthetic construct
  source 1..965: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 22 - DNA, 520 bp, synthetic construct
  source 1..520: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 23 - DNA, 235 bp, Homo sapiens
  source 1..235: mol_type = genomic DNA
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttcc

SEQ ID NO: 24 - DNA, 688 bp, Homo sapiens
  source 1..688: mol_type = genomic DNA
tgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 25 - DNA, 77 bp, Homo sapiens
  source 1..77: mol_type = genomic DNA
cccctgtcagctgctcactcctttccagagacaaaacaggaataatagacatcattaaatatacatagggccccagg

SEQ ID NO: 26 - DNA, 82 bp, Homo sapiens
  source 1..82: mol_type = genomic DNA
tgagccggcagcctggctccccaccccatgtattattcagctcctgagagccagccagctcctgttacactgaccgcagccc

SEQ ID NO: 27 - DNA, 111 bp, Homo sapiens
  source 1..111: mol_type = genomic DNA
cacaggcctcataggcaggcttccccatcctggtaaacaacacccacacactttctactactgctctagggtgaaacccaaggcgctctagaggagatgaattatggatcc

SEQ ID NO: 28 - DNA, 193 bp, synthetic construct
  source 1..193: mol_type = other DNA
tgagccggcagcctggctccccaccccatgtattattcagctcctgagagccagccagctcctgttacactgaccgcagccccacaggcctcataggcaggcttccccatcctggtaaacaacacccacacactttctactactgctctagggtgaaacccaaggcgctctagaggagatgaattatggatcc

SEQ ID NO: 29 - DNA, 193 bp, synthetic construct
  source 1..193: mol_type = other DNA
cacaggcctcataggcaggcttccccatcctggtaaacaacacccacacactttctactactgctctagggtgaaacccaaggcgctctagaggagatgaattatggatcctgagccggcagcctggctccccaccccatgtattattcagctcctgagagccagccagctcctgttacactgaccgcagccc

SEQ ID NO: 30 - DNA, 499 bp, Homo sapiens
  source 1..499: mol_type = genomic DNA
tgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatcc

SEQ ID NO: 31 - DNA, 1262 bp, synthetic construct
  source 1..1262: mol_type = other DNA
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttagcctagagtccttggggtctgtcactggccaccctcgcatcccaggctgcaggaaactgaggcccagagaggacaaggactttcctggacccacacagccagtcagtgacagagcctagggtctgagccaggcctgacccaacctccatttctgcctctctacccctgcccccgccccaacacacacacacacacaagtggagttccactgaaacgcccctccttgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 32 - DNA, 649 bp, synthetic construct
  source 1..649: mol_type = other DNA
gcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcgggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgc

SEQ ID NO: 33 - DNA, 240 bp, Mus musculus
  source 1..240: mol_type = genomic DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 34 - DNA, 95 bp, Mus musculus
  source 1..95: mol_type = genomic DNA
tttatgaggtgggagctgggctctccctgatgtattattcagctccctggagttggccagctcctgttacactggccacagccctgggcatccgc

SEQ ID NO: 35 - DNA, 185 bp, Mus musculus
  source 1..185: mol_type = genomic DNA
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 36 - DNA, 335 bp, synthetic construct
  source 1..335: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 37 - DNA, 425 bp, synthetic construct
  source 1..425: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 38 - DNA, 280 bp, synthetic construct
  source 1..280: mol_type = other DNA
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 39 - DNA, 520 bp, synthetic construct
  source 1..520: mol_type = other DNA
tttatgaggtgggagctgggctctccctgatgtattattcagctccctggagttggccagctcctgttacactggccacagccctgggcatccgcctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 40 - DNA, 520 bp, synthetic construct
  source 1..520: mol_type = other DNA
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 41 - DNA, 520 bp, synthetic construct
  source 1..520: mol_type = other DNA
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 42 - DNA, 964 bp, synthetic construct
  source 1..964: mol_type = other DNA
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 43 - DNA, 519 bp, synthetic construct
  source 1..519: mol_type = other DNA
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 44 - DNA, 943 bp, synthetic construct
  source 1..943: mol_type = other DNA
ggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 45 - DNA, 548 bp, synthetic construct
  source 1..548: mol_type = other DNA
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 46 - DNA, 425 bp, synthetic construct
  source 1..425: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 47 - DNA, 77 bp, synthetic construct
  source 1..77: mol_type = other DNA
gggattttgccgatttcggcctattggttaaaaaatgagctgatttaacaaaaatttaacgcgaattttaacaaaat

SEQ ID NO: 48 - DNA, 865 bp, Homo sapiens
  source 1..865: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 49 - DNA, 437 bp, Homo sapiens
  source 1..437: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtga

SEQ ID NO: 50 - DNA, 428 bp, Homo sapiens
  source 1..428: mol_type = other DNA
gctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 51 - DNA, 287 bp, Homo sapiens
  source 1..287: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 52 - DNA, 290 bp, Homo sapiens
  source 1..290: mol_type = other DNA
cgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcagg

SEQ ID NO: 53 - DNA, 288 bp, Homo sapiens
  source 1..288: mol_type = other DNA
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 54 - DNA, 84 bp, synthetic construct
  source 1..84: mol_type = other DNA
gtaagtatcaaggttacaagacaggtttaaggagaccaatagaaactgggcttgtcgagacagagaagactcttgcgtttctga

SEQ ID NO: 55 - DNA, 49 bp, synthetic construct
  source 1..49: mol_type = other DNA
taggcacctattggtcttactgacatccactttgcctttctctccacag

SEQ ID NO: 56 - DNA, 2406 bp, Homo sapiens
  source 1..2406: mol_type = other DNA
atggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctg

SEQ ID NO: 57 - DNA, 3588 bp, Homo sapiens
  source 1..3588: mol_type = other DNA
gaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatga

SEQ ID NO: 58 - protein, 802 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMREL

SEQ ID NO: 59 - protein, 1195 aa, Homo sapiens
ENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 60 - DNA, 10032 bp, synthetic construct
  source 1..10032: mol_type = other DNA
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 61 - DNA, 10461 bp, synthetic construct
  source 1..10461: mol_type = other DNA
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 62 - DNA, 6678 bp, synthetic construct
  source 1..6678: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccgaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 63 - DNA, 7152 bp, synthetic construct
  source 1..7152: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 64 - DNA, 10005 bp, synthetic construct
  source 1..10005: mol_type = other DNA
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 65 - DNA, 6653 bp, synthetic construct
  source 1..6653: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 66 - DNA, 6986 bp, synthetic construct
  source 1..6986: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 67 - DNA, 7414 bp, synthetic construct
  source 1..7414: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 68 - DNA, 6959 bp, synthetic construct
  source 1..6959: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg